(19) United States
(12) Patent Application Publication
(10) Pub. No.: US 2024/0358767 A1
(43) Pub. Date: Oct. 31, 2024

(54) STEM CELL DIFFERENTIATION AND CHEMICAL COMPOUNDS

(71) Applicant: VERTEX PHARMACEUTICALS INCORPORATED, Boston, MA (US)

(72) Inventors: Chunhui XIE, Boston, MA (US); George HARB, Boston, MA (US); Elizabeth RYU, Boston, MA (US); Nicholas TECENO, Boston, MA (US); Christopher FARRAR, Boston, MA (US)

(73) Assignee: VERTEX PHARMACEUTICALS INCORPORATED, Boston, MA (US)

(21) Appl. No.: 18/707,756

(22) PCT Filed: Nov. 7, 2022
(86) PCT No.: PCT/US2022/079398

§ 371 Date: May 6, 2024

Related U.S. Application Data
(60) Provisional application No. 63/277,092, filed on Nov. 8, 2021.

(51) Int. Cl. A61K 35/39; C12N 5/071

(52) U.S. Cl. CPC A61K 35/39; C12N 5/0676; C12N 2501/117; C12N 2501/16; C12N 2501/385; C12N 2501/72

(57) ABSTRACT

Disclosed herein are compositions and methods related to differentiation of stem cells into pancreatic cells. In some aspects, the methods provided herein relate to generation of pancreatic β cell, α cell, δ cells, and EC cells in vitro in the presence of one or more chemical compounds that inhibit PI3K/Akt/mTOR signaling. In some aspects, the disclosure provides pharmaceutical compositions including the cells generated according to the methods disclosed herein, as well as methods of making use thereof.

### CROSS REFERENCE

This application claims the benefit of U.S. Provisional Application No. 63/277,092, filed Nov. 8, 2021, which is incorporated herein by reference for its entirety.

### BACKGROUND

Generation of stem cell derived β-cells can provide a potentially useful step toward the generation of islets and pancreatic organs. One of the diseases that may be treatable by stem cell derived tissues is diabetes. Type 1 diabetes results from autoimmune destruction of 3-cells in the pancreatic islet. Type 2 diabetes results from peripheral tissue insulin resistance and β-cell dysfunction. Diabetic patients, particularly those suffering from type 1 diabetes, can potentially be cured through transplantation of new 0-cells. Patients transplanted with cadaveric human islets can be made insulin independent for 5 years or longer via this strategy, but this approach is limited because of the scarcity and quality of donor islets. Generation of an unlimited supply of human j-cells from stem cells can extend this therapy to millions of new patients and can be an important test case for translating stem cell biology into the clinic.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

### SUMMARY

In some aspects, provided herein is an in vitro composition comprising Sox17-positive cells and an inhibitor of PI3K/Akt/mTOR signaling. In some cases, the composition further comprises stem cells. In some cases, the composition further comprises a growth factor from the TGF-β superfamily.

In some aspects, provided herein is an in vitro composition comprising stem cells, an inhibitor of PI3K/Akt/mTOR signaling, and a growth factor from TGF-β superfamily.

In some cases, the growth factor from TGF-β superfamily is selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11). In some cases, the growth factor from TGF-β superfamily comprises Activin A, GDF8, or both.

In some cases, the composition provided herein comprises at most about 100 ng/mL, at most about 80 ng/mL, at most about 60 ng/mL, at most about 50 ng/mL, at most about 25 ng/mL, at most about 20 ng/mL, at most about 15 ng/mL, at most about 10 ng/mL, at most about 5 ng/mL, or at most about 2 ng/mL of Activin A. In some cases, the composition comprises from 0.5 ng/mL to 500 ng/mL, 1 ng/mL to 250 ng/mL, 10 ng/mL to 200 ng/mL, 20 ng/mL to 150 ng/mL, 50 ng/mL to 120 ng/mL, 1 ng/mL to 50 ng/mL, 2 ng/mL to 25 ng/mL, or 5 ng/mL to 20 ng/mL of Activin A. In some cases, the composition comprises about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 12 ng/mL, about 14 ng/mL, about 15 ng/mL, about 18 ng/mL, about 20 ng/mL, about 25 ng/mL, about 30 ng/mL, or about 50 ng/mL of Activin A.

In some aspects, provided herein is an in vitro composition comprising stem cells and an inhibitor of PI3K/Akt/mTOR signaling.

In some cases, the inhibitor of PI3K/Akt/mTOR signaling comprises an inhibitor of a PI3K protein, an inhibitor of an Akt protein, an inhibitor of mTOR, or any combination thereof. In some cases, the inhibitor of PI3K/Akt/mTOR signaling comprises one or more of: GSK-690693, IPI-3063, AZD8055, Omipalisib, GNE-477, VS-5584, BYL319, YM201636, PI4KIIIbeta-IN-10, Nemiralisib, BYL719, FT113, or Apitolisib, or any analog or derivative thereof. In some cases, the composition comprises an inhibitor of a PI3K protein and an inhibitor of an Akt protein. In some cases, the composition comprises GSK-690693, an analog or a derivative thereof. In some cases, the composition comprises BYL719, an analog or a derivative thereof. In some cases, the composition comprises BYL319, an analog or a derivative thereof. In some cases, the composition comprises GSK-690693, or an analog or a derivative thereof, and BYL319, or an analog or a derivative thereof. In some cases, the composition comprises GSK-690693, or an analog or a derivative thereof, and BYL719, or an analog or a derivative thereof. In some cases, the composition comprises from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693, or an analog or a derivative thereof. In some cases, the composition comprises about 0.01 μM, about 0.02 μM, about 0.04 μM, about 0.06 μM, about 0.08 μM, about 0.1 μM, about 0.12 μM, about 0.15 μM, about 0.2 μM, about 0.3 μM, about 0.4 μM, about 0.5 μM, about 0.6 μM, about 0.8 μM, or about 1 μM of GSK-690693. In some cases, the composition comprises from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719, or an analog or a derivative thereof. In some cases, the composition comprises about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719. In some cases, the composition comprises about 0.08 μM to about 0.12 μM of GSK-690693 and about 35 nM to about 50 nM of BYL719. In some cases, the composition further comprises an activator of WNT signaling pathway. In some cases, the activator of WNT signaling pathway comprises one or more of Wnt3a, CHIR99021, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC693868, SB 216763, SB 415286, TC-G 24, TCS 2002, TCS 21311, TWS 119, and analogs or derivatives thereof. In some cases, the composition further comprises a GSK3 inhibitor. In some cases, the composition further comprises from 0.5 μM to 50 μM, 0.6 μM to 30 μM, 0.8 μM to 20 μM, 1 μM to 10 μM, or 2 μM to 5 μM of CHIR99021. In some cases, the composition further comprises about 0.5 μM, 0.6 μM, 0.8 μM, 1 μM, 2 μM, 3 μM, 4 μM, 5 μM, 6 μM, 8 μM, 10 μM, 15 μM, 20 μM, 25 μM, or 30 μM of CHIR99021.

In some cases, the stem cells comprise embryonic stem cells. In some cases, the stem cells comprise induced pluripotent stem cells. In some cases, the stem cells are human cells. In some cases, the stem cells are genetically modified. In some cases, the composition comprises a population of cells that comprises Sox17-positive, Oct4-negative cells. In some cases, the population of cells comprises at least about 50%, 60%, 65%, 70%, 75%, 80%, or 85% Sox17-positive, Oct4-negative cells. In some cases, the population of cells comprises from about 50% to about 90%, about 60% to about 90%, about 65% to about 90%, about 70% to about 90%, about 75% to about 90%, about 80% to about 90%, or about 75% to about 85% Sox17-positive, Oct4-negative cells.

In some aspects, provided herein is an in vitro composition comprising a plurality of FOXA2-positive, PDX1-negative cells and an inhibitor of PI3K/Akt/mTOR signaling. In some cases, the composition further comprises one or more agents selected from the group consisting of: a protein kinase C activator, a bone morphogenetic protein signaling pathway inhibitor, a growth factor from fibroblast growth factors (FGF) family, a retinoic acid (RA) signaling pathway activator, a Rho-associated, coiled-coil containing protein kinase (ROCK) inhibitor, and a sonic hedgehog (SHH) pathway inhibitor. In some cases, the composition further comprises: (a) a protein kinase C activator selected from the group consisting of: phorbol 12,13-dibutyrate (PDBU), TPB, phorbol 12-myristate 13-acetate, and bryostatin 1; (b) a bone morphogenetic protein signaling pathway inhibitor comprising LDN193189 or DMH-1; (c) a growth factor from fibroblast growth factors (FGF) family selected from the group consisting of: keratinocyte growth factor (KGF), FGF2, FGF10, FGF21, and FGF8B; (d) a sonic hedgehog pathway inhibitor selected from the group consisting of SANT1, SANT2, SANT4, Cur6l4l4, forskolin, tomatidine, AY9944, triparanol, and cyclopamine; (e) a retinoic acid signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; and/or (f) a ROCK inhibitor selected from the group consisting of Thiazovivin, Y-27632, Fasudil/HA1077, and 14-1152. In some cases, the composition further comprises a growth factor from transformation growth factor β (TGF-β) superfamily. In some cases, the growth factor from the TGF-β superfamily is selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11). In some cases, the growth factor from the TGF-β superfamily comprises Activin A, GDF8, or both. In some cases, the composition comprises at most about 20 ng/mL Activin A. In some cases, the composition comprises at most about 10 ng/mL, at most about 5 ng/mL, at most about 1 ng/mL, at most about 0.5 ng/mL, or at most about 0.1 ng/mL Activin A. In some cases, the composition comprises about 20 ng/mL, about 10 ng/mL, about 5 ng/mL, about 1 ng/mL, about 0.5 ng/mL, or about 0.1 ng/mL Activin A. In some cases, the composition further comprises PDX1-positive and NKX6.1-negative cells.

In some aspects, provided herein is an in vitro composition comprising a plurality of PDX1-positive and NKX6.1-negative cells and an inhibitor of PI3K/Akt/mTOR signaling. In some cases, the composition further comprises one or more agents selected from the group consisting of: a growth factor from fibroblast growth factors (FGF) family, a retinoic acid (RA) signaling pathway activator, a Rho-associated, coiled-coil containing protein kinase (ROCK) inhibitor, a protein kinase C activator, and a sonic hedgehog (SHH) pathway inhibitor. In some cases, the composition further comprises: (a) a growth factor from the transformation growth factor β (TGF-β) superfamily selected from the group consisting of: an Inhibin, an Activin, a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11); (b) a growth factor from fibroblast growth factors (FGF) family selected from the group consisting of: keratinocyte growth factor (KGF), FGF2, FGF10, FGF21, and FGF8B; (c) a retinoic acid (RA) signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; (d) a ROCK inhibitor selected from the group consisting of Thiazovivin, Y-27632, Fasudil/HA1077, and 14-1152; (e) a protein kinase C activator selected from the group consisting of: phorbol 12,13-dibutyrate (PDBU), TPB, phorbol 12-myristate 13-acetate, and bryostatin 1; (f) a sonic hedgehog (SHH) pathway inhibitor selected from the group consisting of SANT1, SANT2, SANT4, Cur6l4l4, forskolin, tomatidine, AY9944, triparanol, and cyclopamine; and/or (g) a FoxO1 inhibitor, optionally wherein the FoxO1 inhibitor is AS1842856. In some cases, the composition further comprises a notch signaling inhibitor, optionally wherein the notch signaling inhibitor is XXI or DAPI. In some cases, the composition further comprises a growth factor from transformation growth factor β (TGF-β) superfamily. In some cases, the growth factor from the TGF-β superfamily is selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11). In some cases, the growth factor from the TGF-β superfamily comprises Activin A, GDF8, or both. In some cases, the composition comprises at most about 5 ng/mL Activin A. In some cases, the composition comprises at most about 2.5 ng/mL, at most about 1 ng/mL, at most about 0.5 ng/mL, at most about 0.1 ng/mL, or at most about 0.05 ng/mL Activin A. In some cases, the composition comprises about 5 ng/mL, about 2.5 ng/mL, about 1 ng/mL, about 0.5 ng/mL, about 0.1 ng/mL, or about 0.05 ng/mL Activin A. In some cases, the composition further comprises PDX1-positive and NKX6.1-positive cells. In some cases, the inhibitor of PI3K/Akt/mTOR signaling comprises an inhibitor of a PI3K protein, an inhibitor of an Akt protein, an inhibitor of mTOR, or any combination thereof. In some cases, the inhibitor of PI3K/Akt/mTOR signaling comprises one or more of: GSK-690693, IPI-3063, AZD8055, Omipalisib, GNE-477, VS-5584, BYL319, YM201636, PI4KIIIbeta-IN-10, Nemiralisib, BYL719, FT113, Apitolisib, or any analog or derivative thereof. In some cases, the composition comprises an inhibitor of a PI3K protein and an inhibitor of an Akt protein. In some cases, the composition comprises GSK-690693, an analog or a derivative thereof. In some cases, the composition comprises BYL719, an analog or a derivative thereof. In some cases, the composition comprises BYL319, an analog or a derivative thereof. In some cases, the composition comprises GSK-690693, or an analog or a derivative thereof, and BYL319, or an analog or a derivative thereof. In some cases, the composition comprises GSK-690693, or an analog or a derivative thereof, and BYL719, or an analog or a derivative thereof.

In some cases, the composition comprises from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693, or an analog or a derivative thereof. In some cases, the composition comprises about 0.01 μM, 0.02 μM, 0.04 μM, 0.06 μM, 0.08 μM, 0.1 μM, 0.12 μM, 0.15 μM, 0.2 μM, 0.3 μM, 0.4 μM, 0.5 μM, 0.6 μM, 0.8 μM, or 1 μM of GSK-690693. In some cases, the composition comprises from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719, or an analog or a derivative thereof. In some cases, the composition comprises about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719. In some cases, the composition comprises about 0.08 μM to about 0.12 μM of GSK-690693 and about 35 nM to about 50 nM of BYL719.

In some cases, the composition provided herein further comprises a water-soluble synthetic polymer. In some cases, the water-soluble synthetic polymer comprises polyvinyl alcohol, poloxamer, polyvinylpyrrolidone, polyethylene glycol (PEG), PEG copolymers, poly(N-isopropylacrylamide), or polyacrylamide. In some cases, the water-soluble synthetic polymer comprises polyvinyl alcohol. In some cases, the water-soluble synthetic polymer is present at a concentration of about 0.005% to about 0.5% (w/v), about 0.01% to about 0.2% (w/v), about 0.02% to about 0.1% (w/v), or about 0.03% to about 0.08% (w/v) in the culture medium. In some cases, the water-soluble synthetic polymer is present at a concentration of about 0.04% to about 0.06% (w/v) in the culture medium. In some cases, the water-soluble synthetic polymer is present at a concentration of about 0.05% (w/v) in the culture medium. In some cases, the water-soluble synthetic polymer com-prises polyvinyl alcohol that is less than 85% hydrolyzed. In some cases, the water-soluble synthetic polymer com-prises polyvinyl alcohol that is about 80% hydrolyzed.

In some embodiments, the composition provided herein has a liquid volume of about 500 mL to about 50 L, about 1 L to about 10 L, about 2 L to about 5 L, about 3 L to about 4 L, about 2 L to about 30 L, or about 10 L to about 20 L. In some embodiments, the composition provided herein has a liquid volume of about 10 mL to about 1000 mL, about 10 mL to about 100 mL, about 20 mL to about 50 mL, about 30 mL to about 40 mL, about 20 mL to about 30 mL, or about 10 mL to about 20 mL.

In some aspects, provided herein is a method, comprising contacting a plurality of stem cells in vitro with an inhibitor of PI3K/Akt/mTOR signaling.

In some cases, the inhibitor of PI3K/Akt/mTOR signaling comprises an inhibitor of a PI3K protein, an inhibitor of an Akt protein, an inhibitor of mTOR, or any combination thereof. In some cases, the inhibitor of PI3K/Akt/mTOR signaling comprises one or more of: GSK-690693, IPI-3063, AZD8055, Omipalisib, GNE-477, VS-5584, BYL319, YM201636, PI4KIIIbeta-IN-10, Nemiralisib, BYL719, FT113, Apitolisib, or any analog or derivative thereof. In some cases, the inhibitor of PI3K/Akt/mTOR signaling comprises an inhibitor of a PI3K protein and an inhibitor an Akt protein. In some cases, the inhibitor of PI3K/Akt/mTOR signaling comprises GSK-690693, an analog or a derivative thereof. In some cases, the inhibitor of PI3K/Akt/mTOR signaling comprises BYL719, an analog or a derivative thereof. In some cases, the inhibitor of PI3K/Akt/mTOR signaling comprises GSK-690693 and BYL719. In some cases, the contacting comprises contacting the plurality of stem cells with from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693. In some cases, the contacting comprises contacting the plurality of stem cells with about 0.01 μM, 0.02 μM, 0.04 μM, 0.06 μM, 0.08 μM, 0.1 μM, 0.12 μM, 0.15 μM, 0.2 μM, 0.3 μM, 0.4 μM, 0.5 μM, 0.6 μM, 0.8 μM, or 1 μM of GSK-690693. In some cases, the contacting comprises contacting the plurality of stem cells with from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719. In some cases, the contacting comprises contacting the plurality of stem cells with about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719. In some cases, the contacting comprises contacting the plurality of stem cells with from about 0.01 μM to about 1 μM, about 0.02 μM to about 0.8 μM, about 0.05 μM to about 0.5 μM, about 0.06 μM to about 0.2 μM, or about 0.07 μM to about 0.15 μM of GSK-690693, and from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719. In some cases, the contacting comprises contacting the plurality of stem cells with about 0.08 μM to about 0.12 μM of GSK-690693 and about 35 nM to about 50 nM of BYL719.

In some cases, the method comprises contacting the plurality of stem cells with the inhibitor of PI3K/Akt/mTOR signaling and a growth factor from TGF-β superfamily. In some cases, the growth factor from TGF-β superfamily comprises Activin A, GDF8, or both. In some cases, the method comprises contacting the plurality of stem cells with from about 0.5 ng/mL to about 500 ng/mL, about 1 ng/mL to about 250 ng/mL, about 10 ng/mL to about 200 ng/mL, about 20 ng/mL to about 150 ng/mL, about 50 ng/mL to about 120 ng/mL, about 1 ng/mL to about 50 ng/mL, about 2 ng/mL to about 25 ng/mL, or about 5 ng/mL to about 20 ng/mL of Activin A. In some cases, the method comprises contacting the plurality of stem cells with about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 12 ng/mL, 14 ng/mL, 15 ng/mL, 18 ng/mL, 20 ng/mL, 25 ng/mL, 30 ng/mL, or 50 ng/mL of Activin A.

In some cases, the method comprises contacting the plurality of stem cells with the inhibitor of PI3K/Akt/mTOR signaling for from about 24 hours to about 96 hours, from about 36 hours to about 84 hours, from about 48 hours to about 84 hours, from about 60 hours to about 84 hours, or about three days.

In some cases, the method comprises contacting the plurality of stem cells also with an activator of WNT signaling pathway. In some cases, the activator of WNT signaling pathway comprises one or more of Wnt3a, CHIR99021, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC693868, SB 216763, SB 415286, TC-G 24, TCS 2002, TCS 21311, TWS 119, and analogs or derivatives of any of these. In some cases, the activator of WNT signaling pathway comprises a GSK3 inhibitor. In some cases, the method comprises contacting the plurality of stem cells with from about 0.5 μM to about 50 μM, about 0.6 μM to about 30 μM, about 0.8 μM to about 20 μM, about 1 μM to about 10 μM, or about 2 μM to about 5 μM of CHIR99021. In some cases, the method comprises contacting the plurality of stem cells with about 0.5 μM, 0.6 μM, 0.8 μM, 1 μM, 2 μM, 3 μM, 4 μM, 5 μM, 6 μM, 8 μM, 10 μM, 15 μM, M, 25 μM, or 30 μM of CHIR99021. In some cases, the method comprises culturing the plurality of stem cells in a first composition comprising the inhibitor of PI3K/Akt/mTOR signaling and the activator of WNT signaling pathway for from 12 hours to 48 hours, from 12 hours to 36 hours, from 18 hours to 30 hours, or about one day.

In some cases, the method further comprises after the culturing in the first composition, culturing at least part of resulting cells in a second composition that comprises the inhibitor of PI3K/Akt/mTOR signaling for from 12 hours to 72 hours, from 24 hours to 72 hours, from 36 hours to 72 hours, or about two days. In some cases, the second composition does not comprise the activator of WNT signaling pathway. In some cases, the second composition comprises the same concentration of the inhibitor of PI3K/Akt/mTOR signaling as the first composition.

In some cases, the stem cells comprise embryonic stem cells. In some cases, the stem cells comprise induced pluripotent stem cells. In some cases, the stem cells are human cells. In some cases, the stem cells are genetically modified.

In some cases, the contacting the plurality of stem cells in vitro with the inhibitor of PI3K/Akt/mTOR signaling results in generation of a population of cells comprising Sox17-positive cells. In some cases, the population of cells comprises at least about 50%, 60%, 65%, 70%, 75%, 80%, or 85% Sox17-positive, Oct4-negative cells. In some cases, the population of cells comprises from about 50% to about 90%, about 60% to about 90%, about 65% to about 90%, about 70% to about 90%, about 75% to about 90%, about 80% to about 90%, or about 75% to about 85% Sox17-positive, Oct4-negative cells.

In some cases, the method results in generation of the population of cells that comprises a percentage of Sox17-positive, Oct4-negative cells that is equivalent to a percentage of Sox17-positive, Oct4-negative cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

In some cases, the method further comprises differentiating the Sox17-positive cells into pancreatic β cells; NKX6.1-positive, ISL1-positive cells; PDX1-positive, NKX6.1-positive cells; PDX1-positive, NKX6.1-negative cells; FOXA2-positive, PDX1-negative cells; or any combination thereof.

In some cases, the method further comprises contacting cells in the population of cells comprising Sox17-positive cells with a growth factor from fibroblast growth factors (FGF) family. In some cases, the growth factor from fibroblast growth factors (FGF) family is selected from the group consisting of: keratinocyte growth factor (KGF), FGF2, FGF10, FGF21, and FGF8B. In some cases, the method comprises culturing cells in the population of cells a third composition that comprises the growth factor from fibroblast growth factors (FGF) family for 1 to 5 days, or 2 to 4 days, or about 1, 2, 3, 4, or 5 days. In some cases, the contacting with the growth factor from fibroblast growth factors (FGF) family results in generation of a population of cells comprising FOXA2-positive, PDX1-negative cells. In some cases, the population of cells comprising FOXA2-positive, PDX1-negative cells has a percentage of FOXA2-positive, PDX1-negative cells that is equivalent to a percentage of FOXA2-positive, PDX1-negative cells in a population of cells generated by a reference method, wherein the reference method comprises contacting the plurality of stem cells with about 100 ng/mL Activin A but not the inhibitor of PI3K/Akt/mTOR signaling, but is otherwise identical to the method. In some cases, the method further comprises contacting cells in the population of cells comprising FOXA2-positive, PDX1-negative cells with one or more agents selected from the group consisting of: a protein kinase C activator, a growth factor from transformation growth factor β (TGF-β) superfamily, a bone morphogenetic protein signaling pathway inhibitor, a growth factor from fibroblast growth factors (FGF) family, a retinoic acid (RA) signaling pathway activator, a Rho-associated, coiled-coil containing protein kinase (ROCK) inhibitor, and a sonic hedgehog (SHH) pathway inhibitor. In some cases, the method comprises contacting the cells in the population of cells comprising FOXA2-positive, PDX1-negative cells with: (a) a protein kinase C activator selected from the group consisting of: phorbol 12,13-dibutyrate (PDBU), TPB, phorbol 12-myristate 13-acetate, and bryostatin 1; (b) a growth factor from the transformation growth factor β (TGF-β) superfamily selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11); (c) a bone morphogenetic protein signaling pathway inhibitor comprising LDN193189 or DMH-1; (d) a growth factor from fibroblast growth factors (FGF) family selected from the group consisting of: keratinocyte growth factor (KGF), FGF2, FGF10, FGF21, and FGF8B; (e) a sonic hedgehog pathway inhibitor selected from the group consisting of SANT1, SANT2, SANT4, Cur6l4l4, forskolin, tomatidine, AY9944, triparanol, and cyclopamine; (f) a retinoic acid signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; and/or (g) a ROCK inhibitor selected from the group consisting of Thiazovivin, Y-27632, Fasudil/HA1077, and 14-1152. In some cases, the method comprises culturing the cells in the population of cells comprising FOXA2-positive, PDX1-negative cells in a fourth composition for 4 to 8 days, or 5 to 7 days, or about 4, 5, 6, 7, or 8 days, and wherein the fourth composition comprises the one or more agents selected from the group consisting of: a protein kinase C activator, a growth factor from transformation growth factor β (TGF-β) superfamily, a bone morphogenetic protein signaling pathway inhibitor, a growth factor from fibroblast growth factors (FGF) family, a retinoic acid (RA) signaling pathway activator, a Rho-associated, coiled-coil containing protein kinase (ROCK) inhibitor, and a sonic hedgehog (SHH) pathway inhibitor.

In some cases, the contacting the cells in the population of cells comprising FOXA2-positive, PDX1-negative cells results in generation of a population of cells comprising PDX1-positive, NKX6.1-negative cells.

In some cases, the population of cells comprising PDX1-positive, NKX6.1-negative cells has a percentage of PDX1-positive, NKX6.1-negative cells that is equivalent to a percentage of PDX1-positive, NKX6.1-negative cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

In some cases, the method further comprises contacting cells in the population of cells comprising PDX1-positive, NKX6.1-negative cells with one or more agents selected from the group consisting of: a growth factor from transformation growth factor β (TGF-β) superfamily, a growth factor from fibroblast growth factors (FGF) family, a retinoic acid (RA) signaling pathway activator, a Rho-associated, coiled-coil containing protein kinase (ROCK) inhibitor, a protein kinase C activator, a FoxO1 inhibitor, a sonic hedgehog (SHH) pathway inhibitor, and a notch signaling inhibitor. In some cases, the method comprises contacting the cells in the population of cells comprising PDX1-positive, NKX6.1-negative cells with: (a) a growth factor from the transformation growth factor β (TGF-β) superfamily selected from the group consisting of: an Inhibin, an Activin, a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11); (b) a growth factor from fibroblast growth factors (FGF) family selected from the group consisting of: keratinocyte growth factor (KGF), FGF2, FGF10, FGF21, and FGF8B; (c) a retinoic acid (RA) signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; (d) a ROCK inhibitor selected from the group consisting of Thiazovivin, Y-27632, Fasudil/HA1077, and 14-1152; (e) a protein kinase C activator selected from the group consisting of: phorbol 12,13-dibutyrate (PDBU), TPB, phorbol 12-myristate 13-acetate, and bryostatin 1; (f) a FoxO1 inhibitor, optionally wherein the FoxO1 inhibitor is AS1842856; (g) a sonic hedgehog (SHH) pathway inhibitor selected from the group consisting of SANT1, SANT2, SANT4, Cur6l4l4, forskolin, tomatidine, AY9944, triparanol, and cyclopamine; and/or (h) a notch signaling inhibitor, optionally wherein the notch signaling inhibitor is XXI or DAPI.

In some cases, the method comprises culturing the cells in the population of cells comprising PDX1-positive, NKX6.1-negative cells in a fifth composition for 4 to 8 days, or 5 to 7 days, or about 4, 5, 6, 7, or 8 days, and wherein the fifth composition comprises the one or more agents selected from the group consisting of: a growth factor from transformation growth factor β (TGF-β) superfamily, a growth factor from fibroblast growth factors (FGF) family, a retinoic acid (RA) signaling pathway activator, a Rho-associated, coiled-coil containing protein kinase (ROCK) inhibitor, a protein kinase C activator, a FoxO1 inhibitor, a sonic hedgehog (SHH) pathway inhibitor, and a notch signaling inhibitor.

In some cases, the contacting the cells in the population of cells comprising PDX1-positive, NKX6.1-negative cells results in differentiation of PDX1-positive, NKX6.1-negative cells into PDX1-positive, NKX6.1-positive cells, thereby generating a population of cells comprising PDX1-positive, NKX6.1-positive cells.

In some cases, the population of cells comprising PDX1-positive, NKX6.1-positive cells has a percentage of PDX1-positive, NKX6.1-positive cells that is equivalent to a percentage of PDX1-positive, NKX6.1-positive cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

In some cases, the first composition, the second composition, the third composition, the fourth composition, or the fifth composition further comprises a water-soluble synthetic polymer. In some cases, the water-soluble synthetic polymer comprises polyvinyl alcohol, poloxamer, polyvinylpyrrolidone, polyethylene glycol (PEG), PEG copolymers, poly(N-isopropylacrylamide), or polyacrylamide. In some cases, the water-soluble synthetic polymer comprises polyvinyl alcohol. In some cases, the water-soluble synthetic polymer is present at a concentration of about 0.005% to about 0.5% (w/v), about 0.01% to about 0.2% (w/v), about 0.02% to about 0.1% (w/v), or about 0.03% to about 0.08% (w/v). In some cases, the water-soluble synthetic polymer is present at a concentration of about 0.05% (w/v) in the culture medium. In some cases, the water-soluble synthetic polymer comprises polyvinyl alcohol that is less than 85% hydrolyzed. In some cases, the water-soluble synthetic polymer comprises polyvinyl alcohol that is about 80% hydrolyzed.

In some cases, the method further comprises contacting cells in the population of cells comprising PDX1-positive, NKX6.1-positive cells with one or more agents selected from the group consisting of: a protein kinase C activator, a TGF-β signaling pathway inhibitor, a thyroid hormone signaling pathway activator, an epigenetic modifying compound, a growth factor from epidermal growth factor (EGF) family, a retinoic acid (RA) signaling pathway activator, a sonic hedgehog (SHH) pathway inhibitor, a γ-secretase inhibitor, a protein kinase inhibitor, a Rho-associated, coiled-coil containing protein kinase (ROCK) inhibitor, a bone morphogenetic protein (BMP) signaling pathway inhibitor, and a Wnt signaling pathway inhibitor. In some cases, the method comprises contacting the cells in the population of cells comprising PDX1-positive, NKX6.1-positive cells with: (a) a TGF-β signaling pathway inhibitor selected from the group consisting of: Alk5i II, A83-01, SB431542, D4476, GW788388, LY364947, LY580276, SB505124, GW6604, SB-525334, SD-208, or SB-505124; (b) a thyroid hormone signaling pathway activator comprising T3 or GC-1; (c) an epigenetic modifying compound selected from the group consisting of: 3-deazaneplanocin A (DZNep), GSK126, EPZ6438, KD5170, MC1568, and TMP195; (d) a growth factor from the epidermal growth factor family comprising betacellulin or EGF; (e) a retinoic acid signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; (f) a sonic hedgehog pathway inhibitor selected from the group consisting of SANT1, SANT2, SANT4, Cur6l4l4, forskolin, tomatidine, AY9944, triparanol, and cyclopamine; (g) a 7-secretase inhibitor comprising XXI or DAPT; (h) a protein kinase inhibitor comprising staurosporine, Ro-31-8220, a bisindolylmaleimide (Bis) compound, 10′-{5″-[(methoxycarbonyl)amino]-2″-methyl}-phenylaminocarbonylstaurosporine, or a staralog; (i) a ROCK inhibitor selected from the group consisting of Thiazovivin, Y-27632, Fasudil/HA1077, and 14-1152; (j) a protein kinase C activator selected from the group consisting of: phorbol 12,13-dibutyrate (PdBU), TPB, phorbol 12-myristate 13-acetate, and bryostatin 1; (k) a bone morphogenetic protein signaling pathway inhibitor comprising LDN193189 or DMH-1; and/or (l) a Wnt signaling pathway inhibitor comprising NVP-TNKS656.

In some cases, the method comprises culturing the cells in the population of cells comprising PDX1-positive, NKX6.1-positive cells in a sixth composition for 5 to 10 days, or 6 to 9 days, or about 5, 6, 7, 8, 9, or 10 days, and wherein the sixth composition comprises the one or more agents selected from the group consisting of: a protein kinase C activator, a TGF-β signaling pathway inhibitor, a thyroid hormone signaling pathway activator, an epigenetic modifying compound, a growth factor from epidermal growth factor (EGF) family, a retinoic acid (RA) signaling pathway activator, a sonic hedgehog (SHH) pathway inhibitor, a γ-secretase inhibitor, a protein kinase inhibitor, a Rho-associated, coiled-coil containing protein kinase (ROCK) inhibitor, a bone morphogenetic protein (BMP) signaling pathway inhibitor, and a Wnt signaling pathway inhibitor.

In some cases, the sixth composition further comprises one or more of an acetyl CoA-related metabolite (e.g., acetate), a vitamin (e.g., biotin), histone deacetylase inhibi-tor (HDACi) (e.g., β-hydroxybutyrate), a redox homeostasis regulator (e.g., taurine), a one car-bon metabolism pathway intermediate (e.g., formate), and/or glutamine (e.g., L-glutamine).

In some cases, the contacting the cells in the population of cells results in generation of a population of cells comprising NKX6.1-positive, ISL1-positive cells.

In some cases, the population of cells comprising NKX6.1-positive, ISL1-positive cells has a percentage of NKX6.1-positive, ISL1-positive cells that is equivalent to a percentage of NKX6.1-positive, ISL1-positive cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

In some cases, the method further comprises differentiating the NKX6.1-positive, ISL1-positive cells into a population of cells comprising pancreatic β cells. In some cases, the method further comprises contacting cells in the population of cells comprising NKX6.1-positive, ISL1-positive cells with a seventh composition comprising one or more agents selected from the group consisting of: a transformation growth factor β (TGF-β) signaling pathway inhibitor, a thyroid hormone signaling pathway activator, an epigenetic modifying compound, a growth factor from epidermal growth factor (EGF) family, a retinoic acid (RA) signaling pathway activator, a sonic hedgehog (SHH) pathway inhibitor, a γ-secretase inhibitor, a protein kinase inhibitor, a Rho-associated, coiled-coil containing protein kinase (ROCK) inhibitor, and a bone morphogenetic protein (BMP) signaling pathway inhibitor.

In some cases, the population of cells comprising pancreatic β cells has a percentage of pancreatic β cells that is equivalent to a percentage of pancreatic β cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

In some cases, the sixth composition or the seventh composition further comprises a water-soluble synthetic polymer. In some cases, the water-soluble synthetic polymer comprises polyvinyl alcohol, poloxamer, polyvinylpyrrolidone, polyethylene glycol (PEG), PEG copolymers, poly(N-isopropylacrylamide), or polyacrylamide. In some cases, the water-soluble synthetic polymer comprises polyvinyl alcohol. In some cases, the water-soluble synthetic polymer is present at a concentration of about 0.005% to about 0.5% (w/v), about 0.01% to about 0.2% (w/v), about 0.02% to about 0.1% (w/v), or about 0.03% to about 0.08% (w/v). In some cases, the water-soluble synthetic polymer is present at a concentration of about 0.05% (w/v) in the culture medium. In some cases, the water-soluble synthetic polymer comprises polyvinyl alcohol that is more than 85% hydrolyzed. In some cases, the water-soluble synthetic polymer comprises polyvinyl alcohol that is about 87% to 89% hydrolyzed.

In some aspects, provided herein is a device comprising the composition or a population of cells obtained from the composition disclosed herein, or cells generated according to the method disclosed herein. In some cases, the device is configured to produce and release insulin when implanted into a subject. In some cases, the cells are encapsulated. In some cases, the device further comprises a semipermeable membrane, wherein the semipermeable membrane is configured to retain the cells in the device and permit passage of insulin.

In some aspects, provided herein is a method of treating a subject with a disease characterized by high blood sugar levels over a prolonged period of time, the method comprising administering the composition or a population of cells obtained from the composition disclosed herein, or cells generated according to the method disclosed herein, or implanting the device disclosed herein, to the subject. In some cases, the disease is diabetes, optionally type I diabetes.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIGS. 1A-1E demonstrate the effects of treatment of stem cells (Stage 1 differentiation) with different concentrations of Activin A on pancreatic β cell differentiation. FIG. 1A shows pictures of cell clusters (bottom) obtained upon completion of Stage 1 (“SiC”) with treatment of 100 ng/mL Activin A (“100% AA”), 10 ng/mL Activin A (“10% AA”), or 5 ng/mL Activin A (“5% AA”), and results of flow cytometry analysis of expression of Oct4 and Sox17 in the cells at S1C. FIG. 1B shows pictures of cell clusters (bottom) obtained upon completion of Stage 3 (“S3C”) with treatment of 100% AA, 10% AA, or 5% AA, and results of flow cytometry analysis of expression of Pdx1 and Cdx2 in the cells at S3C. FIG. 1C shows results of flow cytometry analysis of expression of Pdx1 and Sox2 in the cells at S3C. FIG. 1D shows pictures of cell clusters (bottom) obtained upon completion of Stage 5 (“S5C”) with treatment of 100% AA, 10% AA, or 5% AA, and results of flow cytometry analysis (top) of expression of Nkx6.1 and Isl1 in the cells at S5C. According to the flow cytometry analysis, there was 41.1% Nkx6.1-positive, Isl1-negative cells under 100% AA, and 49.5% Nkx6.1-positive, Isl1-negative cells under 10% AA. FIG. 1E shows a bar graph and a table summarizing the percentage of Sox7-negative, Oct4-positive cells (“Sox17−/Oct4+”), and Sox7-negative, Oct4-negative cells (“double negative”) at S1C with treatment of 100 ng/mL (100%), 20 ng/mL (20%), 10 ng/mL (10%), 5 ng/mL (5%), or 0 ng/mL (0%) Activin A at Stage 1.

FIGS. 2A-2C demonstrate the effects of treatment of stem cells with exemplary compounds upon completion of Stage 1 differentiation. FIG. 2A shows pictures of cell clusters obtained at S1C with treatment of 100% AA, 10% AA, 10% Activin A plus GSK-690693, 10% Activin A plus BYL719, 10% Activin A plus GSK-690693 and BYL719, or 100% Activin A plus GSK-690693 and BYL719 at Stage 1. FIG. 2B shows results of flow cytometry analysis of expression of Oct4 and Sox17 in the cells at S1C, and FIG. 2C shows bar graph summarizing these results.

FIGS. 3A-3E demonstrate the effects of treatment of stem cells with exemplary compounds at Stage 1 upon completion of Stage 3 differentiation. FIG. 3A shows pictures of cell clusters obtained at S3C following Stage 1 treatment with 100% AA, 10% AA, 10% Activin A plus GSK-690693, 10% Activin A plus BYL719, 10% Activin A plus GSK-690693 and BYL719, or 100% Activin A plus GSK-690693 and BYL719. FIG. 3B shows results of flow cytometry analysis of expression of Pdx1 and Cdx2 in the cells at S3C, which are summarized by the bar graph in FIG. 3C. FIG. 3D shows results of flow cytometry analysis of expression of Pdx1 and Sox2 in the cells at S3C, which are summarized by the bar graph in FIG. 3E.

FIGS. 4A-4F demonstrate the effects of treatment of stem cells with exemplary compounds at Stage 1 upon completion of Stage 5 differentiation. FIG. 4A shows pictures of cell clusters obtained at S5C with treatment of 100% AA, 10% AA, 10% Activin A plus GSK-690693, 10% Activin A plus BYL719, 10% Activin A plus GSK-690693 and BYL719, or 100% Activin A plus GSK-690693 and BYL719 at Stage 1. FIG. 4B shows results of flow cytometry analysis of expression of Nkx6.1 and Isl1 in the cells at S5C, which are summarized by the bar graph in FIGS. 4C-4D. FIGS. 4E-4F show bar graphs summarizing total cell yield (FIG. 4E) and yields of Isl1-positive, Nkx6.1-positive cells (Isl1+/Nkx6.1+) and Isl1-positive, Nkx6.1-negative cells (Isl1+/Nkx6.1−) (FIG. 4F) at S5C, respectively.

FIGS. 5A-5E demonstrate the effects of treatment of stem cells with exemplary compounds at Stage 1 on cells upon completion of 7 days of Stage 6 differentiation that followed an exemplary Stage 6 medium regimen (“regimen 1”). FIG. 5A shows results of flow cytometry analysis of expression of Nkx6.1 and Isl1 at day 7 of Stage 6 (“S6d7”) with treatment of 100% AA, 10% AA, 10% Activin A plus GSK-690693, 10% Activin A plus BYL719, 10% Activin A plus GSK-690693 and BYL719, and 100% Activin A plus GSK-690693 and BYL719 at Stage 1. FIG. 5B shows a bar graph summarizing the results in FIG. 5A. FIG. 5C shows results of flow cytometry analysis of expression of C-peptide (“C-pep”) and glucagon (“GCG”) in the cells at S6d7. FIG. 5D shows results of flow cytometry analysis of expression of somatostatin (“SST”) and GCG in the cells at S6d7. FIG. 5E shows results of flow cytometry analysis of expression of SST and C-pep in the cells at S6d7.

FIGS. 6A-6E demonstrate the effects of treatment of stem cells with exemplary compounds at Stage 1 on cells upon completion of 7 days of Stage 6 differentiation that followed a different exemplary Stage 6 medium regimen (“regimen 2”). FIG. 6A shows results of flow cytometry analysis of expression of Nkx6.1 and Isl1 at day 7 of Stage 6 (“S6d7”) with treatment of 100% AA, 10% AA, 10% Activin A plus GSK-690693, 10% Activin A plus BYL719, 10% Activin A plus GSK-690693 and BYL719, and 100% Activin A plus GSK-690693 and BYL719 at Stage 1. FIG. 6B shows a bar graph summarizing the results in FIG. 6A. FIG. 6C shows results of flow cytometry analysis of expression of C-peptide (“C-pep”) and glucagon (“GCG”) in the cells at S6d7. FIG. 6D shows results of flow cytometry analysis of expression of somatostatin (“SST”) and GCG in the cells at S6d7. FIG. 6E shows results of flow cytometry analysis of expression of SST and C-pep in the cells at S6d7.

FIG. 7A shows a graph summarizing the percentage of SC-iselt β cells and the percentage of SC-islet non-β cells in cell compositions at the completion of stage 5 differentiation (“S5C”) under different culture conditions, with either (a) 100 ng/mL Activin A or (b) 0.1 μM GSK-690693 (also referred to as “GSK690693”) and 10 ng/ml Activin A at stage 1. FIG. 7B shows a graph summarizing the percentage of SC-iselt β cells and the percentage of SC-islet non-β cells in the cell composition on day 7 of Stage 6 (“S6d7”) under different culture conditions, with either (a) 100 ng/mL Activin A or (b) 0.1 μM GSK-690693 (also referred to as “GSK690693”) and 10 ng/ml Activin A at stage 1.

### DETAILED DESCRIPTION

Pancreatic islet transplantation is a promising therapy that can achieve significant clinical benefit for diabetic subjects, for example, subjects with type I diabetes. As there is a limited supply of pancreatic islets sources from donor pancreatic tissue, there is a need for improved techniques to generate implantable islets from alternative sources, such as stem cells. Improved methods of generating islet components (e.g., SC-β cells) could result in more effective therapeutic products (e.g., SC-β cells with improved functionality), improved methods of manufacturing SC-islets for human therapeutic use (e.g., lower production costs, and/or higher cell yields), or a combination thereof.

Provided herein are, inter alia, compositions and methods for improved production of SC-β cells in vitro. Certain compositions and combinations of agents disclosed here promote cost-effective, large-scale production of SC-β cells in vitro. For example, the disclosure provides novel formulations and differentiation methods that make use of small molecule compounds in lieu of certain growth factors while maintaining comparable or even improved cell yields, relative percentages of desirable cell populations (e.g., on-target differentiated cells at various differentiation stages and the resulting SC-β cells), function of the SC-β cells in vitro, viability, function, and immunogenicity after transplantation. The disclosed compositions and methods can be particularly advantageous for the large-scale manufacture of SC-islets for human therapeutic use. Small molecule compounds in the compositions and methods disclosed herein include, for example, inhibitors of PI3K/Akt/mTOR signaling.

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

### Definitions

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “diabetes” and its grammatical equivalents as used herein can refer to a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some cases, diabetes can be a form of hereditary diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic α cells, pancreatic β cells, pancreatic δ cells, pancreatic F cells, and/or pancreatic ε cells. Islet cells can also refer to a group of cells, cell clusters, or the like.

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to a stem cell-derived pancreatic cell (e.g., SC-β cell) can refer to any cell that is capable of differentiating into a SC-β cell, including for example, a pluripotent stem cell, a definitive endoderm cell, a primitive gut tube cell, a pancreatic progenitor cell, or endocrine progenitor cell, when cultured under conditions suitable for differentiating the precursor cell into the insulin-positive endocrine cell.

The term “exocrine cell” as used herein can refer to a cell of an exocrine gland, i.e. a gland that discharges its secretion via a duct. In particular embodiments, an exocrine cell can refer to a pancreatic exocrine cell, which is a pancreatic cell that can produce enzymes that are secreted into the small intestine. These enzymes can help digest food as it passes through the gastrointestinal tract. Pancreatic exocrine cells are also known as islets of Langerhans, which can secrete two hormones, insulin and glucagon. A pancreatic exocrine cell can be one of several cell types; α-2 cells (which can produce the hormone glucagon); or β cells (which can manufacture the hormone insulin); and α-1 cells (which can produce the regulatory agent somatostatin). Non-insulin-producing exocrine cells, as the term is used herein, can refer to α-2 cells or α-1 cells.

The terms “stem cell-derived β cell,” “SC-β cell,” “functional β cell,” “functional pancreatic β cell,” “mature SC-β cell,” “SC-islet β cell,” and their grammatical equivalents can refer to cells (e.g., non-native pancreatic β cells) that display at least one marker indicative of a pancreatic β cell (e.g., PDX1 or NKX6.1), expresses insulin, and display a glucose stimulated insulin secretion (GSIS) response characteristic of an endogenous mature β cell. In some embodiments, the terms “SC-β cell” and “non-native β cell” as used herein are interchangeable. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous β cell. In some embodiments, the SC-β cell exhibits an in vitro GSIS response that resembles the GSIS response of an endogenous β cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous β cell. The GSIS response of the SC-β cell can be observed within two weeks of transplantation of the SC-β cell into a host (e.g., a human or animal). In some embodiments, the SC-β cells package insulin into secretory granules. In some embodiments, the SC-β cells exhibit encapsulated crystalline insulin granules. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose.

As used herein, the term “insulin producing cell” and its grammatical equivalent refer to a cell differentiated from a pancreatic progenitor, or precursor thereof, which secretes insulin. An insulin-producing cell can include pancreatic β cell as that term is described herein, as well as pancreatic j-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive, endocrine cells or a precursor thereof into SC-β cells according to the methods of the present disclosure can be pancreatic β cell or (β-like cells (e.g., cells that have at least one, or at least two least two) characteristic of an endogenous β cell and exhibit a glucose stimulated insulin secretion (GSIS) response that resembles an endogenous adult β cell. The population of insulin-producing cells, e.g., produced by the methods as disclosed herein can comprise mature pancreatic β cell or SC-β cells, and can also contain non-insulin-producing cells (e.g., cells of cell like phenotype with the exception they do not produce or secrete insulin).

The terms “insulin-positive j-like cell,” “insulin-positive endocrine cell,” and their grammatical equivalents can refer to cells (e.g., pancreatic endocrine cells) that display at least one marker indicative of a pancreatic β cell and also expresses insulin but lack a glucose stimulated insulin secretion (GSIS) response characteristic of an endogenous β cell.

The term “β cell marker” refers to, without limitation, proteins, peptides, nucleic acids, polymorphism of proteins and nucleic acids, splice variants, fragments of proteins or nucleic acids, elements, and other analyte which are expressed or present in pancreatic β cells. Exemplary β cell markers include, but are not limited to, pancreatic and duodenal homeobox 1 (Pdx1 or PDX1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, and MafA, and those described in Zhang et al., Diabetes. 50(10):2231-6 (2001). In some embodiment, the β cell marker is a nuclear 3-cell marker. In some embodiments, the β cell marker is Pdx1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analyte which are expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g., β cells that produce insulin; a cells that produce glucagon; 6 cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “Pdx1-positive pancreatic progenitor” as used herein can refer to a cell which is a pancreatic endoderm (PE) cell which has the capacity to differentiate into SC-β cells, such as pancreatic β cells. A Pdx1-positive pancreatic progenitor expresses the marker Pdx1. Other markers include, but are not limited to Cdcp1, or Ptf1a, or HNF6 or NRx2.2. The expression of Pdx1 may be assessed by any method known by the skilled person such as immunochemistry using an anti-Pdx1 antibody or quantitative RT-PCR. In some cases, a Pdx1-positive pancreatic progenitor cell lacks expression of NKX6.1 (or Nkx6.1). A Pdx1-positive pancreatic progenitor cell can also be referred to as Pdx1-positive, NKX6.1-negative pancreatic progenitor cell due to its lack of expression of NKX6.1. In some cases, the Pdx1-positive pancreatic progenitor cells can also be termed as “pancreatic foregut endoderm cells.” As used herein, the terms “PDX1,” “Pdx1,” and “PDX-1” are equivalent and interchangeable.

The term “Pdx1-positive, NKX6-1-positive pancreatic progenitor” as used herein can refer to a cell which is a pancreatic endoderm (PE) cell which has the capacity to differentiate into insulin-producing cells, such as pancreatic β cells. A Pdx1-positive, NKX6-1-positive pancreatic progenitor expresses the markers Pdx1 and NKX6-1. Other markers may include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR. As used herein, the terms “Nkx6.1,” “NKX6.1,” and “NKX6-1” are equivalent and interchangeable. In some cases, the Pdx1-positive, NKX6-1-positive pancreatic progenitor cells can also be termed as “pancreatic foregut precursor cells.”

The term “Ngn3-positive endocrine progenitor” as used herein can refer to precursors of pancreatic endocrine cells expressing the transcription factor Neurogenin-3 (Ngn3). Progenitor cells are more differentiated than multipotent stem cells and can differentiate into only few cell types. In particular, Ngn3-positive endocrine progenitor cells have the ability to differentiate into the five pancreatic endocrine cell types (α, β, δ, ε and PP). The expression of Ngn3 may be assessed by any method known by the skilled person such as immunochemistry using an anti-Ngn3 antibody or quantitative RT-PCR.

The terms “NeuroD” and “NeuroD1” are used interchangeably and identify a protein expressed in pancreatic endocrine progenitor cells and the gene encoding it.

The term “epigenetics” refers to heritable changes in gene function that do not involve changes in the DNA sequence. Epigenetics most often denotes changes in a chromosome that affect gene activity and expression, but can also be used to describe any heritable phenotypic change that does not derive from a modification of the genome. Such effects on cellular and physiological phenotypic traits can result from external or environmental factors, or be part of normal developmental program. Epigenetics can also refer to functionally relevant changes to the genome that do not involve a change in the nucleotide sequence. Examples of mechanisms that produce such changes are DNA methylation and histone modification, each of which alters how genes are expressed without altering the underlying DNA sequence. Gene expression can be controlled through the action of repressor proteins that attach to silencer regions of the DNA. These epigenetic changes can last through cell divisions for the duration of the cell's life, and can also last for multiple generations even though they do not involve changes in the underlying DNA sequence of the organism. One example of an epigenetic change in eukaryotic biology is the process of cellular differentiation. During morphogenesis, totipotent stem cells become the various pluripotent cells, which in turn can become fully differentiated cells.

The term “epigenetic modifying compound” refers to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, alter DNA accessibility and chromatin structure, thereby regulating patterns of gene expression. These processes are crucial to normal development and differentiation of distinct cell lineages in the adult organism. They can be modified by exogenous influences, and, as such, can contribute to or be the result of environmental alterations of phenotype or pathophenotype. Importantly, epigenetic modification has a crucial role in the regulation of pluripotency genes, which become inactivated during differentiation. Non-limiting exemplary epigenetic modifying compound include a DNA methylation inhibitor, a histone acetyltransferase inhibitor, a histone deacetylase inhibitor, a histone methyltransferase inhibitor, a bromodomain inhibitor, or any combination thereof.

The term “differentiated cell” or its grammatical equivalents is meant any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell leads to the pancreatic pathway, where ^{˜}98% of the cells become exocrine, ductular, or matrix cells, and ˜2% become endocrine cells. Early endocrine cells are islet progenitors, which can then differentiate further into insulin-producing cells (e.g., functional endocrine cells) which secrete insulin, glucagon, somatostatin, or pancreatic polypeptide. Endoderm cells can also be differentiated into other cells of endodermal origin, e.g., lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of the respiratory and digestive tracts (e.g., the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are develop from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic β cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells include, but are not limited to MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox17 and in some embodiments Sox17 and HNF3B, and do not express significant levels of GATA4, SPARC, APF or DAB. Definitive endoderm cells are not positive for the marker Pdx1 (e.g., they are Pdx1-negative). Definitive endoderm cells have the capacity to differentiate into cells including those of the liver, lung, pancreas, thymus, intestine, stomach and thyroid. The expression of Sox17 and other markers of definitive endoderm may be assessed by any method known by the skilled person such as immunochemistry, e.g., using an anti-Sox17 antibody, or quantitative RT-PCR.

The term “pancreatic endoderm” can refer to a cell of endoderm origin which is capable of differentiating into multiple pancreatic lineages, including pancreatic β cells, but no longer has the capacity to differentiate into non-pancreatic lineages.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNF1-β, HNF3-β or HNF4-α. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “stem cell” as used herein, can refer to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” can refer to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retro-differentiation” by persons of ordinary skill in the art. As used herein, the term “pluripotent stem cell” includes embryonic stem cells, induced pluripotent stem cells, placental stem cells, etc.

The term “pluripotent” as used herein can refer to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g., iPS cells as that term is defined herein) also have the characteristic of the capacity of extended passaging without loss of growth potential, relative to primary cell parents, which generally have capacity for only a limited number of divisions in culture.

As used herein, the terms “iPS cell” and “induced pluripotent stem cell” are used interchangeably and can refer to a pluripotent stem cell artificially derived (e.g., induced or by complete reversal) from a non-pluripotent cell, typically an adult somatic cell, for example, by inducing a forced expression of one or more genes.

The term “phenotype” can refer to one or a number of total biological characteristics that define the cell or organism under a particular set of environmental conditions and factors, regardless of the actual genotype.

The terms “subject,” “patient,” or “individual” are used interchangeably herein, and can refer to an animal, for example, a human from whom cells can be obtained and/or to whom treatment, including prophylactic treatment, with the cells as described herein, is provided. For treatment of those infections, conditions or disease states which are specific for a specific animal such as a human subject, the term subject can refer to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like. “Patient in need thereof” or “subject in need thereof” is referred to herein as a patient diagnosed with or suspected of having a disease or disorder, for instance, but not restricted to diabetes.

The term “administering” used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. In some embodiments, any of the compositions may be administered via the hepatic portal vein. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The terms “treat,” “treating,” “treatment,” and their grammatical equivalents, as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill or injured, or at increased risk of becoming ill relative to an average member of the population and in need of such attention, care, or management.

As used herein, the term “treating” and “treatment” can refer to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. “Treatment” can also mean prolonging survival as compared to expected survival if not receiving treatment. Those in need of treatment include those already diagnosed with a cardiac condition, as well as those likely to develop a cardiac condition due to genetic susceptibility or other factors such as weight, diet and health.

The term “therapeutically effective amount”, therapeutic amount”, or its grammatical equivalents can refer to an amount effective, at dosages and for periods of time necessary, to achieve a desired therapeutic result. The therapeutically effective amount can vary according to factors such as the disease state, age, sex, and weight of the individual and the ability of a composition described herein to elicit a desired response in one or more subjects. The precise amount of the compositions of the present disclosure to be administered can be determined by a physician with consideration of individual differences in age, weight, tumor size, extent of infection or metastasis, and condition of the patient (subject).

Alternatively, the pharmacologic and/or physiologic effect of administration of one or more compositions described herein to a patient or a subject of can be “prophylactic,” e.g., the effect completely or partially prevents a disease or symptom thereof. A “prophylactically effective amount” can refer to an amount effective, at dosages and for periods of time necessary, to achieve a desired prophylactic result (e.g., prevention of disease onset).

Some numerical values disclosed throughout are referred to as, for example, “X is at least or at least about 100; or 200 [or any numerical number].” This numerical value includes the number itself.

All these different combinations are contemplated by the numerical values disclosed throughout. All disclosed numerical values should be interpreted in this manner, whether it refers to an administration of a therapeutic agent or referring to days, months, years, weight, dosage amounts, etc., unless otherwise specifically indicated to the contrary.

The ranges disclosed throughout are sometimes referred to as, for example, “X is administered on or on about day 1 to 2; or 2 to 3 [or any numerical range].” This range includes the numbers themselves (e.g., the endpoints of the range) and all individual numbers within the recited range.

All these different combinations are contemplated by the ranges disclosed throughout. All disclosed ranges should be interpreted in this manner, whether it refers to an administration of a therapeutic agent or referring to days, months, years, weight, dosage amounts, etc., unless otherwise specifically indicated to the contrary.

### Inhibitor of PI3K/Akt/Mtor Signaling

In some embodiments, the compositions and methods disclosed herein relate to use of one or more inhibitors of PI3K/Akt/mTOR signaling during differentiation of cells in the pancreatic lineage (e.g., Sox17-positive definitive endoderm cells, FOXA2-positive primitive gut tube cells, PDX1-positive, NKX6.1-negative pancreatic progenitor 1 cells, PDX1-positive, NKX6.1-negative pancreatic progenitor 2 cells, insulin-positive pancreatic endocrine cells, or SC-pancreatic β cells).

Without wishing to be bound by a certain theory, activation of a phosphoinositide 3-kinase (PI3K) phosphorylates and activates protein kinase B (PKB, also known as Akt). The phosphorylation and activation of Akt can have a number of downstream effects, such as activating cAMP response element-binding protein (CREB), inhibiting p27, localizing forkhead box O (FOXO) in the cytoplasm, activating PtdIns-3ps, and activating mechanistic target of rapamycin (mTOR, also known as mammalian target of rapamycin). As used herein, the term “inhibitor of PI3K/Akt/mTOR signaling” refers to an agent that inhibits activity of a PI3K protein, activity of an Akt protein, activity of mTOR protein, or any combination thereof. The term “inhibitor of PI3K/Akt/mTOR signaling” is not intended to be limited to only the agents that have an inhibitory effect on the signal transduction cascade that takes place inside a cell from PI3K to Akt and to mTOR. Instead, in some cases, an inhibitor of PI3K/Akt/mTOR signaling disclosed herein has an inhibitory effect on the activity of a PI3K protein without any immediate effect on the activation of Akt or mTOR, or on the activity of an Akt protein without any immediate effect on the activation of mTOR or activity of a PI3K protein. In some cases, an inhibitor of PI3K/Akt/mTOR signaling disclosed herein directly inhibits activity of mTOR protein, without any immediate effect on the activity of an Akt protein or activity of a PI3K protein. In some cases, an inhibitor of PI3K/Akt/mTOR signaling directly inhibits activity of a PI3K protein, activity of an Akt protein, activity of mTOR protein, or any combination thereof. In some cases, an inhibitor of PI3K/Akt/mTOR signaling indirectly inhibits activity of a PI3K protein, activity of an Akt protein, activity of mTOR protein, or any combination thereof.

In some embodiments, an inhibitor of PI3K/Akt/mTOR signaling disclosed herein includes an inhibitor of a PI3K protein, such as, a class I PI3K, a class II PI3K, or a class III PI3K. In some embodiments, an inhibitor of PI3K/Akt/mTOR signaling disclosed herein inhibits more than one PI3K protein, e.g., more than one class I, II, or III PI3K, or PI3Ks in more than one classes. In some embodiments, an inhibitor of PI3K/Akt/mTOR signaling disclosed herein comprises a pan-PI3K inhibitor, e.g., an agent that inhibits virtually all types of PI3Ks. Class I PI3Ks discussed herein can include PIK3CA, PIK3CB, PIK3CG, PIK3CD, PIK3R1, PIK3R2, PIK3R3, PIK3R4, PIK3R5, PIK3R6. Class II PI3Ks discussed herein can include PIK3C2A, PIK3C2B, PIK3C2G. Class III PI3Ks discussed herein can include PIK3C3.

In some embodiments, an inhibitor of PI3K/Akt/mTOR signaling disclosed herein includes an inhibitor of an Akt protein, such as, Akt1, Akt2, or Akt3. In some embodiments, an inhibitor of PI3K/Akt/mTOR signaling disclosed herein inhibits more than one Akt protein, e.g., Akt1 and Akt2, Akt1 and Akt3, or Akt2 and Akt3. In some embodiments, an inhibitor of PI3K/Akt/mTOR signaling disclosed herein inhibits Akt1, but has minimal or no effect on Akt2 or Akt3. In some embodiments, an inhibitor of PI3K/Akt/mTOR signaling disclosed herein inhibits Akt2, but has minimal or no effect on Akt1 or Akt3. In some embodiments, an inhibitor of PI3K/Akt/mTOR signaling disclosed herein inhibits Akt3, but has minimal or no effect on Akt1 or Akt2. In some embodiments, an inhibitor of PI3K/Akt/mTOR signaling disclosed herein comprises a pan-Akt inhibitor, e.g., an agent that inhibits virtually all three types of Akt proteins.

In some embodiments, an inhibitor of PI3K/Akt/mTOR signaling disclosed herein includes an inhibitor of mTOR. Without wishing to be bound by a certain theory, activated mTOR protein can associate with other proteins and serve as a core component of two distinct protein complexes, mTOR complex 1 (mTORC1) and mTOR complex 2 (mTORC2), which can have different downstream cellular signaling pathways and regulate different cellular processes. In some embodiments, an inhibitor of PI3K/Akt/mTOR signaling disclosed herein inhibits the formation of mTORC1. In some embodiments, an inhibitor of PI3K/Akt/mTOR signaling disclosed herein inhibits the activity of mTORC1 and thus at least some of the target proteins and/or cellular signaling pathways that are activated by mTORC1. In some embodiments, an inhibitor of PI3K/Akt/mTOR signaling disclosed herein inhibits the formation of mTORC2. In some embodiments, an inhibitor of PI3K/Akt/mTOR signaling disclosed herein inhibits the activity of mTORC2 and thus at least some of the target proteins and/or cellular signaling pathways that are activated by mTORC2. In some embodiments, an inhibitor of PI3K/Akt/mTOR signaling disclosed herein inhibits the formation of both mTORC1 and mTORC2. In some embodiments, an inhibitor of PI3K/Akt/mTOR signaling disclosed herein inhibits the activity of both mTORC1 and mTORC2, and thus at least some of the target proteins and/or cellular signaling pathways that are activated by mTORC1 or mTORC2.

In some embodiments, an inhibitor of PI3K/Akt/mTOR signaling disclosed herein includes, but not limited to, GSK-690693, IPI-3063, AZD8055, Omipalisib, GNE-477, VS-5584, BYL319, YM201636, PI4KIIIbeta-IN-10, Nemiralisib, BYL719, FT113, Apitolisib, and any analog or derivative thereof. In some embodiments, an inhibitor of PI3K/Akt/mTOR signaling disclosed herein is one or more of GSK-690693, IPI-3063, AZD8055, Omipalisib, GNE-477, VS-5584, BYL319, YM201636, PI4KIIIbeta-IN-10, Nemiralisib, BYL719, FT113, Apitolisib, or any analog or derivative thereof.

In some embodiments, an inhibitor of PI3K/Akt/mTOR signaling disclosed herein is of Formula (I):

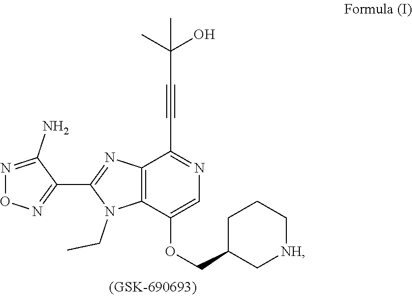 [SMILES: CCn1c(-c2nonc2N)nc2c(C#CC(C)(C)O)ncc(OC[C@H]3CCCNC3)c21]

or a pharmaceutically acceptable salt, solvate, hydrate, polymorph, co-crystal, tautomer, stereoisomer, isotopically labeled derivative, prodrug, composition, or mixture thereof.

In some embodiments, the disclosure provides for an inhibitor of PI3K/Akt/mTOR, wherein the inhibitor is any of the molecules disclosed in US2008076763, which is incorporated herein in its entirety. For example, in some embodiments, the disclosure provides for an inhibitor of PI3K/Akt/mTOR comprising the structure of Formula (II):

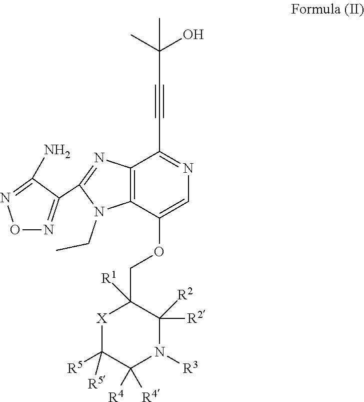

wherein: X is absent or selected from the group consisting of: O, S and CR²⁰R²¹,
where R²⁰R²¹ are independently selected from: hydrogen, fluorine, cyclopropyl, substituted cyclopropyl, cyclobutyl, substituted cyclobutyl, cyclopentyl, substituted cyclopentyl, —C₁-C₄alkyl, and substituted —C₁-C₄alkyl,
or R²⁰R²¹ taken together with the carbon to which they are attached form cyclopropyl, substituted cyclopropyl, cyclobutyl, substituted cyclobutyl, cyclopentyl or substituted cyclopentyl;
R²R^{2′} are independently selected from: hydrogen, fluorine, cyclopropyl, substituted cyclopropyl, cyclobutyl, substituted cyclobutyl, cyclopentyl, substituted cyclopentyl, —C₁-C₄alkyl, and substituted —C₁-C₄alkyl,
or R²R^{2′} taken together with the carbon to which they are attached form cyclopropyl, substituted cyclopropyl, cyclobutyl, substituted cyclobutyl, cyclopentyl or substituted cyclopentyl;
R³ is selected from the group consisting of: hydrogen, cyclopropyl, substituted cyclopropyl, cyclobutyl, substituted cyclobutyl, cyclopentyl, substituted cyclopentyl, cyclopropylmethyl, substituted cyclopropylmethyl, —C₁-C₄alkyl, and substituted —C₁-C₄alkyl;
R⁴R^{4′} are independently selected from: hydrogen, fluorine, cyclopropyl, substituted cyclopropyl, cyclobutyl, substituted cyclobutyl, cyclopentyl, substituted cyclopentyl, —C₁-C₄alkyl, and substituted —C₁-C₄alkyl, or R⁴R^{4′} taken together with the carbon to which they are attached form cyclopropyl, substituted cyclopropyl, cyclobutyl, substituted cyclobutyl, cyclopentyl or substituted cyclopentyl;
R⁵R^{5′} are independently selected from: hydrogen, fluorine, cyclopropyl, substituted cyclopropyl, cyclobutyl, substituted cyclobutyl, cyclopentyl, substituted cyclopentyl, —C₁-C₄alkyl, and substituted —C₁-C₄alkyl,
or R⁵R^{5′} taken together with the carbon to which they are attached form cyclopropyl, substituted cyclopropyl, cyclobutyl, substituted cyclobutyl, cyclopentyl or substituted cyclopentyl; and R¹ is selected from the group consisting of: hydrogen, —C₁-C₄alkyl and substituted —C₁-C₄alkyl; and
when X is absent or R²⁰R²¹, R¹ can additionally be fluorine;
and/or pharmaceutically acceptable salts, hydrates, solvates and pro-drugs thereof.

By the term “—C₁-C₄alkyl” as used herein, is meant a linear or branched, saturated or unsaturated hydrocarbon chain, containing from 1 to 4 carbon atoms. Examples of —C₁-C₄alkyl as used herein include: —CH₃, —CH₂—CH₃, —CH₂—CH₂—CH₃, —CH(CH₃)₂, —CH₂—CF₃, —C(CH₃), —(CH₂)₃—CH₃, —CH₂—CH(CH₃)₂, —CH(CH₃)—CH₂—CH₃, —CH—CH₂, and —C[identical to]C—CH₃.

In some embodiments, an inhibitor of PI3K/Akt/mTOR signaling disclosed herein is of Formula (III):

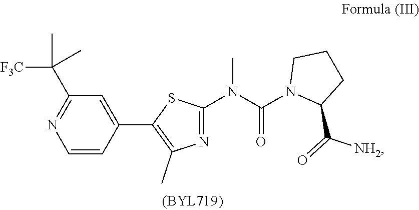 [SMILES: Cc1nc(N(C)C(=O)N2CCC[C@H]2C(N)=O)sc1-c1ccnc(C(C)(C)C(F)(F)F)c1]

or a pharmaceutically acceptable salt, solvate, hydrate, polymorph, co-crystal, tautomer, stereoisomer, isotopically labeled derivative, prodrug, composition, or mixture thereof.

In some embodiments, the disclosure provides for an inhibitor of PI3K/Akt/mTOR, wherein the inhibitor is any of the molecules disclosed in US2010105711, which is incorporated herein in its entirety. For example, in some embodiments, the disclosure provides for an inhibitor of PI3K/Akt/mTOR comprising the structure of Formula (IV):

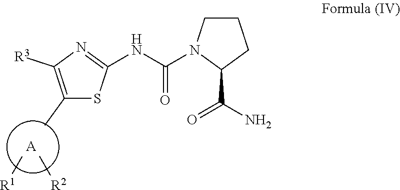

wherein A represents heteroaryl;
R¹ represents (1) optionally substituted alkyl; (2) optionally substituted cycloalkyl; (3) optionally substituted aryl; (4) optionally substituted amine; (5) optionally substituted sulfonyl; (6) halo;
R² represents hydrogen, deuterium or a substituent as defined for R¹;
R³ represents hydrogen, halo, optionally substituted alkyl;
with the exception of (S)-Pyrrolidine-1,2-dicarboxylic acid 2-amide 1-({5-[2-(tert-butyl)-pyrimidin-4-yl]-4-methyl-thiazol-2-yl}-amide).

The prefix “C₁-C₇” denotes a radical having up to and including a maximum of 7, especially up to and including a maximum of 4 carbon atoms, the radicals in question being either linear or branched with single or multiple branching.

“Alkyl” refers to a straight-chain or branched-chain alkyl group, preferably represents a straight-chain or branched-chain C₁₋₁₂ alkyl, particularly preferably represents a straight-chain or branched-chain C₁₋₇ alkyl; for example, methyl, ethyl, n- or iso-propyl, n-, iso-, sec- or tert-butyl, n-pentyl, n-hexyl, n-heptyl, n-octyl, n-nonyl, n-decyl, n-undecyl, n-dodecyl, with particular preference given to methyl, ethyl, n-propyl, iso-propyl and n-butyl and iso-butyl. Alkyl may be unsubstituted or substituted. Exemplary substituents include, but are not limited to deuterium, hydroxy, alkoxy, halo and amino. An example of a substituted alkyl is trifluoromethyl. Cycloalkyl may also be a substituent to alkyl. An example of such a case is the moiety (alkyl)-cyclopropyl or alkandiyl-cycloproyl, e.g., —CH2-cyclopropyl. C1-C7-alkyl is preferably alkyl with from and including 1 up to and including 7, preferably from and including 1 to and including 4, and is linear or branched; preferably, lower alkyl is butyl, such as n-butyl, sec-butyl, isobutyl, tert-butyl, propyl, such as n-propyl or isopropyl, ethyl or preferably methyl.

Each alkyl part of other groups like “alkoxy”, “alkoxyalkyl”, “alkoxycarbonyl”, “alkoxycarbonylalkyl”, “alkylsulfonyl”, “alkylsulfoxyl”, “alkylamino”, “haloalkyl” shall have the same meaning as described in the above-mentioned definition of “alkyl”.

“Alkandiyl” refers to a straight-chain or branched-chain alkandiyl group bound by two different Carbon atoms to the moiety, it preferably represents a straight-chain or branched-chain C1-12 alkandiyl, particularly preferably represents a straight-chain or branched-chain C1-6 alkandiyl; for example, methandiyl (—CH₂—), 1,2-ethanediyl (—CH₂—CH₂—), 1,1-ethanediyl ((—CH(CH₃)—), 1,1-, 1,2-, 1,3-propanediyl and 1,1-, 1,2-, 1,3-, 1,4-butanediyl, with particular preference given to methandiyl, 1,1-ethanediyl, 1,2-ethanediyl, 1,3-propanediyl, 1,4-butanediyl.

“Alkendiyl” refers to a straight-chain or branched-chain alkendiyl group bound by two different Carbon atoms to the molecule, it preferably represents a straight-chain or branched-chain C2-6 alkandiyl; for example, —CH═CH—, —CH═C(CH₃)—, —CH═CH—CH₂—, —C(CH₃)=CH—CH₂—, —CH═C(CH₃)—CH₂—, —CH═CH—C(CH₃)H, —CH═CH—CH═CH—, —C(CH₃)=CH—CH═CH—, —CH═C(CH₃)—CH═CH—, with particular preference given to —CH═CH—CH₂—, —CH═CH—CH═CH—. Alkendiyl may be substituted or unsubstituted

“Cycloalkyl” refers to a saturated or partially saturated, monocyclic, fused polycyclic, or Spiro polycyclic, carbocycle having from 3 to 12 ring atoms per carbocycle. Illustrative examples of cycloalkyl groups include the following moieties: cyclopropyl, cyclobutyl, cyclpentyl and cylclohexyl. Cycloalkyl may be unsubstituted or substituted; exemplary substituents are provided in the definition for alkyl and also include alkyl itself (e.g., methyl). A moiety like —(CH₃)cyclopropyl is considered substituted cycloalkyl.

“Aryl” refers to an aromatic homocyclic ring system (i.e. only Carbon as ring forming atoms) with 6 or more carbon atoms; aryl is preferably an aromatic moiety with 6 to 14 ring carbon atoms, more preferably with 6 to 10 ring carbon atoms, such as phenyl or naphthyl, preferably phenyl. Aryl may be unsubstituted or substituted by one or more, preferably up to three, more preferably up to two substituents independently selected from the group consisting of unsubstituted or substituted heterocyclyl as described below, especially pyrrolidinyl, such as pyrrolidino, oxopyrrolidinyl, such as oxopyrrolidino, C₁-C₇-alkyl-pyrrolidinyl, 2,5-di-(C₁-C₇alkyl)pyrrolidinyl, such as 2,5-di-(C₁-C₇alkyl)-pyrrolidino, tetrahydrofuranyl, thiophenyl, C₁-C₇-alkylpyrazolidinyl, pyridinyl, C₁-C₇-alkylpiperidinyl, piperidino, piperidino substituted by amino or N-mono- or N,N-di-[lower alkyl, phenyl, C₁-C₇-alkanoyl and/or phenyl-lower alkyl)-amino, unsubstituted or N-lower alkyl substituted piperidinyl bound via a ring carbon atom, piperazino, lower alkylpiperazino, morpholino, thiomorpholino, S-oxo-thiomorpholino or S,S-dioxothiomorpholino; C₁-C₇-alkyl, amino-C₁-C₇-alkyl, N—C₁-C₇-alkanoylamino-C₁-C₇-alkyl, N—C₁-C₇-alkanesulfonyl-amino-C₁-C₇-alkyl, carbamoyl-C₁-C₇-alkyl, [N-mono- or N,N-di-(C₁-C₇-alkyl)-carbamoyl] C₁-C₇-alkyl, C₁-C₇-alkanesulfinyl-C₁-C₇-alkyl, C₁-C₇-alkanesulfonyl-C₁-C₇-alkyl, phenyl, naphthyl, mono- to tri-[C₁-C₇-alkyl, halo and/or cyano]-phenyl or mono- to tri-[C₁-C₇-alkyl, halo and/or cyano]-naphthyl; C₃-C₈-cycloalkyl, mono- to tri-[C₁-C₇-alkyl and/or hydroxy]-C3-C8-cycloalkyl; halo, hydroxy, lower alkoxy, lower-alkoxy-lower alkoxy, (lower-alkoxy)-lower alkoxy-lower alkoxy, halo-C₁-C₇-alkoxy, phenoxy, naphthyloxy, phenyl- or naphthyl-lower alkoxy; amino-C₁-C₇-alkoxy, lower-alkanoyloxy, ben-zoyloxy, naphthoyloxy, formyl (CHO), amino, N-mono- or N,N-di-(C₁-C₇-alkyl)-amino, C₁-C₇-alkanoylamino, C₁-C₇-alkanesulfonylamino, carboxy, lower alkoxy carbonyl, e.g.; phenyl- or naphthyl-lower alkoxycarbonyl, such as benzyloxycarbonyl; C₁-C₇-alkanoyl, such as acetyl, benzoyl, naphthoyl, carbamoyl, N-mono- or N,N-disubstituted carbamoyl, such as N-mono- or N,N-di-substituted carbamoyl wherein the substitutents are selected from lower alkyl, (lower-alkoxy)-lower alkyl and hydroxy-lower alkyl; amidino, guanidino, ureido, mercapto, lower alkylthio, phenyl- or naphthylthio, phenyl- or naphthyl-lower alkylthio, lower alkyl-phenylthio, lower alkyl-naphthylthio, halo-lower alkylmercapto, sulfo (—SO₃H), lower alkanesulfonyl, phenyl- or naphthyl-sulfonyl, phenyl- or naphthyl-lower alkylsulfonyl, alkylphenylsulfonyl, halo-lower alkylsulfonyl, such as trifluoromethanesulfonyl; sulfonamido, benzosulfonamido, azido, azido-C₁-C₇-alkyl, especially azidomethyl, C₁-C₇-alkanesulfonyl, sulfamoyl, N-mono- or N,N-di-(C₁-C₇-alkyl)-sulfamoyl, morpholinosulfonyl, thiomorpholinosulfonyl, cyano and nitro; where each phenyl or naphthyl (also in phenoxy or naphthoxy) mentioned above as substituent or part of a substituent of substituted alkyl (or also of substituted aryl, heterocyclyl etc. mentioned herein) is itself unsubstituted or substituted by one or more, e.g., up to three, preferably 1 or 2, substituents independently selected from halo, halo-lower alkyl, such as trifluoromethyl, hydroxy, lower alkoxy, azido, amino, N-mono- or N,N-di-(lower alkyl and/or C₁-C₇-alkanoyl)-amino, nitro, carboxy, lower-alkoxycarbonyl, carbamoyl, cyano and/or sulfamoyl.

“Heterocyclyl” refers to a heterocyclic radical that is unsaturated (=carrying the highest possible number of conjugated double bonds in the ring(s)), saturated or partially saturated and is preferably a monocyclic or in a broader aspect of the invention bicyclic, tricyclic or spirocyclic ring; and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The term heterocyclyl also includes heteroaryl. The heterocyclic radical (heterocyclyl) may be unsubstituted or substituted by one or more, especially 1 to 3, substituents independently selected from the group consisting of the substituents defined above for substituted alkyl and/or from one or more of the following substituents: oxo (═O), thiocarbonyl imino (═NH), imino-lower alkyl. Further, heterocyclyl is especially a heterocyclyl radical selected from the group consisting of oxiranyl, azirinyl, aziridinyl, 1,2-oxathiolanyl, thienyl (=thiophenyl), furanyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidinyl, piperazinyl, pyridazinyl, morpholinyl, thiomorpholinyl, (S-oxo or S,S-dioxo)-thiomorpholinyl, indolizinyl, azepanyl, diazepanyl, especially 1,4-diazepanyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, benzo[1,3]dioxol-5-yl and 2,3-dihydro-benzo[1,4]dioxin-6-yl, each of these radicals being unsubstituted or substituted by one or more, preferably up to three, substituents selected from those mentioned above for substituted aryl and/or from one or more of the following substituents: oxo 0), thiocarbonyl S), imino (═NH), imino-lower alkyl.

“Arylalkyl” refers to an aryl group bound to the molecule via an alkyl group, such as a methyl or ethyl group, preferably phenethyl or benzyl, in particular benzyl. Similarly, cycloalkyl-alkyl and heterocyclyl-alkyl represents a cycloalkyl group bound to the molecule via an alkyl group or a heterocyclyl group bound to the molecule via an alkyl group. In each instance, aryl, heterocyclyl, cycloalkyl and alkyl may be substituted as defined above.

In some embodiments, a composition comprising inhibitors of PI3K/Akt/mTOR signaling disclosed herein comprises GSK-690693 and BYL719, or derivatives, analogues, or variants thereof.

### Stages of Differentiation

In some embodiments, pancreatic differentiation as disclosed herein is carried out in a step-wise manner. In the step-wise progression, “Stage 1” or “S1” refers to the first step in the differentiation process, the differentiation of pluripotent stem cells into cells expressing markers characteristic of definitive endoderm cells (“DE”, “Stage 1 cells” or “S1 cells”). “Stage 2” refers to the second step, the differentiation of cells expressing markers characteristic of definitive endoderm cells into cells expressing markers characteristic of gut tube cells (“GT”, “Stage 2 cells” or “S2 cells”). “Stage 3” refers to the third step, the differentiation of cells expressing markers characteristic of gut tube cells into cells expressing markers characteristic of pancreatic progenitor 1 cells (“PP1”, “Stage β cells” or “S3 cells”). “Stage 4” refers to the fourth step, the differentiation of cells expressing markers characteristic of pancreatic progenitor 1 cells into cells expressing markers characteristic of pancreatic progenitor 2 cells (“PP2”, “Stage 4 cells” or “S4 cells”). “Stage 5” refers to the fifth step, the differentiation of cells expressing markers characteristic of pancreatic progenitor 2 cells (e.g., PDX.1⁺, NKX6.1⁺) into cells expressing markers characteristic of pancreatic endoderm cells and/or pancreatic endocrine progenitor cells (e.g., insulin⁺) (“EN”, “Stage 5 cells” or “S5 cells”). “Stage 6” refers to the differentiation of cells expressing markers characteristic of pancreatic endocrine progenitor cells (e.g., insulin) into cells expressing markers characteristic of pancreatic endocrine β cells (“SC-β cells”) or pancreatic endocrine α cells (“SC-α cells”). It should be appreciated, however, that not all cells in a particular population progress through these stages at the same rate, i.e., some cells may have progressed less, or more, down the differentiation pathway than the majority of cells present in the population. For example, in some embodiments, SC-β cells can be identified during stage 5, at the conclusion of stage 5, at the beginning of stage 6, etc. Examples of methods of making cells of any one of stages 1-6 are provided in, for example, U.S. Pat. Nos. 10,030,229; 10,443,042; U.S. Patent Publication Nos. US 20200332262, US20210198632A1, and US20220090020, each of which is incorporated by reference in its entirety.

### Reprogramming

The term “reprogramming” as used herein refers to the process that alters or reverses the differentiation state of a somatic cell. The cell can either be partially or terminally differentiated prior to the reprogramming. Reprogramming encompasses complete reversion of the differentiation state of a somatic cell to a pluripotent cell. Such complete reversal of differentiation produces an induced pluripotent (iPS) cell. Reprogramming as used herein also encompasses partial reversion of a cell's differentiation state, for example to a multipotent state or to a somatic cell that is neither pluripotent or multipotent, but is a cell that has lost one or more specific characteristics of the differentiated cell from which it arises, e.g., direct reprogramming of a differentiated cell to a different somatic cell type. Reprogramming generally involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult.

As used herein, the term “reprogramming factor” is intended to refer to a molecule that is associated with cell “reprogramming”, that is, differentiation, and/or de-differentiation, and/or transdifferentiation, such that a cell converts to a different cell type or phenotype. Reprogramming factors generally affect expression of genes associated with cell differentiation, de-differentiation and/or transdifferentiation. Transcription factors are examples of reprogramming factors.

The term “differentiation” and their grammatical equivalents as used herein refers to the process by which a less specialized cell (i.e., a more naive cell with a higher cell potency) becomes a more specialized cell type (i.e., a less naive cell with a lower cell potency); and that the term “de-differentiation” refers to the process by which a more specialized cell becomes a less specialized cell type (i.e., a more naive cell with a higher cell potency); and that the term “transdifferentiation” refers to the process by which a cell of a particular cell type converts to another cell type without significantly changing its “cell potency” or “naivety” level. Without wishing to be bound by theory, it is thought that cells “transdifferentiate” when they convert from one lineage-committed cell type or terminally differentiated cell type to another lineage-committed cell type or terminally differentiated cell type, without significantly changing their “cell potency” or “naivety” level.

As used herein, the term “cell potency” is to be understood as referring to the ability of a cell to differentiate into cells of different lineages. For example, a pluripotent cell (e.g., a stem cell) has the potential to differentiate into cells of any of the three germ layers, that is, endoderm (interior stomach lining, gastrointestinal tract, the lungs), mesoderm (muscle, bone, blood, urogenital), or ectoderm (epidermal tissues and nervous system), and accordingly has high cell potency; a multipotent cell (e.g., a stem cell or an induced stem cell of a certain type) has the ability to give rise to cells from a multiple, but limited, number of lineages (such as hematopoietic stem cells, cardiac stem cells, or neural stem cells, etc.) comparatively has a lower cell potency than pluripotent cells. Cells that are committed to a particular lineage or are terminally differentiated can have yet a lower cell potency. Specific examples of transdifferentiation known in the art include the conversion of e.g., fibroblasts beta cells or from pancreatic exocrine cells to beta cells etc.

Accordingly, the cell may be caused to differentiate into a more naïve cell (e.g., a terminally differentiated cell may be differentiated to be multipotent or pluripotent); or the cell may be caused to de-differentiate into a less naive cell (e.g., a multipotent or pluripotent cell can be differentiated into a lineage-committed cell or a terminally differentiated cell). However, in an embodiment, the cell may be caused to convert or transdifferentiate from one cell type (or phenotype) to another cell type (or phenotype), for example, with a similar cell potency level. Accordingly, in an embodiment of the present disclosure, the inducing steps of the present disclosure can reprogram the cells of the present disclosure to differentiate, de-differentiate and/or transdifferentiate. In an embodiment of the present disclosure, the inducing steps of the present disclosure may reprogram the cells to transdifferentiate.

Methods of reprogramming or inducing a particular type of cell to become another type of cell, for example, by differentiation, de-differentiation and/or transdifferentiation using one or more exogenous polynucleotide or polypeptide reprogramming factors are known to the person skilled in the art. Such methods may rely on the introduction of genetic material encoding one or more transcription factor(s) or other polypeptide(s) associated with cell reprogramming. For example, Pdx1, Ngn3 and MafA, or functional fragments thereof are all known to encode peptides that can induce cell differentiation, de-differentiation and/or transdifferentiation of the cells of the present disclosure. In some methods known to the person skilled in the art, exogenous polypeptides (e.g., recombinant polypeptides) encoded by reprogramming genes (such as the above genes) are contacted with the cells to induce, for example, cells of the present disclosure. The person skilled in the art will appreciate that other genes may be associated with reprogramming of cells, and exogenous molecules encoding such genes (or functional fragments thereof) and the encoded polypeptides are also considered to be polynucleotide or polypeptide reprogramming factors (e.g., polynucleotides or polypeptides that in turn affect expression levels of another gene associated with cell reprogramming). For example, it has been shown that the introduction of exogenous polynucleotide or polypeptide epigenetic gene silencers that decrease p53 inactivation increase the efficiency of inducing induced pluripotent stem cells (iPSC). Accordingly, exogenous polynucleotides or polypeptides encoding epigenetic silencers and other genes or proteins that may be directly or indirectly involved in cell reprogramming or increasing cell programming efficiency would be considered to constitute an exogenous polynucleotide or polypeptide reprogramming factor. The person skilled in the art will appreciate that other methods of influencing cell reprogramming exist, such as introducing RNAi molecules (or genetic material encoding RNAi molecules) that can knock down expression of genes involved in inhibiting cell reprogramming. Accordingly, any exogenous polynucleotide molecule or polypeptide molecule that is associated with cell reprogramming, or enhances cell reprogramming, is to be understood to be an exogenous polynucleotide or polypeptide reprogramming factor as described herein.

In some embodiments of the present disclosure, the method excludes the use of reprogramming factor(s) that are not small molecules. However, it will be appreciated that the method may utilize tissue culture components such as culture media, serum, serum substitutes, supplements, antibiotics, etc., such as RPMI, Renal Epithelial Basal Medium (REBM), Dulbecco's Modified Eagle Medium (DMEM), MCDB131 medium, CMRL 1066 medium, F12, fetal calf serum (FCS), fetal bovine serum (FBS), bovine serum albumin (BSA), D-glucose, L-glutamine, GlutaMAX™-1 (dipeptide, L-alanine-L-glutamine), B27, heparin, progesterone, putrescine, laminin, nicotinamide, insulin, transferrin, sodium selenite, selenium, ethanolamine, human epidermal growth factor (hEGF), basic fibroblast growth factor (bFGF), hydrocortisone, epinephrine, normacin, penicillin, streptomycin, gentamicin and amphotericin, etc. It is to be understood that these tissue culture components (and other similar tissue culture components that are routinely used in tissue culture) are not small molecule reprogramming molecules for the purposes of the present disclosure. Indeed, these components are either not small molecules as defined herein and/or are not reprogramming factors as defined herein. Cell culture components and metabolites disclosed herein can be used, however, to enhance the cell reprogramming and differentiation methods disclosed herein. For example, combinations of cell culture components/additives and metabolites disclosed herein can improve the efficiency of generation of SC-β cells, and their functions.

Accordingly, in an embodiment, the present disclosure does not involve a culturing step of the cell(s) with one or more exogenous polynucleotide or polypeptide reprogramming factor(s). Accordingly, in an embodiment, the method of the present disclosure does not involve the introduction of one or more exogenous polynucleotide or polypeptide reprogramming factor(s), e.g., by introducing transposons, viral transgenic vectors (such as retroviral vectors), plasmids, mRNA, miRNA, peptides, or fragments of any of these molecules, that are involved in producing induced beta cells or, otherwise, inducing cells of the present disclosure to differentiate, de-differentiation and/or transdifferentiate.

That is, in an embodiment, the method occurs in the absence of one or more exogenous polynucleotide or polypeptide reprogramming factor(s) (e.g., activin A). Accordingly, it is to be understood that in an embodiment, the method of the present disclosure utilizes small molecules to reprogram cells, without the addition of polypeptide transcription factors; other polypeptide factors specifically associated with inducing differentiation, de-differentiation, and/or transdifferentiation; polynucleotide sequences encoding polypeptide transcription factors, polynucleotide sequences encoding other polypeptide factors specifically associated with inducing differentiation, de-differentiation, and/or transdifferentiation; mRNA; interference RNA; microRNA and fragments thereof.

In some embodiments, the disclosure provides for a method in which one or more small molecules supplements, replaces, and/or reduces the use of activin A in a differentiation protocol.

### Stem Cells

The term “stem cell” is used herein to refer to a cell (e.g., plant stem cell, vertebrate stem cell) that has the ability both to self-renew and to generate a differentiated cell type (Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., beta cell progenitors), which can differentiate into end-stage cells (i.e., terminally differentiated cells, e.g., beta cells, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. al, Science. 1998 Nov. 6; 282(5391):1145-7) whereas induced pluripotent stem cells (iPSCs) are derived from somatic cells (Takahashi et. al, Cell. 2007 Nov. 30; 131(5):861-72; Takahashi et. al, Nat Protoc. 2007; 2(12):3081-9; Yu et. al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell.

By “embryonic stem cell” (ESC) is meant a PSC that is isolated from an embryo, typically from the inner cell mass of the blastocyst. ESC lines are listed in the NIH Human Embryonic Stem Cell Registry, e.g., hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and H1, H7, H9, H13, H14 (Wisconsin Alumni Research Foundation (WiCell Research Institute)). Stem cells of interest also include embryonic stem cells from other primates, such as Rhesus stem cells and marmoset stem cells. The stem cells can be obtained from any mammalian species, e.g., human, equine, bovine, porcine, canine, feline, rodent, e.g., mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, each of which is incorporated herein by its entirety. Methods for proliferating hESCs in the undifferentiated form are described in WO 99/20741, WO 01/51616, and WO 03/020920, each of which is incorporated herein by its entirety.

By “embryonic germ stem cell” (EGSC) or “embryonic germ cell” or “EG cell”, it is meant a PSC that is derived from germ cells and/or germ cell progenitors, e.g., primordial germ cells, i.e., those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which is incorporated herein by its entirety.

By “induced pluripotent stem cell” or “iPSC”, it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

In some embodiments, the population of cells is derived from stem cells in vitro. In some embodiments, the stem cells are genetically modified. In some embodiments, the stem cells have reduced expression of one or more of beta-2 microglobulin, ABO, FUT1, CXCL10, renalase, CIITA, HLA-A, HLA-B, HLA-C, HLA-DP, HLA-DQ, and HLA-DR, relative to stem cells that are not genetically modified. In some embodiments, the stem cells have increased expression of one or more of CD47, PDL1, HLA-G, CD46, CD55, CD59 and/or CTLA, relative to stem cells that are not genetically modified.

In certain examples, the stem cells can be undifferentiated (e.g., a cell not committed to a specific lineage) prior to exposure to at least one β cell maturation factor according to the methods as disclosed herein, whereas in other examples it may be desirable to differentiate the stem cells to one or more intermediate cell types prior to exposure of the at least one cell maturation factor (s) described herein. For example, the stems cells may display morphological, biological, or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be by themselves (for example, without substantially any undifferentiated cells being present) or may be used in the presence of differentiated cells. In certain examples, the stem cells may be cultured in the presence of) suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present invention can be any cells derived from any kind of tissue (for example embryonic tissue such as fetal or pre-fetal tissue, or adult tissue), which stem cells have the characteristic of being capable under appropriate conditions of producing progeny of different cell types, e.g., derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g., hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, FISF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically induced differentiation into mature, insulin positive cells did not involve destroying a human embryo.

In another embodiment, the stem cells can be isolated from tissue including solid tissue. In some embodiments, the tissue is skin, fat tissue (e.g., adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al, (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al, (2001) Blood 98:2615-2625; Eisenberg & Bader (1996) Circ Res. 78(2):205-16; etc.) The stem cells may be obtained from any mammalian species, e.g., human, equine, bovine, porcine, canine, feline, rodent, e.g., mice, rats, hamster, primate, etc. In some embodiments, a human embryo was not destroyed for the source of pluripotent cell used on the methods and compositions as disclosed herein.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. In an embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the SC-β cell can be derived from one or more of trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glia (e.g., oligodendrocyte astrocytes), ependymocytes, pinealocytes, pneumocytes (e.g., type I pneumocytes, and type II pneumocytes), clara cells, goblet cells, G cells, D cells, ECL cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells (e.g., PP cells), pancreatic ε cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, splenocytes (e.g., T lymphocytes, B lymphocytes, dendritic cells, microphages, leukocytes), trophoblast stem cells, or any combination thereof.

### Use of Small Molecule Compounds for Differentiation of β Cells

Provided herein are compositions and methods related to differentiation of stem cells into cells of pancreatic lineage that involve small molecule compounds, for instance, those that can substitute or supplement the use of growth factor(s) from TGF-β superfamily. The composition and methods disclosed herein, in some cases, relate to differentiation of stem cells into, for instance, Sox17-positive cells (e.g., definitive endoderm cells), FOXA2-positive cells (e.g., primitive gut tube cells), Pdx1-positive cells (pancreatic progenitor cells, e.g., Pdx1-positive, Nkx6.1-negative pancreatic progenitor 1 cells, or Pdx1-positive, Nkx6.1-positive pancreatic progenitor 2 cells), insulin-positive pancreatic endocrine cells, or β cells (e.g., stem cell-derived β cells, or non-native pancreatic β cells). In some embodiments, the small molecule compounds comprise an inhibitor of PI3K/Akt/mTOR signaling, such as GSK690693 or an analog thereof.

### Differentiation of Stem Cells

In some embodiments, a method provided herein relates to differentiation of stem cells by contacting a plurality of stem cells (e.g., pluripotent stem cells, e.g., iPSCs or hESCs) with an inhibitor of PI3K/Akt/mTOR signaling. In some embodiments, contacting the stem cells with one or more inhibitors of PI3K/Akt/mTOR signaling results in generation of a population of cells comprising Sox17-positive cells (e.g., definitive endoderm cells).

In some cases, the method disclosed herein includes contacting the plurality of stem cells with the inhibitor of PI3K/Akt/mTOR signaling and a growth factor from TGF-β superfamily (e.g., Activin A). In some cases, the method disclosed herein also includes contacting the stem cells with an activator of WNT signaling pathway in addition to the inhibitor of PI3K/Akt/mTOR signaling. In some cases, the method disclosed herein includes contacting the stem cells with an activator of WNT signaling pathway, an inhibitor of PI3K/Akt/mTOR signaling, and a growth factor from the TGF-β superfamily.

The method disclosed herein can make use of a reduced amount of growth factor from TGF-β superfamily as compared to a reference method that does not involve the inhibitor of PI3K/Akt/mTOR signaling. For instance, in the presence of the inhibitor of PI3K/Akt/mTOR signaling, the growth factor from TGF-β superfamily (e.g., Activin A) can be applied at a concentration that is at most 90%, 80%, 70%, 60%, 50%, 40%, 30%, 20%, 10%, or 5% of the concentration that is applied in the absence of the inhibitor of PI3K/Akt/mTOR signaling for differentiation of at least 95%, 90%, 85%, 80%, 75%, 70%, 65%, 60%, 55%, 50%, 45% or 40% of stem cells into Sox17-positive cells (e.g., definitive endoderm cells). In some cases, the growth factor from TGF-β superfamily (e.g., Activin A) can be applied at a concentration that is about 50%, 40%, 30%, 20%, 10%, 5%, 1%, or 0% of the concentration that is applied in the absence of the inhibitor of PI3K/Akt/mTOR signaling for differentiation of at least 95%, 90%, 85%, 80%, 75%, 70%, 65%, 60%, 55%, 50%, 45% or 40% of stem cells into Sox17-positive cells (e.g., definitive endoderm cells).

Contacting stem cells with an inhibitor of PI3K/Akt/mTOR signaling according to the present disclosure can result in generation of a population of cells that has cell constituent comparable to a population of cells generated by a reference method, wherein the reference method comprises contacting the stem cells with about 100 ng/mL Activin A but not the inhibitor of PI3K/Akt/mTOR signaling, but is otherwise identical to the method. For instance, the population of cells generated according to the present disclosure can have a percentage of Sox17-positive cells that is equivalent to a percentage (e.g., at least 60%, 65%, 70%, 75%, 80%, 85%, 90%, or 95%) of Sox17-positive cells in a population of cells generated by the reference method. In some cases, the population of cells generated according to the present disclosure can have a percentage of Sox17-positive, Oct4-negative cells that is equivalent to a percentage of Sox17-positive, Oct4-negative cells in a population of cells generated by the reference method.

In some cases, contacting stem cells with an inhibitor of PI3K/Akt/mTOR signaling according to the present disclosure can result in generation of a population of cells that comprises at least about 50%, 60%, 65%, 70%, 75%, 80%, or 85% Sox17-positive, Oct4-negative cells. In some cases, contacting stem cells with an inhibitor of PI3K/Akt/mTOR signaling can result in generation of a population of cells that comprises from about 50% to about 90%, about 60% to about 90%, about 65% to about 90%, about 70% to about 90%, about 75% to about 90%, about 80% to about 90%, or about 75% to about 85% Sox17-positive, Oct4-negative cells. In some embodiments, contacting stem cells with an inhibitor of PI3K/Akt/mTOR signaling according to the present disclosure can result in generation of a population of cells that comprises at 50-90%, 50-85%, 50-75%, 50-65%, 60-90%, 60-85%, 60-75%, 70-90%, 70-85%, 80-85%, or 80-90% Sox17-positive, Oct4-negative cells.

In some cases, Sox17-positive cells (e.g., definitive endoderm cells) can be obtained by contacting a population of stem cells with i) at least one growth factor from the TGF-β superfamily, ii) a WNT signaling pathway activator, and optionally iii) any one or more of any of the inhibitors of PI3K/Akt/mTOR signaling disclosed herein (e.g., GSK-690693 and/or BYL719), to induce the differentiation of at least some of the stem cells into definitive endoderm cells, wherein the definitive endoderm cells express at least one marker characteristic of definitive endoderm, for instance, Sox17.

The inhibitor of PI3K/Akt/mTOR signaling that can be used in the differentiation of stem cells into Sox17-positive cells includes an inhibitor of PI3K, an inhibitor of an Akt protein, an inhibitor of mTOR, or any combination thereof. For instance, small molecule compounds such as GSK-690693, IPI-3063, AZD8055, Omipalisib, GNE-477, VS-5584, BYL319, YM201636, PI4KIIIbeta-IN-10, Nemiralisib, BYL719, FT113, Apitolisib, or any analog or derivative thereof, can be used for the differentiation of stem cells into Sox17-positive cells. In some cases, the method involves contacting the stem cells with both an inhibitor of a PI3K protein and an inhibitor of an Akt protein, for instance, BYL719 and GSK-690693. In some cases, the method involves contacting the stem cells with an inhibitor of an Akt protein (e.g., GSK-690693) and a growth factor from TGF-β superfamily (e.g., Activin A).

In some embodiments, the method comprises differentiating stem cells into Sox17-positive cells (e.g., definitive endoderm cells) by contacting a population of stem cells with a suitable concentration of the inhibitor of PI3K/Akt/mTOR signaling. For instance, in some cases, the method comprises differentiating stem cells into Sox17-positive cells (e.g., definitive endoderm cells) by contacting a population of stem cells with from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693, an analog or a derivative thereof. In some cases, the method comprises differentiating stem cells into Sox17-positive cells (e.g., definitive endoderm cells) by contacting a population of stem cells with about 0.01 μM, 0.02 μM, 0.04 μM, 0.06 μM, 0.08 μM, 0.1 μM, 0.12 μM, 0.15 μM, 0.2 μM, 0.3 μM, 0.4 μM, 0.5 μM, 0.6 μM, 0.8 μM, or 1 μM of GSK-690693, an analog or a derivative thereof.

In some cases, the method comprises differentiating stem cells into Sox17-positive cells (e.g., definitive endoderm cells) by contacting a population of stem cells with from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719, an analog or a derivative thereof. In some cases, the method comprises differentiating stem cells into Sox17-positive cells (e.g., definitive endoderm cells) by contacting a population of stem cells with about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719, an analog or a derivative thereof.

In some cases, the method comprises differentiating stem cells into Sox17-positive cells (e.g., definitive endoderm cells) by contacting a population of stem cells with from about 0.01 μM to about 1 μM, about 0.02 μM to about 0.8 μM, about 0.05 μM to about 0.5 μM, about 0.06 μM to about 0.2 μM, or about 0.07 μM to about 0.15 μM of GSK-690693, and from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719, for instance, with about 0.08 μM to about 0.12 μM of GSK-690693 and about 35 nM to about 50 nM of BYL719.

In some examples, the method comprises differentiating stem cells into Sox17-positive cells (e.g., definitive endoderm cells) by contacting a population of stem cells with a suitable concentration of the WNT signaling pathway activator (e.g., CHIR99021), such as, about 0.01 μM, about 0.05 μM, about 0.1 μM, about 0.2 μM, about 0.5 μM, about 0.8 μM, about 1 μM, about 1.5 μM, about 2 μM, about 2.5 μM, about 3 μM, about 3.5 μM, about 4 μM, about 5 μM, about 8 μM, about 10 μM, about 12 μM, about 15 μM, about 20 μM, about 30 μM, about 50 μM, about 100 μM, or about 200 μM. In some cases, the method comprises use of about 2 μM CHIR99021 for differentiation of stem cells into definitive endoderm cells. In some cases, the method comprises use of about 3 μM CHIR99021 for differentiation of stem cells into definitive endoderm cells. In some examples, the method comprises differentiating stem cells into Sox17-positive cells (e.g., definitive endoderm cells) by contacting a population of stem cells with a suitable concentration of the WNT signaling pathway activator (e.g., CHIR99021), with 0.5-10 μM, 1-10 μM, 1-7 μM, 1-5 μM, 2-4 μM, or 2.5-3.5 μM.

Any growth factor from the TGF-β superfamily capable of inducing the stem cells to differentiate into definitive endoderm cells (e.g., alone, or in combination with a WNT signaling pathway activator and/or an inhibitor of PI3K/Akt/mTOR signaling) can be used in the method provided herein. In some cases, the growth factor from the TGF-β superfamily comprises Activin A. In some cases, the growth factor from the TGF-β superfamily comprises growth differentiating factor 8 (GDF8). Any WNT signaling pathway activator capable of inducing the pluripotent stem cells to differentiate into definitive endoderm cells (e.g., alone, or in combination with a growth factor from the TGF-β superfamily and/or an inhibitor of PI3K/Akt/mTOR signaling) can be used in the method provided herein. In some cases, the WNT signaling pathway activator comprises CHIR99021, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC 693868, SB 216763, SB 415286, TC-G 24, TCS 2002, TCS 21311, or TWS 119. In some embodiments, the WNT signaling pathway activator comprises CHIR99021. In some cases, the WNT signaling pathway activator comprises Wnt3a recombinant protein, or a functional variant thereof.

In some examples, the method comprises differentiating stem cells into definitive endoderm cells by contacting a population of stem cells with a suitable concentration of the growth factor from the TGF-β superfamily (e.g., Activin A), such as, about 5 ng/mL, about 10 ng/mL, about 20 ng/mL, about 50 ng/mL, about 75 ng/mL, about 80 ng/mL, about 90 ng/mL, about 95 ng/mL, or about 100 ng/mL, or about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 12 ng/mL, about 14 ng/mL, about 15 ng/mL, about 18 ng/mL, or about 25 ng/mL. In some cases, the method comprises use of about 10 ng/mL Activin A for differentiation of stem cells into definitive endoderm cells. In some cases, the method comprises use of about 100 ng/mL Activin A for differentiation of stem cells into definitive endoderm cells. In some cases, the method comprises use of 10-200 ng/mL, 10-400 ng/mL, 10-150 ng/mL, 10-120 ng/mL, 90-120 ng/mL, 95-105 ng/mL, 1-20 ng/mL, 5-25 ng/mL, 5-50 ng/mL, 10-50 ng/mL, 5-15 ng/mL, or 8-12 ng/mL Activin A for differentiation of stem cells into definitive endoderm cells.

In some cases, Sox17-positive cells (e.g., definitive endoderm cells) can be obtained by culturing stem cells in a composition that includes an inhibitor of PI3K/Akt/mTOR signaling for from about 24 hours to about 96 hours, from about 36 hours to about 84 hours, from about 48 hours to about 84 hours, from about 60 hours to about 84 hours, for instance, for about one day, about two days, or about three days. In some cases, Sox17-positive cells (e.g., definitive endoderm cells) can be obtained by culturing stem cells in a composition that includes an inhibitor of PI3K/Akt/mTOR and a growth factor from TGF-β superfamily (e.g., Activin A) for from about 24 hours to about 96 hours, from about 36 hours to about 84 hours, from about 48 hours to about 84 hours, from about 60 hours to about 84 hours, for instance, for about one day, about two days, or about three days.

In some cases, the method includes a two-stage protocol of treating the stem cells. For instance, the method can include culturing the stem cells in a first composition comprising an inhibitor of PI3K/Akt/mTOR signaling and an activator of WNT signaling pathway for from 12 hours to 48 hours, from 12 hours to 36 hours, from 18 hours to 30 hours, or about one day. The method can further include following the culturing in the first composition, culturing the resulting cell population in a second composition that comprises the inhibitor of PI3K/Akt/mTOR signaling for from 12 hours to 72 hours, from 24 hours to 72 hours, or from 36 hours to 72 hours, for instance, about one day, or about two days.

In some cases, differentiating at least some stem cells in a population into definitive endoderm cells is achieved by a process of contacting a population of stem cells with i) an inhibitor of PI3K/Akt/mTOR and ii) CHIR99021 for a suitable period of time, e.g., about one day, and then contacting the resulting population of cells with an inhibitor of PI3K/Akt/mTOR for a suitable period of time, e.g., about one day, about 2 days, about 3 days, about 4 days, or about 5 days to induce the differentiation of at least some of the stem cells in the population into definitive endoderm cells, wherein the definitive endoderm cells express at least one marker characteristic of definitive endoderm, for instance, Sox17.

In some cases, differentiating at least some stem cells in a population into definitive endoderm cells is achieved by a process of contacting a population of stem cells with i) an inhibitor of PI3K/Akt/mTOR, ii) CHIR99021, and iii) Activin A for a suitable period of time, e.g., about one day, and then contacting the resulting population of cells with i) an inhibitor of PI3K/Akt/mTOR, and ii) Activin A for a suitable period of time, e.g., about one day, about 2 days, about 3 days, about 4 days, or about 5 days to induce the differentiation of at least some of the stem cells in the population into definitive endoderm cells, wherein the definitive endoderm cells express at least one marker characteristic of definitive endoderm, for instance, Sox17.

In some cases, a definitive endoderm cell produced by the methods as disclosed herein expresses at least one marker selected from the group consisting of: Nodal, Tmprss2, Tmem30b, St14, Spink3, Sh3gl2, Ripk4, Rab1S, Npnt, Clic6, Cldn5, Cacna1b, Bnip1, Anxa4, Emb, FoxA1, Sox17, and Rbm35a, wherein the expression of at least one marker is upregulated by a statistically significant amount in the definitive endoderm cell relative to the pluripotent stem cell from which it was derived. In some cases, a definitive endoderm cell produced by the methods as disclosed herein does not express by a statistically significant amount at least one marker selected the group consisting of: Gata4, SPARC, AFP and Dab2 relative to the pluripotent stem cell from which it was derived. In some cases, a definitive endoderm cell produced by the methods as disclosed herein does not express a statistically significant amount at least one marker selected the group consisting of: Zic1, Pax6, Flk1 and CD31 relative to the pluripotent stem cell from which it was derived. In some cases, a definitive endoderm cell produced by the methods as disclosed herein has a higher level of phosphorylation of Smad2 by a statistically significant amount relative to the pluripotent stem cell from which it was derived. In some cases, a definitive endoderm cell produced by the methods as disclosed herein has the capacity to form gut tube in vivo. In some cases, a definitive endoderm cell produced by the methods as disclosed herein can differentiate into a cell with morphology characteristic of a gut cell, and wherein a cell with morphology characteristic of a gut cell expresses FoxA2 and/or Claudin6. In some cases, a definitive endoderm cell produced by the methods as disclosed herein can be further differentiated into a cell of endoderm origin.

In some cases, a population of pluripotent stem cells are cultured in the presence of at least one β cell differentiation factor prior to any differentiation or during the first stage of differentiation. One can use any pluripotent stem cell, such as a human pluripotent stem cell, or a human iPS cell or any of pluripotent stem cell as discussed herein or other suitable pluripotent stem cells. In some cases, a β cell differentiation factor as described herein can be present in the culture medium of a population of pluripotent stem cells or may be added in bolus or periodically during growth (e.g., replication or propagation) of the population of pluripotent stem cells. In certain examples, a population of pluripotent stem cells can be exposed to at least one β cell differentiation factor prior to any differentiation. In other examples, a population of pluripotent stem cells may be exposed to at least one β cell differentiation factor during the first stage of differentiation.

### Differentiation of FOXA2-Positive, PDX1-Negative Cells

In some embodiments, a method provided herein relates to differentiation of FOXA2-positive, PDX1-negative cells (e.g., primitive gut tube cells) by contacting a plurality of FOXA2-positive, PDX1-negative cells with an inhibitor of PI3K/Akt/mTOR signaling. In some embodiments, contacting the FOXA2-positive, PDX1-negative cells with an inhibitor of PI3K/Akt/mTOR signaling results in generation of a population of cells comprising PDX1-positive cells (e.g., PDX1-positive cells, NKX6.1-negative cells, e.g., pancreatic progenitor 1 cells).

In some cases, the method disclosed herein includes contacting the plurality of FOXA2-positive, PDX1-negative cells with the inhibitor of PI3K/Akt/mTOR signaling and i) at least one BMP signaling pathway inhibitor, ii) at least one growth factor from the FGF family, iii) at least one SHH pathway inhibitor, iv) at least one retinoic acid (RA) signaling pathway activator; v) at least one protein kinase C activator, vi) ROCK inhibitor, and vii) a growth factor from TGF-β superfamily. In some cases, the method disclosed herein includes contacting the plurality of FOXA2-positive, PDX1-negative cells with the inhibitor of PI3K/Akt/mTOR signaling and i) at least one BMP signaling pathway inhibitor, ii) at least one growth factor from the FGF family, iii) at least one SHH pathway inhibitor, iv) at least one retinoic acid (RA) signaling pathway activator; v) at least one protein kinase C activator, and vi) ROCK inhibitor, without a growth factor from TGF-β superfamily.

In some aspects, PDX1-positive, NKX6.1-negative pancreatic progenitor cells can be obtained by differentiating at least a portion of primitive gut tube cells in a population into PDX1-positive, NKX6.1-negative pancreatic progenitor cells, e.g., by contacting the primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; vi) at least one protein kinase C activator, and vii) ROCK inhibitor to induce the differentiation of at least some of the primitive gut tube cells into PDX1-positive, NKX6.1-negative pancreatic progenitor cells.

In some aspects, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; and vi) at least one protein kinase C activator, to induce the differentiation of at least some of the primitive gut tube cells into PDX1-positive, NKX6.1-negative pancreatic progenitor cells.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one BMP signaling pathway inhibitor, ii) at least one growth factor from the FGF family, iii) at least one SHH pathway inhibitor, iv) at least one retinoic acid (RA) signaling pathway activator; and v) at least one protein kinase C activator, to induce the differentiation of at least some of the primitive gut tube cells into PDX1-positive, NKX6.1-negative pancreatic progenitor cells.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one SHH pathway inhibitor, ii) at least one retinoic acid (RA) signaling pathway activator; and iii) at least one protein kinase C activator.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with i) at least one growth factor from the FGF family, and ii) at least one retinoic acid (RA) signaling pathway activator, to induce the differentiation of at least some of the primitive gut tube cells into PDX1-positive, NKX6.1-negative pancreatic progenitor cells.

In some embodiments, the method disclosed herein makes use of a reduced amount of growth factor from TGF-β superfamily as compared to a reference method that does not include the inhibitor of PI3K/Akt/mTOR signaling for differentiation of FOXA2-positive, PDX1-negative cells (e.g., primitive gut tube cells) into PDX1-positive cells. For instance, in the presence of the inhibitor of PI3K/Akt/mTOR signaling, the growth factor from TGF-β superfamily (e.g., Activin A) can be applied at a concentration that is at most 90%, 80%, 70%, 60%, 50%, 40%, 30%, 20%, 10%, or 5% of the concentration that is applied in the absence of the inhibitor of PI3K/Akt/mTOR signaling for differentiation of at least 50%, 60%, 70%, 80%, 90%, or 95% (e.g., 50-90%, 50-80%, 50-70%, or 50-60%) of FOXA2-positive, PDX1-negative cells (e.g., primitive gut tube cells) in a culture into PDX1-positive cells. In some cases, the growth factor from TGF-β superfamily (e.g., Activin A) can be applied at a concentration that is about 50%, 40%, 30%, 20%, 10%, 5%, 1%, or 0% of the concentration that is applied in the absence of the inhibitor of PI3K/Akt/mTOR signaling for differentiation of at least 50%, 60%, 70%, 80%, 90%, or 95% (e.g., 50-90%, 50-80%, 50-70%, or 50-60%) of FOXA2-positive, PDX1-negative cells (e.g., primitive gut tube cells) in a culture into PDX1-positive cells.

Any growth factor from the TGF-β superfamily capable of inducing primitive gut tube cells to differentiate into PDX1-positive, NKX6.1-negative pancreatic progenitor cells (e.g., alone, or with any combination of at least one BMP signaling pathway inhibitor, a growth factor from the FGF family, at least one SHH pathway inhibitor, at least one retinoic acid signaling pathway activator, at least one protein kinase C activator, and ROCK inhibitor) can be used. In some cases, the growth factor from TGF-β family comprises Activin A. In some cases, the growth factor from TGF-β family comprises Activin A or GDF8. In some examples, the method comprises contacting primitive gut tube cells with a concentration of a growth factor from TGF-β superfamily (e.g., Activin A), such as, about 5 ng/mL, about 7.5 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 35 ng/mL, about 40 ng/mL, about 50 ng/mL, or about 100 ng/mL. In some embodiments, the concentration of the growth factor from TGF-β superfamily (e.g., Activin A) is 5-50 ng/mL, 15-30 ng/mL, 12-28 ng/mL, 15-25 ng/mL, or 18-22 ng/mL.

In some embodiments, in the presence of an inhibitor of PI3K/Akt/mTOR pathway, the method comprises contacting primitive gut tube cells with a reduced concentration of a growth factor from TGF-β superfamily (e.g., Activin A), such as, at most about 5 ng/mL, at most about at most about 2.5 ng/mL, 1 ng/mL, 0.5 ng/mL, 0.1 ng/mL, or 0.05 ng/mL, e.g., about 2.5 ng/mL, 1 ng/mL, 0.5 ng/mL, 0.1 ng/mL, or 0.05 ng/mL. In some embodiments, the concentration of the growth factor from TGF-β superfamily (e.g., Activin A) is 0.5-5 ng/mL, 1.5-3 ng/mL, 1.2-2.8 ng/mL, 1.5-2.5 ng/mL, or 1.8-2.2 ng/mL.

Any BMP signaling pathway inhibitor capable of inducing primitive gut tube cells to differentiate into PDX1-positive, NKX6.1-negative pancreatic progenitor cells (e.g., alone, or with any combination of a growth factor from TGF-β superfamily, at least one growth factor from the FGF family, at least one SHH pathway inhibitor, at least one retinoic acid signaling pathway activator, at least one protein kinase C activator, and ROCK inhibitor) can be used in the method provided herein. In some cases, the BMP signaling pathway inhibitor comprises LDN193189 or DMH-1. In some examples, the method comprises contacting primitive gut tube cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 μM. In some examples, the method comprises contacting primitive gut tube cells with a concentration of BMP signaling pathway inhibitor (e.g., DMH-1), such as, about 0.01 μM, about 0.02 μM, about 0.05 μM, about 0.1 μM, about 0.2 μM, about 0.5 μM, about 0.8 μM, about 1 μM, about 1.2 μM, about 1.5 μM, about 1.75 μM, about 2 μM, about 2.2 μM, about 2.5 μM, about 2.75 μM, about 3 μM, about 3.25 μM, about 3.5 μM, about 3.75 μM, about 4 μM, about 4.5 μM, about 5 μM, about 8 μM, about 10 μM, about 15 μM, about 20 μM, about 30 μM, about 40 μM, about 50 μM, or about 100 μM. In some examples, the method comprises contacting primitive gut tube cells with a concentration of BMP signaling pathway inhibitor (e.g., DMH-1), such as, 50-1000 nM, 50-500 nM, 50-300 nM, 100-300 nM, 200-300 nM, 200-500 nM, or 225-275 nM.

Any growth factor from the FGF family capable of inducing primitive gut tube cells to differentiate into PDX1-positive, NKX6.1-negative pancreatic progenitor cells (e.g., alone, or with any combination of at least one BMP signaling pathway inhibitor, a growth factor from TGF-β superfamily, at least one SHH pathway inhibitor, at least one retinoic acid signaling pathway activator, at least one protein kinase C activator, and ROCK inhibitor) can be used. In some cases, the at least one growth factor from the FGF family comprises keratinocyte growth factor (KGF). In some cases, the at least one growth factor from the FGF family is selected from the group consisting of FGF2, FGF8B, FGF 10, and FGF21. In some examples, the method comprises contacting primitive gut tube cells with a concentration of a growth factor from FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some examples, the method comprises contacting primitive gut tube cells with a concentration of a growth factor from FGF family (e.g., KGF), such as, 10-200 ng/mL, 10-150 ng/mL, 10-100 ng/mL, 25-75 ng/mL, 40-60 ng/mL, or 45-55 ng/mL.

Any SHH pathway inhibitor capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, a growth factor from TGF-β superfamily, at least one retinoic acid signaling pathway activator, at least one protein kinase C activator, and ROCK inhibitor) can be used. In some cases, the SHH pathway inhibitor comprises Sant1. In some examples, the method comprises contacting primitive gut tube cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting primitive gut tube cells with a concentration of a SHH pathway inhibitor (e.g., Sant1), 50-1000 nM, 50-500 nM, 50-300 nM, 100-300 nM, 200-300 nM, 200-500 nM, or 225-275 nM.

Any RA signaling pathway activator capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, at least one SHH pathway inhibitor, at least one protein kinase C activator, and ROCK inhibitor) can be used. In some cases, the RA signaling pathway activator comprises retinoic acid. In some examples, the method comprises contacting primitive gut tube cells with a concentration of an RA signaling pathway activator (e.g., retinoic acid), such as, about 0.02 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting primitive gut tube cells with a concentration of an RA signaling pathway activator (e.g., retinoic acid), such as, 0.2-5 μM, 0.8-3 μM, 0.8-2.5 μM, 1-2.5 μM, 1.5-2.5 μM, 1.8-2.2 μM, or 1.9-2.1 μM.

Any PKC activator capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, at least one SHH pathway inhibitor, at least one RA signaling pathway activator, and ROCK inhibitor) can be used. In some cases, the PKC activator comprises PdBU. In some cases, the PKC activator comprises TPB. In some examples, the method comprises contacting primitive gut tube cells with a concentration of a PKC activator (e.g., PdBU), such as, about 10 nM, 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 10 μM, about 20 μM, about 50 μM, about 75 μM, about 80 μM, about 100 μM, about 120 μM, about 140 μM, about 150 μM, about 175 μM, about 180 μM, about 200 μM, about 210 μM, about 220 μM, about 240 μM, about 250 μM, about 260 μM, about 280 μM, about 300 μM, about 320 μM, about 340 μM, about 360 μM, about 380 μM, about 400 μM, about 420 μM, about 440 μM, about 460 μM, about 480 μM, about 500 μM, about 520 μM, about 540 μM, about 560 μM, about 580 μM, about 600 μM, about 620 μM, about 640 μM, about 660 μM, about 680 μM, about 700 μM, about 750 μM, about 800 μM, about 850 μM, about 900 μM, about 1 mM, about 2 mM, about 3 mM, about 4 mM, or about 5 mM. In some embodiments, the method comprises contacting primitive gut tube cells with a concentration of a PKC activator (e.g., PdBU) of 10 nM-1 mM, 10 nM-500 μM, 10 nM-1 μM, 10-800 nM, 100-900 nM, 300-800 nM, 300-600 nM, 400-600 nM, 450-550 nM, or about 500 nM. In some embodiments, primitive gut tube cells are not treated with a PKC activator (e.g., PDBU).

Any ROCK inhibitor capable of inducing primitive gut tube cells to differentiate into PDX1-positive, NKX6.1-negative pancreatic progenitor cells (e.g., alone, or with any combination of at least one BMP signaling pathway inhibitor, at least one growth factor from the FGF family, at least one SHH pathway inhibitor, PKC activator, and at least one RA signaling pathway activator) can be used. In some cases, the ROCK inhibitor comprises Thiazovivin, Y-27632, Fasudil/HA1077, or H-1152. In some cases, the ROCK inhibitor comprises Y-27632. In some cases, the ROCK inhibitor comprises Thiazovivin. In some examples, the method comprises contacting primitive gut tube cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some examples, the method comprises contacting primitive gut tube cells with a concentration of a ROCK inhibitor (e.g., Y-27632 or Thiazovivin), such as, 0.2-5 μM, 0.8-3 μM, 1-4 μM, 1.5-4 μM, 1.8-3.5 μM, 2-3 μM, 2.4-2.6 μM.

In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with retinoic acid, KGF, Sant1, DMH-1, PdBU, thiazovivin, and Activin A, for a suitable period of time, e.g., about 1 day, about 2 days, about 3 days, about 4 days, 18-72 hours, 36-60 hours, 40-54 hours, or 44-52 hours. In some cases, PDX1-positive, NKX6.1-negative pancreatic progenitor cells can be obtained by differentiating at least some primitive gut tube cells in a population into PDX1-positive, NKX6.1-negative pancreatic progenitor cells, e.g., by contacting primitive gut tube cells with retinoic acid, KGF, Sant1, DMH-1, PdBU, thiazovivin, and Activin A, for about 2 days. In some cases, PDX1-positive, NKX6.1-negative pancreatic progenitor cells can be obtained by differentiating at least some primitive gut tube cells in S3 medium.

### Differentiation of PDX1-Positive, NKX6.1-Negative Pancreatic Progenitor Cells

In some embodiments, a method provided herein relates to differentiation of PDX1-positive, NKX6.1-negative cells (e.g., pancreatic progenitor 1 cells) by contacting a plurality of PDX1-positive, NKX6.1-negative cells with an inhibitor of PI3K/Akt/mTOR signaling. In some embodiments, contacting the PDX1-positive, NKX6.1-negative cells with an inhibitor of PI3K/Akt/mTOR signaling results in generation of a population of cells comprising PDX1-positive, NKX6.1-positive cells (e.g., pancreatic progenitor 2 cells).

In some cases, the method disclosed herein includes contacting the plurality of PDX1-positive, NKX6.1-negative cells (e.g., pancreatic progenitor 1 cells) with the inhibitor of PI3K/Akt/mTOR signaling and i) at least one growth factor from the FGF family, ii) at least one SHH pathway inhibitor, and optionally iii) a RA signaling pathway activator, iv) a ROCK inhibitor, and v) at least one growth factor from the TGF-β superfamily, optionally vi) a protein kinase C activator. In some cases, the method disclosed herein includes contacting the plurality of PDX1-positive, NKX6.1-negative cells (e.g., pancreatic progenitor 1 cells) with the inhibitor of PI3K/Akt/mTOR signaling and i) at least one growth factor from the FGF family, ii) at least one SHH pathway inhibitor, and optionally iii) a RA signaling pathway activator, iv) ROCK inhibitor, and optionally v) a protein kinase C activator, without a growth factor from TGF-β superfamily.

In some embodiments, the method disclosed herein makes use of a reduced amount of growth factor from TGF-β superfamily as compared to a reference method that does not involve the inhibitor of PI3K/Akt/mTOR signaling for differentiation of PDX1-positive, NKX6.1-negative cells (e.g., pancreatic progenitor 1 cells) into PDX1-positive, NKX6.1-positive cells (e.g., pancreatic progenitor 2 cells). For instance, in the presence of the inhibitor of PI3K/Akt/mTOR signaling, the growth factor from TGF-β superfamily (e.g., Activin A) can be applied at a concentration that is at most 90%, 80%, 70%, 60%, 50%, 40%, 30%, 20%, 10%, or 5% of the concentration that is applied in the absence of the inhibitor of PI3K/Akt/mTOR signaling for differentiation of at least 30%, 40%, 50%, 60%, 70%, 80%, 90% or 95% (e.g., 40-90%, 40-80%, 40-70%, 40-60%, 40-50%, 60-90%, 60-80%, or 70-90%) of PDX1-positive, NKX6.1-negative cells (e.g., pancreatic progenitor 1 cells) in a culture into PDX1-positive, NKX6.1-positive cells (e.g., pancreatic progenitor 2 cells). In some cases, the growth factor from TGF-β superfamily (e.g., Activin A) can be applied at a concentration that is about 50%, 40%, 30%, 20%, 10%, 5%, 1%, or 0% of the concentration that is applied in the absence of the inhibitor of PI3K/Akt/mTOR signaling for differentiation of at least 30%, 40%, 50%, 60%, 70%, 80%, 90% or 95% (e.g., 40-90%, 40-80%, 40-70%, 40-60%, 40-50%, 60-90%, 60-80%, or 70-90%) of PDX1-positive, NKX6.1-negative cells (e.g., pancreatic progenitor 1 cells) in a culture into PDX1-positive, NKX6.1-positive cells (e.g., pancreatic progenitor 2 cells).

In some aspects, a method of producing a PDX1-positive, NKX6.1-positive pancreatic progenitor cell from a PDX1-positive, NKX6.1-negative pancreatic progenitor cell comprises contacting a population of cells (e.g., under conditions that promote cell clustering and/or promoting cell survival) comprising PDX1-positive, NKX6.1-negative pancreatic progenitor cells with at least two β cell-differentiation factors comprising a) at least one growth factor from the fibroblast growth factor (FGF) family, b) a sonic hedgehog pathway inhibitor, and optionally c) a retinoic acid (RA) signaling pathway activator, to induce the differentiation of at least one PDX1-positive, NKX6.1-negative pancreatic progenitor cell in the population into PDX1-positive, NKX6.1-positive pancreatic progenitor cells.

In some cases, the PDX1-positive, NKX6.1-positive pancreatic progenitor cells are obtained by contacting PDX1-positive, NKX6.1-negative pancreatic progenitor cells with i) at least one growth factor from the FGF family, ii) at least one SHH pathway inhibitor, and optionally iii) a RA signaling pathway activator, to induce the differentiation of at least some of the PDX1-positive, NKX6.1-negative pancreatic progenitor cells into PDX1-positive, NKX6.1-positive pancreatic progenitor cells.

In some cases, the PDX1-positive, NKX6.1-positive pancreatic progenitor cells are obtained by contacting PDX1-positive, NKX6.1-negative pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily, to induce the differentiation of at least a portion (e.g., at least 30%, 40%, 50%, 60%, 70%, 80%, 90% or 95% (e.g., 40-90%, 40-80%, 40-70%, 40-60%, 40-50%, 60-90%, 60-80%, or 70-90%) of the PDX1-positive, NKX6.1-negative pancreatic progenitor cells in a culture into PDX1-positive, NKX6.1-positive pancreatic progenitor cells. In some cases, the PDX1-positive, NKX6.1-positive pancreatic progenitor cells are obtained by contacting PDX1-positive, NKX6.1-negative pancreatic progenitor cells under conditions that promote cell clustering with at least one growth factor from the FGF family. In some embodiments, the PDX1-positive, NKX6.1-negative pancreatic progenitor cells are contacted with a PKC activator (e.g., PDBU). See, e.g., U.S. Patent Publication No. US20210238553A1, and US20220143374A1, which are incorporated by reference herein in their entireties.

Any growth factor from the FGF family capable of inducing PDX1-positive, NKX6.1-negative pancreatic progenitor cells to differentiate into PDX1-positive, NKX6.1-positive pancreatic progenitor cells (e.g., alone, or with any combination of at least one SHH pathway inhibitor, a ROCK inhibitor, a growth factor from the TGF-β superfamily, and at least one retinoic acid signaling pathway activator) can be used in the method provided herein. In some cases, the at least one growth factor from the FGF family comprises keratinocyte growth factor (KGF). In some cases, the at least one growth factor from the FGF family is selected from the group consisting of FGF2, FGF8B, FGF10, and FGF21. In some examples, the method comprises contacting PDX1-positive, NKX6.1-negative pancreatic progenitor cells with a concentration of a growth factor from FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some examples, the method comprises contacting PDX1-positive, NKX6.1-negative pancreatic progenitor cells with a concentration of a growth factor from FGF family (e.g., KGF), such as, 10-200 ng/mL, 10-150 ng/mL, 10-100 ng/mL, 25-75 ng/mL, 40-60 ng/mL, or 45-55 ng/mL.

Any SHH pathway inhibitor capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one retinoic acid signaling pathway activator, ROCK inhibitor, and at least one growth factor from the TGF-β superfamily) can be used in the method provided herein. In some cases, the SHH pathway inhibitor comprises Sant1. In some examples, the method comprises contacting PDX1-positive, NKX6.1-negative pancreatic progenitor cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive, NKX6.1-negative pancreatic progenitor cells with a concentration of a SHH pathway inhibitor (e.g., Sant1), such as, 50-1000 nM, 50-500 nM, 50-300 nM, 100-300 nM, 200-300 nM, 200-500 nM, or 225-275 nM.

Any RA signaling pathway activator capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one SHH pathway inhibitor, ROCK inhibitor, and at least one growth factor from the TGF-β superfamily) can be used. In some cases, the RA signaling pathway activator comprises retinoic acid. In some examples, the method comprises contacting PDX1-positive, NKX6.1-negative pancreatic progenitor cells with a concentration of an RA signaling pathway activator (e.g., retinoic acid), such as, about 0.02 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting PDX1-positive, NKX6.1-negative pancreatic progenitor cells with a concentration of an RA signaling pathway activator (e.g., retinoic acid), such as, 1-500 nM, 50-400 nM, 50-250 nM, 50-150 nM, 80-200 nM, 75-125 nM, or 90-110 nM.

Any ROCK inhibitor capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one SHH pathway inhibitor, a RA signaling pathway activator, and at least one growth factor from the TGF-β superfamily) can be used. In some cases, the ROCK inhibitor comprises Thiazovivin, Y-27632, Fasudil/HA1077, or 14-1152. In some examples, the method comprises contacting PDX1-positive, NKX6.1-negative pancreatic progenitor cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some examples, the method comprises contacting PDX1-positive, NKX6.1-negative pancreatic progenitor cells with a concentration of a ROCK inhibitor (e.g., Y-27632 or Thiazovivin), such as, 0.2-5 μM, 0.8-3 μM, 1-4 μM, 1.5-4 μM, 1.8-3.5 μM, 2-3 μM, 2.4-2.6 μM.

Any activator from the TGF-β superfamily capable of inducing PDX1-positive, NKX6.1-negative pancreatic progenitor cells to differentiate into PDX1-positive, NKX6.1-positive pancreatic progenitor cells (e.g., alone, or with any combination of at least one growth factor from the FGF family, at least one SHH pathway inhibitor, a RA signaling pathway activator, and ROCK inhibitor) can be used. In some cases, the activator from the TGF-β superfamily comprises Activin A or GDF8. In some examples, the method comprises contacting PDX1-positive, NKX6.1-negative pancreatic progenitor cells with a concentration of a growth factor from TGF-β superfamily (e.g., Activin A), such as, about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 1 ng/mL, about 1.2 ng/mL, about 1.4 ng/mL, about 1.6 ng/mL, about 1.8 ng/mL, about 2 ng/mL, about 2.2 ng/mL, about 2.4 ng/mL, about 2.6 ng/mL, about 2.8 ng/mL, about 3 ng/mL, about 3.2 ng/mL, about 3.4 ng/mL, about 3.6 ng/mL, about 3.8 ng/mL, about 4 ng/mL, about 4.2 ng/mL, about 4.4 ng/mL, about 4.6 ng/mL, about 4.8 ng/mL, about 5 ng/mL, about 5.2 ng/mL, about 5.4 ng/mL, about 5.6 ng/mL, about 5.8 ng/mL, about 6 ng/mL, about 6.2 ng/mL, about 6.4 ng/mL, about 6.6 ng/mL, about 6.8 ng/mL, about 7 ng/mL, about 8 ng/mL, about 9 ng/mL, about 10 ng/mL, about 20 ng/mL, about 30 ng/mL, or about 50 ng/mL. In some examples, the method comprises contacting PDX1-positive, NKX6.1-negative pancreatic progenitor cells with a concentration of a growth factor from TGF-β superfamily (e.g., Activin A), such as, about 5 ng/mL. In some embodiments, the concentration of the growth factor from TGF-β superfamily (e.g., Activin A) is 1-15 ng/mL, 3-12 ng/mL, 5-12 ng/mL, 5-20 ng/mL, 8-20 ng/mL, 8-15 ng/mL, 9-11 ng/mL, or 8-12 ng/mL.

In some embodiments, in the presence of an inhibitor of PI3K/Akt/mTOR pathway, the method comprises contacting primitive gut tube cells with a reduced concentration of a growth factor from TGF-β superfamily (e.g., Activin A), such as, at most about 20 ng/mL, at most about 10 ng/mL, 5 ng/mL, 1 ng/mL, 0.5 ng/mL, or 0.1 ng/mL, e.g., about 10 ng/mL, 5 ng/mL, 1 ng/mL, 0.5 ng/mL, or 0.1 ng/mL. In some embodiments, the concentration of the growth factor from TGF-β superfamily (e.g., Activin A) is 0.1-1.5 ng/mL, 0.3-1.2 ng/mL, 0.5-1.2 ng/mL, 0.5-2.0 ng/mL, 0.8-2.0 ng/mL, 0.8-1.5 ng/mL, 0.9-1.1 ng/mL, or 0.8-1.2 ng/mL.

In some cases, the PDX1-positive, NKX6.1-positive pancreatic progenitor cells are obtained by contacting PDX1-positive, NKX6.1-negative pancreatic progenitor cells under conditions that promote cell clustering with KGF, Sant1, and RA and optionally an inhibitor of PI3K/Akt/mTOR signaling, for a period of 5 days or 6 days or 96-170 hours, 120-170 hours, 130-160 hours, or 140-150 hours. In some cases, the PDX1-positive, NKX6.1-positive pancreatic progenitor cells are obtained by contacting PDX1-positive, NKX6.1-negative pancreatic progenitor cells under conditions that promote cell clustering with KGF, Sant1, RA, thiazovivin, and Activin A and optionally an inhibitor of PI3K/Akt/mTOR signaling, for a period of 5 or 6 days or 96-170 hours, 120-170 hours, 130-160 hours, or 140-150 hours. In some cases, the PDX1-positive, NKX6.1-positive pancreatic progenitor cells are obtained by contacting PDX1-positive, NKX6.1-negative pancreatic progenitor cells under conditions that promote cell clustering with KGF for a period of 5 or 6 days or 96-170 hours, 120-170 hours, 130-160 hours, or 140-150 hours. In some cases, the PDX1-positive, NKX6.1-positive pancreatic progenitor cells are obtained by contacting PDX1-positive, NKX6.1-negative pancreatic progenitor cells in a S4 medium.

In some cases, the PDX1-positive, NKX6.1-positive pancreatic progenitor cells are obtained by contacting PDX1-positive, NKX6.1-negative pancreatic progenitor cells under conditions that promote cell clustering with KGF, Sant1, RA, thiazovivin, and Activin A and optionally an inhibitor of PI3K/Akt/mTOR signaling, for a period of 5 or 6 days or 96-170 hours, 120-170 hours, 130-160 hours, or 140-150 hours.

### Methods of Generating Stem Cell Derived β Cells

Provided herein are methods of generating SC-β cells (e.g., non-native pancreatic β cells). Examples of detailed protocols of generating endocrine cells from the stem cells to provide at least one SC-β cell are described in U.S. Patent Application Publication Nos. US20150240212, US20150218522, US20210198632A1, US20210238553A1, and US20220143374A1, each of which is herein incorporated by reference in its entirety.

The endoderm can give rise to digestive and respiratory tracts, thyroid, liver, and pancreas. Representative disease of endoderm lineages is type 1 diabetes resulting from destruction of the insulin-producing β cells. Generation of functional β cells from human pluripotent stem cells (hPSC) in vitro can be a practical, renewable cell source for replacement cell therapy for type 1 diabetes. The embryotic stem (ES) cells that are generated from the inner cell mass of blastocyst-stage embryos represent a promising source of cells for transplantation or cell-based therapy of any damaged cells. They can be maintained in culture, renew for themselves, and proliferate unlimitedly as undifferentiated ES cells. The ES cells are capable of differentiating into all cell types of the body as the ectoderm, mesoderm, and endoderm lineage cells or tissues. The major benefit of ES cells is stable self-renewal in culture and the potential to differentiate.

The definitive endoderm can be generated in vivo from the inner cell mass by the process of gastrulation of embryogenesis, in which epiblast cells are instructed to form the three germ layers. Definitive endoderm can give rise to diverse cells and tissues that contribute to vital organs as the pancreatic β cells, liver hepatocytes, lung alveolar cells, thyroid, thymus, and the epithelial lining of the alimentary and respiratory tract. It is different from the primitive endoderm of extraembryonic tissues, which can give rise to the visceral and parietal endoderm. The definitive endoderm derived from ES cells is theoretically capable of becoming any endoderm derivatives, and directing ES cells into the endoderm lineage is a prerequisite for generating therapeutic endoderm derivatives.

Precise patterning of anterior-posterior axis of the definitive endoderm can eventually form the primitive gut tube. The definitive endoderm-derived primitive gut tube induces the pharynx, esophagus, stomach, duodenum, small and large intestine along the anterior-posterior axis as well as associated organs, including pancreas, lung, thyroid, thymus, parathyroid, and liver. The anterior portion of the foregut of the primitive gut tube becomes lung, thyroid, esophagus, and stomach. The pancreas, liver, and duodenum originate from the posterior portion of the foregut. The midgut and hindgut of primitive gut tube gives rise to the small and large intestine. The anterior foregut expresses developmental markers, NK2 homeobox 1 (NKX2-1) and SRY (sex determining region Y)-box 2 (SOX2); the posterior foregut expresses hematopoietically expressed homeobox (HHEX), pancreatic and duodenal homeobox 1 (PDX1), one cut homeobox 1 (ONECUT1, known as HNF6), and hepatocyte nuclear factor 4 alpha (HNF4A); and the midgut/hindgut expresses caudal type homeobox 1 (CDX1), caudal type homeobox 2 (CDX2), and motor neuron and pancreas homeobox 1 (MNX1).

The successful differentiation to pancreatic β cells should require that differentiated cells synthesize and secrete physiologically appropriate amounts of insulin. An exemplary stepwise protocol directing hPSC cell differentiation is developed, which entails differentiation processes that recapitulates the major stages of normal pancreatic endocrine development. The differentiation of hPSC cells to hormone-expressing pancreatic endocrine cells is conducted by transitioning hPSC cells through major stages of embryonic development; differentiation to mesendoderm and definitive endoderm, establishment of the primitive gut endoderm, patterning of the posterior foregut, and specification and maturation of pancreatic endoderm and endocrine precursors. Through these stages, hPSC cells can obtain pancreatic endocrine phenotype and ability of glucose responsive insulin secretion in vitro.

Generally, the at least one pancreatic α, β and/or δ cell or precursor thereof, e.g., pancreatic progenitors produced according to the methods disclosed herein, can comprise a mixture or combination of different cells, e.g., for example a mixture of cells such as a PDX1-positive, NKX6.1-negative pancreatic progenitors, pancreatic progenitors co-expressing PDX1 and NKX6-1, a Ngn3-positive endocrine progenitor cell, an insulin-positive endocrine cell (e.g., NKX6.1-positive, ISL1-positive cells, or 3-like cells), and/or other pluripotent or stem cells.

The at least one pancreatic α, β and/or δ cell or precursor thereof can be produced according to any suitable culturing protocol to differentiate a stem cell or pluripotent cell to a desired stage of differentiation. In some embodiments, the at least one pancreatic α, β and/or δ cell or the precursor thereof are produced by culturing at least one pluripotent cell for a period of time and under conditions suitable for the at least one pluripotent cell to differentiate into the at least one pancreatic α, β and/or δ cell or the precursor thereof.

In some embodiments, the at least one pancreatic α, β and/or δ cell or precursor thereof is a substantially pure population of pancreatic α, β and/or δ cells or precursors thereof. In some embodiments, a population of pancreatic α, β and/or δ cells or precursors thereof comprises a mixture of pluripotent cells or differentiated cells. In some embodiments, a population pancreatic α, β and/or δ cells or precursors thereof are substantially free or devoid of embryonic stem cells or pluripotent cells or iPS cells.

In some embodiments, a somatic cell, e.g., a fibroblast, can be isolated from a subject, for example as a tissue biopsy, such as, for example, a skin biopsy, and reprogrammed into an induced pluripotent stem cell for further differentiation to produce the at least one SC-β cell or precursor thereof for use in the compositions and methods described herein. In some embodiments, a somatic cell, e.g., a fibroblast, is maintained in culture by methods known by one of ordinary skill in the art, and in some embodiments, propagated prior to being converted into pancreatic α, β and/or δ cells by the methods as disclosed herein.

In some embodiments, the at least one pancreatic α, β and/or δ cell or precursor thereof are maintained in culture by methods known by one of ordinary skill in the art, and in some embodiments, propagated prior to being converted into pancreatic α, β and/or δ cells by the methods as disclosed herein.

Further, at least one pancreatic α, β and/or δ cell or precursor thereof, e.g., pancreatic progenitor can be from any mammalian species, with non-limiting examples including a murine, bovine, simian, porcine, equine, ovine, or human cell. For clarity and simplicity, the description of the methods herein refers to a mammalian at least one pancreatic α, β and/or δ cell or precursor thereof but it should be understood that all of the methods described herein can be readily applied to other cell types of at least one pancreatic α, β and/or δ cell or precursor thereof. In some embodiments, the at least one pancreatic α, β and/or δ cell or precursor thereof is derived from a human individual.

### Definitive Endoderm Cells

Aspects of the disclosure involve definitive endoderm cells. Definitive endoderm cells of use herein can be derived from any source or generated in accordance with any suitable protocol, including a method disclosed herein involving the use of a small molecule compound, such as an inhibitor of PI3K/Akt/mTOR signaling.

The definitive endoderm can be generated in vivo from the inner cell mass by the process of gastrulation of embryogenesis, in which epiblast cells are instructed to form the three germ layers. Definitive endoderm can give rise to diverse cells and tissues that contribute to vital organs as the pancreatic β cells, liver hepatocytes, lung alveolar cells, thyroid, thymus, and the epithelial lining of the alimentary and respiratory tract. It is different from the primitive endoderm of extraembryonic tissues, which can give rise to the visceral and parietal endoderm. The definitive endoderm derived from ES cells is theoretically capable of becoming any endoderm derivatives.

Precise patterning of anterior-posterior axis of the definitive endoderm can eventually form the primitive gut tube. The definitive endoderm-derived primitive gut tube induces the pharynx, esophagus, stomach, duodenum, small and large intestine along the anterior-posterior axis as well as associated organs, including pancreas, lung, thyroid, thymus, parathyroid, and liver. The anterior portion of the foregut of the primitive gut tube becomes lung, thyroid, esophagus, and stomach. The pancreas, liver, and duodenum originate from the posterior portion of the foregut. The midgut and hindgut of primitive gut tube gives rise to the small and large intestine. The anterior foregut expresses developmental markers, NK2 homeobox 1 (NKX2-1) and SRY (sex determining region Y)-box 2 (SOX2); the posterior foregut expresses hematopoietically expressed homeobox (HHEX), pancreatic and duodenal homeobox 1 (PDX1), one cut homeobox 1 (ONECUT1, known as HNF6), and hepatocyte nuclear factor 4 alpha (HNF4A); and the midgut/hindgut expresses caudal type homeobox 1 (CDX1), caudal type homeobox 2 (CDX2), and motor neuron and pancreas homeobox 1 (MNX1) (3, 19, 20).

As described herein definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells (stage 1) are further differentiated, e.g., to primitive gut tube cells (stage 2), PDX1-positive, NKX6.1-negative pancreatic progenitor cells (stage 3), PDX1-positive, NKX6.1-positive pancreatic progenitor cells (stage 4), or Ngn3-positive endocrine progenitor cells or insulin-positive endocrine cells (stage 5), followed by induction or maturation to SC-β cells (stage 6).

In some embodiments, definitive endoderm cells can be obtained by differentiating at least some pluripotent cells in a population into definitive endoderm cells, e.g., by contacting a population of pluripotent cells with i) at least one growth factor from the TGF-β superfamily, and ii) a WNT signaling pathway activator, to induce the differentiation of at least some of the pluripotent cells into definitive endoderm cells, wherein the definitive endoderm cells express at least one marker characteristic of definitive endoderm.

Any growth factor from the TGF-β superfamily capable of inducing the pluripotent stem cells to differentiate into definitive endoderm cells (e.g., alone, or in combination with a WNT signaling pathway activator) can be used in the method provided herein. In some embodiments, the growth factor from the TGF-β superfamily comprises Activin A. In some embodiments, the growth factor from the TGF-β superfamily comprises growth differentiating factor 8 (GDF8). Any WNT signaling pathway activator capable of inducing the pluripotent stem cells to differentiate into definitive endoderm cells (e.g., alone, or in combination with a growth factor from the TGF-β superfamily) can be used in the method provided herein. In some embodiments, the WNT signaling pathway activator comprises CHIR99021. In some embodiments, the WNT signaling pathway activator comprises Wnt3a recombinant protein.

In some embodiments, differentiating at least some pluripotent cells in a population into definitive endoderm cells is achieved by a process of contacting a population of pluripotent cells with i) Activin A, and ii) CHIR99021 for a suitable period of time, e.g., about 2 days, about 3 days, about 4 days, or about 5 days to induce the differentiation of at least some of the pluripotent cells in the population into definitive endoderm cells, wherein the definitive endoderm cells express at least one marker characteristic of definitive endoderm. In some embodiments, the process comprises contacting a population of pluripotent cells with activin A and CHIR99021 for 1 day, and then with activin A (in the absence of CHIR99021) for a further 1 or 2 days.

In some examples, the method comprises differentiating pluripotent cells into definitive endoderm cells by contacting a population of pluripotent cells with a suitable concentration of the growth factor from the TGF-β superfamily (e.g., Activin A), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some embodiments, the method comprises use of about 70-130 ng·ml, 80-120 ng/ml, or 90-110 ng/ml Activin A for differentiation of pluripotent cells into definitive endoderm cells. In some embodiments, the method comprises use of about 100 ng/mL Activin A for differentiation of pluripotent cells into definitive endoderm cells. In some embodiments, the method comprises use of about 200 ng/mL Activin A for differentiation of pluripotent cells into definitive endoderm cells.

In some examples, the method comprises differentiating pluripotent cells into definitive endoderm cells by contacting a population of pluripotent cells with a suitable concentration of the WNT signaling pathway activator (e.g., CHIR99021), such as, about 0.01 μM, about 0.05 μM, about 0.1 μM, about 0.2 μM, about 0.5 μM, about 0.8 μM, about 1 μM, about 1.5 μM, about 2 μM, about 2.5 μM, about 3 μM, about 3.5 μM, about 4 μM, about 5 μM, about 8 μM, about 10 μM, about 12 μM, about 15 μM, about 20 μM, about 30 μM, about 50 μM, about 100 μM, or about 200 μM. In some embodiments, the method comprises use of about 1-5 μM or 2-4 μM CHIR99021 for differentiation of pluripotent cells into definitive endoderm cells. In some embodiments, the method comprises use of about 2 μM CHIR99021 for differentiation of pluripotent cells into definitive endoderm cells. In some embodiments, the method comprises use of about 3 μM CHIR99021 for differentiation of pluripotent cells into definitive endoderm cells. In some embodiments, the method comprises use of about 5 μM CHIR99021 for differentiation of pluripotent cells into definitive endoderm cells.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, a definitive endoderm cell produced by the methods as disclosed herein expresses at least one marker selected from the group consisting of. Nodal, Tmprss2, Tmem30b, St14, Spink3, Sh3gl2, Ripk4, Rab1S, Npnt, Clic6, Cldn5, Cacna1b, Bnip1, Anxa4, Emb, FoxA1, Sox17, and Rbm35a, wherein the expression of at least one marker is upregulated to by a statistically significant amount in the definitive endoderm cell relative to the pluripotent stem cell from which it was derived. In some embodiments, a definitive endoderm cell produced by the methods as disclosed herein does not express by a statistically significant amount at least one marker selected the group consisting of. Gata4, SPARC, AFP and Dab2 relative to the pluripotent stem cell from which it was derived. In some embodiments, a definitive endoderm cell produced by the methods as disclosed herein does not express by a statistically significant amount at least one marker selected the group consisting of: Zic1, Pax6, Flk1 and CD31 relative to the pluripotent stem cell from which it was derived. In some embodiments, a definitive endoderm cell produced by the methods as disclosed herein has a higher level of phosphorylation of Smad2 by a statistically significant amount relative to the pluripotent stem cell from which it was derived. In some embodiments, a definitive endoderm cell produced by the methods as disclosed herein has the capacity to form gut tube in vivo. In some embodiments, a definitive endoderm cell produced by the methods as disclosed herein can differentiate into a cell with morphology characteristic of a gut cell, and wherein a cell with morphology characteristic of a gut cell expresses FoxA2 and/or Claudin6. In some embodiments, a definitive endoderm cell produced by the methods as disclosed herein can be further differentiated into a cell of endoderm origin.

In some embodiments, a population of pluripotent stem cells are cultured in the presence of at least one β cell differentiation factor prior to any differentiation or during the first stage of differentiation. One can use any pluripotent stem cell, such as a human pluripotent stem cell, or a human iPS cell or any of pluripotent stem cell as discussed herein or other suitable pluripotent stem cells. In some embodiments, a β cell differentiation factor as described herein can be present in the culture medium of a population of pluripotent stem cells or may be added in bolus or periodically during growth (e.g., replication or propagation) of the population of pluripotent stem cells. In certain examples, a population of pluripotent stem cells can be exposed to at least one β cell differentiation factor prior to any differentiation. In other examples, a population of pluripotent stem cells may be exposed to at least one β cell differentiation factor during the first stage of differentiation.

### Primitive Gut Tube Cells

Aspects of the disclosure involve primitive gut tube cells. Primitive gut tube cells of use herein can be derived from any source or generated in accordance with any suitable protocol. In some aspects, definitive endoderm cells are differentiated to primitive gut tube cells. In some aspects, the primitive gut tube cells are further differentiated, e.g., to PDX1-positive, NKX6.1-negative pancreatic progenitor cells, PDX1-positive, NKX6.1-positive pancreatic progenitor cells, Ngn3-positive endocrine progenitor cells, insulin-positive endocrine cells, followed by induction or maturation to SC-β cells.

In some embodiments, primitive gut tube cells can be obtained by differentiating at least some definitive endoderm cells in a population into primitive gut tube cells, e.g., by contacting definitive endoderm cells with at least one growth factor from the fibroblast growth factor (FGF) family, to induce the differentiation of at least some of the definitive endoderm cells into primitive gut tube cells, wherein the primitive gut tube cells express at least one marker characteristic of primitive gut tube cells.

Any growth factor from the FGF family capable of inducing definitive endoderm cells to differentiate into primitive gut tube cells (e.g., alone, or in combination with other factors) can be used in the method provided herein. In some embodiments, the at least one growth factor from the FGF family comprises keratinocyte growth factor (KGF). In some embodiments, the at least one growth factor from the FGF family comprises FGF2. In some embodiments, the at least one growth factor from the FGF family comprises FGF8B. In some embodiments, the at least one growth factor from the FGF family comprises FGF10. In some embodiments, the at least one growth factor from the FGF family comprises FGF21.

In some embodiments, primitive gut tube cells can be obtained by differentiating at least some definitive endoderm cells in a population into primitive gut tube cells, e.g., by contacting definitive endoderm cells with KGF for a certain period of time, e.g., about 1 day, about 2 days, about 3 days, about 4 days, 24-96 hours, 50-80 hours, 60-80 hours, or 65-75 hours, to induce the differentiation of at least some of the definitive endoderm cells into primitive gut tube cells.

In some embodiments, the method comprises differentiating definitive endoderm cells into primitive gut tube cells by contacting definitive endoderm cells with a suitable concentration of the growth factor from the FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some embodiments, the method comprises use of about 20-80 ng/ml, 30-70 ng/ml, or 40-60 ng/mL KGF for differentiation of definitive endoderm cells into primitive gut tube cells. In some embodiments, the method comprises use of about 50 ng/mL KGF for differentiation of definitive endoderm cells into primitive gut tube cells. In some embodiments, the method comprises use of about 100 ng/mL KGF for differentiation of definitive endoderm cells into primitive gut tube cells.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, the cells are contacted with any of the inhibitors of PI3K/Akt/mTOR signaling as disclosed herein.

### PDX1-Positive Pancreatic Progenitor Cells

Aspects of the disclosure involve PDX1-positive, NKX6.1-negative pancreatic progenitor cells. PDX1-positive, NKX6.1-negative pancreatic progenitor cells of use herein can be derived from any source or generated in accordance with any suitable protocol, including a method disclosed herein involving the use of a small molecule compound, such as an inhibitor of PI3K/Akt/mTOR signaling.

In some aspects, primitive gut tube cells are differentiated to PDX1-positive pancreatic progenitor cells (e.g., PDX1-positive, NKX6.1-negative cells). In some aspects, the PDX1-positive pancreatic progenitor cells are NKX6.1 negative, and can be further differentiated to, e.g., NKX6.1-positive pancreatic progenitor cells, Ngn3-positive endocrine progenitor cells, insulin-positive endocrine cells, followed by induction or maturation to SC-β cells.

In some aspects, PDX1-positive pancreatic progenitor cells can be obtained by differentiating at least some primitive gut tube cells in a population into PDX1-positive pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; vi) at least one protein kinase C activator, and vii) a ROCK inhibitor to induce the differentiation of at least some of the primitive gut tube cells into PDX1-positive pancreatic progenitor cells, wherein the PDX1-positive pancreatic progenitor cells express PDX1.

In some aspects, PDX1-positive pancreatic progenitor cells can be obtained by differentiating at least some primitive gut tube cells in a population into PDX1-positive pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; and vi) at least one protein kinase C activator, to induce the differentiation of at least some of the primitive gut tube cells into PDX1-positive pancreatic progenitor cells, wherein the PDX1-positive pancreatic progenitor cells express PDX1.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with i) at least one BMP signaling pathway inhibitor, ii) at least one growth factor from the FGF family, iii) at least one SHH pathway inhibitor, iv) at least one retinoic acid (RA) signaling pathway activator; and v) at least one protein kinase C activator, to induce the differentiation of at least some of the primitive gut tube cells into PDX1-positive pancreatic progenitor cells, wherein the PDX1-positive pancreatic progenitor cells express PDX1.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with i) at least one SHH pathway inhibitor, ii) at least one retinoic acid (RA) signaling pathway activator; and iii) at least one protein kinase C activator, wherein the PDX1-positive pancreatic progenitor cells express PDX1.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with i) at least one growth factor from the FGF family, and ii) at least one retinoic acid (RA) signaling pathway activator, to induce the differentiation of at least some of the primitive gut tube cells into PDX1-positive pancreatic progenitor cells, wherein the PDX1-positive pancreatic progenitor cells express PDX1.

Any BMP signaling pathway inhibitor capable of inducing primitive gut tube cells to differentiate into PDX1-positive pancreatic progenitor cells (e.g., alone, or with any combination of a growth factor from TGF-β superfamily, at least one growth factor from the FGF family, at least one SHH pathway inhibitor, at least one retinoic acid signaling pathway activator, at least one protein kinase C activator, and ROCK inhibitor) can be used in the method provided herein. In some embodiments, the BMP signaling pathway inhibitor comprises LDN193189 or DMH-1. In some examples, the method comprises contacting primitive gut tube cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 μM. In some examples, the method comprises contacting primitive gut tube cells with a concentration of BMP signaling pathway inhibitor (e.g., DMH-1), such as, about 0.01 μM, about 0.02 μM, about 0.05 μM, about 0.1 μM, about 0.2 μM, about 0.5 μM, about 0.8 μM, about 1 μM, about 1.2 μM, about 1.5 μM, about 1.75 μM, about 2 μM, about 2.2 μM, about 2.5 μM, about 2.75 μM, about 3 μM, about 3.25 μM, about 3.5 μM, about 3.75 μM, about 4 μM, about 4.5 μM, about 5 μM, about 8 μM, about 10 μM, about 15 μM, about 20 μM, about 30 μM, about 40 μM, about 50 μM, or about 100 μM. In some examples, the method comprises contacting primitive gut tube cells with a concentration of BMP signaling pathway inhibitor (e.g., DMH-1), such as, about 220-280 nM, about 230-270 nM, about 240-260 nM, or about 245-255 nM. In some examples, the method comprises contacting primitive gut tube cells with a concentration of BMP signaling pathway inhibitor (e.g., DMH-1) about 250 nM.

Any growth factor from the TGF-β superfamily capable of inducing primitive gut tube cells to differentiate into PDX1-positive pancreatic progenitor cells (e.g., alone, or with any combination of at least one BMP signaling pathway inhibitor, a growth factor from the FGF family, at least one SHH pathway inhibitor, at least one retinoic acid signaling pathway activator, at least one protein kinase C activator, and ROCK inhibitor) can be used. In some embodiments, the growth factor from TGF-β family comprises Activin A. In some embodiments, the growth factor from TGF-β family comprises GDF8. In some examples, the method comprises contacting primitive gut tube cells with a concentration of a growth factor from TGF-β superfamily (e.g., Activin A), such as, about 5 ng/mL, about 7.5 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 35 ng/mL, about 40 ng/mL, about 50 ng/mL, or about 100 ng/mL. In some examples, the method comprises contacting primitive gut tube cells with a concentration of a growth factor from TGF-β superfamily (e.g., Activin A), such as, about 17-23 ng/ml, about 18-22 ng/ml, or about 19-21 ng/ml. In some examples, the method comprises contacting primitive gut tube cells with a concentration of a growth factor from TGF-β superfamily (e.g., Activin A) of about 20 ng/ml.

Any growth factor from the FGF family capable of inducing primitive gut tube cells to differentiate into PDX1-positive pancreatic progenitor cells (e.g., alone, or with any combination of at least one BMP signaling pathway inhibitor, a growth factor from TGF-β superfamily, at least one SHH pathway inhibitor, at least one retinoic acid signaling pathway activator, at least one protein kinase C activator, and ROCK inhibitor) can be used. In some embodiments, the at least one growth factor from the FGF family comprises keratinocyte growth factor (KGF). In some embodiments, the at least one growth factor from the FGF family is selected from the group consisting of FGF2, FGF8B, FGF10, and FGF21. In some examples, the method comprises contacting primitive gut tube cells with a concentration of a growth factor from FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some examples, the method comprises contacting primitive gut tube cells with a concentration of a growth factor from FGF family (e.g., KGF), such as, about 20-80 ng/ml, about 30-70 ng/ml, about 40-60 ng/ml, or about 45-55 ng/ml. In some examples, the method comprises contacting primitive gut tube cells with a concentration of a growth factor from FGF family (e.g., KGF) of about 50 ng/ml.

Any SHH pathway inhibitor capable of inducing primitive gut tube cells to differentiate into PDX1-positive pancreatic progenitor cells (e.g., alone, or with any combination of at least one BMP signaling pathway inhibitor, at least one growth factor from the FGF family, a growth factor from TGF-β superfamily, at least one retinoic acid signaling pathway activator, at least one protein kinase C activator, and ROCK inhibitor) can be used. In some embodiments, the SHH pathway inhibitor comprises Sant1. In some examples, the method comprises contacting primitive gut tube cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting primitive gut tube cells with a concentration of a SHH pathway inhibitor (e.g., Sant1), such as, about 220-280 nM, about 230-270 nM, about 240-260 nM, or about 245-255 nM. In some examples, the method comprises contacting primitive gut tube cells with a concentration of a SHH pathway inhibitor (e.g., Sant1) of about 250 nM.

Any RA signaling pathway activator capable of inducing primitive gut tube cells to differentiate into PDX1-positive pancreatic progenitor cells (e.g., alone, or with any combination of at least one BMP signaling pathway inhibitor, at least one growth factor from the FGF family, at least one SHH pathway inhibitor, at least one protein kinase C activator, and ROCK inhibitor) can be used. In some embodiments, the RA signaling pathway activator comprises retinoic acid. In some examples, the method comprises contacting primitive gut tube cells with a concentration of an RA signaling pathway activator (e.g., retinoic acid), such as, about 0.02 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting primitive gut tube cells with a concentration of an RA signaling pathway activator (e.g., retinoic acid), such as, about 1.7-2.3 μM, about 1.8-2.2 μM, or about 1.9-2.1 μM. In some examples, the method comprises contacting primitive gut tube cells with a concentration of an RA signaling pathway activator (e.g., retinoic acid) of about 2 μM.

Any PKC activator capable of inducing primitive gut tube cells to differentiate into PDX1-positive pancreatic progenitor cells (e.g., alone, or with any combination of at least one BMP signaling pathway inhibitor, at least one growth factor from the FGF family, at least one SHH pathway inhibitor, at least one RA signaling pathway activator, and ROCK inhibitor) can be used. In some embodiments, the PKC activator comprises PdBU. In some embodiments, the PKC activator comprises TPPB. In some examples, the method comprises contacting primitive gut tube cells with a concentration of a PKC activator (e.g., PdBU or TPPB), such as, about 10 nM, 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 10 μM, about 20 μM, about 50 μM, about 75 μM, about 80 μM, about 100 μM, about 120 μM, about 140 μM, about 150 μM, about 175 μM, about 180 μM, about 200 μM, about 210 μM, about 220 μM, about 240 μM, about 250 μM, about 260 μM, about 280 μM, about 300 μM, about 320 μM, about 340 μM, about 360 μM, about 380 μM, about 400 μM, about 420 μM, about 440 μM, about 460 μM, about 480 μM, about 500 μM, about 520 μM, about 540 μM, about 560 μM, about 580 μM, about 600 μM, about 620 μM, about 640 μM, about 660 μM, about 680 μM, about 700 μM, about 750 μM, about 800 μM, about 850 μM, about 900 μM, about 1 mM, about 2 mM, about 3 mM, about 4 mM, or about 5 mM. In some embodiments, the method comprises contacting primitive gut tube cells with a concentration of a PKC activator (e.g., PdBU or TPPB) of 10 nM-1 mM, 10 nM-500 μM, 10 nM-1 μM, 10-800 nM, 100-900 nM, 300-800 nM, 300-600 nM, 400-600 nM, 450-550 nM, or about 500 nM. In some examples, the method comprises contacting primitive gut tube cells with a concentration of a PKC activator (e.g., PdBU or TPPB), such as, about 450-550 mM, about 475-525 nM, about 490-510 nM, or about 495-505 nM. In some examples, the method comprises contacting primitive gut tube cells with a concentration of a PKC activator (e.g., PdBU or TPPB) of about 500 nM. In some embodiments, primitive gut tube cells are not treated with a PKC activator (e.g., PDBU).

Any ROCK inhibitor capable of inducing primitive gut tube cells to differentiate into PDX1-positive pancreatic progenitor cells (e.g., alone, or with any combination of at least one BMP signaling pathway inhibitor, at least one growth factor from the FGF family, at least one SHH pathway inhibitor, PKC activator, and at least one RA signaling pathway activator) can be used. In some embodiments, the ROCK inhibitor comprises Thiazovivin, Y-27632, Fasudil/HA1077, or H-1152. In some embodiments, the ROCK inhibitor comprises Y-27632. In some embodiments, the ROCK inhibitor comprises Thiazovivin. In some examples, the method comprises contacting primitive gut tube cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some examples, the method comprises contacting primitive gut tube cells with a concentration of a ROCK inhibitor (e.g., Y-27632 or Thiazovivin), such as, about 2.2-2.8 μM, about 2.3-2.7 μM, or about 2.4-2.6 μM. In some examples, the method comprises contacting primitive gut tube cells with a concentration of a ROCK inhibitor (e.g., Y-27632 or Thiazovivin) of about 2.5 μM.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with retinoic acid, KGF, Sant1, DMH-1, PdBU, thiazovivin, and Activin A, for a suitable period of time, e.g., about 1 day, about 2 days, about 3 days, or about 4 days. In some embodiments, PDX1-positive pancreatic progenitor cells can be obtained by differentiating at least some primitive gut tube cells in a population into PDX1-positive pancreatic progenitor cells, e.g., by contacting primitive gut tube cells with retinoic acid, KGF, Sant1, DMH-1, PdBU, thiazovivin, and Activin A, for about 2 days. In some embodiments, PDX1-positive pancreatic progenitor cells can be obtained by differentiating at least some primitive gut tube cells in a population into PDX1-positive pancreatic progenitor cells, e.g., by contacting primitive gut tube cells with retinoic acid, KGF, Sant1, DMH-1, PdBU, thiazovivin, and Activin A for 1 day, followed by contacting the cells with retinoic acid, KGF, Sant1, PdBU, thiazovivin, and Activin A for 1 day (in the absence of DMH-1).

### NKX6.1-Positive Pancreatic Progenitor Cells

Aspects of the disclosure involve NKX6.1-positive pancreatic progenitor cells. NKX6.1-positive pancreatic progenitor cells of use herein can be derived from any source or generated in accordance with any suitable protocol, including a method disclosed herein involving the use of a small molecule compound, such as an inhibitor of PI3K/Akt/mTOR signaling. In some aspects, PDX1-positive, NKX6.1-negative pancreatic progenitor cells are differentiated to PDX1-positive, NKX6.1-positive pancreatic progenitor cells. In some aspects, the PDX1-positive, NKX6.1-positive pancreatic progenitor cells are further differentiated, e.g., to Ngn3-positive endocrine progenitor cells, or insulin-positive endocrine cells, followed by induction or maturation to SC-β cells.

In some aspects, a method of producing a NKX6.1-positive pancreatic progenitor cell from a PDX1-positive pancreatic progenitor cell comprises contacting a population of cells (e.g., under conditions that promote cell clustering and/or promoting cell survival) comprising PDX1-positive pancreatic progenitor cells with at least two β cell-differentiation factors comprising a) at least one growth factor from the fibroblast growth factor (FGF) family, b) a sonic hedgehog pathway inhibitor, and optionally c) a low concentration of a retinoic acid (RA) signaling pathway activator, to induce the differentiation of at least one PDX1-positive pancreatic progenitor cell in the population into NKX6.1-positive pancreatic progenitor cells, wherein the NKX6.1-positive pancreatic progenitor cells expresses NKX6.1.

In some embodiments, the PDX1-positive, NKX6.1-positive pancreatic progenitor cells are obtained by contacting PDX1-positive pancreatic progenitor cells with i) at least one growth factor from the FGF family, ii) at least one SHH pathway inhibitor, and optionally iii) a RA signaling pathway activator, to induce the differentiation of at least some of the PDX1-positive pancreatic progenitor cells into PDX1-positive, NKX6.1-positive pancreatic progenitor cells, wherein the PDX1-positive, NKX6.1-positive pancreatic progenitor cells express PDX1 and NKX6.1.

In some embodiments, the PDX1-positive, NKX6.1-positive pancreatic progenitor cells are obtained by contacting PDX1-positive pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily, to induce the differentiation of at least some of the PDX1-positive pancreatic progenitor cells into PDX1-positive, NKX6.1-positive pancreatic progenitor cells. In some embodiments, following 3, 4, or 5 days of contacting the PDX1-positive, NKX6.1-positive pancreatic progenitor cells are obtained by contacting PDX1-positive pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily; the cells are then contacted with i) at least one growth factor from the FGF family, ii) at least one SHH pathway inhibitor, and optionally iii) a RA signaling pathway activator, iv) ROCK inhibitor, and v) at least one growth factor from the TGF-β superfamily, and vi) a PKC activator and optionally a gamma-secretase inhibitor. In some embodiments, the PDX1-positive, NKX6.1-positive pancreatic progenitor cells are obtained by contacting PDX1-positive pancreatic progenitor cells under conditions that promote cell clustering with at least one growth factor from the FGF family. In some embodiments, the growth factor from the FGF family is KGF.

In some embodiments, the disclosure provides for a method in which a first population of cells comprising PDX1-positive, NKX6.1-negative cells is cultured in a media comprising any one or combination of: i) at least one growth factor from the FGF family, ii) at least one SHH pathway inhibitor, iii) a RA signaling pathway activator, iv) a ROCK inhibitor, and v) a growth factor from the TGF-β superfamily for a period of about 1, 2, 3, 4 or 5 days (e.g., 2-4, 3-4, or 4-5 days); thereby generating a second population of cells. In some embodiments, the second population of cells is then incubated in a composition comprising any one or combination of: i) at least one growth factor from the FGF family, ii) at least one SHH pathway inhibitor, iii) a RA signaling pathway activator, iv) a ROCK inhibitor, v) a growth factor from the TGF-β superfamily, vi) a PKC activator, vii) a FoxO1 inhibitor, and optionally viii) a notch signaling inhibitor for about 1, 2, or 3 days (e.g., 1-2, 1-3, or 2-3 days).

In some embodiments, in the media for culturing the first population of cells, the growth factor from the FGF family is present at a concentration of about 45-55 ng/ml, about 46-54 ng/ml, about 47-53 ng/ml, about 48-52 ng/ml, or about 49-51 ng/ml, the SHH pathway inhibitor is present at a concentration of about 200-300 nM, about 220-280 nM, or about 240-260 nM, the RA signaling pathway activator is present at a concentration of about 1.7-2.3 μM, about 1.8-2.2 μM, or about 1.9-2.1 μM, the ROCK inhibitor is present at a concentration of about 2-3 μM, about 2.2-2.8 μM, or about 2.4-2.6 μM, and/or the growth factor from the TGF-β superfamily is present at a concentration of about 2-8 ng/ml, about 3-7 ng/ml or about 4-6 ng/ml.

In some embodiments, in the media for culturing the second population of cells, the growth factor from the FGF family is present at a concentration of about 45-55 ng/ml, about 46-54 ng/ml, about 47-53 ng/ml, about 48-52 ng/ml, or about 49-51 ng/ml, the SHH pathway inhibitor is present at a concentration of about 200-300 nM, about 220-280 nM, or about 240-260 nM, the RA signaling pathway activator is present at a concentration of about 1.7-2.3 μM, about 1.8-2.2 μM, or about 1.9-2.1 μM, the ROCK inhibitor is present at a concentration of about 2-3 μM, about 2.2-2.8 μM, or about 2.4-2.6 μM, the growth factor from the TGF-β superfamily is present at a concentration of 2 about −8 ng/ml, about 3-7 ng/ml or about 4-6 ng/ml, the PKC activator is present at a concentration of about 0.2-0.8 μM, about 0.3-0.7 μM, or about 0.4-0.6 μM, and the FoxO1 inhibitor is present at a concentration of about 0.7-1.3 μM, about 0.8-1.2 μM, or about 0.9-1.1 μM, and optionally the notch signaling inhibitor is present at a concentration of about 1.7-2.3 μM, about 1.8-2.2 μM, or about 1.9-2.1 μM.

In some embodiments, the PDX1-positive pancreatic progenitor cells are produced from a population of pluripotent cells. In some embodiments, the PDX1-positive pancreatic progenitor cells are produced from a population of iPS cells. In some embodiments, the PDX1-positive pancreatic progenitor cells are produced from a population of ESC cells. In some embodiments, the PDX1-positive pancreatic progenitor cells are produced from a population of definitive endoderm cells. In some embodiments, the PDX1-positive pancreatic progenitor cells are produced from a population of primitive gut tube cells.

Any growth factor from the FGF family capable of inducing PDX1-positive pancreatic progenitor cells to differentiate into NKX6.1-positive pancreatic progenitor cells (e.g., alone, or with any combination of at least one SHH pathway inhibitor, a ROCK inhibitor, a growth factor from the TGF-β superfamily, and at least one retinoic acid signaling pathway activator) can be used in the method provided herein. In some embodiments, the at least one growth factor from the FGF family comprises keratinocyte growth factor (KGF). In some embodiments, the at least one growth factor from the FGF family is selected from the group consisting of FGF8B, FGF 10, and FGF21. In some examples, the method comprises contacting PDX1-positive pancreatic progenitor cells with a concentration of a growth factor from FGF family (e.g., KGF), such as, about 10 ng/mL, about 20 ng/mL, about 50 ng/mL, about 75 ng/mL, about 80 ng/mL, about 90 ng/mL, about 95 ng/mL, about 100 ng/mL, about 110 ng/mL, about 120 ng/mL, about 130 ng/mL, about 140 ng/mL, about 150 ng/mL, about 175 ng/mL, about 180 ng/mL, about 200 ng/mL, about 250 ng/mL, or about 300 ng/mL. In some examples, the method comprises contacting PDX1-positive pancreatic progenitor cells with a concentration of a growth factor from FGF family (e.g., KGF), such as, about 20-80 ng/ml, about 30-70 ng/ml, about 40-60 ng/ml, or about 45-55 ng/ml. In some examples, the method comprises contacting PDX1-positive pancreatic progenitor cells with a concentration of a growth factor from FGF family (e.g., KGF) of about 50 ng/ml.

Any SHH pathway inhibitor capable of inducing PDX1-positive pancreatic progenitor cells to differentiate into NKX6.1-positive pancreatic progenitor cells (e.g., alone, or with any combination of at least one growth factor from the FGF family, a retinoic acid signaling pathway activator, ROCK inhibitor, and at least one growth factor from the TGF-β superfamily) can be used in the method provided herein. In some embodiments, the SHH pathway inhibitor comprises Sant1. In some examples, the method comprises contacting PDX1-positive pancreatic progenitor cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a SHH pathway inhibitor (e.g., Sant1), such as, about 220-280 nM, about 230-270 nM, about 240-260 nM, or about 245-255 nM. In some examples, the method comprises contacting PDX1-positive pancreatic progenitor cells with a concentration of a SHH pathway inhibitor (e.g., Sant1) of about 250 nM.

Any RA signaling pathway activator capable of inducing PDX1-positive pancreatic progenitor cells to differentiate into NKX6.1-positive pancreatic progenitor cells (e.g., alone, or with any combination of at least one growth factor from the FGF family, at least one SHH pathway inhibitor, ROCK inhibitor, and at least one growth factor from the TGF-β superfamily) can be used. In some embodiments, the RA signaling pathway activator comprises retinoic acid. In some examples, the method comprises contacting PDX1-positive pancreatic progenitor cells with a concentration of an RA signaling pathway activator (e.g., retinoic acid), such as, about 0.02 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting PDX1-positive pancreatic progenitor cells with a concentration of an RA signaling pathway activator (e.g., retinoic acid), such as, about 70-130 nM, about 80-120 nM, about 90-110 nM, or about 95-105 nM. In some examples, the method comprises contacting PDX1-positive pancreatic progenitor cells with a concentration of an RA signaling pathway activator (e.g., retinoic acid) of about 100 nM.

Any ROCK inhibitor capable of inducing PDX1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and at least one growth factor from the TGF-β superfamily) can be used. In some embodiments, the ROCK inhibitor comprises Thiazovivin, Y-27632, Fasudil/HA1077, or 14-1152. In some examples, the method comprises contacting PDX1-positive pancreatic progenitor cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some examples, the method comprises contacting PDX1-positive pancreatic progenitor cells with a concentration of a ROCK inhibitor (e.g., Y-27632 or Thiazovivin), such as, about 2.2-2.8 μM, about 2.3-2.7 μM, or about 2.4-2.6 μM. In some examples, the method comprises contacting PDX1-positive pancreatic progenitor cells with a concentration of a ROCK inhibitor (e.g., Y-27632 or Thiazovivin) of about 2.5 μM.

Any activator from the TGF-β superfamily capable of inducing PDX1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and ROCK inhibitor) can be used. In some embodiments, the activator from the TGF-β superfamily comprises Activin A or GDF8. In some examples, the method comprises contacting PDX1-positive pancreatic progenitor cells with a concentration of a growth factor from TGF-β superfamily (e.g., Activin A), such as, about 0.1 ng/mL, about 0.2 ng/mL, about 0.3 ng/mL, about 0.4 ng/mL, about 0.5 ng/mL, about 0.6 ng/mL, about 0.7 ng/mL, about 0.8 ng/mL, about 1 ng/mL, about 1.2 ng/mL, about 1.4 ng/mL, about 1.6 ng/mL, about 1.8 ng/mL, about 2 ng/mL, about 2.2 ng/mL, about 2.4 ng/mL, about 2.6 ng/mL, about 2.8 ng/mL, about 3 ng/mL, about 3.2 ng/mL, about 3.4 ng/mL, about 3.6 ng/mL, about 3.8 ng/mL, about 4 ng/mL, about 4.2 ng/mL, about 4.4 ng/mL, about 4.6 ng/mL, about 4.8 ng/mL, about 5 ng/mL, about 5.2 ng/mL, about 5.4 ng/mL, about 5.6 ng/mL, about 5.8 ng/mL, about 6 ng/mL, about 6.2 ng/mL, about 6.4 ng/mL, about 6.6 ng/mL, about 6.8 ng/mL, about 7 ng/mL, about 8 ng/mL, about 9 ng/mL, about 10 ng/mL, about 20 ng/mL, about 30 ng/mL, or about 50 ng/mL. In some examples, the method comprises contacting PDX1-positive pancreatic progenitor cells with a concentration of a growth factor from TGF-β superfamily (e.g., Activin A), such as, about 2-8 ng/ml, about 3-7 ng/ml, about 4-6 ng/ml, or about 4.5-5.5 ng/ml. In some examples, the method comprises contacting PDX1-positive pancreatic progenitor cells with a concentration of a growth factor from TGF-β superfamily (e.g., Activin A), such as, about 5 ng/mL.

Any FoxO1 inhibitor capable of inducing PDX1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, at least one growth factor from the TGF-β superfamily, PKC activator, and Notch signaling inhibitor) can be used in the method provided herein. In some embodiments, the FoxO1 inhibitor is AS1842856. In some examples, the method comprises contacting PDX1-positive pancreatic progenitor cells with a concentration of a FoxO1 inhibitor (e.g., AS1842856), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a FoxO1 inhibitor (e.g., AS1842856), such as, about 0.7-1.3 μM, about 0.8-1.2 μM, about or 0.9-1.1 μM. In some examples, the method comprises contacting PDX1-positive pancreatic progenitor cells with a concentration of a FoxO1 inhibitor (e.g., AS1842856), such as, about 1 μM.

Any PKC activator capable of inducing PDX1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, at least one growth factor from the TGF-β superfamily, FoxO1 inhibitor, and Notch signaling inhibitor) can be used in the method provided herein. In some embodiments, the PKC activator is PDBU. In some examples, the method comprises contacting PDX1-positive pancreatic progenitor cells with a concentration of a PKC activator (e.g., PDBU), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a PKC activator (e.g., PDBU), such as, about 0.2-0.8 μM, about 0.3-0.7 μM, about 0.4-0.6 μM. In some examples, the method comprises contacting PDX1-positive pancreatic progenitor cells with a concentration of a PKC activator (e.g., PDBU), such as, about 0.5 μM.

Any Notch signaling inhibitor capable of inducing PDX1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, at least one growth factor from the TGF-β superfamily, FoxO1 inhibitor, and PKC activator) can be used in the method provided herein. In some embodiments, the Notch signaling inhibitor is XXI. In some examples, the method comprises contacting PDX1-positive pancreatic progenitor cells with a concentration of a Notch signaling inhibitor (e.g., XXI), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a Notch signaling inhibitor (e.g., XXI), such as, about 1.7-2.3 μM, about 1.8-2.2 μM, or about 1.9-2.1 μM. In some examples, the method comprises contacting PDX1-positive pancreatic progenitor cells with a concentration of a Notch signaling inhibitor (e.g., XXI), such as, about 2 μM.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, the PDX1-positive, NKX6.1-positive pancreatic progenitor cells are obtained by contacting PDX1-positive pancreatic progenitor cells under conditions that promote cell clustering with KGF, Sant1, and RA, for a period of 5 days or 6 days. In some embodiments, the PDX1-positive, NKX6.1-positive pancreatic progenitor cells are obtained by contacting PDX1-positive pancreatic progenitor cells under conditions that promote cell clustering with KGF, Sant1, RA, thiazovivin, and Activin A, for a period of 5 or 6 days. In some embodiments, the PDX1-positive, NKX6.1-positive pancreatic progenitor cells are obtained by contacting PDX1-positive pancreatic progenitor cells under conditions that promote cell clustering with KGF for a period of 5 days. In some embodiments, the PDX1-positive, NKX6.1-positive pancreatic progenitor cells are obtained by contacting PDX1-positive pancreatic progenitor cells under conditions that promote cell clustering with KGF for a period of 6 days. In some embodiments, the PDX1-positive, NKX6.1-positive pancreatic progenitor cells are obtained by: a) contacting PDX1-positive pancreatic progenitor cells with KGF, Sant1, RA, thiazovivin, and Activin A, for a period of 3, 4 or 5 days (e.g., 4 days), followed by; b) contacting the cells of a) with PDBU, XXI, KGF, Sant1, RA, thiazovivin, and Activin A and optionally AS1842856 for a period of 1, 2 or 3 days (e.g., 2 days).

### Insulin-Positive Endocrine Cells

Aspects of the disclosure involve insulin-positive endocrine cells (e.g., NKX6.1-positive, ISL1-positive cells, or β-like cells) and additional methods of generating insulin-positive endocrine cells. Insulin-positive endocrine cells of use herein can be derived from any source or generated in accordance with any suitable protocol. In some aspects, NKX6.1-positive pancreatic progenitor cells are differentiated to insulin-positive endocrine cells (e.g., NKX6.1-positive, ISL1-positive cells, or β-like cells). In some aspects, the insulin-positive endocrine cells are further differentiated, e.g., by induction or maturation to SC-β cells.

In some aspects, a method of producing an insulin-positive endocrine cell from an NKX6.1-positive pancreatic progenitor cell comprises contacting a population of cells (e.g., under conditions that promote cell clustering) comprising NKX6-1-positive pancreatic progenitor cells with a) a TGF-β signaling pathway inhibitor, b) a thyroid hormone signaling pathway activator, c) a BMP pathway inhibitor, and/or d) a protein kinase inhibitor to induce the differentiation of at least one NKX6.1-positive pancreatic progenitor cell in the population into an insulin-positive endocrine cell, wherein the insulin-positive endocrine ceil expresses insulin. In some embodiments, insulin-positive endocrine cells express PDX1, NKX6.1, ISL1, NKX2.2, Mafb, glis3, Sur1, Kir6.2, Znt8, SLC2A1, SLC2A3 and/or insulin.

Any TGF-β signaling pathway inhibitor capable of inducing the differentiation of NKX6.1-positive pancreatic progenitor cells to differentiate into insulin-positive endocrine cells (e.g., alone, or in combination with other β cell-differentiation factors, e.g., a thyroid hormone signaling pathway activator) can be used. In some embodiments, the TGF-β signaling pathway comprises TGF-β receptor type I kinase signaling. In some embodiments, the TGF-β signaling pathway inhibitor comprises Alk5 inhibitor II. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 0.1 μM, about 0.5 μM, about 1 μM, about 1.5 μM, about 2 μM, about 2.5 μM, about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, about 20 μM, about 25 μM, about 30 μM, about 35 μM, about 40 μM, about 45 μM, or about 50 μM. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 7-13 μM, about 8-12 μM, about 9-11 μM. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 10 μM.

Any thyroid hormone signaling pathway activator capable of inducing the differentiation of NKX6.1-positive pancreatic progenitor cells to differentiate into insulin-positive endocrine cells (e.g., alone, or in combination with other β cell-differentiation factors, e.g., a TGF-β signaling pathway inhibitor) can be used. In some embodiments, the thyroid hormone signaling pathway activator comprises triiodothyronine (T3). In some embodiments, the thyroid hormone signaling pathway activator comprises GC-1. In some examples, the method comprises contacting NKX6.1-positive pancreatic progenitor cells with a concentration of thyroid hormone signaling pathway activator (e.g., GC-1), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting NKX6.1-positive pancreatic progenitor cells with a concentration of thyroid hormone signaling pathway activator (e.g., GC-1), such as, about 0.7-1.3 μM, about 0.8-1.2 μM, or about 0.9-1.1 μM. In some examples, the method comprises contacting NKX6.1-positive pancreatic progenitor cells with a concentration of thyroid hormone signaling pathway activator (e.g., GC-1), such as, about 1 μM.

In some embodiments, the method comprises contacting the population of cells (e.g., NKX6.1-positive pancreatic progenitor cells) with at least one additional factor. In some embodiments, the method comprises contacting the PDX1-positive NKX6.1-positive pancreatic progenitor cells with at least one of i) a SHH pathway inhibitor, ii) a γ-secretase inhibitor, iii) at least one growth factor from the epidermal growth factor (EGF) family, iv) a TGF-β signaling pathway inhibitor, or vii) a thyroid hormone signaling pathway activator. In some embodiments, the method comprises contacting the population of cells (e.g., NKX6.1-positive pancreatic progenitor cells) with at least one additional factor. In some embodiments, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) a protein kinase inhibitor, vi) a TGF-β signaling pathway inhibitor, vii) a thyroid hormone signaling pathway activator, viii) a wnt signaling pathway inhibitor, or ix) a PKC activator.

In some embodiments, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) a protein kinase inhibitor, or ix) a ROCK inhibitor.

In some embodiments, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, or x) a ROCK inhibitor. In some embodiments, the method comprises contacting the PDX1-positive, NKX6.1-positive pancreatic progenitor cells in a culture with a i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, x) a ROCK inhibitor, xi) a PKC activator and xii) a Wnt signaling pathway inhibitor for 1, 2, or 3 days (e.g., 1-2, 1-3, or 2-3 days), and then contacting the cells in the culture with i) a γ-secretase inhibitor, ii) at least one growth factor from the epidermal growth factor (EGF) family, iii) at least one bone morphogenetic protein (BMP) signaling pathway inhibitor, iv) a TGF-β signaling pathway inhibitor, v) a thyroid hormone signaling pathway activator, vi) an epigenetic modifying compound, vii) a protein kinase inhibitor, and viii) a ROCK inhibitor for a period of 1, 2, 3, 4, 5, 6, or 7 days (e.g., 1-7, 1-5, 1-3, 3-7, 3-5, 5-7, or 4-6 days) in the absence of a SHH pathway inhibitor, a RA signaling pathway activator, a Wnt signaling pathway inhibitor, PKC activator, and/or growth factor from the epidermal growth factor (EGF) family.

In some embodiments, in the method of generating the insulin-positive endocrine cells from the PDX1-positive NKX6.1-postive pancreatic progenitor cells, some of the differentiation factors are present only for the first 1, 2, 3, 4, or 5 days during the differentiation step. In some embodiments, some of the differentiation factors, such as the SHH pathway inhibitor, the RA signaling pathway activator, the PKC activator, and the at least one growth factor from the EGF family are removed from the culture medium after the first 1, 2, or 3 days of incubation.

Any γ-secretase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the γ-secretase inhibitor comprises XXI. In some embodiments, the γ-secretase inhibitor comprises DAPT. In some examples, the method comprises contacting NKX6.1-positive pancreatic progenitor cells with a concentration of a γ-secretase inhibitor (e.g., XXI), such as, about 0.01 μM, about 0.02 μM, about 0.05 μM, about 0.075 μM, about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 2.9 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.2 μM, about 5.4 μM, about 5.6 μM, about 5.8 μM, about 6 μM, about 6.2 μM, about 6.4 μM, about 6.6 μM, about 6.8 μM, about 7 μM, about 8 μM, about 9 μM, about 10 μM, about 20 μM, about 30 μM, or about 50 μM. In some examples, the method comprises contacting NKX6.1-positive pancreatic progenitor cells with a concentration of a γ-secretase inhibitor (e.g., XXI), such as, about 1.7-2.3 μM, about 1.8-2.2 μM, or about 1.9-2.1 μM. In some examples, the method comprises contacting NKX6.1-positive pancreatic progenitor cells with a concentration of a γ-secretase inhibitor (e.g., XXI), such as about 2 μM.

Any growth factor from the EGF family capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the at least one growth factor from the EGF family comprises betacellulin. In some embodiments, at least one growth factor from the EGF family comprises EGF. In some examples, the method comprises contacting NKX6.1-positive pancreatic progenitor cells with a concentration of a growth factor from EGF family (e.g., betacellulin), such as, about 1 ng/mL, about 2 ng/mL, about 4 ng/mL, about 6 ng/mL, about 8 ng/mL, about 10 ng/mL, about 12 ng/mL, about 14 ng/mL, about 16 ng/mL, about 18 ng/mL, about 20 ng/mL, about 22 ng/mL, about 24 ng/mL, about 26 ng/mL, about 28 ng/mL, about 30 ng/mL, about 40 ng/mL, about 50 ng/mL, about 75 ng/mL, about 80 ng/mL, about 90 ng/mL, about 95 ng/mL, about 100 ng/mL, about 150 ng/mL, about 200 ng/mL, about 250 ng/mL, or about 300 ng/mL. In some examples, the method comprises contacting NKX6.1-positive pancreatic progenitor cells with a concentration of a growth factor from EGF family (e.g., betacellulin), such as, about 17-23 ng/ml, about 18-22 ng/ml, or about 19-21 ng/ml. In some examples, the method comprises contacting NKX6.1-positive pancreatic progenitor cells with a concentration of a growth factor from EGF family (e.g., betacellulin), such as, about 20 ng/ml.

Any RA signaling pathway activator capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some embodiments, the RA signaling pathway activator comprises RA. In some examples, the method comprises contacting NKX6.1-positive pancreatic progenitor cells with a concentration of an RA signaling pathway activator (e.g., retinoic acid), such as, about 0.02 μM, about 0.05 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting NKX6.1-positive pancreatic progenitor cells with a concentration of an RA signaling pathway activator (e.g., retinoic acid), such as, about 20-80 nM, about 30-70 nM, or about 40-60 nM. In some examples, the method comprises contacting NKX6.1-positive pancreatic progenitor cells with a concentration of an RA signaling pathway activator (e.g., retinoic acid), such as, about 50 nM.

Any SHH pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used in the method provided herein. In some embodiments, the SHH pathway inhibitor comprises Sant1. In some examples, the method comprises contacting NKX6.1-positive pancreatic progenitor cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting NKX6.1-positive pancreatic progenitor cells with a concentration of a SHH pathway inhibitor (e.g., Sant1), such as, about 220-280 nM, about 230-270 nM, about 240-260 nM, or about 245-255 nM. In some examples, the method comprises contacting NKX6.1-positive pancreatic progenitor cells with a concentration of a SHH pathway inhibitor (e.g., Sant1), such as, about 250 nM.

Any BMP signaling pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some embodiments, the BMP signaling pathway inhibitor comprises LDN193189 or DMH-1. In some examples, the method comprises contacting NKX6.1-positive pancreatic progenitor cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 μM. In some examples, the method comprises contacting NKX6.1-positive pancreatic progenitor cells with a concentration of BMP signaling pathway inhibitor (e.g., LDN1931189), such as, about 70-130 nM, about 80-120 nM, about 90-110 nM. In some examples, the method comprises contacting NKX6.1-positive pancreatic progenitor cells with a concentration of BMP signaling pathway inhibitor (e.g., LDN1931189), such as, about 100 nM.

Any ROCK inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the ROCK inhibitor comprises Thiazovivin, Y-27632, Fasudil/HA1077, or H-1152. In some embodiments, the ROCK inhibitor comprises Y-27632. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 2.2-2.8 μM, about 2.3-2.7 μM, or about 2.4-2.6 μM. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 2.5 μM.

Any epigenetic modifying compound that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the epigenetic modifying compound comprises a histone methyltransferase inhibitor or a HDAC inhibitor. In some embodiments, the epigenetic modifying compound comprises a histone methyltransferase inhibitor, e.g., DZNep. In some embodiments, the epigenetic modifying compound comprises a HDAC inhibitor, e.g., KD5170. In some examples, the method comprises contacting PDX1-positive, NKX6.1-positive pancreatic progenitor cells with a concentration of an epigenetic modifying compound (e.g., DZNep or KD5170), such as, about 0.01 μM, about 0.025 μM, about 0.05 μM, about 0.075 μM, about 0.1 μM, about 0.15 μM, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, about 20 μM, about 25 μM, about 30 μM, about 35 μM, about 40 μM, about 50 μM, or about 100 μM. In some examples, the method comprises contacting PDX1-positive, NKX6.1-positive pancreatic progenitor cells with a concentration of an epigenetic modifying compound (e.g., DZNep or KD5170), such as, about 70-130 nM, about 80-120 nM, or about 90-110 nM. In some examples, the method comprises contacting PDX1-positive, NKX6.1-positive pancreatic progenitor cells with a concentration of an epigenetic modifying compound (e.g., DZNep or KD5170), such as, about 100 nM.

Any Wnt signaling pathway inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the Wnt signaling pathway inhibitor comprises a tankyrase inhibitor. In some embodiments, the tankyrase inhibitor is NVP-TNKS656. In some examples, the method comprises contacting PDX1-positive, NKX6.1-positive pancreatic progenitor cells with a concentration of a Wnt signaling pathway inhibitor (e.g., a tankyrase inhibitor such as NVP-TNKS656), such as, about 0.1 μM, about 0.15 μM, about 0.2 μM, about 0.25 μM, about 0.3 μM, about 0.35 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 0.95 μM, about 1 μM, about 1.5 μM, about 2 μM, about 2.5 μM, about 3 μM, about 3.5 μM, about 4 μM, about 4.5 μM, or about 5 μM. In some examples, the method comprises contacting PDX1-positive, NKX6.1-positive pancreatic progenitor cells with a concentration of a Wnt signaling pathway inhibitor (e.g., a tankyrase inhibitor such as NVP-TNKS656), such as, about 1.7-2.3 μM, about 1.8-2.2 μM, or about 1.9-2.1 μM. In some examples, the method comprises contacting PDX1-positive, NKX6.1-positive pancreatic progenitor cells with a concentration of a Wnt signaling pathway inhibitor (e.g., a tankyrase inhibitor such as NVP-TNKS656), such as, about 2 μM.

Any PKC activator that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the PKC activator is TPB or PDBU. In some examples, the method comprises contacting PDX1-positive, NKX6.1-positive pancreatic progenitor cells with a concentration of a PKC activator (TPB or PDBU), such as, about 0.01 μM, about 0.025 μM, about 0.05 μM, about 0.075 μM, about 0.1 μM, about 0.15 μM, about 0.2 μM, about 0.25 μM, about 0.3 μM, about 0.35 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 0.95 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, or about 20 μM. In some examples, the method comprises contacting PDX1-positive, NKX6.1-positive pancreatic progenitor cells with a concentration of a PKC activator (TPB or PDBU), such as, about 450-550 mM, about 475-525 nM, about 490-510 nM, or about 495-505 nM. In some examples, the method comprises contacting PDX1-positive, NKX6.1-positive pancreatic progenitor cells with a concentration of a PKC activator (TPB or PDBU), such as, about 500 nM.

In some embodiments, the population of cells is optionally contacted with a protein kinase inhibitor. In some embodiments, the population of cells is not contacted with the protein kinase inhibitor. In some embodiments, the population of cells is contacted with the protein kinase inhibitor. Any protein kinase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some embodiments, the protein kinase inhibitor comprises staurosporine. In some examples, the method comprises contacting NKX6.1-positive pancreatic progenitor cells with a concentration of a protein kinase inhibitor (e.g., staurosporine), such as, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.1 nM, about 1.2 nM, about 1.3 nM, about 1.4 nM, about 1.5 nM, about 1.6 nM, about 1.7 nM, about 1.8 nM, about 1.9 nM, about 2.0 nM, about 2.1 nM, about 2.2 nM, about 2.3 nM, about 2.4 nM, about 2.5 nM, about 2.6 nM, about 2.7 nM, about 2.8 μM, about 2.9 nM, about 3 nM, about 3.1 nM, about 3.2 nM, about 3.3 nM, about 3.4 nM, about 3.5 nM, about 3.6 nM, about 3.7 nM, about 3.8 nM, about 3.9 nM, about 4.0 nM, about 4.1 nM, about 4.2 nM, about 4.3 nM, about 4.4 nM, about 4.5 nM, about 4.6 nM, about 4.7 nM, about 4.8 μM, about 4.9 nM, or about 5 nM. In some examples, the method comprises contacting NKX6.1-positive pancreatic progenitor cells with a concentration of a protein kinase inhibitor (e.g., staurosporine), such as, about 1-5 nM, about 2-4 nM, or about 2.5-3.5 nM. In some examples, the method comprises contacting NKX6.1-positive pancreatic progenitor cells with a concentration of a protein kinase inhibitor (e.g., staurosporine), such as, about 3 nM.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 89% hydrolyzed.

In some embodiments, the method comprises contacting the population of cells (e.g., NKX6.1-positive pancreatic progenitor cells) with XXI, Alk5i, T3 or GC-1, RA, Sant1, and betacellulin, PDBU, and NVP-TNKS656 for a period of 7 days, to induce the differentiation of at least one NKX6.1-positive pancreatic progenitor cell in the population into an insulin-positive endocrine cell, wherein the insulin-positive endocrine cell expresses insulin. In some embodiments, the method comprises contacting the population of cells (e.g., NKX6.1-positive pancreatic progenitor cells) with XXI, Alk5i, T3 or GC-1, RA, Sant1, betacellulin, and LDN193189 for a period of 7 days, to induce the differentiation of at least one NKX6.1-positive pancreatic progenitor cell in the population into an insulin-positive endocrine cell, wherein the insulin-positive endocrine cell expresses insulin. In some embodiments, one or more differentiation factors are added in a portion of the Stage 5, for instance, only the first 1, 2, 3, 4, 5, or 6 days of the period of time for Stage 5, or the last 1, 2, 3, 4, 5, or 6 days of the period of time for Stage 5. In one example, the cells are contacted with SHH signaling pathway inhibitor the PKC activator, the retinoic acid, and/or the wnt signaling pathway inhibitor for only the first 2, 3, 4, or 5 days during Stage 5, after which the SHH signaling pathway inhibitor, the PKC activator, the retinoic acid, and/or the wnt signaling pathway inhibitor are not included in or removed from the culture medium. In another example, the cells are contacted with BMP signaling pathway inhibitor for only the first 1, 2, or 3 days during Stage 5, after which the BMP signaling pathway inhibitor is removed from the culture medium.

In some embodiments, the method comprises contacting the population of cells (e.g., NKX6.1-positive pancreatic progenitor cells) with one or more metabolites. In some embodiments, the method comprises contacting the population of cells (e.g., NKX6.1-positive pancreatic progenitor cells) with one or more of an acetyl CoA-related metabolite, a vitamin, histone deacetylase inhibitor (HDACi), a redox homeostasis regulator, a one carbon metabolism pathway intermediate, and/or glutamine. Examples of metabolites include glutamine, taurine, acetate, beta-hydroxybutyrate, biotin, and formate.

In some embodiments, a composition (e.g., medium) of the disclosure comprises an acetyl CoA-related metabolite. Exemplary acetyl CoA-related metabolites include, but are not limited to acetate, pyruvate, ketogenic amino acids, valine, leucine, isoleucine, phenylalanine, tyrosine, lysine, tryptophan, fatty acids, CoA, Isovaleryl-CoA, and β-hydroxybutyrate. In some embodiments, the acetyl CoA-related metabolite is acetate. In some embodiments, the acetyl CoA-related metabolite is present in or is added to a composition of the disclosure at a concentration of about 10 nM, about 50 nM, about 80 nM, about 100 nM, about 120 nM, about 140 nM, about 150 nM, about 200 nM, about 300 nM, about 500 nM, about 800 nM, about 1 μM, about 10 μM, about 100 μM, about 500 μM, about 800 μM, about 900 μM, about 1 mM, about 2 mM, about 3 mM, about 5 mM, or about 10 mM. In some embodiments, the acetyl CoA-related metabolite is present in or is added to a composition of the disclosure at a concentration of about 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM. In some embodiments, the acetyl CoA-related metabolite is acetate present at a concentration of about 1 mM. In some embodiments, the acetyl CoA-related metabolite is acetate present at a concentration of about 50-1000 nM, 50-800 nM, 50-500 nM, 50-300 nM, 50-250 nM, 100-200 nM, or 125-175 nM. In some embodiments, the acetyl CoA-related metabolite is acetate present at a concentration of about 160 nM.

In some embodiments, a composition (e.g., medium) of the disclosure comprises one or more vitamins. Exemplary vitamins include, but are not limited to biotin, vitamin B1 (thiamine), vitamin B2 (riboflavin), vitamin B3 (niacin), vitamin B6 (pyridoxine) and vitamin B12 (cyanocobalamin). In some embodiments the vitamin modulates fatty acid synthesis. In some embodiments the vitamin modulates branched-chain amino acid metabolism. In some embodiments the vitamin modulates or participates as a co-factor in the TCA cycle, e.g., as a cofactor for pyruvate carboxylase. In some embodiments, the vitamin is biotin. In some embodiments, the vitamin is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the vitamin is biotin present at a concentration of about 800 nM. In some embodiments, the vitamin is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, a composition (e.g., medium) of the disclosure comprises a histone deacetylase inhibitor (HDACi). Exemplary histone deacetylase inhibitors (HDACi) include, but are not limited to β-Hydroxybutyrate, butyric acid, class I HDACi, class IIA HDACi, class IIB HDACi, class III HDACi, class IV HDACi, HDAC-1, HDAC-2, HDAC-3, HDAC-4, HDAC-5, HDAC-6, HDAC-7, HDAC-8, HDAC-9, HDAC-10, HDAC-11, sirtuins, SIRT1, SIRT2, SIRT3, SIRT4, SIRT5, SIRT6, SIRT7, Vorinostat (suberoylanilide hydroxamic acid, SAHA, MK0683), Entinostat (MS-275, SNDX-275), Panobinostat (LBH589, NVP-LBH589), Trichostatin A (TSA), Mocetinostat (MGCD0103, MG0103), GSK3117391 (GSK3117391A, HDAC-IN-3), BRD3308, BRD3308, Tubastatin A TFA (Tubastatin A trifluoroacetate salt), Tubastatin A, SIS17, NKL 22, BML-210 (CAY10433), TC-H 106, SR-4370, Belinostat (PXD101, NSC726630, PX-105684), Romidepsin (FK228, Depsipeptide, FR 901228, NSC 630176), MC1568, Givinostat (ITF2357), Dacinostat (LAQ824, NVP-LAQ824), CUDC-101, Quisinostat (JNJ-26481585), Pracinostat (SB939), PCI-34051, Droxinostat (NS 41080), Abexinostat (PCI-24781), Abexinostat (PCI-24781, CRA-024781), RGFP966, AR-42 (HDAC-42), Ricolinostat (ACY-1215, Rocilinostat), Valproic acid sodium salt (Sodium valproate), Tacedinaline (CI994, PD-123654, GOE-5549, Acetyldinaline), Fimepinostat (CUDC-907), Sodium butyrate (NaB), Curcumin, Diferuloylmethane, M344, Tubacin, RG2833 (RGFP109), RG2833 (RGFP109), Resminostat (RAS2410), Divalproex Sodium, Scriptaid (GCK 1026), Sodium Phenylbutyrate, Sinapinic acid (Sinapic acid), TMP269, Santacruzamate A (CAY10683), TMP195 (TFMO 2), Valproic acid (VPA), UFO10, Tasquinimod (ABR-215050), SKLB-23bb, Isoguanosine, Sulforaphane, BRD73954, Citarinostat (ACY-241, HDAC-IN-2), Suberohydroxamic acid, Splitomicin, HPOB, LMK-235, Biphenyl-4-sulfonyl chloride (p-Phenylbenzenesulfonyl, 4-Phenylbenzenesulfonyl, p-Biphenylsulfonyl), Nexturastat A, TH34, Tucidinostat (Chidamide, HBI-8000, CS-055), (−)-Parthenolide, WT161, CAY10603, CAY10603, ACY-738, Raddeanin A, Tinostamustine (EDO-S101), Domatinostat (4SC-202), and BG45. In some embodiments, the HDACi is β-Hydroxybutyrate. In some embodiments, the HDACi is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the HDACi is β-Hydroxybutyrate present at a concentration of about 200 nM. In some embodiments, the HDACi is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, a composition (e.g., medium) of the disclosure comprises a redox homeostasis regulator. Exemplary redox homeostasis regulators include, but are not limited to taurine, respiratory chain regulators, free radical scavengers, regulators of mitochondrial protein synthesis, allium sulphur compounds, anthocyanins, beta-carotene, catechins, copper, cryptoxanthins, flavonoids, indoles, isoflavonoids, lignans, lutein, lycopene, alpha lipoic acid, ellagic acid, manganese, polyphenols, selenium, glutathione, vitamin A, vitamin C, vitamin E, zinc, superoxide disutases, GSHPx, Prx-I, catalase, and co-enzyme Q10. In some embodiments, the redox homeostasis regulator is taurine. In some embodiments, the redox homeostasis regulator is present in or is added to a composition of the disclosure at a concentration of about 100 nM, about 500 nM, 1 μM, about 10 μM, about 20 μM, about 30 μM, about 40 μM, about 50 μM, about 60 μM, about 70 μM, about 80 μM, about 90 μM, about 100 μM, about 110 μM, about 110 μM, about 150 μM, or about 200 μM. In some embodiments, the redox homeostasis regulator is taurine. In some embodiments, the redox homeostasis regulator is taurine present at a concentration of about 90 μM. In some embodiments, the redox homeostasis regulator intermediate is present or is added at a concentration of about 100 nM to 1 mM, 500 nM to 1 mM, 1 μM to 1 mM, 10 μM to 1 mM, 20 μM to 1 mM, 30 μM to 1 mM, 30 μM to 1 mM, 40 μM to 1 mM, 50 μM to 1 mM, 60 μM to 1 mM, 70 μM to 1 mM, 80 μM to 1 mM, 100 nM to 250 μM, 500 nM to 250 μM, 1 μM to 250 μM, 10 μM to 250 μM, 20 μM to 250 μM, 30 μM to 250 μM, 30 μM to 250 μM, 40 μM to 250 μM, 50 μM to 250 μM, 60 μM to 250 μM, 70 μM to 250 μM, 100 nM to 100 μM, 500 nM to 100 μM, 1 μM to 100 μM, 10 μM to 100 μM, 20 μM to 100 μM, 30 μM to 100 μM, 40 μM to 100 μM, 50 μM to 100 μM, 60 μM to 100 μM, 70 μM to 100 μM, or 80 μM to 100 μM.

In some embodiments, a composition (e.g., medium) of the disclosure comprises a one carbon metabolism pathway intermediate. Exemplary one carbon metabolism pathway intermediates include, but are not limited to formate, tetrahydrofolate (THF), 10-formylTHF; 5,10-meTHF; 5,10-meTHF; and 10-formylTHF. In some embodiments, the one carbon metabolism pathway intermediate is formate present at a concentration of about 50 μM. In some embodiments, the one carbon metabolism pathway intermediate is present or is added at a concentration of about 100 nM to 1 mM, 500 nM to 1 mM, 1 μM to 1 mM, 10 μM to 1 mM, 20 μM to 1 mM, 30 μM to 1 mM, 100 nM to 250 μM, 500 nM to 250 μM, 1 μM to 250 μM, 10 μM to 250 μM, 20 μM to 250 μM, 30 μM to 250 μM, 100 nM to 100 μM, 500 nM to 100 μM, 1 μM to 100 μM, 10 μM to 100 μM, 20 μM to 100 μM, 30 μM to 100 μM, 100 nM to 60 μM, 500 nM to 60 μM, 1 μM to 60 μM, 10 μM to 60 μM, 20 μM to 60 μM, 30 μM to 60 μM, 40 μM to 60 μM, or 45 μM to 55 μM.

In some embodiments, a composition (e.g., medium) of the disclosure comprises glutamine. Thus in some embodiments, compositions and methods of the disclosure utilize glutamine in a form with increased bioavailability, such as a free glutamine form, such as a non-dipeptide form, a non-alanine-glutamine dipeptide form (e.g., a non-alanyl-1-glutamine form), a non-glycine-glutamine dipeptide form (e.g., a non-glycyl-1-glutamine form), a form that in which glutamine is not conjugated to another amino acid or stabilizing moiety, a monomeric form, a free form, or a combination thereof. In some embodiments, glutamine is provided as a protein hydrolysate. In some embodiments, glutamine is present or is added to a composition of the disclosure at a concentration of from 0.5-20 mM, 0.5-10 mM, 0.5-5 mM, 1-5 mM, 2-5 mM, or 1 mM to 10 mM. In some embodiments, glutamine is present or is added to a composition of the disclosure at a concentration of 3.8-4.2 mM. In some embodiments, glutamine is present or is added to a composition of the disclosure at a concentration of 1-10, 1-7, 1-8, 1-6, 1-5, 1-4, 2-10, 2-7, 2-8, 2-6, 2-5, 2-4, 3-10, 3-7, 3-8, 3-6, 3-5, 3-4, 3.5-4.5, 3.8-4.2, or 3.9-4.1 mM. In some embodiments, glutamine is present or is added to a composition of the disclosure at a concentration of about 4 mM. In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a dipeptide form. In some embodiments, at least 500 μM, at least 750 μM, at least 1 mM, at least 1.5 mM, at least 2 mM, at least 2.5 mM, at least 2.6 mM, at least 2.7 mM, at least 2.8 mM, at least 2.9 mM, at least 3 mM, at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, at least 5 mM, at least 5.5 mM, at least 6 mM, at least 6.5 mM, at least 7 mM, at least 7.5 mM, at least 8 mM, at least 8.5 mM, at least 9 mM, at least 9.5 mM, or at least 10 mM of the glutamine is in a free form.

In some embodiments, the method comprises culturing the population of cells (e.g., NKX6.1-positive pancreatic progenitor cells) in a medium, to induce the differentiation of at least one NKX6.1-positive pancreatic progenitor cell in the population into an insulin-positive endocrine cell, wherein the insulin-positive endocrine cell expresses insulin.

Aspects of the disclosure involve treatment of cell population comprising PDX1-positive, NKX6.1-positive pancreatic progenitor cells with PKC activator and/or Wnt signaling pathway inhibitor, which can lead to increase in percentage of pancreatic α cells, increase in percentage of pancreatic δ cells, increase in percentage of pancreatic β cells, reduction in percentage of EC cells, or any combination thereof, in the cell population of pancreatic endocrine cells generated according to the method disclosed herein.

In some embodiments, the method comprises contacting a population of cells comprising PDX1-positive, NKX6.1-positive pancreatic progenitor cells with a first composition comprising a FOXO1 inhibitor, notch signaling inhibitor, a PKC activator, a ROCK inhibitor, a growth factor from TGFβ superfamily, a growth factor from FGF family, a RA signaling pathway activator, and a SHH pathway inhibitor, for one to two days, thereby obtaining a first transformation cell population comprising PDX1-positive, NKX6.1-positive pancreatic progenitor cells; and contacting the first transformation cell population comprising PDX1-positive, NKX6.1-positive pancreatic progenitor cells with a second composition comprising the PKC activator, notch signaling inhibitor, a TGF-β signaling pathway inhibitor, a TH signaling pathway activator, BMP pathway inhibitor, ROCK inhibitor, retinoic acid, and EGF-family growth factor, wnt signaling pathway inhibitor, and/or an epigenetic modifying compound, for one to two days, thereby obtaining a second transformation cell population comprising NKX6.1-positive, ISL1-positive endocrine cells.

### Pancreatic β Cells

Aspects of the disclosure involve generating pancreatic β cells (e.g., non-native pancreatic β cells). Non-native pancreatic β cells, in some cases, resemble endogenous mature R cells in form and function, but nevertheless are distinct from native β cells.

In some cases, the insulin-positive pancreatic endocrine cells generated using the method provided herein can form a cell cluster, alone or together with other types of cells, e.g., precursors thereof, e.g., stem cell, definitive endoderm cells, primitive gut tube cell, PDX1-positive, NKX6.1-negative pancreatic progenitor cells, or PDX1-positive, NKX6.1-positive pancreatic progenitor cells.

In some cases, the cell population comprising the insulin-positive endocrine cells can be directly induced to mature into SC-β cells without addition of any exogenous differentiation factors (such as inhibitor of TGF-β signaling pathway, thyroid hormone signaling pathway activator, PKC activator, growth factors from TGF-β superfamily, FGF family, or EGF family, SHH signaling pathway inhibitor, γ-secretase inhibitor, ROCK inhibitor, or BMP signaling pathway inhibitor). In some embodiments, the method provided herein comprises contacting a cell population comprising NKX6.1-positive, ISL1-positive endocrine cells with a TGF-β signaling pathway inhibitor, a SHH pathway inhibitor, a thyroid hormone signaling pathway activator, a protein kinase inhibitor, a ROCK inhibitor, a BMP signaling pathway inhibitor, and/or an epigenetic modifying compound. In some embodiments, the method provided herein comprises contacting a cell population comprising NKX6.1-positive, ISL1-positive endocrine cells with human serum albumin protein. In some embodiments, the method provided herein comprises contacting a cell population comprising NKX6.1-positive, ISL1-positive endocrine cells with a PKC activator.

In some examples, insulin-positive endocrine cells can be matured in a NS-GFs medium, MCDB131 medium, DMEM medium, or CMRL medium. In some cases, the insulin-positive endocrine cells can be matured in a CMRLs medium supplemented with 10% FBS. In some cases, the insulin-positive endocrine cells can be matured in a DMEM/F12 medium supplemented with 0.01-1% HSA (e.g., 0.05% HSA). In some cases, the HSA is substituted with a water-soluble synthetic polymer. In some cases, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 89% hydrolyzed. In other cases, SC-β cells can be obtained by culturing the population of cells containing the insulin-positive endocrine cells in a MCDB131 medium that can be supplemented by 2% BSA. In some cases, the MCDB131 medium with 2% BSA for maturation of insulin-positive endocrine cells into SC-β cells can be comprise no small molecule factors as described herein. In some case, the MCDB131 medium with 2% BSA for maturation of insulin-positive endocrine cells into SC-β cells can comprise no serum (e.g., no FBS). In other cases, SC-β cells can be obtained by culturing the population of cells containing the insulin-positive endocrine cells in a MCDB131 medium that can be supplemented by 0.05% HSA and vitamin C. In some cases, SC-β cells can be obtained by culturing the population of cells containing the insulin-positive endocrine cells in a MCDB131 medium that can be supplemented by 0.05% HSA, ITS-X, vitamin C, and glutamine (Gln, e.g., 4 mM). In some cases, the type of culture medium may be changed during S6. For instance, the S6 cells are cultured in a MCDB131 medium that can be supplemented by 0.05% HSA and vitamin C for the first two to four days, and then followed by a DMEM/F12 medium supplemented with 1% HSA. In some cases, additional factors are introduced into the culture medium. For instance, S6 cells can be cultured in a MCDB131 medium that can be supplemented by 0.05% HSA, ITS-X, vitamin C, and glutamine (Gln, e.g., 4 mM) throughout the 4-12 days, during which ZnSO₄ is introduced from day 4 of S6.

In some aspects, the disclosure provides a method of generating SC-β cells from pluripotent cells, the method comprising: a) differentiating pluripotent stem cells in a population into definitive endoderm cells by contacting the pluripotent stem cells with at least a WNT signaling pathway activator for 1 day and one factor from TGFβ superfamily and optionally one or more inhibitors of PI3K/Akt/mTOR signaling for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive pancreatic progenitor cells by a process of contacting the primitive gut tube cells with a BMP signaling pathway inhibitor (e.g., DMH-1 or LDN193189) for one day and with i) retinoic acid signaling pathway activator, ii) at least one factor from the FGF family, iii) a SHH pathway inhibitor, iv) a PKC activator, v) a ROCK inhibitor, vi) one factor from TGFβ superfamily and vii) optionally one or more inhibitors of PI3K/Akt/mTOR signaling for 2 days; d) differentiating at least some of the PDX1-positive pancreatic progenitor cells into PDX1-positive, NKX6.1-positive pancreatic progenitor cells by a process of contacting the PDX1-positive pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, and optionally vi) one or more inhibitors of PI3K/Akt/mTOR signaling for a period of 6 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) at least one SHH pathway inhibitor, ii) a RA signaling pathway activator, iii) at least one growth factor from the epidermal growth factor (EGF) family, iv) a PKC activator for a period of 2-3 days, and v) a TGF-β signaling pathway inhibitor, vi) a TH signaling pathway activator, vii) a γ-secretase inhibitor, optionally viii) a BMP signaling pathway inhibitor, ix) a ROCK inhibitor, x) a protein kinase inhibitor (e.g., staurosporine), and xi) an epigenetic modifier (e.g., DZNEP), for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium), for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor, v) a PKC activator, vi) a ROCK inhibitor, and vii) a growth factor from TGFβ superfamily, for a period of 2 days; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a TGF-β signaling pathway inhibitor, ii) a TH signaling pathway activator, iii) at least one SHH pathway inhibitor, iv) a RA signaling pathway activator, v) a γ-secretase inhibitor, optionally vi) at least one growth factor from the epidermal growth factor (EGF) family, and optionally vii) a BMP signaling pathway inhibitor, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium) without exogenous differentiation factors, for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a PKC activator, and v) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a TGF-β signaling pathway inhibitor, ii) a TH signaling pathway activator, iii) at least one SHH pathway inhibitor, iv) a RA signaling pathway activator, v) a γ-secretase inhibitor, and optionally vi) at least one growth factor from the epidermal growth factor (EGF) family, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium) without exogenous differentiation factors, for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 or 6 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound (e.g., DZNep or KD5170), ix) a protein kinase inhibitor, and x) a ROCK inhibitor, for a period of between five and seven days; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium) without exogenous differentiation factors, for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells under conditions that promote cell clustering with i) at least one growth factor from the FGF family, ii) at least one SHH pathway inhibitor, and optionally iii) a RA signaling pathway activator, and optionally iv) ROCK inhibitor and v) at least one factor from TGFβ superfamily, for a period of 5 or 6 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a γ-secretase inhibitor, ii) at least one bone morphogenetic protein (BMP) signaling pathway inhibitor, iii) a TGF-β signaling pathway inhibitor, iv) a thyroid hormone signaling pathway activator, v) an epigenetic modifying compound (e.g., DZNep or KD5170), vi) a protein kinase inhibitor, and vii) a ROCK inhibitor, for a period of between five and seven days, and within first three days of the period of between five and seven days, contacting the PDX1-positive, NKX6.1-positive pancreatic progenitor cells with a SHH pathway inhibitor, a RA signaling pathway, and at least one growth factor from the EGF family, which are removed from the PDX1-positive, NKX6.1-positive pancreatic progenitor cells thereafter; and f) differentiating at least some of the PDX1-positive, NKX6.1-positive, insulin-positive endocrine cells into SC-β cells by a process of culturing the PDX1-positive, NKX6.1-positive, insulin-positive endocrine cells in a medium (e.g., NS-GFs medium, MCDB medium supplemented with BSA, MCDB131 medium, or DMEM/F12 medium) without exogenous differentiation factors, for a period of between 7 and 14 days to induce the in vitro maturation of at least some of the PDX1-positive, NKX6.1-positive, insulin-positive endocrine cells into SC-β cells, wherein the SC-β cells exhibit a GSIS response in vitro and/or in vivo. In some cases, the GSIS response resembles the GSIS response of an endogenous mature β cells.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 89% hydrolyzed.

In some aspects, the disclosure provides a method of generating SC-β cells from pluripotent cells, the method comprising: a) differentiating pluripotent stem cells in a population into definitive endoderm cells by contacting the pluripotent stem cells with at least one factor from TGFβ superfamily and a WNT signaling pathway activator for a period of 3 days; b) differentiating at least some of the definitive endoderm cells into primitive gut tube cells by a process of contacting the definitive endoderm cells with at least one factor from the FGF family for a period of 3 days; c) differentiating at least some of the primitive gut tube cells into PDX1-positive, NKX6.1-negative pancreatic progenitor cells by a process of contacting the primitive gut tube cells with i) retinoic acid signaling pathway activator, ii) at least one factor from the FGF family, iii) a SHH pathway inhibitor, iv) a BMP signaling pathway inhibitor (e.g., DMH-1 or LDN193189), v) a PKC activator, and vi) a ROCK inhibitor; d) differentiating at least some of the PDX1-positive, NKX6.1-negative pancreatic progenitor cells into PDX1-positive, NKX6.1-positive pancreatic progenitor cells by a process of contacting the PDX1-positive, NKX6.1-negative pancreatic progenitor cells under conditions that promote cell clustering with i) at least one SHH pathway inhibitor, and optionally ii) a RA signaling pathway activator, and optionally iii) ROCK inhibitor and v) at least one factor from TGFβ superfamily, for a period of 5 or 6 days; e) differentiating at least some of the PDX1-positive, NKX6.1-positive pancreatic progenitor cells into PDX1-positive, NKX6.1-positive, insulin-positive endocrine cells by a process of contacting the PDX1-positive, NKX6.1-positive pancreatic progenitor cells with i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound (e.g., DZNep or KD5170), ix) a protein kinase inhibitor, and x) a ROCK inhibitor, for a period of between five and seven days; and f) differentiating at least some of the PDX1-positive, NKX6.1-positive, insulin-positive endocrine cells into SC-β cells.

The medium used to culture the cells dissociated from the first cell cluster can be xeno-free. A xeno-free medium for culturing cells and/or cell clusters of originated from an animal can have no product from other animals. In some cases, a xeno-free medium for culturing human cells and/or cell clusters can have no products from any non-human animals. For example, a xeno-free medium for culturing human cells and/or cell clusters can comprise human platelet lysate (PLT) instead of fetal bovine serum (FBS). For example, a medium can comprise from about 1% to about 20%, from about 5% to about 15%, from about 8% to about 12%, from about 9 to about 11% serum. In some cases, medium can comprise about 10% of serum. In some cases, the medium can be free of small molecules and/or FBS. For example, a medium can comprise MCDB131 basal medium supplemented with 2% BSA. In some cases, the medium is serum-free. In some examples, a medium can comprise no exogenous small molecules or signaling pathway agonists or antagonists, such as, growth factor from fibroblast growth factor family (FGF, such as FGF2, FGF8B, FGF 10, or FGF21), Sonic Hedgehog Antagonist (such as Sant1, Sant2, Sant4, Sant4, Cur61414, forskolin, tomatidine, AY9944, triparanol, cyclopamine, or derivatives thereof), Retinoic Acid Signaling agonist (e.g., retinoic acid, CD1530, AM580, TTHPB, CD437, Ch55, BMS961, AC261066, AC55649, AM80, BMS753, tazarotene, adapalene, or CD2314), inhibitor of Rho-associated, coiled-coil containing protein kinase (ROCK) (e.g., Thiazovivin, Y-27632, Fasudil/HA1077, or 14-1152), activator of protein kinase C (PKC) (e.g., phorbol 12,13-dibutyrate (PDBU), TPB, phorbol 12-myristate 13-acetate, bryostatin 1, or derivatives thereof), antagonist of TGF R super family (e.g., Alk5 inhibitor II (CAS 446859-33-2), A83-01, SB431542, D4476, GW788388, LY364947, LY580276, SB505124, GW6604, SB-525334, SD-208, SB-505124, or derivatives thereof), inhibitor of Bone Morphogenetic Protein (BMP) type 1 receptor (e.g., LDN193189 or derivatives thereof), thyroid hormone signaling pathway activator (e.g., T3, GC-1 or derivatives thereof), gamma-secretase inhibitor (e.g., XXI, DAPT, or derivatives thereof), activator of TGF-β signaling pathway (e.g., WNT3a or Activin A) growth factor from epidermal growth factor (EGF) family (e.g., betacellulin or EGF), broad kinase (e.g., staurosporine or derivatives thereof), non-essential amino acids, vitamins or antioxidants (e.g., cyclopamine, vitamin D, vitamin C, vitamin A, or derivatives thereof), or other additions like N-acetyl cysteine, zinc sulfate, or heparin. In some cases, the reaggregation medium can comprise no exogenous extracellular matrix molecule. In some cases, the reaggregation medium does not comprise Matrigel™. In some cases, the reaggregation medium does not comprise other extracellular matrix molecules or materials, such as, collagen, gelatin, poly-L-lysine, poly-D-lysine, vitronectin, laminin, fibronectin, PLO laminin, fibrin, thrombin, and RetroNectin and mixtures thereof, for example, or lysed cell membrane preparations.

A person of ordinary skill in the art will appreciate that that the concentration of serum albumin supplemented into the medium may vary. For example, a medium (e.g., MCDB131) can comprise about 0.01%, 0.05%, 0.1%, 1%, about 2%, about 3%, about 4%, about 5%, about 10%, or about 15% BSA. In other cases, a medium can comprise about 0.01%, 0.05%, 0.1%, 1%, about 2%, about 3%, about 4%, about 5%, about 10%, or about 15% HSA. The medium used (e.g., MCDB131 medium) can contain components not found in traditional basal media, such as trace elements, putrescine, adenine, thymidine, and higher levels of some amino acids and vitamins. These additions can allow the medium to be supplemented with very low levels of serum or defined components. The medium can be free of proteins and/or growth factors, and may be supplemented with EGF, hydrocortisone, and/or glutamine. The medium can comprise one or more extracellular matrix molecules (e.g., extracellular proteins). Non-limiting exemplary extracellular matrix molecules used in the medium can include collagen, placental matrix, fibronectin, laminin, merosin, tenascin, heparin, heparin sulfate, chondroitin sulfate, dermatan sulfate, aggrecan, biglycan, thrombospondin, vitronectin, and decorin. In some cases, the medium comprises laminin, such as LN-332. In some cases, the medium comprises heparin.

The medium can be changed periodically in the culture, e.g., to provide optimal environment for the cells in the medium. When culturing the cells dissociated from the first cell cluster for re-aggregation, the medium can be changed at least or about every 4 hours, 12 hours, 24 hours, 48 hours, 3 days or 4 days. For example, the medium can be changed about every 48 hours.

In some cases, cells can be cultured under dynamic conditions (e.g., under conditions in which the cells are subject to constant movement or stirring while in the suspension culture). For dynamic culturing of cells, the cells can be cultured in a container (e.g., an non-adhesive container such as a spinner flask (e.g., of 200 ml to 3000 ml, for example 250 ml; of 100 ml; or in 125 ml Erlenmeyer), or a dynamic large-scale bioreactor (e.g., 500 mL to 50 L, e.g., 1 L to 10 L, 2 L to 5 L, 3 L to 4 L, 2 L to 30 L, or 10 L to 20 L), which can be connected to a control unit and thus present a controlled culturing system. In some cases, cells can be cultured under non-dynamic conditions (e.g., a static culture) while preserving their proliferative capacity. For non-dynamic culturing of cells, the cells can be cultured in an adherent culture vessel. An adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, PLO laminin, fibrin, thrombin, and RetroNectin and mixtures thereof, for example, Matrigel™, and lysed cell membrane preparations.

In some cases, the cell differentiation process disclosed herein is conducted in suspension cell culture that has a liquid volume of about 500 mL to about 50 L, e.g., about 1 L to about 10 L, about 2 L to about 5 L, about 3 L to about 4 L, about 2 L to about 30 L, or about 10 L to about 20 L. In some cases, the cell differentiation process disclosed herein is conducted in suspension cell culture that has a liquid volume of about 10 mL to about 1000 mL, e.g., about 10 mL to about 100 mL, about 20 mL to about 50 mL, about 30 mL to about 40 mL, about 20 mL to about 30 mL, or about 10 mL to about 20 mL.

Medium in a dynamic cell culture vessel (e.g., a spinner flask) can be stirred (e.g., by a stirrer). The spinning speed can correlate with the size of the re-aggregated second cell cluster. The spinning speed can be controlled so that the size of the second cell cluster can be similar to an endogenous pancreatic islet. In some cases, the spinning speed is controlled so that the size of the second cell cluster can be from about 75 μm to about 250 μm. The spinning speed of a dynamic cell culture vessel (e.g., a spinner flask) can be about 20 rounds per minute (rpm) to about 100 rpm, e.g., from about 30 rpm to about 90 rpm, from about 40 rpm to about 60 rpm, from about 45 rpm to about 50 rpm. In some cases, the spinning speed can be about 50 rpm.

Stage 6 cells as provided herein may or may not be subject to the dissociation and reaggregation process as described herein. In some cases, the cell cluster comprising the insulin-positive endocrine cells can be reaggregated. The reaggregation of the cell cluster can enrich the insulin-positive endocrine cells. In some cases, the insulin-positive endocrine cells in the cell cluster can be further matured into pancreatic β cells. For example, after reaggregation, the second cell cluster can exhibit in vitro GSIS, resembling native pancreatic islet. For example, after reaggregation, the second cell cluster can comprise non-native pancreatic β cell that exhibits in vitro GSIS. In some embodiments, the reaggregation process can be performed according to the disclosure of U.S. Patent Publication No. US20200332262A1, which is incorporated herein by reference in its entirety.

Stage 6 cells obtained according to methods provided herein can have high recovery yield after cryopreservation and reaggregation procedures. In some cases, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a higher recovery yield after cryopreservation post stage 5, as compared to a corresponding cell population without such treatment. In some cases, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a higher recovery yield after cryopreservation post stage 5, as compared to a corresponding cell population without treatment of a BMP signaling pathway inhibitor (e.g., DMH-1 or LDN) and a growth factor from TGF-β superfamily (e.g., Activin A) at stage 3. In some cases, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a recovery yield after cryopreservation post stage 5 that is at least about 35%, 37.5%, 40%, 42.5%, 45%, 47.5%, 48%, 49%, or 50%. The recovery yield can be calculated as a percentage of cells that survive and form reaggregated cell clusters after cryopreservation, thawing and recovery, and reaggregation procedures, as compared to the cells before the cryopreservation.

In some embodiments, the present disclosure relates to cryopreservation of the non-native pancreatic β cells or precursors thereof (e.g., NKX6.1-positive, PDX1-positive, insulin-positive cells obtained following stage 5) obtained using the methods provided herein. In some embodiments, the cell population comprising non-native pancreatic β cells can be stored via cryopreservation. For instances, the cell population comprising non-native β cells, e.g., Stage 6 cells in some cases, can be dissociated into cell suspension, e.g., single cell suspension, and the cell suspension can be cryopreserved, e.g., frozen in a cryopreservation solution. The dissociation of the cells can be conducted by any of the technique provided herein, for example, by enzymatic treatment. The cells can be frozen at a temperature of at highest −20° C., at highest −30° C., at highest −40° C., at highest −50° C., at highest −60° C., at highest −70° C., at highest −80° C., at highest −90° C., at highest −100° C., at highest −110° C., at highest −120° C., at highest −130° C., at highest −140° C., at highest −150° C., at highest −160° C., at highest −170° C., at highest −180° C., at highest −190° C., or at highest −200° C. In some cases, the cells are frozen at a temperature of about −80° C. In some cases, the cells are frozen at a temperature of about −195° C. Any cooling methods can be used for providing the low temperature needed for cryopreservation, such as, but not limited to, electric freezer, solid carbon dioxide, and liquid nitrogen. In some cases, any cryopreservation solution available to one skilled in the art can be used for incubating the cells for storage at low temperature, including both custom made and commercial solutions. For example, a solution containing a cryoprotectant can be used. The cryoprotectant can be an agent that is configured to protect the cell from freezing damage. For instance, a cryoprotectant can be a substance that can lower the glass transition temperature of the cryopreservation solution. Exemplary cryoprotectants that can be used include DMSO (dimethyl sulfoxide), glycols (e.g., ethylene glycol, propylene glycol and glycerol), dextran (e.g., dextran-40), and trehalose. Additional agents can be added into the cryopreservation solution for other effects. In some cases, commercially available cryopreservation solutions can be used in the method provided herein, for instance, FrostaLife™, pZerve™, Prime-XV®, Gibco Synth-a-Freeze Cryopreservation Medium, STEM-CELLBANKER®, CryoStor® Freezing Media, HypoThermosol® FRS Preservation Media, and CryoDefend® Stem Cells Media.

During the differentiation process, the cells can be subject to irradiation treatment as provided herein. In some cases, the cell population at Stage 6, e.g., the cell population or cell cluster that has cells being differentiated from insulin-positive endocrine cells into pancreatic β cells, is irradiated for a period of time. In some cases, the cell population at Stage 6 after reaggregation following the recovery from cryopreservation is irradiated for a period of time. In some cases, the cryopreserved cells (e.g., the cells that are cryopreserved at the end of Stage 5) are irradiated for a certain period of time prior to thawing and recovery for subsequent differentiation process.

In some embodiments, the stage 6 cells comprise NKX6.1-positive, insulin-positive cells. In some embodiments, the stage 6 cells comprise NKX6.1-positive, insulin-negative cells. In some embodiments, the stage 6 cells comprise C-peptide positive cells. In some embodiments, Stage 6 cells or cells that have characteristics of stage 6 cells are incubated in NS-GFs medium, MCDB131 medium, DMEM medium, or CMRL medium. In some embodiments, the stage 6 cells or cells that have characteristics of stage 6 cells are contacted with any one or more of a vitamin or anti-oxidant (e.g., vitamin C), a human serum albumin protein, a TGF-beta pathway inhibitor (e.g., an ALK5 inhibitor II), a bone morphogenic protein (BMP) type 1 receptor inhibitor (e.g., LDN193189), a Rho-associated coiled-coil containing protein kinase (ROCK) inhibitor (e.g., thiazovivin), a histone methyltransferase inhibitor (e.g., DZNEP), and a protein kinase inhibitor (e.g., staurosporine). In some embodiments, the stage 6 cells are contacted with a PKC activator (see, e.g., U.S. Patent Publication No. US20210214690A1, which is incorporated by reference herein in its entirety). In some embodiments, the stage 6 cells are not contacted with a PKC activator.

In some embodiments, the disclosure provides for a composition comprising a population of insulin-positive cells and a lipid. In some embodiments, the disclosure provides for a method of contacting a population of insulin-positive cells with a lipid. In some embodiments, the lipid is a saturated fatty acid. In some embodiments, the saturated fatty acid is palmitate. In some embodiments, the lipid is a unsaturated fatty acid. In some embodiments, the non-saturated fatty acid is oleic acid, linoleic acid, or palmitoleic acid.

In some embodiments, the disclosure provides for a composition comprising a population of insulin-positive cells and MCDB 131. In some embodiments, the disclosure provides for a method of contacting a population of insulin-positive cells with MCDB 131. In some embodiments, the disclosure provides for a composition comprising a population of insulin-positive cells and DMEM/F12. In some embodiments, the disclosure provides for a method of contacting a population of insulin-positive cells with DMEM/F12. In some embodiments, the disclosure provides for a composition comprising a population of insulin-positive cells and zinc. In some embodiments, the disclosure provides for a method of contacting a population of insulin-positive cells with zinc. In some embodiments, the disclosure provides for a composition comprising a population of insulin-positive cells and ZnSO₄. In some embodiments, the disclosure provides for a method of contacting a population of insulin-positive cells with ZnSO₄.

In some embodiments, the disclosure provides for a composition comprising a population of insulin-positive cells and at least one metabolite. In some embodiments, the disclosure provides for a method of contacting a population of insulin-positive cells with at least one metabolite. In some embodiments, the at least one metabolite is glutamate, acetate, b-hydroxybutarate, L-carnitine, taurine, formate, or biotin. In some embodiments, the disclosure provides for a composition comprising a population of insulin-positive cells and one, two, three, four, five, six, or seven of glutamate, acetate, β-hydroxybutarate, L-carnitine, taurine, formate, or biotin. In some embodiments, the disclosure provides for a method of contacting a population of insulin-positive cells with one, two, three, four, five, six, or seven of glutamate, acetate, β-hydroxybutarate, L-carnitine, taurine, formate, or biotin.

In some embodiments, the disclosure provides for a composition comprising a population of insulin-positive cells and at least one amino acid. In some embodiments, the disclosure provides for a method of contacting a population of insulin-positive cells with at least one amino acid. In some embodiments, the at least one amino acid is alanine, glutamate, glycine, proline, threonine, or tryptophan. In some embodiments, the at least one amino acid is arginine, histidine, lysine, aspartic acid, glutamic acid, serine, asparagine, glutamine, cysteine, selenocysteine, alanine, valine, isoleucine, leucine, methionine, phenylalanine, tyrosine, glutamate, glycine, proline, threonine, or tryptophan. In some embodiments, the disclosure provides for a composition comprising a population of insulin-positive cells and at least one vitamin. In some embodiments, the disclosure provides for a method of contacting a population of insulin-positive cells with at least one vitamin. In some embodiments, the at least one vitamin is biotin or riboflavin.

In some embodiments, the disclosure provides for a composition comprising a population of insulin-positive cells and a monoglyceride lipase (MGLL) inhibitor. In some embodiments, the disclosure provides for a method of contacting a population of insulin-positive cells with at least one vitamin. In some embodiments, the MGLL inhibitor is any of JJKK048, KML29, NF1819, JW642, JZL184, JZL195, JZP361, pristimerin, or URB602, or derivatives thereof.

### Differentiation Factors

Aspects of the disclosure relate to contacting progenitor cells (e.g., stem cells, e.g., iPS cells, definitive endoderm cells, primitive gut tube cells, PDX1-positive, NKX6.1-negative pancreatic progenitor cells, PDX1-positive, NKX6.1-positive pancreatic progenitor cells, insulin-positive endocrine cells) with one or more β cell differentiation factors, for example, to induce the maturation of the insulin-positive endocrine cells or differentiation of other progenitor cells into SC-β cells (e.g., mature pancreatic β cells). In some embodiments, the differentiation factor(s) can induce the differentiation of pluripotent cells (e.g., iPSCs or hESCs) into definitive endoderm cells, e.g., in accordance with a method described herein. In some embodiments, the differentiation factor(s) can induce the differentiation of definitive endoderm cells into primitive gut tube cells, e.g., in accordance with a method described herein. In some embodiments, the differentiation factor(s) can induce the differentiation of primitive gut tube cells into PDX1-positive, NKX6.1-negative pancreatic progenitor cells, e.g., in accordance with a method described herein. In some embodiments, the differentiation factor(s) can induce the differentiation of PDX1-positive, NKX6.1-negative pancreatic progenitor cells into NKX6-1-positive pancreatic progenitor cells, e.g., in accordance with a method described herein. In some embodiments, the differentiation factor(s) can induce the differentiation of NKX6-1-positive pancreatic progenitor cells into insulin-positive endocrine cells, e.g., in accordance with a method described herein. In some embodiments, the differentiation factor(s) can induce the maturation of insulin-positive endocrine cells into SC-β cells, e.g., in accordance with a method described herein.

At least one differentiation factor described herein can be used alone, or in combination with other differentiation actors, to generate SC-β cells according to the methods as disclosed herein. In some embodiments, at least two, at least three, at least four, at least five, at least six, at least seven, at least eight, at least nine, or at least ten differentiation factors described herein are used in the methods of generating SC-β cells.

### Transforming Growth Factor-β (TGF-β) Superfamily

Aspects of the disclosure relate to the use of growth factors from the transforming growth factor-β (TGF-β) superfamily as differentiation factors. The “TGF-β superfamily” means proteins having structural and functional characteristics of known TGFβ family members. The TGFβ family of proteins can include the TGFβ series of proteins, the Inhibins (including Inhibin A and Inhibin B), the Activins (including Activin A, Activin B, and Activin AB), MIS (Müllerian inhibiting substance), BMP (bone morphogenetic proteins), dpp (decapentaplegic), Vg-1, MNSF (monoclonal nonspecific suppressor factor), and others. Activity of this family of proteins can be based on specific binding to certain receptors on various cell types. Members of this family can share regions of sequence identity, particularly at the C-terminus, that correlate to their function. The TGFβ family can include more than one hundred distinct proteins, all sharing at least one region of amino acid sequence identity. Members of the family that can be used in the method disclosed herein can include, but are not limited to, the following proteins, as identified by their GenBank accession numbers: P07995, P18331, P08476, Q04998, P03970, P43032, P55102, P27092, P42917, P09529, P27093, P04088, Q04999, P17491, P55104, Q9WUK5, P55103, O88959, O08717, P58166, O61643, P35621, P09534, P48970, Q9NR23, P25703, P30884, P12643, P49001, P21274, O46564, O19006, P22004, P20722, Q04906, Q07104, P30886, P18075, P23359, P22003, P34821, P49003, Q90751, P21275, Q06826, P30885, P34820, Q29607, P12644, Q90752, O46576, P27539, P48969, Q26974, P07713, P91706, P91699, P27091, O42222, Q24735, P20863, O18828, P55106, Q9PTQ2, O14793, O08689, O42221, O18830, O18831, O18836, O35312, O42220, P43026, P43027, P43029, O95390, Q9R229, O93449, Q9Z1W4, Q9BDW8, P43028, Q7Z4P5, P50414, P17246, P54831, P04202, P01137, P09533, P18341, O19011, Q9Z1Y6, P07200, Q9Z217, O95393, P55105, P30371, Q9MZE2, Q07258, Q96S42, P97737, AAA97415.1, NP-776788.1, NP-058824.1, EAL24001.1, 1 S4Y, NP-001009856.1, NP-1-032406.1, NP-999193.1, XP-519063.1, AAG17260.1, CAA40806.1, NP-1-001009458.1, AAQ55808.1, AAK40341.1, AAP33019.1, AAK21265.1, AAC59738.1, CA146003.1, B40905, AAQ55811.1, AAK40342.1, XP-540364.1, P55102, AAQ55810.1, NP-990727.1, CAA51163.1, AAD50448.1, JC4862, PN0504, BAB17600.1, AAH56742.1, BAB17596.1, CAG06183.1, CAG05339.1, BAB17601.1, CAB43091.1, A36192, AAA49162.1, AAT42200.1, NP-789822.1, AAA59451.1, AAA59169.1, XP-541000.1, NP-990537.1, NP-1-002184.1, AAC14187.1, AAP83319.1, AAA59170.1, BAB16973.1, AAM66766.1, WFPGBB, 1201278C, AAH30029.1, CAA49326.1, XP-344131.1, AA-148845.1, XP-1-148966.3, 148235, B41398, AAH77857.1, AAB26863.1, 1706327A, BAA83804.1, NP-571143.1, CAG00858.1, BAB17599.1, BAB17602.1, AAB61468.1, PN0505, PN0506, CAB43092.1, BAB17598.1, BAA22570.1, BAB16972.1, BAC81672.1, BAA12694.1, BAA08494.1, B36192, C36192, BAB16971.1, NP-034695.1, AAA49160.1, CAA62347.1, AAA49161.1, AAD30132.1, CAA58290.1, NP-005529.1, XP-522443.1, AAM27448.1, XP-538247.1, AAD30133.1, AAC36741.1, AAH10404.1, NP-032408.1, AAN03682.1, XP-509161.1, AAC32311.1, NP-651942.2, AAL51005.1, AAC39083.1, AAH85547.1, NP-571023.1, CAF94113.1, EAL29247.1, AAW30007.1, AAH90232.1, A29619, NP-001007905.1, AAH73508.1, AADO2201.1, NP-999793.1, NP-990542.1, AAF19841.1, AAC97488.1, AAC60038.1, NP 989197.1, NP-571434.1, EAL41229.1, AAT07302.1, CA119472.1, NP-031582.1, AAA40548.1, XP-535880.1, NP-1-037239.1, AAT72007.1, XP-418956.1, CAA41634.1, BAC30864.1, CAA38850.1, CAB81657.2, CAA45018.1, CAA45019.1, BAC28247.1, NP-031581.1, NP-990479.1, NP-999820.1, AAB27335.1, S45355, CAB82007.1, XP-534351.1, NP-058874.1, NP-031579.1, 1REW, AAB96785.1, AAB46367.1, CAA05033.1, BAA89012.1, IES7, AAP20870.1, BAC24087.1, AAG09784.1, BAC06352.1, AAQ89234.1, AAM27000.1, AAH30959.1, CAGO1491.1, NP-571435.1, 1REU, AAC60286.1, BAA24406.1, A36193, AAH55959.1, AAH54647.1, AAH90689.1, CAG09422.1, BAD16743.1, NP-032134.1, XP-532179.1, AAB24876.1, AAH57702.1, AAA82616.1, CAA40222.1, CAB90273.2, XP-342592.1, XP-534896.1, XP-534462.1, 1LXI, XP-417496.1, AAF34179.1, AAL73188.1, CAF96266.1, AAB34226.1, AAB33846.1, AAT12415.1, AA033819.1, AAT72008.1, AAD38402.1, BAB68396.1, CAA45021.1, AAB27337.1, AAP69917.1, AAT12416.1, NP-571396.1, CAA53513.1, AA033820.1, AAA48568.1, BAC02605.1, BAC02604.1, BAC02603.1, BAC02602.1, BAC02601.1, BAC02599.1, BAC02598.1, BAC02597.1, BAC02595.1, BAC02593.1, BAC02592.1, BAC02590.1, AAD28039.1, AAP74560.1, AAB94786.1, NP-001483.2, XP-528195.1, NP-571417.1, NP-001001557. I, AAH43222.1, AAM33143.1, CAG10381.1, BAA31132.1, EAL39680.1, EAA12482.2, P34820, AAP88972.1, AAP74559.1, CA116418.1, AAD30538.1, XP-345502.1, NP-1-038554.1, CAG04089.1, CAD60936.2, NP-031584.1, B55452, AAC60285.1, BAA06410.1, AAH52846.1, NP-031580.1, NP-1-036959.1, CAA45836.1, CAA45020.1, Q29607, AAB27336.1, XP-547817.1, AAT12414.1, AAM54049.1, AAH78901.1, AA025745.1, NP-570912.1, XP-392194.1, AAD20829.1, AAC97113.1, AAC61694.1, AAH60340.1, AAR97906.1, BAA32227.1, BAB68395.1, BAC02895.1, AAWS 1451.1, AAF82188.1, XP-544189.1, NP-990568.1, BAC80211.1, AAW82620.1, AAF99597.1, NP-571062.1, CAC44179.1, AAB97467.1, AAT99303.1, AAD28038.1, AAH52168.1, NP-001004122.1, CAA72733.1, NP-032133.2, XP-394252.1, XP-224733.2, JH0801, AAP97721.1, NP-989669.1, S43296, P43029, A55452, AAH32495.1, XP-542974.1, NP-032135.1, AAK30842.1, AAK27794.1, BAC30847.1, EAA12064.2, AAP97720.1, XP-525704.1, AAT07301.1, BAD07014.1, CAF94356.1, AAR27581.1, AAG13400.1, AAC60127.1, CAF92055.1, XP-540103.1, AA020895.1, CAF97447.1, AAS01764.1, BAD08319.1, CAA10268.1, NP-998140.1, AAR03824.1, AAS48405.1, AAS48403.1, AAK53545.1, AAK84666.1, XP-395420.1, AAK56941.1, AAC47555.1, AAR88255.1, EAL33036.1, AAW47740.1, AAW29442.1, NP-722813.1, AAR08901.1, AAO 15420.2, CAC59700.1, AAL26886.1, AAK71708.1, AAK71707.1, CAC51427.2, AAK67984.1, AAK67983.1, AAK28706.1, P07713, P91706, P91699, CAG02450.1, AAC47552.1, NP-005802.1, XP-343149.1, AW34055.1, XP-538221.1, AAR27580.1, XP-125935.3, AAF21633.1, AAF21630.1, AAD05267.1, Q9Z1 W4, NP-1-031585.2, NP-571094.1, CAD43439.1, CAF99217.1, CAB63584.1, NP-722840.1, CAE46407.1, XP-1-417667.1, BAC53989.1, BAB19659.1, AAM46922.1, AAA81169.1, AAK28707.1, AAL05943.1, AAB17573.1, CAH25443.1, CAG10269.1, BAD16731.1, EAA00276.2, AAT07320.1, AAT07300.1, AAN15037.1, CAH25442.1, AAK08152.2, 2009388A, AAR12161.1, CAGO1961.1, CAB63656.1, CAD67714.1, CAF94162.1, NP-477340.1, EAL24792.1, NP-1-001009428.1, AAB86686.1, AAT40572.1, AAT40571.1, AAT40569.1, NP-033886.1, AAB49985.1, AAG39266.1, Q26974, AAC77461.1, AAC47262.1, BAC05509.1, NP-055297.1, XP-546146.1, XP-525772.1, NP-060525.2, AAH33585.1, AAH69080.1, CAG12751.1, AAH74757.2, NP-034964.1, NP-038639.1, 042221, AAF02773.1, NP-062024.1, AAR18244.1, AAR14343.1, XP-228285.2, AAT40573.1, AAT94456.1, AAL35278.1, AAL35277.1, AAL17640.1, AAC08035.1, AAB86692.1, CAB40844.1, BAC38637.1, BAB16046.1, AAN63522.1, NP-571041.1, AAB04986.2, AAC26791.1, AAB95254.1, BAA11835.1, AAR18246.1, XP-538528.1, BAA31853.1, AAK18000.1, XP-1-420540.1, AAL35276.1, AAQ98602.1, CAE71944.1, AAW50585.1, AAV63982.1, AAW29941.1, AAN87890.1, AAT40568.1, CAD57730.1, AAB81508.1, AAS00534.1, AAC59736.1, BAB79498.1, AAA97392.1, AAP85526.1, NP-999600.2, NP-878293.1, BAC82629.1, CAC60268.1, CAG04919.1, AAN10123.1, CAA07707.1 AAK20912.1, AAR88254.1, CAC34629.1, AAL35275.1, AAD46997.1, AAN03842.1, NP-571951.2, CAC50881.1, AAL99367.1, AAL49502.1, AAB71839.1, AAB65415.1, NP-624359.1, NP-990153.1, AAF78069.1, AAK49790.1, NP-919367.2, NP-001192.1, XP-544948.1, AAQ18013.1, AAV38739.1, NP-851298.1, CAA67685.1, AAT67171.1, AAT37502.1, AAD27804.1, AAN76665.1, BAC11909.1, XP-1-421648.1, CAB63704.1, NP-037306.1, A55706, AAF02780.1, CAG09623.1, NP-067589.1, NP-035707.1, AAV30547.1, AAP49817.1, BAC77407.1, AAL87199.1, CAG07172.1, B36193, CAA33024.1, NP-1-001009400.1, AAP36538.1, XP-512687.1, XP-510080.1, AAH05513.1, 1KTZ, AAH14690.1, AAA31526.1.

The growth factor from the TGF-β superfamily in the methods and compositions provided herein can be naturally obtained or recombinant. In some embodiments, the growth factor from the TGF-β superfamily comprises Activin A. The term “Activin A” can include fragments and derivatives of Activin A. Non-limiting exemplary sequences of Activin A are listed in Table 1. In some embodiments, the growth factor from the TGF-β superfamily can comprise a polypeptide having an amino acid sequence at least 30%, at least 40%, at least 50%, at least 60%, at least 70%, at least 80%, at least 90%, at least 95%, or at least 99%, or greater identical to the sequence of SEQ ID NO: 1. In some embodiments, the growth factor from the TGF-β superfamily can comprise a polypeptide having the amino acid sequence of SEQ ID NO: 1. In some embodiments, the growth factor from the TGF-β superfamily can comprise a polypeptide having an amino acid sequence at least 30%, at least 40%, at least 50%, at least 60%, at least 70%, at least 80%, at least 90%, at least 95%, or at least 99%, or greater identical to the sequence of any one of SEQ ID NOs: 2-15.

In some embodiments, the growth factor from the TGF-β superfamily comprises growth differentiation factor 8 (GDF8). The term “GDF8” can include fragments and derivatives of GDF8. The sequences of GDF8 polypeptides are available to the skilled artisan. In some embodiments, the growth factor from the TGF-β superfamily comprises a polypeptide having an amino acid sequence at least 30%, at least 40%, at least 50%, at least 60%, at least 70%, at least 80%, at least 90%, at least 95%, or at least 99%, or greater identical to the human GDF8 polypeptide sequence (GenBank Accession EAX10880).

In some embodiments, the growth factor from the TGF-β superfamily comprises a growth factor that is closely related to GDF8, e.g., growth differentiation factor 11 (GDF11). In some embodiments, the growth factor from the TGF-β superfamily comprises a polypeptide having an amino acid sequence at least 30%, at least 40%, at least 50%, at least 60%, at least 70%, at least 80%, at least 90%, at least 95%, or at least 99%, or greater identical to the human GDF11 polypeptide sequence (GenBank Accession AAF21630).

In some embodiments, the growth factor from the TGF-β superfamily can be replaced with an agent mimics the at least one growth factor from the TGF-β superfamily. Exemplary agents that mimic the at least one growth factor from the TGF-β superfamily, include, without limitation, IDE1 and IDE2.

### Bone Morphogenetic Protein (BMP) Signaling Pathway Inhibitors

Aspects of the disclosure relate to the use of BMP signaling pathway inhibitors as R cell differentiation factors. The BMP signaling family is a diverse subset of the TGF-β superfamily (Sebald et al. Biol. Chem. 385:697-710, 2004). Over twenty known BMP ligands are recognized by three distinct type II (BMPRII, ActRIIa, and ActRIIb) and at least three type I (ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR—) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner (Nohe et al. Cell Signal 16:291-299, 2004). Soluble BMP antagonists such as noggin, chordin, gremlin, and follistatin limit BMP signaling by ligand sequestration.

In some embodiments, the BMP signaling pathway inhibitor in the methods and composition provided herein comprises DMH-1, or a derivative, analogue, or variant thereof. In some embodiments, the BMP signaling pathway inhibitor in the methods and composition provided herein comprises the following compound or a derivative, analogue, or variant of the following compound:

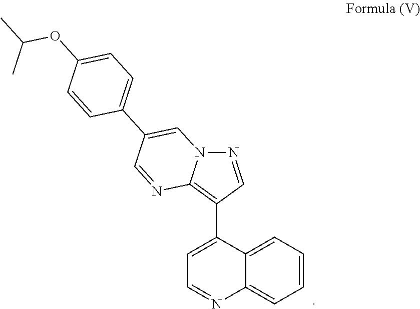 [SMILES: CC(C)Oc1ccc(-c2cnc3c(-c4ccnc5ccccc45)cnn3c2)cc1]

In some embodiments, the BMP signaling pathway inhibitor in the methods and composition provided herein comprises LDN193189 (also known as LDN193189, 1062368-24-4, LDN-193189, DM 3189, DM-3189, IUPAC Name: 4-[6-(4-piperazin-1-ylphenyl)pyrazolo[1,5-a]pyrimidin-3-yl]quinolone). In some embodiments, the BMP signaling pathway inhibitor in the methods and composition provided herein comprises the following compound or a derivative, analogue, or variant of the following compound:

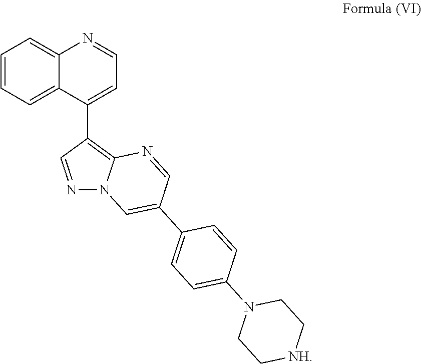 [SMILES: c1ccc2c(-c3cnn4cc(-c5ccc(N6CCNCC6)cc5)cnc34)ccnc2c1]

In some cases, DMH-1 can be more selective as compared to LDN193189. In some embodiments of the present disclosure, DMH-1 can be particularly useful for the methods provided herein. In some embodiments, the methods and compositions provided herein exclude use of LDN193189. In some embodiments, the methods and compositions provided herein exclude use of LDN193189, or a derivative, analogue, or variant thereof for generating PDX1-positive, NKX6.1-negative pancreatic progenitor cells from primitive gut tube cells. In some embodiments, the methods and compositions provided herein relate to use of DMH-1, or a derivative, analogue, or variant thereof for generating PDX1-positive, NKX6.1-negative pancreatic progenitor cells from primitive gut tube cells.

In some embodiments, the BMP signaling pathway inhibitor in the methods and composition provided herein comprise an analog or derivative of LDN193189, e.g., a salt, hydrate, solvent, ester, or prodrug of LDN193189. In some embodiments, a derivative (e.g., salt) of LDN193189 comprises LDN193189 hydrochloride.

In some embodiments, the BMP signaling pathway inhibitor in the methods and composition provided herein comprises a compound of Formula I from U.S. Patent Publication No. 2011/0053930.

### TGF-β Signaling Pathway Inhibitors

Aspects of the disclosure relate to the use of TGF-β signaling pathway inhibitors as R cell differentiation factors.

In some embodiments, the TGF-β signaling pathway comprises TGF-β receptor type I kinase (TGF-β RI) signaling. In some embodiments, the TGF-β signaling pathway inhibitor comprises ALK5 inhibitor II (CAS 446859-33-2, an ATP-competitive inhibitor of TGF-B RI kinase, also known as RepSox, IUPAC Name: 2-[5-(6-methylpyridin-2-yl)-1H-pyrazol-4-yl]-1,5-naphthyridine. In some embodiments, the TGF-β signaling pathway inhibitor is an analog or derivative of ALK5 inhibitor II.

In some embodiments, the analog or derivative of ALK5 inhibitor II (also named “ALK5i”) is a compound of Formula I as described in U.S. Patent Publication No. 2012/0021519, incorporated by reference herein in its entirety.

In some embodiments, the TGF-β signaling pathway inhibitor in the methods and compositions provided herein is a TGF-β receptor inhibitor described in U.S. Patent Publication No. 2010/0267731. In some embodiments, the TGF-β signaling pathway inhibitor in the methods and compositions provided herein comprises an ALK5 inhibitor described in U.S. Patent Publication Nos. 2009/0186076 and 2007/0142376. In some embodiments, the TGF-β signaling pathway inhibitor in the methods and compositions provided herein is A 83-01. In some embodiments, the TGF-β signaling pathway inhibitor in the methods and compositions provided herein is not A 83-01. In some embodiments, the compositions and methods described herein exclude A 83-01. In some embodiments, the TGF-β signaling pathway inhibitor in the methods and compositions provided herein is SB 431542. In some embodiments, the TGF-β signaling pathway inhibitor is not SB 431542. In some embodiments, the compositions and methods described herein exclude SB 431542. In some embodiments, the TGF-β signaling pathway inhibitor in the methods and compositions provided herein is D 4476. In some embodiments, the TGF-β signaling pathway inhibitor is not D 4476. In some embodiments, the compositions and methods described herein exclude D 4476. In some embodiments, the TGF-β signaling pathway inhibitor in the methods and compositions provided herein is GW 788388. In some embodiments, the TGF-β signaling pathway inhibitor is not GW 788388. In some embodiments, the compositions and methods described herein exclude GW 788388. In some embodiments, the TGF-β signaling pathway inhibitor in the methods and compositions provided herein is LY 364947. In some embodiments, the TGF-β signaling pathway inhibitor is not LY 364947. In some embodiments, the compositions and methods described herein exclude LY 364947. In some embodiments, the TGF-β signaling pathway inhibitor in the methods and compositions provided herein is LY 580276. In some embodiments, the TGF-β signaling pathway inhibitor is not LY 580276. In some embodiments, the compositions and methods described herein exclude LY 580276. In some embodiments, the TGF-β signaling pathway inhibitor in the methods and compositions provided herein is SB 525334. In some embodiments, the TGF-β signaling pathway inhibitor is not SB 525334. In some embodiments, the compositions and methods described herein exclude SB 525334. In some embodiments, the TGF-β signaling pathway inhibitor in the methods and compositions provided herein is SB 505124. In some embodiments, the TGF-β signaling pathway inhibitor is not SB 505124. In some embodiments, the compositions and methods described herein exclude SB 505124. In some embodiments, the TGF-β signaling pathway inhibitor in the methods and compositions provided herein is SD 208. In some embodiments, the TGF-β signaling pathway inhibitor is not SD 208. In some embodiments, the compositions and methods described herein exclude SD 208. In some embodiments, the TGF-β signaling pathway inhibitor in the methods and compositions provided herein is GW 6604. In some embodiments, the TGF-β signaling pathway inhibitor is not GW 6604. In some embodiments, the compositions and methods described herein exclude GW 6604. In some embodiments, the TGF-β signaling pathway inhibitor in the methods and compositions provided herein is GW 788388. In some embodiments, the TGF-β signaling pathway inhibitor in the methods and compositions provided herein is not GW 788388. In some embodiments, the compositions and methods described herein exclude GW 788388.

From the collection of compounds described above, the following can be obtained from various sources: LY-364947, SB-525334, SD-208, and SB-505124 available from Sigma, P.O. Box 14508, St. Louis, Mo., 63178-9916; 616452 and 616453 available from Calbiochem (EMD Chemicals, Inc.), 480 S. Democrat Road, Gibbstown, N.J., 08027; GW788388 and GW6604 available from GlaxoSmithKline, 980 Great West Road, Brentford, Middlesex, TW8 9GS, United Kingdom; LY580276 available from Lilly Research, Indianapolis, Ind. 46285; and SM16 available from Biogen Idec, P.O. Box 14627, 5000 Davis Drive, Research Triangle Park, N.C., 27709-4627.

### WNT Signaling Pathway

Aspects of the disclosure relate to the use of activators of the WNT signaling pathway as cell differentiation factors.

In some embodiments, the WNT signaling pathway activator in the methods and compositions provided herein comprises CHIR99021. In some embodiments, the WNT signaling pathway activator in the methods and compositions provided herein comprises a derivative of CHIR99021, e.g., a salt of CHIR99021, e.g., trihydrochloride, a hydrochloride salt of CHIR99021. In some embodiments, the WNT signaling pathway activator in the methods and compositions provided herein comprises Wnt3a recombinant protein. In some embodiments, the WNT signaling pathway activator in the methods and compositions provided herein comprises a glycogen synthase kinase 3 (GSK3) inhibitor. Exemplary GSK3 inhibitors include, without limitation, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC 693868, SB 216763, SB 415286, TC-G 24, TCS 2002, TCS 21311, TWS 119, and analogs or derivatives of any of these. In certain embodiments, the methods, compositions, and kits disclosed herein exclude a WNT signaling pathway activator.

In some embodiments, a medium described herein does not comprise a Wnt signaling pathway activator.

Aspects of the disclosure relate to the use of inhibitors of the WNT signaling pathway as β cell differentiation factors.

In some embodiments, the WNT signaling inhibitor is a tankyrase inhibitor that inhibits expression or activity of at least one tankyrase (TNKS) protein. In some embodiments, the at least one tankyrase protein is tankyrase 1 or tankyrase 2. In some embodiments, the WNT signaling inhibitor inhibits binding of a substrate to a nicotinamide subsite or an adenosine subsite, or both, of a tankyrase protein. In some embodiments, the tankyrase inhibitor is AZ 6102, JW55, MN64, IWR-1-endo, TC-E5001, WIKI4, TNKS 22, TNKS 49, 2X-121 (E7449), XAV-939 (XAV), G007-LK, NVP-TNKS656, decernotinib, (VX-509), vismodegib (GDC-0449), IM-12, GSK429286A, INO-1001, Ofloxacin, TG101209, FG-4592, 1-BET-762, LY2157299, MK-0752, Wnt-C59 (C59), MC1568, Pacritinib (SB 1518), SB415286, Drocinostat, IWR-1-endo, Norfloxacin, SH-4-54, Nexturastat A, SB216763, UNCO 79, dephnetin, GF109203X, RepSox, Sotrastaurin, SB431542, tofacitinib (CP-690550, Tasocitinib), AG-14361, CI994 (tacedinaline), Ro 31-8220 mesylate, resveratrol, NVP-TNKS656, or YO-01027. In some embodiments, said tankyrase inhibitor is AZ 6102, NVP-TNKS656, or IWR-1-endo. In some embodiments, the tankyrase inhibitor is NVP-TNKS656 (NVP). In some embodiments, the tankyrase inhibitor selectively inhibits tankyrase 1 over tankyrase 2. In some embodiments, the tankyrase inhibitor selectively inhibits tankyrase 2 over tankyrase 1.

In some embodiments, a medium described herein does not comprise a Wnt signaling pathway inhibitor.

### Fibroblast Growth Factor (FGF) Family

Aspects of the disclosure relate to the use of growth factors from the FGF family as R cell differentiation factors.

In some embodiments, the growth factor from the FGF family in the methods and compositions provided herein comprises keratinocyte growth factor (KGF). The polypeptide sequences of KGF are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human KGF polypeptide sequence (GenBank Accession AAB21431). In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the sequence of SEQ ID NO: 16.

In some embodiments, the growth factor from the FGF family in the methods and composition provided herein comprises FGF2. The polypeptide sequences of FGF2 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF2 polypeptide sequence (GenBank Accession NP-001997).

In some embodiments, the at least one growth factor from the FGF family in the methods and composition provided herein comprises FGF8B. The polypeptide sequences of FGF8B are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF8B polypeptide sequence (GenBank Accession AAB40954).

In some embodiments, the at least one growth factor from the FGF family in the methods and composition provided herein comprises FGF10. The polypeptide sequences of FGF10 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF10 polypeptide sequence (GenBank Accession CAG46489).

In some embodiments, the at least one growth factor from the FGF family in the methods and composition provided herein comprises FGF21. The polypeptide sequences of FGF21 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF21 polypeptide sequence (GenBank Accession AAQ89444.1).

### Sonic Hedgehog (SHH) Signaling Pathway

Aspects of the disclosure relate to the use of SHH signaling pathway inhibitors as R cell differentiation factors.

In some embodiments, the SHH signaling pathway inhibitor in the methods and composition provided herein comprises Sant1. In some embodiments, the SHH signaling pathway inhibitor in the methods and composition provided herein comprises SANT2. In some embodiments, the SHH signaling pathway inhibitor in the methods and composition provided herein comprises SANT3. In some embodiments, the SHH signaling pathway inhibitor in the methods and composition provided herein comprises SANT4. In some embodiments, the SHH signaling pathway inhibitor comprises Cur61414. In some embodiments, the SHH signaling pathway inhibitor in the methods and composition provided herein comprises forskolin. In some embodiments, the SHH signaling pathway inhibitor in the methods and composition provided herein comprises tomatidine. In some embodiments, the SHH signaling pathway inhibitor in the methods and composition provided herein comprises AY9944. In some embodiments, the SHH signaling pathway inhibitor in the methods and composition provided herein comprises triparanol. In some embodiments, the SHH signaling pathway inhibitor in the methods and composition provided herein comprises compound A or compound B (as disclosed in U.S. Pub. No. 2004/0060568). In some embodiments, the SHH signaling pathway inhibitor in the methods and composition provided herein comprises a steroidal alkaloid that antagonizes hedgehog signaling (e.g., cyclopamine or a derivative thereof) as disclosed in U.S. Pub. No. 2006/0276391. In certain embodiments, the methods, compositions, and kits disclosed herein exclude a SHH signaling pathway inhibitor.

### Rho Kinase (ROCK) Signaling Pathway

Aspects of the disclosure relate to the use of ROCK signaling pathway inhibitors (ROCK inhibitors) as β cell differentiation factors.

In some embodiments, the ROCK inhibitor in the methods and composition provided herein comprises Y-27632 or Thiazovivin. In some embodiments, the ROCK inhibitor in the methods and composition provided herein comprises Thiazovivin. In some embodiments, the ROCK inhibitor in the methods and composition provided herein comprises Y-27632. In some cases, the ROCK inhibitor in the methods and composition provided herein comprises the following compound or a derivative thereof:

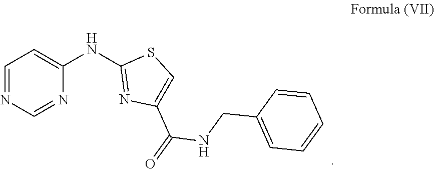 [SMILES: O=C(NCc1ccccc1)c1csc(Nc2ccncn2)n1]

In some cases, the ROCK inhibitor in the methods and composition provided herein comprises the following compound or a derivative thereof:

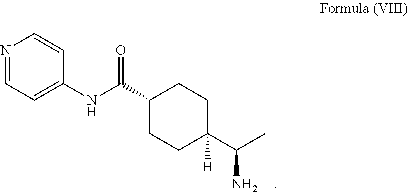 [SMILES: C[C@@H](N)[C@H]1CC[C@H](C(=O)Nc2ccncc2)CC1]

Non-limiting examples of ROCK inhibitor that can be used in the methods and compositions provided herein include Thiazovivin, Y-27632, Fasudil/HA1077, H-1152, Ripasudil, Y39983, Wf-536, SLx-2119, Azabenzimidazole-aminofurazans, DE-104, Olefins, Isoquinolines, Indazoles, and pyridinealkene derivatives, ROKa inhibitor, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, and quinazoline.

### Retinoic Acid Signaling Pathway

Aspects of the disclosure relate to the use of modulators of retinoic acid signaling as β cell differentiation factors.

In some embodiments, the modulator of retinoic acid signaling in the methods and composition provided herein comprises an activator of retinoic acid signaling. In some embodiments, the RA signaling pathway activator in the methods and composition provided herein comprises retinoic acid. In some embodiments, the RA signaling pathway activator in the methods and composition provided herein comprises a retinoic acid receptor agonist. Exemplary retinoic acid receptor agonists in the methods and composition provided herein include, without limitation, CD 1530, AM 580, TTNPB, CD 437, Ch 55, BMS 961, AC 261066, AC 55649, AM 80, BMS 753, tazarotene, adapalene, and CD 2314.

In some embodiments, the modulator of retinoic acid signaling in the methods and composition provided herein comprises an inhibitor of retinoic acid signaling. In some embodiments, the retinoic acid signaling pathway inhibitor comprises DEAB (IUPAC Name: 2-[2-(diethylamino)ethoxy]-3-prop-2-enylbenzaldehyde). In some embodiments, the retinoic acid signaling pathway inhibitor comprises an analog or derivative of DEAB.

In some embodiments, the retinoic acid signaling pathway inhibitor in the methods and composition provided herein comprises a retinoic acid receptor antagonist. In some embodiments, the retinoic acid receptor antagonist in the methods and composition provided herein comprises (E)-4-[2-(5,6-dihydro-5,5-dimethyl-8-phenyl-2-naphthalenyl)ethenyl]benzoic acid, (E)-4-[[(5,6-dihydro-5,5-dimethyl-8-phenylethynyl)-2-naphthalenyl]ethenyl]benzoic acid, (E)-4-[2-[5,6-dihydro-5,5-dimethyl-8-(2-naphthalenyl)-2-naphthalenyl]ethenyl]-benzoic acid, and (E)-4-[2-[5,6-dihydro-5,5-dimethyl-8-(4-methoxyphenyl)-2-naphthalenyl]ethenyl]benzoic acid. In some embodiments, the retinoic acid receptor antagonist comprises BMS 195614 (CAS #253310-42-8), ER 50891 (CAS #187400-85-7), BMS 493 (CAS #170355-78-9), CD 2665 (CAS #170355-78-9), LE 135 (CAS #155877-83-1), BMS 453 (CAS #166977-43-1), or MM 11253 (CAS #345952-44-5).

In certain embodiments, the methods, compositions, and kits disclosed herein exclude a modulator of retinoic acid signaling. In certain embodiments, the methods, compositions, and kits disclosed herein exclude a retinoic acid signaling pathway activator. In certain embodiments, the methods, compositions, and kits disclosed herein exclude a retinoic acid signaling pathway inhibitor.

### Protein Kinase C Activators

Aspects of the disclosure relate to the use of protein kinase C activators as β cell differentiation factors. Protein kinase C is one of the largest families of protein kinase enzymes and is composed of a variety of isoforms. Conventional isoforms include a, βI, βII, γ; novel isoforms include δ, ε, η, Θ; and atypical isoforms include ξ, and ι/λ. PKC enzymes are primarily cytosolic but translocate to the membrane when activated. In the cytoplasm, PKC is phosphorylated by other kinases or autophosphorylated. In order to be activated, some PKC isoforms (e.g., PKC-ε) require a molecule to bind to the diacylglycerol (“DAG”) binding site or the phosphatidylserine (“PS”) binding site. Others are able to be activated without any secondary binding messengers at all. PKC activators that bind to the DAG site include, but are not limited to, bryostatin, picologues, phorbol esters, aplysiatoxin, and gnidimacrin. PKC activators that bind to the PS site include, but are not limited to, polyunsaturated fatty acids and their derivatives. It is contemplated that any protein kinase C activator that is capable, either alone or in combination with one or more other β cell differentiation factors, of inducing the differentiation of at least one insulin-producing, endocrine cell or precursor thereof into a SC-β cell can be used in the methods, compositions, and kits described herein.

In some embodiments, the PKC activator in the methods and composition provided herein comprises PdBU. In some embodiments, the PKC activator in the methods and composition provided herein comprises TPB. In some embodiments, the PKC activator in the methods and composition provided herein comprises cyclopropanated polyunsaturated fatty acids, cyclopropanated monounsaturated fatty acids, cyclopropanated polyunsaturated fatty alcohols, cyclopropanated monounsaturated fatty alcohols, cyclopropanated polyunsaturated fatty acid esters, cyclopropanated monounsaturated fatty acid esters, cyclopropanated polyunsaturated fatty acid sulfates, cyclopropanated monounsaturated fatty acid sulfates, cyclopropanated polyunsaturated fatty acid phosphates, cyclopropanated monounsaturated fatty acid phosphates, macrocyclic lactones, DAG derivatives, isoprenoids, octylindolactam V, gnidimacrin, iripallidal, ingenol, napthalenesulfonamides, diacylglycerol kinase inhibitors, fibroblast growth factor 18 (FGF-18), insulin growth factor, hormones, and growth factor activators, as described in U.S. Patent Publication No. US20140315990A1. In some embodiments, the bryostain comprises bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18. In certain embodiments, the methods, compositions, and kits disclosed herein exclude a protein kinase C activator.

### γ-Secretase Inhibitors

Aspects of the disclosure relate to the use of γ-secretase inhibitors as β cell differentiation factors.

In some embodiments, the γ-secretase inhibitor in the methods and composition provided herein comprises XXI. In some embodiments, the γ-secretase inhibitor in the methods and composition provided herein comprises DAPT. Additional exemplary γ-secretase inhibitors in the methods and composition provided herein include, without limitation, the γ-secretase inhibitors described in U.S. Pat. Nos. 7,049,296, 8,481,499, 8,501,813, and U.S. Patent Publication No. US20140243374A1. In certain embodiments, the methods, compositions, and kits disclosed herein exclude a γ-secretase inhibitor.

### Thyroid Hormone Signaling Pathway Activators

Aspects of the disclosure relate to the use of thyroid hormone signaling pathway activators as β cell differentiation factors.

In some embodiments, the thyroid hormone signaling pathway activator in the methods and composition provided herein comprises triiodothyronine (T3). In some embodiments, the thyroid hormone signaling pathway activator in the methods and composition provided herein comprises GC-1. In some embodiments, the thyroid hormone signaling pathway activator in the methods and composition provided herein comprises an analog or derivative of T3 or GC-1. Exemplary analogs of T3 in the methods and composition provided herein include, but are not limited to, selective and non-selective thyromimetics, TRO selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-2115 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine [T4] and triiodothyronine [T3] alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3′-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA).

In some embodiments, the thyroid hormone signaling pathway activator in the methods and composition provided herein comprises a prodrug or prohormone of T3, such as T4 thyroid hormone (e.g., thyroxine or L-3,5,3′,5′-tetraiodothyronine).

In some embodiments, the thyroid hormone signaling pathway activator in the methods and composition provided herein is an iodothyronine composition described in U.S. Pat. No. 7,163,918, which is incorporated herein by reference in its entirety.

### Epidermal Growth Factor (EGF) Family

Aspects of the disclosure relate to the use of growth factors from the EGF family as β cell differentiation factors.

In some embodiments, the at least one growth factor from the EGF family in the methods and compositions provided herein comprises betacellulin. In some embodiments, at least one growth factor from the EGF family in the methods and composition provided herein comprises EGF. Epidermal growth factor (EGF) is a 53 amino acid cytokine which is proteolytically cleaved from a large integral membrane protein precursor. In some embodiments, the growth factor from the EGF family in the methods and composition provided herein comprises a variant EGF polypeptide, for example an isolated epidermal growth factor polypeptide having at least 90% amino acid identity to the human wild-type EGF polypeptide sequence, as disclosed in U.S. Pat. No. 7,084,246. In some embodiments, the growth factor from the EGF family in the methods and composition provided herein comprises an engineered EGF mutant that binds to and agonizes the EGF receptor, as is disclosed in U.S. Pat. No. 8,247,531. In some embodiments, the at least one growth factor from the EGF family in the methods and composition provided herein is replaced with an agent that activates a signaling pathway in the EGF family. In some embodiments, the growth factor from the EGF family in the methods and composition provided herein comprises a compound that mimics EGF. In certain embodiments, the methods, compositions, and kits disclosed herein exclude a growth factor from the EGF family. In some embodiments, the growth factor from the EGF family used in the compositions and methods described herein comprises an amino acid sequence that is at least 30%, at least 40%, at least 50%, at least 60%, at least 70%, at least 75%, at least 80%, at least 85%, at least 90%, at least 95%, at least 98% or at least 99% identical to Human betacellulin amino acid sequence (GenBank: AAB25452.1). In some embodiments, the growth factor from the EGF family used in the compositions and methods described herein comprises an amino acid sequence that is at least 30%, at least 40%, at least 50%, at least 60%, at least 70%, at least 75% at least 80%, at least 85%, at least 90%, at least 95%, at least 98% or at least 99% identical to the amino acid sequence of SEQ ID NO: 17, or a functional fragment thereof. In some embodiments, the growth factor from the EGF family used in the compositions and methods described herein comprises the amino acid sequence of SEQ ID NO: 17.

TABLE 1
Exemplary amino acid sequences of differentiation factors

| SEQ | | | |
|---|---|---|---|
| ID | Molecular | | |
| NO | Type | Organism | AminQ acid sequence |
| 1 | Protein | Homo sapiens | GLECDGKVNICCKKQFFVSFKDIGWNDWIIAPSGYHA |
| | | | NYCEGECPSHIAGTSGSSLSFHSTVINHYRMRGHSPF |
| | | | ANLKSCCVPTKLRPMSMLYYDDGQNIIKKDIQNMIVE |
| | | | ECGCS |
| 2 | Protein | Homo sapiens | MPLLWLRGFLLASCWIIVRSSPTPGSEGHSAAPDCPS |
| | | | CALAALPKDVPNSQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEVWLFLKVPKANRTRTKVTIRLFQQQKHPQGS |
| | | | LDTGEEAEEVGLKGERSELLLSEKVVDARKSTWHVFP |
| | | | VSSSIQRLLDQGKSSLDVRIACEQCQESGASLVLLGK |
| | | | KKKKEEEGEGKKKGGGEGGAGADEEKEQSHRPFLMLQ |
| | | | ARQSEDHPHRRRRRGLECDGKVNICCKKQFFVSFKDI |
| | | | GWNDWIIAPSGYHANYCEGECPSHIAGTSGSSLSFHS |
| | | | TVINHYRMRGHSPFANLKSCCVPTKLRPMSMLYYDDG |
| | | | QNIIKKDIQNMIVEECGCS |
| 3 | Protein | Homo sapiens | ARQSEDHPHRRRRRGLECDGKVNICCKKQFFVSFKDI |
| | | | GWNDWIIAPSGYHANYCEGECPSHIAGTSGSSLSFHS |
| | | | TVINHYRMRGHSPFANLKSCCVPTKLRPMSMLYYDDG |
| | | | QNIIKKDIQNMIVEECGCS |
| 4 | Protein | Unidentified | GLECDGKVNICCKKQFFVSFKDIGWNDWIIAPSGYHA |
| | | | NYCEGECPSHIAGTSGSSLSFHSTVINHYACGHSPFA |
| | | | NLKSCCVPTKLRPMSMLYYDDGQNIIKKDIQNMIVEE |
| | | | CGCS |
| 5 | Protein | Homo sapiens | MPLLWLRGFLLASCWIIVRSSPTPGSEGHSAAPDCPS |
| | | | CALAALPKDVPNSQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEVWLFLKVPKANRTRTKVTIRLFQQQKHPQGS |
| | | | LDTGEEAEEVGLKGERSELLLSEKVVDARKSTWHVFP |
| | | | VSSSIQRLLDQGKSSLDVRIACEQCQESGASLVLLGK |
| | | | KKKKEEEGEGKKKGGGEGGAGADEEKEQSHRPFLMLQ |
| | | | ARQSEDHPHRRRRRGLECDGKVNICCKKQFFVSFKDI |
| | | | GWNDWIIAPSGYHANYCEGECPSHIAGTSGSSLSFHS |
| | | | TVINHYRMRGHSPFANLKSCCVPTKLRPMSMLYYDDG |
| | | | QNIIKKDIQNMIVEECGCS |
| 6 | Protein | Unidentified | MPLLWLRGFLLASCWIIVRSSPTPGSEGHGSAPDCPS |
| | | | CALATLPKDGPNSQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEVWLFLKVPKANRTRTKVTIRLFQQQKHPQGS |
| | | | LDTGDEAEEMGLKGERSELLLSEKVVDARKSTWHIFP |
| | | | VSSSIQRLLDQGKSSLDVRIACEQCQESGASLVLLGK |
| | | | KKKKEVDGDGKKKDGSDGGLEEEKEQSHRPFLMLQAR |
| | | | QSEDHPHRRRRRGLECDGKVNICCKKQFFVSFKDIGW |
| | | | NDWIIAPSGYHANYCEGECPSHIAGTSGSSLSFHSTV |
| | | | INHYRMRGHSPFANLKSCCVPTKLRPMSMLYYDDGQN |
| | | | IIKKDIQNMIVEECGCS |
| 7 | Protein | Unidentified | MPLLWLRGFLLASCWIIVRSSPTPGSEGHGAAPDCPS |
| | | | CALATLPKDGPNSQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEVWLFLKVPKANRTRTKVTIRLFQQQKHPQGS |
| | | | LDMGDEAEEMGLKGERSELLLSEKVVDARKSTWHIFP |
| | | | VSSSIQRLLDQGKSSLDVRIACEQCQESGASLVLLGK |
| | | | KKKKEVDGDGKKKDGSDGGLEEEKEQSHRPFLMLQAR |
| | | | QSEDHPHRRRRRGLECDGKVNICCKKQFFVSFKDIGW |
| | | | NDWIIAPSGYHANYCEGECPSHIAGTSGSSLSFHSTV |
| | | | INHYRMRGHSPFANLKSCCVPTKLRPMSMLYYDDGQN |
| | | | IIKKDIQNMIVEECGCS |
| 8 | Protein | Unidentified | MPLLWKRGFLLVICWIIVRSSPTPGSEGHSSVADCPS |
| | | | CALTTLSKDVPSSQPEMVEAVKKHILNMLHLRDRPNI |
| | | | TQPVPKAALLNATKKLHVGKVGDDGYVEIEDDVGRRA |
| | | | EMNEVVEQTSEIITFAESGTPKKTLHFEISKEGSELS |
| | | | VVEHAEVWLFLKVSKANRSRTKVTIRLFQQQRQPKGN |
| | | | SEAAEDMEDMGLKGERSETLISEKAVDARKSTWHIFP |
| | | | ISSSVQRLLDQGQSSLDVRIACDLCQETGASLVLLGK |
| | | | KKKKEDDGEGKEKDGGELTGEEEKEQSHRPFLMMLAR |
| | | | HSEDRQHRRRERGLECDGKVNICCKKQFFVSFKDIGW |
| | | | SDWIIAPTGYHANYCEEECPSHIAGTSGSSLSFHSTV |
| | | | INHYRMRGHSPFANLKSCCVPTKLRPMSMLYYDDGQN |
| | | | IIKKDIQNMIVEECGCS |
| 9 | Protein | Unidentified | MPLLWLRGFLLASCWIIVRSSPTPGSEGHSAAPDCPS |
| | | | CALATLPKDVPNSQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEIWLFLKVPKANRTRSKVTIRLFQQQKHLQGS |
| | | | LDAGEEAEEVGLKGEKSEMLISEKVVDARKSTWHIFP |
| | | | VSSCIQRLLDQGKSSLDIRIACEQCQETGASLVLLGK |
| | | | KKKKEEEGEGKKRDGEGGAGGDEEKEQSHRPFLMLQA |
| | | | RQSEDHPHRRRRRGLECDGKVNICCKKQFFVSFKDIG |
| | | | WNDWIIAPSGYHANYCEGECPSHIAGTSGSSLSFHST |
| | | | VINHYRMRGHSPFANLKSCCVPTKLRPMSMLYYDDGQ |
| | | | NIIKKDIQNMIVEECGCS |
| 10 | Protein | Unidentified | MPLLWLRGFLLASCWIIVKSSPTPGSEGHSAAPNCPS |
| | | | CALATLPKDVPNAQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEVWLFLKVPKANRTRSKVTIRLLQQQKHPQGS |
| | | | SDTREEAEEADLMEERSEQLISEKVVDARKSTWHIFP |
| | | | VSSSIQRLLDQGKSSLDIRIACDQCHETGASLVLLGK |
| | | | KKKKEEEGEGKKKDGGEAGAGVDEEKEQSHRPFLMLQ |
| | | | ARQSEDHPHRRRRRGLECDGKVNICCKKQFFVSFKDI |
| | | | GWNDWIIAPSGYHANYCEGECPSHIAGTSGSSLSFHS |
| | | | TVINQYRLRGHNPFANLKSCCVPTKLRPMSMLYYDDG |
| | | | QNIIKKDIQNMIVEECGCS |
| 11 | Protein | Unidentified | MPLLWLRGFLLASCWIIVRSSPTPGSGGHSAAPDCPS |
| | | | CALATLPKDVPNSQPEMVEAVKKHILNMLHLKKRPDV |
| | | | TQPVPKAALLNAIRKLHVGKVGENGYVELEDDIGRRA |
| | | | EMNELMEQTSEIITFAEAGTARKTLRFEISKEGSDLS |
| | | | VVERAEIWLFLKVPKANRTRTKVSIRLFQQQRRPQGS |
| | | | ADAGEEAEDVGFPEEKSEVLISEKVVDARKSTWHIFP |
| | | | VSSSIQRLLDQGKSALDIRTACEQCHETGASLVLLGK |
| | | | KKKKEEEAEGRKRDGEGAGVDEEKEQSHRPFLMLQAR |
| | | | QSEEHPHRRRRRGLECDGKVNICCKKQFFVSFKDIGW |
| | | | NDWIIAPSGYHANYCEGECPSHIAGTSGSSLSFHSTV |
| | | | INHYRMRGHSPFANLKSCCVPTKLRPMSMLYYDDGQN |
| | | | IIKKDIQNMIVEECGCS |
| 12 | Protein | Unidentified | MPLLWLRGFLLASCWIIVRSSPTPGSEGHSAAPDCPS |
| | | | CALATLPKDVPNSQPEMVEAVKKHILNMLHLKKRPDV |
| | | | TQPVPKAALLNAIRKLHVGKVGENGYVEIEDDIGRRA |
| | | | EMNELMEQTSEIITFAESGTARKTLHFEISQEGSDLS |
| | | | VVERAEIWLFLKVPKANRTRSKVTIRLFQQQKHLQGS |
| | | | LDAGEEAEEVGLKGEKSEMLISEKVVDARKSTWHIFP |
| | | | VSSCIQRLLDQGKSSLDIRIACEQCQETGASLVLLGK |
| | | | KKRKEEEGEGKKRDGEGGAGGDEEKEQSHRPFLMLQA |
| | | | RQSEDHPHRRRRRGLECDGKVNICCKKQFYVSFKDIG |
| | | | WNDWIIAPSGYHANYCEGECPSHIAGTSGSSLSFHST |
| | | | VINHYRMRGHSPFANLKSCCVPTKLRPMSMLYYDDGQ |
| | | | NIIKKDIQNMIVEECGCS |
| 13 | Protein | Unidentified | MPLLWLRGFLLASCWIIVRSSPTPGSEGPGAAPDCPS |
| | | | CALATLPKDVPNSQPEMVEAVKKHILNMLHLKKRPEV |
| | | | TQPVPKAALLNAIRKLHVGKVGENGYVEIEDDIGRRA |
| | | | EMNELMEQTSEIITFAESGTARKTLHFEISKEGSDLS |
| | | | VVERAEVWLFLKVPKANRTRTKVTIQLLQKQPQGGVD |
| | | | AGEEAEEMGLMEERNEVLISEKVVDARKSTWHIFPVS |
| | | | SSIQRLLDQGKSSLDVRIACEQCHETGASLVLLGKKK |
| | | | KKEEEGEGKKKDGGDGGAGADEDKEQSHRPFLMLQAR |
| | | | QSEDHPHRRRRRGLECDGKVNICCKKQFFVSFKDIGW |
| | | | NDWIIAPSGYHANYCEGECPSHIAGTSGSSLSFHSTV |
| | | | INHYRMRGHSPFANLKSCCVPTKLRPMSMLYYDDGQN |
| | | | IIKKDIQNMIVEECGCS |
| 14 | Protein | Unidentified | MSPLPLLSGILLLLIRSCSLSAMVTKGSLPMSEQQAG |
| | | | ATVCPSCALARFRKGVSESEDEGAQQDVVEAVKRHIL |
| | | | NMLHLQERPNITHPVPRAALLNAIRKVHVGRVAKDGS |
| | | | VLIEDEASNRAETEQAEQTEIITFAETGEAPGIVNEL |
| | | | ISKEGGEMSVVDQANVWIFLRLPKGNRTRANVNIRLL |
| | | | LQQGAGEKILAEKSVDTRRSGWHTFPASESVQSLLQR |
| | | | GGSTLSLRVSCPLCADARATPVLVSPGGSEREQSHRP |
| | | | FLMAVVRQMDELSLRRRRKRGLECDGKARVCCKRQFY |
| | | | VNFKDIGWNDWIIAPSGYHANYCEGDCASNVASITGN |
| | | | SLSFHSTVISHYRIRGYSPFTNIKSCCVPTRLRAMSM |
| | | | LYYNEEQKIVKKDIQNMIVEECGCS |
| 15 | Protein | Unidentified | MSSLTLVNRGTAALRLFVRGLLTHSSREWLSGDGEPD |
| | | | DPVTPCPSCALAQRQKDSEEQTDMVEAVKRHILNMLH |
| | | | LNTRPNVTHPVPRAALLNAIRRLHVGRVGEDGTVEME |
| | | | EDGGGLGEHREQSEEQPFEIITFAEPGDAPDIMKEDI |
| | | | SMEGNTLSVVEQANVWLLLKVAKGSRGKGKVSVQLLQ |
| | | | HGKADPGSADGPQEAVVSEKTVDTRRSGWHTLPVSRT |
| | | | VQTLLDGDSSMLSLRVSCPMCAEAGAVPILVPTESNK |
| | | | GKEREQSHRPFLMVVLKPAEEHPHRRSKRGLECDGKI |
| | | | RVCCKRQFYVNFKDIGWSDWIIAPSGYHANYCEGDCP |
| | | | SHVASITGSALSFHSTVINHYRMRGYSPENNIKSCCV |
| | | | PTRLRAMSMLYYNEEQKIIKKDIQNMIVEECGCS |
| 16 | Protein | Unidentified | MHKWILTWILPTLLYRSCFHIICLVGTISLACNDMTP |
| | | | EQMATNVNCSSPERHTRSYDYMEGGDIRVRRLFCRTQ |
| | | | WYLRIDKRGKVKGTQEMKNNYNIMEIRTVAVGIVAIK |
| | | | GVESEFYLAMNKEGKLYAKKECNEDCNFKELILENHY |
| | | | NTYASAKWTHNGGEMFVALNQKGIPVRGKKTKKEQKT |
| | | | AHFLPMAIT |
| 17 | Protein | Homo sapiens | MDRAARCSGASSLPLLLALALGLVILHCVVADGNSTR |
| | | | SPETNGLLCGDPEENCAATTTQSKRKGHFSRCPKQYK |
| | | | HYCIKGRCRFVVAEQTPSCVCDEGYIGARCERVDLFY |
| | | | LRGDRGQILVICLIAVMVVFIILVIGVCTCCHPLRKR |
| | | | RKRKKKEEEMETLGKDITPINEDIEETN |

### Epigenetic Modifying Compounds

Aspects of the disclosure relate to the use of epigenetic modifying compound as β cell differentiation factors.

The term “epigenetic modifying compound” can refer to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, can alter DNA accessibility and chromatin structure, thereby regulating patterns of gene expression. These processes can be crucial to normal development and differentiation of distinct cell lineages in the adult organism. They can be modified by exogenous influences, and, as such, can contribute to or be the result of environmental alterations of phenotype or pathophenotype. Importantly, epigenetic modification can have a crucial role in the regulation of pluripotency genes, which become inactivated during differentiation. Non-limiting exemplary epigenetic modifying compound include a DNA methylation inhibitor, a histone acetyltransferase inhibitor, a histone deacetylase inhibitor, a histone methyltransferase inhibitor, a bromodomain inhibitor, or any combination thereof.

In an embodiment, the histone methyltransferase inhibitor is an inhibitor of enhancer of zeste homolog 2 (EZH2). EZH2 is a histone-lysine N-methyltransferase enzyme. Non-limiting examples of an EZH2 inhibitor that can be used in the methods provided herein include 3-deazaneplanocin A (DZNep), EPZ6438, EPZ005687 (an S-adenosylmethionine (SAM) competitive inhibitor), EI1, GSK126, and UNC1999. DZNep can inhibit the hydrolysis of S-adenosyl-L-homocysteine (SAH), which is a product-based inhibitor of all protein methyltransferases, leading to increased cellular concentrations of SAH which in turn inhibits EZH2. DZNep may not be specific to EZH2 and can also inhibit other DNA methyltransferases. GSK126 is a SAM-competitive EZH2 inhibitor that has 150-fold selectivity over EZH1. UNC1999 is an analogue of GSK126, and it is less selective than its counterpart GSK126.

In an embodiment, the histone methyltransferase inhibitor is DZNep. In an embodiment, the HDAC inhibitor is a class I HDAC inhibitor, a class II HDAC inhibitor, or a combination thereof. In an embodiment, the HDAC inhibitor is KD5170 (mercaptoketone-based HDAC inhibitor), MC1568 (class IIa HDAC inhibitor), TMP195 (class IIa HDAC inhibitor), or any combination thereof. In some embodiments, HDAC inhibitor is vorinostat, romidepsin (Istodax), chidamide, panobinostat (farydak), belinostat (PXD101), panobinostat (LBH589), valproic acid, mocetinostat (MGCD0103), abexinostat (PCI-24781), entinostat (MS-275), SB939, resminostat (4SC-201), givinostat (ITF2357), quisinostat (JNJ-26481585), HBI-8000, (a benzamide HDI), kevetrin, CUDC-101, AR-42, CHR-2845, CHR-3996, 4SC-202, CG200745, ACY-1215, ME-344, sulforaphane, or any variant thereof.

Exemplary histone methyltransferase inhibitors can include, but are not limited to, e.g., 3-Deazaneplanocin A hydrochloride (DZNep—(1S,2R,5R)-5-(4-amino-1H-imidazo[4,5-c]pyridin-1-yl)-3-(hydroxymethyl)cyclopent-3-ene-1,2-diol); Bix-01294, UNC0638, BRDD4770, EPZ004777, AZ505, PDB4e47, alproic acid, vorinostat, romidepsin, entinostat abexinostat, givinostat, and mocetinostat, butyrate, a serine protease inhibitor (serpin) family member. In some embodiments, the histone methyltransferase inhibitor is DZNep. In some embodiments, the histone methyltransferase inhibitor has the following structure:

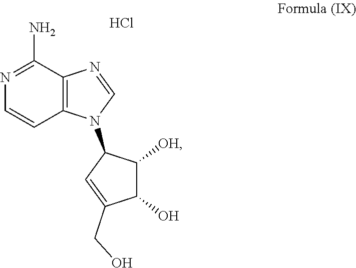 [SMILES: Cl ; Nc1nccc2c1ncn2[C@@H]1C=C(CO)[C@@H](O)[C@H]1O]

In some embodiments, the concentration of the histone methyltransferase inhibitor is from about 0.1-110 nM, 0.1-100 nM, or 0.1-50 nM. In some embodiments, the concentration of the histone methyltransferase inhibitor is about 100 nM. In some embodiments, the histone methyltransferase inhibitor is DZNep and the concentration of the inhibitor is about 100 nM.

In some embodiments, the concentration of the histone methyltransferase inhibitor can be about 0.01 μM, about 0.025 μM, about 0.05 μM, about 0.075 μM, about 0.1 μM, about 0.15 μM, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, about 20 μM, about 25 μM, about 30 μM, about 35 μM, about 40 μM, about 50 μM, or about 100 μM.

### Protein Kinase Inhibitors

Aspects of the disclosure relate to the use of protein kinase inhibitors as β cell differentiation factors.

In some embodiments, the protein kinase inhibitor in the methods and composition provided herein comprises staurosporine. In some embodiments, the protein kinase inhibitor in the methods and composition provided herein comprises an analog of staurosporine. Exemplary analogs of staurosporine in the methods and composition provided herein include, without limitation, Ro-31-8220, a bisindolylmaleimide (Bis) compound, 10′-{5″-[(methoxycarbonyl)amino]-2″-methyl}-phenylaminocarbonylstaurosporine, a staralog (see, e.g., Lopez et al., “Staurosporine-derived inhibitors broaden the scope of analog-sensitive kinase technology”, J. Am. Chem. Soc. 2013; 135(48):18153-18159), and, cgp41251.

In some embodiments, the protein kinase inhibitor in the methods and composition provided herein is an inhibitor of PKCβ. In some embodiments, the protein kinase inhibitor in the methods and composition provided herein is an inhibitor of PKCβ with the following structure or a derivative, analogue or variant of the compound as follows:

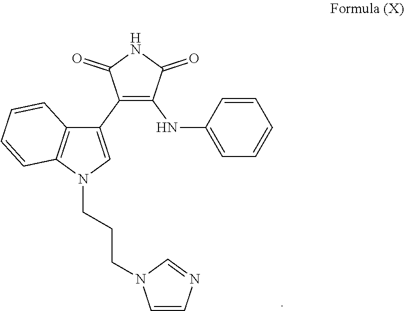 [SMILES: O=C1NC(=O)C(c2cn(CCCn3ccnc3)c3ccccc23)=C1Nc1ccccc1]

In some embodiments, the inhibitor of PKCβ is a GSK-2 compound with the following structure or a derivative, analogue or variant of the compound as follows:

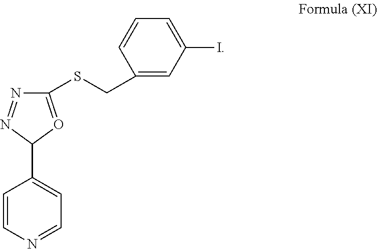

In some embodiments, the inhibitor of PKC in the methods and composition provided herein is a bisindolylmaleimide. Exemplary bisindolylmaleimides include, without limitation, bisindolylmaleimide I, bisindolylmaleimide II, bisindolylmaleimide Ill, hydrochloride, or a derivative, analogue or variant thereof.

In some embodiments, the PKC inhibitor in the methods and composition provided herein is a pseudohypericin, or a derivative, analogue, or variant thereof. In some embodiments, the PKC inhibitor in the methods and composition provided herein is indorublin-3-monoximc, 5-Iodo or a derivative, analogue or variant thereof. In certain embodiments, the methods, compositions, and kits disclosed herein exclude a protein kinase inhibitor.

### Forkhead Box O1 (FoxO1) Inhibitor

Aspects of the disclosure relate to the use of Forkhead Box O1 (FoxO1) inhibitors as differentiation factors. In some embodiments, the FoxO1 inhibitor used in the compositions and methods described herein is a compound of Formula (XII):

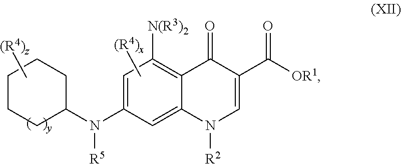

or a pharmaceutically acceptable salt, solvate, hydrate, polymorph, co-crystal, tautomer, stereoisomer, isotopically labeled derivative, prodrug, composition, or mixture thereof, wherein:
R¹ is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
R² is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
each instance of R³ is independently optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R³ are taken together with their intervening atoms to form a substituted or unsubstituted heterocyclic or substituted or unsubstituted heteroaryl ring;
each instance of R⁴ is independently halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN;
R⁵ is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
each instance of R⁶ is independently halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN;
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
x is 0, 1, or 2;
y is 0 or 1; and
z is 0, 1, 2, 3, 4, 5, 6, 7, 8, 9, or 10, as valency permits.

In some embodiments, the compound is of Formula (XII-A):

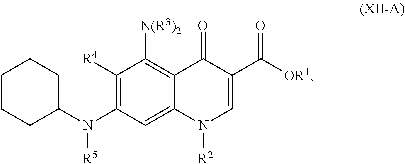

or a pharmaceutically acceptable salt, solvate, hydrate, polymorph, co-crystal, tautomer, stereoisomer, isotopically labeled derivative, prodrug, composition, or mixture thereof, wherein:
R¹ is hydrogen, optionally substituted alkyl, optionally substituted alkenyl, or optionally substituted alkynyl;
R² is hydrogen, optionally substituted alkyl, optionally substituted alkenyl, or optionally substituted alkynyl;
each instance of R³ is independently hydrogen, optionally substituted alkyl, optionally substituted alkenyl, or optionally substituted alkynyl;
R⁴ is halogen, optionally substituted acyl, optionally substituted alkyl, optionally substituted heteroalkyl, optionally substituted alkenyl; and
R⁵ is hydrogen, optionally substituted alkyl, optionally substituted alkenyl, or optionally substituted alkynyl.

In some embodiments, R¹ is hydrogen. In some embodiments, R² is optionally substituted alkyl. In some embodiments, R² is ethyl. In some embodiments, at least one instance of R³ is hydrogen. In some embodiments, both instances of R³ are hydrogen. In some embodiments, at least one instance of R⁴ is halogen. In some embodiments, at least one instance of R⁴ is fluorine. In some embodiments, x is 1. In some embodiments, R⁵ is hydrogen. In some embodiments, y is 1. In some embodiments, z is 0.

In some embodiments, the compound is of Formula (XII-B):

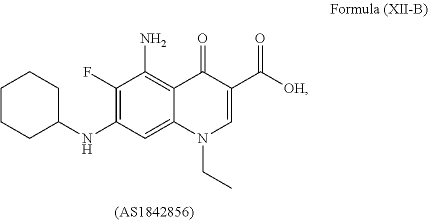 [SMILES: CCn1cc(C(=O)O)c(=O)c2c(N)c(F)c(NC3CCCCC3)cc21]

or a pharmaceutically acceptable salt, solvate, hydrate, polymorph, co-crystal, tautomer, stereoisomer, isotopically labeled derivative, prodrug, composition, or mixture thereof.

In some embodiments, the compound is AS1842856.

In some embodiments, a medium described herein does not comprise a FoxO1 inhibitor.

### Acetyl CoA-Related Metabolite

In some embodiments, a composition (e.g., medium) of the disclosure comprises an acetyl CoA-related metabolite. Metabolism of acetyl-coenzyme A (acetyl-CoA) can confer numerous metabolic functions, including energy production, lipid synthesis, and protein acetylation.

Exemplary acetyl CoA-related metabolites include, but are not limited to acetate, pyruvate, ketogenic amino acids, valine, leucine, isoleucine, phenylalanine, tyrosine, lysine, tryptophan, fatty acids, CoA, Isovaleryl-CoA, and β-hydroxybutyrate. In some embodiments, the acetyl CoA-related metabolite is acetate. In some embodiments, a composition of the disclosure contains two or more different acetyl CoA related metabolites, for example, 2, 3, 4, 5, 6, 7, 8, 9, 10, or more different acetyl CoA-related metabolites. In some embodiments, the acetyl CoA-related metabolite is acetate.

In some embodiments, a medium described herein does not include an acetyl CoA-related metabolite (e.g., does not include acetate).

### Histone Deacetylase Inhibitor (HDACi)

In some embodiments, a composition (e.g., medium) of the disclosure comprises a histone deacetylase inhibitor (HDACi). Histone deacetylase inhibitors (HDACi) are a class of compounds that increase acetylation of lysine residues on histone proteins as well as other, nonhistone, proteins by inhibiting the activity of HDAC enzymes.

Exemplary histone deacetylase inhibitors (HDACi) include, but are not limited to (3-Hydroxybutyrate, butyric acid, class I HDACi, class IIA HDACi, class IIB HDACi, class III HDACi, class IV HDACi, HDAC-1, HDAC-2, HDAC-3, HDAC-4, HDAC-5, HDAC-6, HDAC-7, HDAC-8, HDAC-9, HDAC-10, HDAC-11, sirtuins, SIRT1, SIRT2, SIRT3, SIRT4, SIRT5, SIRT6, SIRT7, Vorinostat (suberoylanilide hydroxamic acid, SAHA, MK0683), Entinostat (MS-275, SNDX-275), Panobinostat (LBH589, NVP-LBH589), Trichostatin A (TSA), Mocetinostat (MGCD0103, MG0103), GSK3117391 (GSK3117391A, HDAC-IN-3), BRD3308, BRD3308, Tubastatin A TFA (Tubastatin A trifluoroacetate salt), Tubastatin A, SIS17, NKL 22, BML-210 (CAY10433), TC-H 106, SR-4370, Belinostat (PXD101, NSC726630, PX-105684), Romidepsin (FK228, Depsipeptide, FR 901228, NSC 630176), MC1568, Givinostat (ITF2357), Dacinostat (LAQ824, NVP-LAQ824), CUDC-101, Quisinostat (JNJ-26481585), Pracinostat (SB939), PCI-34051, Droxinostat (NS 41080), Abexinostat (PCI-24781), Abexinostat (PCI-24781, CRA-024781), RGFP966, AR-42 (HDAC-42), Ricolinostat (ACY-1215, Rocilinostat), Valproic acid sodium salt (Sodium valproate), Tacedinaline (CI994, PD-123654, GOE-5549, Acetyldinaline), Fimepinostat (CUDC-907), Sodium butyrate (NaB), Curcumin, Diferuloylmethane, M344, Tubacin, RG2833 (RGFP109), RG2833 (RGFP109), Resminostat (RAS2410), Divalproex Sodium, Scriptaid (GCK 1026), Sodium Phenylbutyrate, Sinapinic acid (Sinapic acid), TMP269, Santacruzamate A (CAY10683), TMP195 (TFMO 2), Valproic acid (VPA), UF010, Tasquinimod (ABR-215050), SKLB-23bb, Isoguanosine, lforaphane, BRD73954, Citarinostat (ACY-241, HDAC-IN-2), Suberohydroxamic acid, plitomicin, HPOB, LMK-235, Biphenyl-4-sulfonyl chloride (p-Phenylbenzenesulfonyl, 4-henylbenzenesulfonyl, p-Biphenylsulfonyl), Nexturastat A, TH34, Tucidinostat (Chidamide, HBI-8000, CS-055), (−)-Parthenolide, WT161, CAY10603, CAY10603, ACY-738, RaddeaninA, Tinostamustine (EDO-S101), Domatinostat (4SC-202), and BG45.

In some embodiments, the HDACi is β-Hydroxybutyrate. β-Hydroxybutyric acid is a ketone body that, along with butyric acid, is an agonist of hydroxycarboxylic acid receptor 2 (HCA2), a Gi/o-coupled GPCR. In some embodiments, an HDACi inhibitor is an agonist of hydroxycarboxylic acid receptor 2.

In some embodiments, a medium described herein does not comprise an HDACi (e.g., does not include β-Hydroxybutyrate).

### Redox Homeostasis Regulator

In some embodiments, a composition (e.g., medium) of the disclosure comprises a redoxhomeostasis regulator.

Exemplary redox homeostasis regulators include, but are not limited to taurine, respiratory chain regulators, free radical scavengers, regulators of mitochondrial protein synthesis, allium sulphur compounds, anthocyanins, beta-carotene, catechins, copper, cryptoxanthins, flavonoids, indoles, isoflavonoids, lignans, lutein, lycopene, alpha lipoic acid, ellagic acid, manganese, polyphenols, selenium, glutathione, vitamin A, vitamin C, vitamin E, zinc, superoxide disutases, GSHPx, Prx-I, catalase, and co-enzyme Q10.

In some embodiments, the redox homeostasis regulator is taurine.

In some embodiments, a medium described herein does not comprise a redox homeostasis regulator.

Taurine is a non-proteinogenic ß-aminosulfonic acid that can be derived from methionine and cysteine metabolism. In some embodiments, taurine can inhibit ROS generation within the respiratory chain.

In some embodiments, a medium described herein does not comprise a redox homeostasis regulator (e.g., does not include taurine).

### One Carbon Metabolism Pathway Intermediate

In some embodiments, a composition (e.g., medium) of the disclosure comprises a one carbon metabolism pathway intermediate. One-carbon metabolism mediated by folate cofactors, supports multiple physiological processes including amino acid homeostasis (methionine, glycine and serine), biosynthesis of nucleotides (purines, thymidine), epigenetic maintenance, and redox defense.

Exemplary one carbon metabolism pathway intermediates include, but are not limited to formate, tetrahydrofolate (THF), 10-formylTHF; 5,10-meTHF; 5,10-meTHF; and 10-formylTHF.

In some embodiments, a medium described herein does not comprise a one carbon metabolism pathway intermediate (e.g., does not include formate).

### Glutamine

In some embodiments, a composition (e.g., medium) of the disclosure comprises glutamine. Glutamine (Gln or Q) is an alpha-amino acid. Glutamine can be an essential amino acid within in vitro cell cultures. Glutamine supports the growth of cells, including cells that have high energy demands and synthesize large amounts of proteins and nucleic acids. It is an alternative energy source for rapidly dividing cells and cells that use glucose inefficiently.

In some embodiments, compositions and methods of the disclosure utilize glutamine in a form with increased bioavailability. Because of its chemical instability and importance for cell growth and function, it is important that delivery of L-glutamine be tailored to each unique cell culture process. Glutamine (e.g., L-glutamine) in a free form can be unstable at physiological pH in liquid media, breaking down to ammonium and pyroglutamate at rates that make it a problem in many cell culture and biomanufacturing applications. Therefore, many cell culture media contain stabilized forms of glutamine, including dipeptide forms, such as alanyl-1-glutamine and glycyl-1-glutamine. However, these more stable forms of L-glutamine can also have limited bioavailability, for example, due to a requirement for processing by enzymes, such as cell surface peptidases. Thus in some embodiments, compositions and methods of the disclosure utilize glutamine in a form with increased bioavailability, such as a free glutamine form, such as a non-dipeptide form, a non-alanine-glutamine dipeptide form (e.g., a non-alanyl-1-glutamine form), a non-glycine-glutamine dipeptide form (e.g., a non-glycyl-1-glutamine form), a form that in which glutamine is not conjugated to another amino acid or stabilizing moiety, a monomeric form, a free form, or a combination thereof. In some embodiments, glutamine is provided as a protein hydrolysate.

In some embodiments, a basal media contains glutamine. In some embodiments, glutamine in a form as disclosed herein is added to a media that already contains glutamine. In some embodiments, glutamine in a form as disclosed herein is added to a basal media that contains no glutamine or only low levels of glutamine to increase the bioavailability of glutamine.

In some embodiments, a medium described herein does not comprise glutamine.

### Glutamate

In some embodiments, a composition (e.g., medium) of the disclosure comprises glutamate (e.g., L-glutamate). Glutamate can be converted into, for example, g-amino butyric acid (GABA), ornithine, 2-oxoglutarate, glucose or glutathione. Glutamate and metabolites generated therefrom can contribute to, for example, redox homeostasis, cell signaling, nitrogen assimilation, amine catabolism, amino acid biosynthesis, nucleoside biosynthesis, and cofactor production.

In some embodiments, contacting cells with glutamate can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks glutamate, or contains a lower concentration of glutamate.

In some embodiments, a medium described herein does not comprise glutamate. Vitamins

In some embodiments, a composition (e.g., medium) of the disclosure comprises one or more vitamins.

Exemplary vitamins include, but are not limited to biotin, vitamin B1 (thiamine), vitamin B2 (riboflavin), vitamin B3 (niacin), vitamin B6 (pyridoxine) and vitamin B12 (cyanocobalamin). In some embodiments the vitamin modulates fatty acid synthesis. In some embodiments the vitamin modulates branched-chain amino acid metabolism. In some embodiments the vitamin modulates or participates as a co-factor in the TCA cycle, e.g., as a cofactor for pyruvate carboxylase. In some embodiments, the vitamin is biotin. In some embodiments, a composition of the disclosure contains two or more different vitamins, for example, 2, 3, 4, 5, 6, 7, 8, 9, 10, or more different vitamins.

In some embodiments, a medium described herein does not comprise a vitamin.

### Water-Soluble Synthetic Polymer

Water-soluble polymer described herein can refer to any polymer that has hydrophilic property and is soluble in aqueous solution at room temperature. The water-soluble polymer can be either naturally occurring or synthetic. In some embodiments, a water-soluble polymer is an albumin protein (e.g., human serum albumin or bovine serum albumin). In some embodiments, the water-soluble polymer is a water-soluble synthetic polymer. Water-soluble synthetic polymers described herein can refer to any synthetic polymer that has hydrophilic property and is soluble in aqueous solution at room temperature. Water-soluble synthetic polymers applicable in the subject methods and compositions include, but not limited to, poloxamer, polyvinyl alcohol, polyvinylpyrrolidone, polyethylene glycol (PEG), PEG copolymers, poly(Nisopropylacrylamide), and polyacrylamide. The water-soluble synthetic polymer can refer to a polymer compound or a mixture of polymer compounds that may have an idealized chemical formula but a variety of derivatives and/or precursors of the idealized formula, depending on the applicable manufacturing method. In some embodiments, the water-soluble synthetic polymer is used to replace at least partially serum or serum albumin, e.g., BSA or HSA, that is typically utilized in cell differentiation, e.g., differentiation of pancreatic β cells or precursor cells thereof. In some embodiments, the water-soluble synthetic polymer replaces 100% of serum albumin, e.g., BSA or HSA, that is typically utilized in cell differentiation, e.g., differentiation of pancreatic β cells or precursor cells thereof. In some embodiments, the water-soluble synthetic polymer reduces the amount of serum albumin, e.g., BSA or HSA, by at least 20%, 30%, 40%, 50%, 60%, 80%, 90%, 95%, or 99% of that is typically utilized in cell differentiation, e.g., differentiation of pancreatic β cells or precursor cells thereof. In some embodiments, the disclosure provides for a composition comprising a population of any of the cells disclosed herein (e.g., pluripotent stem cells; endoderm cells; primitive gut cells; PDX1-positive, NKX6.1-negative pancreatic progenitor cells; PDX1-positive, NKX6.1-positive pancreatic progenitor cells; insulin-positive cells; and/or pancreatic beta cells) and water soluble polymers, wherein at least 20%, 30%, 40%, 50%, 60%, 80%, 90%, 95%, or 99% of the water soluble polymers in the composition are water-soluble synthetic polymers (e.g., any of the PVA molecules disclosed herein) and wherein the remainder of the water soluble polymers are human serum albumin polypeptides. In some embodiments, the disclosure provides for a composition comprising a population of any of the cells disclosed herein (e.g., pluripotent stem cells; endoderm cells; primitive gut cells; PDX1-positive, NKX6.1-negative pancreatic progenitor cells; PDX1-positive, NKX6.1-positive pancreatic progenitor cells; insulin-positive cells; and/or pancreatic beta cells) and water soluble polymers, wherein no more than 1%, 2%, 3%, 4%, 5%, 6%, 7%, 8%, 9%10%, 15%, 20%, 30%, 40%, 50%, 60%, 80%, 90%, 95%, or 99% of the water soluble polymers are naturally occurring water-soluble polymers (e.g., HSA or BSA). In some embodiments, more than 90%, 95%, 99%, and up to 100% of the water soluble polymers in the composition are water-soluble synthetic polymers (e.g., PVA).

In some embodiments, the water-soluble synthetic polymer applicable to the subject compositions and methods includes polyvinyl alcohol (PVA). Polyvinyl alcohol described herein can refer to a water-soluble synthetic polymer that has an idealized formula [CH2CH(OH)]n, which can be either partially or completed hydrolyzed. In some embodiments, the polyvinyl alcohol is manufactured by either partial or complete hydrolysis of polyvinyl acetate to remove acetate groups. In some embodiments, the polyvinyl alcohol is at most 85% hydrolyzed, e.g., 80% hydrolyzed. The percentage of hydrolyzation measures the approximate percentage (e.g., average percentage) of acetate residue that is hydrolyzed in the polyvinyl acetate precursor polymer. In some embodiments, the polyvinyl alcohol is at least 85% hydrolyzed, e.g., 87-89% hydrolyzed, 87-90% hydrolyzed, or 99% hydrolyzed. In some embodiments, the polyvinyl alcohol is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. Without wishing to be bound by a certain theory, the polyvinyl alcohol can assume a function of carrier-molecule in the culture medium, which is typically carried out by serum or serum albumin, e.g., HSA. The percentage of hydrolyzation of polyvinyl alcohol can be determined by the manufacturing method utilized to produce the polyvinyl alcohol, e.g., how polyvinyl acetate precursor polymer is converted into polyvinyl alcohol, e.g., conversion by base-catalyzed transesterification with ethanol. In some embodiments, the water-soluble synthetic polymer preparation, e.g., polyvinyl alcohol, that is used in the subject method or present in the subject composition has purity of at least 90%, such as at least 92%, at least 95%, at least 98%, at least 99%, at least 99.5%, at least 99.9%, or nearly 100%. Purity of polyvinyl alcohol measures the percentage of synthetic polymer that has the idealized formula [CH2CH(OH)]n in the preparation, which includes polyvinyl alcohol of any percentage of hydrolyzation. Impurity of polyvinyl alcohol preparation can include other polymer materials that do not have the idealized formula [CH2CH(OH)]n, or other organic inorganic materials.

In some embodiments, a medium described herein does not comprise a water-soluble synthetic polymer.

### Cell Clusters of Stem Cell Derived Beta Cells

In some aspects, provided herein are cell clusters that resemble the functions and characteristics of endogenous pancreatic islets. Such cell clusters can mimic the function of endogenous pancreatic islets in regulating metabolism, e.g., glucose metabolism in a subject. Thus, the cell clusters can be transplanted to a subject for treating disease resulting from insufficient pancreatic islet function, e.g., diabetes. The terms “cluster” and “aggregate” can be used interchangeably, and refer to a group of cells that have close cell-to-cell contact, and in some cases, the cells in a cluster can be adhered to one another.

A cell cluster comprises a plurality of cells. In some embodiments, a cell cluster comprises at least 10, at least 50, at least 200, at least 500, at least 750, at least 1000, at least 1500, at least 2000, at least 2500, at least 3000, at least 3500, at least 4000, at least 4500, at least 5000, at least 6000, at least 7000, at least 8000, at least 9000, at least 10,000, at least 20,000, at least 30,000, or at least 50,000 cells. In some embodiments, a cell cluster comprises between 10-10,000 cells, between 50-10,000, between 100-10,000, between 100-10,000, between 1,000-10,000, between 500 and 10,000, between 500 and 5,000, between 500 and 2,500, between 500 and 2,000, between 1,000 and 100,000, between 1,000 and 50,000, between 1,000 and 40,000, between 1,000 and 20,000, between 1,000 and 10,000, between 1,000 and 5,000 and between 1,000 and 3,000 cells. In some embodiments, a cell cluster comprises at least 500 cells. In some embodiments, a cell cluster comprises at least 1,000 cells. In some embodiments, a cell cluster comprises at least 2,000 cells. In some embodiments, a cell cluster comprises at least 5,000 cells. In some embodiments, a cell cluster comprises no more than 100,000, no more than 90,000, no more than 80,000, no more than 70,000, no more than 60,000, no more than 50,000, no more than 40,000, no more than 30,000, no more than 20,000, no more than 10,000, no more than 7,000, no more than 5,000, no more than 3,000, no more than 2,000 cells, or no more than 1,000 cells.

A cell cluster herein can comprise at least one non-native cell, e.g., a non-native pancreatic β cell. A non-native cell (e.g., a non-native pancreatic β cell) can share characteristics of an endogenous cell (e.g., an endogenous mature pancreatic β cell), but is different in certain aspects (e.g., gene expression profiles). A non-native cell can be a genetically modified cell. A non-native cell can be a cell differentiated from a progenitor cell, e.g., a stem cell. The stem cell can be an embryonic stem cell (ESC) or induced pluripotent stem cell (iPSC). In some cases, the non-native cell can be a cell differentiated from a progenitor cell in vitro. In some cases, the non-native cell can be a cell differentiated from a progenitor cell in in vivo. For example, a cell cluster can comprise at least one non-native pancreatic β cell. The non-native pancreatic β cells can be those described in U.S. Patent Publication Nos. US20150240212A1 and US20150218522A1, which are incorporated herein in their entireties. A cell cluster can comprise a plurality of non-native pancreatic β cells. In some cases, at least about 20%, 30%, 40%, 50%, 60%, 70%, 80%, 90%, 99% cells in a cell cluster are non-native pancreatic β cells. A cell cluster can comprise one or more native cells. For example, a cell cluster can comprise one or more primary cells, e.g., primary cells from an endogenous pancreatic islet.

A cell cluster can comprise one or more cells expressing at least one marker of an endogenous cell, e.g., an endogenous mature pancreatic β cell. The term “marker” can refer to a molecule that can be observed or detected. For example, a marker can include, but is not limited to, a nucleic acid, such as a transcript of a specific gene, a polypeptide product of a gene, a non-gene product polypeptide, a glycoprotein, a carbohydrate, a glycolipid, a lipid, a lipoprotein, or a small molecule. In many cases, a marker can refer to a molecule that can be characteristic of a particular type of cell, so that the marker can be called as a marker of the type of cell. For instance, Insulin gene can be referred to as a marker of β cells. In some cases, a marker is a gene. Non-limiting of markers of an endogenous mature pancreatic β cell include insulin, C-peptide, PDX1, NKX6.1, CHGA, MAFA, ZNT8, PAX6, NEUROD1, glucokinase (GCK), SLC2A, PCSK1, KCNJ11, ABCC8, SLC30A8, SNAP25, RAB3A, GAD2, and PTPRN.

A cell cluster can comprise one more cells expressing one or multiple markers of an endogenous cell, e.g., an endogenous mature pancreatic β cell. For example, a cell cluster can comprise one or more cells co-expressing at least 2, 3, 4, 5, 6, 7, 8, 9, 10, 15, or 20 marker(s) of an endogenous cell, e.g., an endogenous mature pancreatic β cell. In some cases, a cell cluster comprises cells that express NKX6.1 and C-peptide, both of which can be markers of a β cell.

A cell cluster can comprise a plurality of cells expressing at least one marker of an endogenous cell. For example, at least about 1%, 5%, 10%, 20%, 30%, 40%, 50%, 60%, 70%, 80%, 90%, 95%, 99% cells in a cell cluster can express at least one marker of an endogenous cell. In some cases, all cells in a cell cluster can express a marker of an endogenous cell. In some cases, the endogenous cell can be a pancreatic cell, e.g., a pancreatic β cell, pancreatic α cells, pancreatic β cells, pancreatic δ cells, or pancreatic γ cells. A cell cluster as provided herein can comprise a heterogeneous group of cells, e.g., cells of different types. For example, the cell cluster can comprise a cell expressing insulin/C-peptide, which can be a marker of a pancreatic R cell, a cell expressing glucagon, which can be a marker of a pancreatic α cell, a cell expressing somatostatin, which can be a marker of a pancreatic δ cell, a cell expressing pancreatic polypeptides, or any combination thereof.

For example, the cell cluster herein can comprise a plurality of cells expressing one or more markers of an endogenous mature pancreatic β cell. For example, at least about 10%, 20%, 30%, 40%, 50%, 60%, 70%, 80%, 90%, 95%, or 99% cells in the cell cluster can express one or more markers of an endogenous mature pancreatic β cell.

The cell cluster can comprise a plurality of cells expressing CHGA. In some cases, at least about 30%, 40%, 50%, 60%, 70%, 80%, 90%, 95%, or 99% cells in the cell cluster express CHGA. In some cases, at least about 85% cells in a cell cluster can express CHGA. In some cases, a cell cluster can comprise about 90% cell expressing CHGA. In some cases, a cell cluster can comprise about 95% cells expressing CHGA. In certain cases, all cells in a cell cluster can express CHGA.

The cell cluster can comprise a plurality of cells expressing NKX6.1. For example, at least about 30%, 40%, 50%, 60%, 70%, 80%, 90%, 95%, 99% cells in a cell cluster can express NKX6.1. In some cases, at least about 50% cells in a cell cluster can express NKX6.1. In some cases, all cells in a cell cluster can express NKX6.1.

The cell cluster can comprise a plurality of cells expressing ISL1. For example, at least about 30%, 40%, 50%, 60%, 70%, 80%, 90%, 95%, 99% cells in a cell cluster can express NKX6.1. In some cases, at least about 50% cells in a cell cluster can express ISL1. In some cases, all cells in a cell cluster can express ISL1.

The cell cluster can comprise a plurality of cells expressing C-peptide. For example, at least about 30%, 40%, 50%, 60%, 70%, 80%, 90%, 95%, or 99% cells in a cell cluster can express C-peptide. In some cases, at least about 60% cells in a cell cluster can express C-peptide. In some cases, all cells in a cell cluster can express C-peptide.

The cell cluster can comprise a plurality of cells expressing both NKX6.1 and C-peptide. For example, at least about 10%, 20%, 30%, 40%, 50%, 60%, 70%, 80%, 90%, 95%, or 99% cells in a cell cluster can express C-peptide. In some cases, at least about 35% cells in a cell cluster can express NKX6.1 and C-peptide. In some cases, at least about 40% cells in a cell cluster can express NKX6.1 and C-peptide. In some cases, at least about 35% cells in a cell cluster can express NKX6.1 and C-peptide. In some cases, a cell cluster can comprise about 60% cells expressing NKX6.1 and C-peptide. In some cases, a cell cluster can comprise about 75% cell expressing NKX6.1 and C-peptide. In some cases, all cells in a cell cluster can express NKX6.1 and C-peptide.

The cell cluster can comprise a plurality of cells expressing both NKX6.1 and ISL1. For example, at least about 10%, 20%, 30%, 35%, 38%, 40%, 42%, 44%, 46%, 50%, 60%, 70%, 80%, 90%, 95%, or 99% cells in a cell cluster can express NKX6.1 and ISL1. In some cases, at least about 35% cells in a cell cluster can express NKX6.1 and ISL1. In some cases, at least about 40% cells in a cell cluster can express NKX6.1 and ISL1. In some cases, at least about 35% cells in a cell cluster can express NKX6.1 and ISL1. In some cases, a cell cluster can comprise about 60% cells expressing NKX6.1 and ISL1. In some cases, a cell cluster can comprise about 75% cell expressing NKX6.1 and ISL1. In some cases, all cells in a cell cluster can express NKX6.1 and ISL1.

The cell cluster can comprise a limited proportion of cells that are negative for both NKX6.1 and ISL1. For example, a cell cluster can comprise at most 2%, at most 4%, at most 6%, at most 8%, at most 10%, at most 12%, at most 14%, at most 16%, at most 18%, at most 20%, at most 22%, at most 22%, at most 25%, or at most 30% ISL1-negative, NKX6.1-negative cells.

The cell cluster can comprise very few to none of stem cells or progenitor cells, e.g., pancreatic progenitor cells. For example, a cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing LIN28. In some examples, a cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing Ki67.

In some cases, a cell cluster can comprise at most 3% cells, at most about 2% cells, at most about 1% cells, at most about 0.5% cells, at most about 0.1% cells, at most about 0.05% cells, at most about 0.01% cells, or no cells expressing SOX2. In some cases, a cell cluster can comprise about 1% cells expressing SOX2. In some cases, a cell cluster can comprise about 0.6% cells expressing SOX2. In some cases, a cell cluster can comprise about 0.3% cells expressing SOX2. In some cases, a cell cluster can comprise about 0.1% cells expressing SOX2.

In some examples, a cell cluster can comprise at most 10% cells, at most about 8% cells, at most about 6% cells, at most about 5% cells, at most about 2% cells, at most about 1% cells, at most about 0.5% cells, at most about 0.1% cells, at most about 0.05% cells, at most about 0.01% cells, or no cells expressing SOX9. In some cases, a cell cluster can comprise about 2% cells expressing SOX9. In some cases, a cell cluster can comprise about 6% cells expressing SOX9. In some cases, a cell cluster can comprise about 1.2% cells expressing SOX9.

A cell cluster herein can exhibit one or multiple glucose stimulated insulin secretion (GSIS) response(s) in vitro when exposed to glucose challenge(s). The GSIS responses can resemble the GSIS responses of an endogenous pancreatic islet. In some cases, the cell cluster exhibits an in vitro GSIS response to a glucose challenge. In some cases, the cell cluster exhibits in vitro GSIS responses to multiple glucose challenges, such as sequential glucose challenges. For example, the cell cluster can exhibit in vitro GSIS responses to at least 2, 3, 4, 5, 6, 7, 8, 9, 10 sequential glucose challenges.

A cell cluster as provided herein can comprise at least one cell exhibiting in vitro GSIS. For example, at least one cell in the cell cluster can be referred to as a mature pancreatic β cell. In some cases, the at least one cell is a non-native pancreatic β cell. In some cases, the at least one cell is a pancreatic β cell resembling a native/endogenous β cell. In some cases, the cell exhibits an in vitro glucose stimulated insulin secretion (GSIS) response. In some cases, the at least one cell exhibits a GSIS response to at least one glucose challenge. In some cases, the cell exhibits a GSIS response to at least two sequential glucose challenges. In some cases, the cell exhibits a GSIS response to at least three sequential glucose challenges.

As provided herein, a cell cluster can exhibit GSIS stimulation index similar to an endogenous pancreatic islet. Stimulation index of a cell cluster or a cell can be characterized by the ratio of insulin secreted in response to high glucose concentrations compared to low glucose concentrations. For example, a stimulation index of a cell cluster or a cell as provided herein can be calculated as a ratio of insulin secreted in response to 20 mM glucose stimulation versus insulin secreted in response to 2.8 mM glucose stimulation. In some examples, the stimulation index of a cell cluster or a cell as provided herein is greater than or equal to 1, or greater than or equal to 1.1, or greater than or equal to 1.3, or greater than or equal to 2, or greater than or equal to 2.3, or greater than or equal to 2.6. In some instances, the cell cluster or the cell exhibits cytokine-induced apoptosis in response to a cytokine. In some cases, the cytokine comprises interleukin-β (IL-β), interferon-γ (INF-γ), tumor necrosis factor-α (TNF-α), or any combination thereof. In some cases, insulin secretion from the cell cluster or the cell is enhanced in response to an anti-diabetic agent. In some cases, the anti-diabetic agent comprises a secretagogue selected from the group consisting of an incretin mimetic, a sulfonylurea, a meglitinide, and combinations thereof. In some cases, the cell cluster or the cell is monohormonal. In some cases, the cell cluster or the cell exhibits a morphology that resembles the morphology of an endogenous mature pancreatic β cell. In some cases, the cell cluster or the cell exhibits encapsulated crystalline insulin granules under electron microscopy that resemble insulin granules of an endogenous mature pancreatic β cell. In some cases, the cell cluster or the cell exhibits a low rate of replication. In some cases, the cell cluster or the cell exhibits a glucose stimulated Ca²⁺ flux (GSCF) that resembles the GSCF of an endogenous mature pancreatic R cell. In some cases, the cell cluster or the cell exhibits a GSCF response to at least one glucose challenge. In some cases, the cell cluster or the cell exhibits a GSCF response to at least two glucose challenges. In some cases, the cell cluster or the cell exhibits a GSCF response to at least three glucose challenges. In some cases, the cell cluster or the cell exhibits an increased calcium flux. In some cases, the increased calcium flux comprises an increased amount of influx or a ratio of influx at low relative to high glucose concentrations.

A cell cluster as provided herein can exhibit biphasic insulin secretion in response to a high glucose concentration stimulation similar to an endogenous pancreatic islet, e.g., a human pancreatic islet. A biphasic insulin secretion can be a phenomenon characteristic of an endogenous pancreatic islet, e.g., human islet. In some embodiments, response to a high glucose concentration challenge, e.g., 10 mM, 15 mM, 20 mM, or 30 mM, a cell cluster as provided herein, e.g., a reaggregated pancreatic cell cluster, can exhibit a transient increase in insulin secretion to a peak value followed by a rapid decrease to a relatively elevated insulin secretion level, e.g., a level that is higher than an insulin secretion level in response to a lower glucose concentration, e.g., 2.8 mM glucose. Such a transient increase and decrease process can be termed as a first phase of the biphasic insulin secretion pattern. With a persistent high glucose challenge, the first phase can be thus followed by a second phase, in which the insulin secretion by the cell cluster can be maintained at the relatively elevated level. The second phase can last for an extended period, e.g., as long as the high glucose concentration challenge lasts, or relatively longer than the first phase. Such a biphasic insulin secretion pattern can be due to intrinsic cellular signaling changes that are characteristic of a mature native pancreatic β cell.

In some embodiments, any of the cell populations and/or cell clusters disclosed herein comprises NKX6.1-positive, ISL1-positive cells that express lower levels of MAFA than NKX6.1-positive, ISL1-positive cells from the pancreas of a healthy control adult subject. In some embodiments, the population comprises NKX6.1-positive, ISL1-positive cells that express higher levels of MAFB than NKX6.1-positive, ISL1-positive cells from the pancreas of a healthy control adult subject. In some embodiments, the population comprises NKX6.1-positive, ISL1-positive cells that express higher levels of SIX2, HOPX, IAPP and/or UCN3 than NKX6.1-positive, ISL1-positive cells from the pancreas of a healthy control adult subject. In some embodiments, the population comprises NKX6.1-positive, ISL1-positive cells that do not express MAFA. In some embodiments, the population comprises NKX6.1-positive, ISL1-positive cells that express MAFB.

In some embodiments, any of the cell populations and/or cell clusters disclosed herein comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, said cells comprise a genomic disruption in at least one gene sequence, wherein said disruption reduces or eliminates expression of a protein encoded by said gene sequence. In some embodiments, any of the cells disclosed herein (e.g., any of the SC-derived beta cells or cells in any of the clusters disclosed herein) comprise a genomic disruption in at least one gene sequence, wherein said disruption reduces or eliminates expression of a protein encoded by said gene sequence. In some embodiments, said at least one gene sequence encodes an MHC-Class I gene. In some embodiments, said MHC-Class I gene encodes beta-2 microglobulin (B2M), HLA-A, HLA-B, or HLA-C. In some embodiments, said at least one gene sequence encodes CIITA. In some embodiments, the cells comprise a genomic disruption in the genes encoding HLA-A and HLA-B, but do not comprise a genomic disruption in the gene encoding HLA-C. In some embodiments, said cells comprise a genomic disruption in a natural killer cell activating ligand gene. In some embodiments, said natural killer cell activating ligand gene encodes intercellular adhesion molecule 1 (ICAM1), CD58, CD155, carcinoembryonic antigen-related cell adhesion molecule 1 (CEACAM1), cell adhesion molecule 1 (CADM1), MHC-Class I polypeptide-related sequence A (MICA), or MHC-Class I polypeptide-related sequence B (MICB). In some embodiments, the cells have reduced expression of one or more of beta-2 microglobulin, CIITA, HLA-A, HLA-B, HLA-C, HLA-DP, HLA-DQ, and HLADR, relative to stem cells that are not genetically modified. In some embodiments, the cells have increased expression of CD47, PDL1, HLA-G, CD46, CD55, CD59 and CTLA, relative to stem cells that are not genetically modified. In particular embodiments, the pancreatic islet cells disclosed herein (e.g., the SC-beta cells) have increased expression of PDL1 as compared to endogenous pancreatic islet cells from a healthy control subject. In particular embodiments, the pancreatic islet cells disclosed herein (e.g., the SC-beta cells) have increased expression of CD47 as compared to endogenous pancreatic islet cells from a healthy control subject. In some embodiments, the genomic disruption is induced by use of a gene editing system, e.g., CRISPR Cas technology.

In some embodiments, any of the cells disclosed herein (e.g., any of the stem cells disclosed herein) comprises a “safety switch.” In some embodiments, the safety switches are nucleic acid constructs encoding a switch protein that inducibly causes cell death or stops cell proliferation. In some embodiments, the safety switch is inserted at a defined, specific target locus (e.g., a safe harbor locus) in the genome of an engineered cell, usually at both alleles of the target locus. In some embodiments, the target locus is a safe harbor locus, such as ActB or CLYBL. In some embodiments, the switch protein is activated by contacting with an effective dose of a clinically acceptable orthologous small molecule. In some embodiments, when activated, the safety switch causes the cell to stop proliferation, in some embodiments by activating apoptosis of the cell. In some embodiments, the switch protein comprises herpes-simplex-thymidine-kinase. In some embodiments the switch protein comprises a human caspase protein, e.g., caspase 1, caspase 2, caspase 3, caspase 4, caspase 5, caspase 6, caspase 7, caspase 8, caspase 9, caspase 10, caspase 14, etc. In certain embodiments the protein is human caspase 9. In some embodiments, the caspase protein is fused to a sequence that provides for chemically induced dimerization (CID), in which dimerization occurs only in the presence of the orthologous activating agent. One or more CID domains may be fused to the caspase protein, e.g., two different CID domains may be fused to the caspase protein. In some embodiments the CID domain is a dimerization domain of FKBP or FRB (FKBP-rapamycin-binding) domain of mTOR, which are activated with rapamycin analogs. In some embodiments, the safety switch is any of the safety switches described in WO2021173449 and Jones et al., 2014, Frontiers in Pharmacology, 5(254):1-8, each of which is incorporated herein in its entirety.

When transplanted to a subject, a cell cluster can exhibit one or more in vivo GSIS responses when exposed to glucose challenge(s). The cell cluster herein can be capable of exhibiting an in vivo GSIS response within a short period of time after transplanted to a subject. For example, the cell cluster can exhibit an in vivo GSIS within about 6, 12, or 24 hours after transplantation. In some cases, the cell cluster exhibits an in vivo GSIS within about 2 days, 4 days, 6 days, 8 days, 10 days, 12 days, 14 days, 21 days, 28 days, 35 days, or 42 days after transplantation. The amount of insulin secreted by the cell cluster can be similar or higher than an endogenous pancreatic islet. The term “about” in relation to a reference numerical value as used through the application can include a range of values plus or minus 10% from that value. For example, the amount “about 10” includes amounts from 9 to 11. For example, the term “about” in relation to a reference numerical value can also include a range of values plus or minus 10%, 9%, 8%, 7%, 6%, 5%, 4%, 3%, 2%, or 1% from that value.

The cell cluster can maintain the ability of exhibiting in vivo GSIS responses for a period of time after transplanted into a subject. For example, an in vivo GSIS response of the cell cluster can be observed up to at least 2 weeks, 3 weeks, 4 weeks, 5 weeks, 10 weeks, 15 weeks, 20 weeks, 6 months, 7 months, 8 months, 9 months, 10 months, 11 months, 1 year, 2 years, 3 years, 4 years, 5 years, 10 years, 20 years, 30 years, 40 years, 60 years, 80 years, or 100 years after transplantation of the cell cluster into a subject (e.g., a human).

The GSIS of a cell cluster can be measured by a stimulation index. A stimulation index of a cell cluster can equal to the ratio of insulin secreted in response to a high glucose concentration compared to insulin secreted in response to a low glucose concentration. A cell cluster can have a stimulation index similar to an endogenous pancreatic islet. In some cases, a cell cluster has a stimulation index of at least 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

The amount of insulin secreted by a cell cluster in response to a glucose challenge (e.g., a high concentration, such as 20 mM, of glucose) can range from about 0.1 μIU/10³ cells to about 5 μIU/10³ cells, from about 0.2 μIU/10³ cells to about 4 μIU/10³ cells, from about 0.2 μIU/10³ cells to about 3 μIU/10³ cells, or from about 0.23 μIU/10³ cells to about 2.7 μIU/10³ cells. In some cases, the amount of insulin secreted by a cell cluster in response to a glucose challenge (e.g., a high concentration, such as 20 mM, of glucose) is at least 0.05, 0.1, 0.15, 0.2, 0.21, 0.22, 0.23, 0.24, 0.25, 0.26, 0.27, 0.28, 0.29, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 μIU/10³ cells.

A cell cluster can secrete both pro-insulin and insulin. For example, a cell cluster can secrete pro-insulin and insulin at a proinsulin-to-insulin ratio substantially the same as the ratio of pro-insulin to insulin secreted by an endogenous pancreatic islet. In some cases, a cell cluster secretes pro-insulin and insulin at a proinsulin-to-insulin ratio of from about 0.01 to about 0.05, from about 0.02 to about 0.04, from about 0.02 to about 0.03, or from 0.029 to about 0.031. In some cases, a cell cluster secretes pro-insulin and insulin at a proinsulin-to-insulin ratio of about 0.02, 0.021, 0.022, 0.023, 0.024, 0.025, 0.026, 0.027, 0.028, 0.029, 0.03, 0.031, 0.032, 0.033, 0.034, 0.035, 0.036, 0.037, 0.038, 0.039, or 0.04.

A cell cluster can be in a size similar to an endogenous pancreatic islet. For example, a cell cluster can have a diameter similar to an endogenous pancreatic islet. A diameter of a cell cluster can refer to the largest linear distance between two points on the surface of the cell cluster. In some cases, the diameter of a cell cluster is at most 300 μm, 200 μm, 150 μm, 100 μm, 90 μm, 80 μm, 70 μm, 60 μm, 50 μm, or 40 μm. The diameter of a cell cluster can be from about 75 μm to about 250 μm. The diameter of a cell cluster can be at most 100 μm.

In some embodiments, at least about 40%, 50%, 60%, 70%, 80%, or 90% of cell clusters have a diameter of from about 50 μm to about 250 μm, from about 75 μm to about 250 μm, or from about 100 μm to about 200 μm.

In some embodiments, at least about 40%, 50%, 60%, 70%, 80%, or 90% of the cell clusters have a diameter of about 80-150, about 100-150, about 120-150, about 140-150, about 80-130, about 100-130, about 120-130, about 80-120, about 90-120, or about 100-120 μm.

In some embodiments, the cell clusters have a mean or median diameter of at most 120, at most 130, at most 140, at most 150, at most 160, or at most 170 μm.

In some embodiments, the cell clusters have a mean or median diameter of about 80-150, about 100-150, about 120-150, about 140-150, about 80-130, about 100-130, about 120-130, about 80-120, about 90-120, or about 100-120 μm.

In some embodiments, at least about 50%, at least about 60%, at least about 70%, or at least about 80% of the cell clusters have a diameter of less than 150 μm.

In some embodiments, at least about 50%, at least about 60%, or at least about 70% of the cell clusters have a diameter of less than 140 μm.

In some embodiments, at least about 50%, at least about 60%, or at least about 70% of the cell clusters have a diameter of less than 130 μm.

A cell cluster can comprise very few or no dead cells. The cell cluster can be in a size that allows effective diffusion of molecules (e.g., nutrition and gas) from surrounding environment into the core of the cell cluster. The diffused molecule can be important for the survival and function of the cells in the core. In some cases, the cell cluster can have less than about 1%, 2%, 3%, 4%, 5%, 6%, 7%, 8%, 9%, 10% of dead cells, e.g., dead cells in its core. In some cases, a cell cluster can have no dead cell. The dead cells can be apoptotic cells, narcotic cells or any combination thereof.

A cell cluster can comprise one or multiple types of cells. In some cases, a cell cluster comprises one or more types of pancreatic cells. For example, the cell cluster can comprise one or more pancreatic β cell, pancreatic α cells, pancreatic Δ cells, pancreatic γ cells, and any combination thereof. In some cases, the pancreatic cells can be non-native pancreatic cells, e.g., cells derived from stem cells, such as ESCs and/or iPSCs. In some cases, the cell cluster can also comprise one or more progenitor cells of mature pancreatic cells, including iPSCs, ESCs, definitive endoderm cells, primitive gut tube cells, Pdx1-positive pancreatic progenitor cells, Pdx1-positive/NKX6.1-positive pancreatic progenitor cells, Ngn3-positive endocrine progenitor cells, and any combination thereof.

In some embodiments, a cell cluster can exhibit cytokine-induced apoptosis in response to cytokines. For example, the cell cluster may exhibit cytokine-induced apoptosis in response to a cytokine such as interleukin-1β (IL-β), interferon-γ (INF-γ), tumor necrosis factor-α (TNF-α), and combinations thereof.

Insulin secretion from a cell cluster herein can be enhanced by an anti-diabetic drug (e.g., an anti-diabetic drug acting on pancreatic β cells ex vivo, in vitro, and/or in vivo). The disclosure can contemplate any known anti-diabetic drug. In some cases, insulin secretion from a cell cluster can be enhanced by a secretagogue. The secretagogue can be an incretin mimetic, a sulfonylurea, a meglitinide, and combinations thereof.

A cell cluster can comprise a monohormonal. For example, the cell cluster can comprise a pancreatic cell (e.g., a pancreatic β cell, pancreatic α cells, pancreatic β cells, pancreatic Δ cells, or pancreatic γ cells) that is monohormonal. In some cases, the cell cluster comprises an insulin-secreting non-native pancreatic cell that is monohormonal. A cell cluster can comprise a polyhormonal. In some case, a cell cluster comprises a monohormonal cell and a polyhormonal cell.

A cell cluster can comprise a cell (e.g., a non-native pancreatic cell) having a morphology that resembles the morphology of an endogenous mature pancreatic β cell. In some cases, the cell cluster can comprise cell encapsulating crystalline insulin granules that resemble insulin granules of an endogenous mature pancreatic β cell, e.g., as detected by electron microscopy. A cell cluster can comprise a plurality cells having a morphology that resembles the morphology of an endogenous mature pancreatic β cell. For example, at least about 10%, 20%, 30%, 40%, 50%, 60%, 70%, 80%, 90% cells in a cell cluster can encapsulate crystalline insulin granules that resemble insulin granules of an endogenous mature pancreatic β cell. In some cases, 100% cells in a cell cluster encapsulate crystalline insulin granules that resemble insulin granules of an endogenous mature pancreatic β cell.

A cell cluster can exhibit glucose-stimulated calcium (Ca²⁺) flux to one or more glucose challenges. In some cases, a cell cluster exhibits a glucose-stimulated Ca²⁺ flux (GSCF) that resembles the GSCF of an endogenous pancreatic islet. In some cases, a cell cluster exhibits a GSCF response to at least 1, 2, 3, 4, 5, 6, 8, or 10 sequential glucose challenges in a manner that resembles the GSCF response of an endogenous pancreatic islet to multiple glucose challenges. A cell cluster can exhibit an in vitro and/or in vivo GSCF response when exposed to a glucose challenge.

A cell cluster can comprise cells originated from any species. For example, a cell cluster can comprise cells from a mammalian species, with non-limiting examples including a murine, bovine, simian, porcine, equine, ovine, or human cell. In some cases, at least one cell in the cell cluster is a human cell.

Provided herein also include compositions comprising a cell clusters disclosed through the application. In addition to the cell cluster, the compositions can further comprise a scaffold or matrix that can be used for transplanting the cell clusters to a subject. A scaffold can provide a structure for the cell cluster to adhere to. The cell cluster can be transplanted to a subject with the scaffold. The scaffold can be biodegradable. In some cases, a scaffold comprises a biodegradable polymer. The biodegradable polymer can be a synthetic polymer, such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, and biodegradable polyurethanes. The biodegradable polymer can also be a natural polymer, such as albumin, collagen, fibrin, polyamino acids, prolamines, and polysaccharides (e.g., alginate, heparin, and other naturally occurring biodegradable polymers of sugar units). Alternatively, the scaffold can be non-biodegradable. For example, a scaffold can comprise a non-biodegradable polymer, such as polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, and polyethylene oxide.

### Dissociated Cell Compostions and Methods for Making Cell Clusters of Stem Cell Derived Beta Cells

Further disclosed herein are methods for making cell clusters that resemble the function and characteristics of an endogenous tissue or cell cluster, e.g., an endogenous pancreatic islet.

The methods can comprise dissociating a first cell cluster and re-aggregating the dissociated cells to a second cell cluster, where the second cell cluster more closely resembles the function and characteristics of an endogenous tissue or cell cluster, e.g., an endogenous pancreatic islet, compared to the first cell cluster. The term “re-aggregating” and its grammatical equivalences as used herein can refer to, when clusters are dissociated into smaller clusters or single cells, the dissociated cells then form new cell-to-cell contacts and form new clusters. The methods can be used for producing a cell cluster in vitro by a) dissociating a plurality of cells from a first cell cluster; and b) culturing the plurality of cells from a) in a medium, thereby allowing the plurality of cells to form a second cell cluster. In some cases, the second cell cluster is an in vitro cell cluster. The first cell cluster can be an in vitro cell cluster, e.g., a cluster formed by a suspension of single cells in vitro in a culture medium. In some cases, the first cell cluster can be an ex vivo cell cluster, e.g., a cell cluster that is formed in a body of a live organism and isolated from said organism. For example, a first cell cluster that the method provided herein is applicable to can be a human pancreatic islet. In some cases, the first cell cluster can be a cadaveric pancreatic islet. In some embodiments, the dissociated cells were previously frozen.

A method provided herein can enrich pancreatic cells in a cell cluster, e.g., a pancreatic β cell, an endocrine cell, or an endocrine progenitor cell. In some examples, the method can reduce or eliminate stem cells or pancreatic progenitor cells from a cell cluster. In some cases, the second cell cluster comprises a higher percentage of cells that express chromogranin A as compared the first cell cluster. In some cases, the second cell cluster comprises a higher percentage of cells that express NKX6.1 and C-peptide as compared the first cell cluster. In some cases, the second cell cluster comprises a higher percentage of cells that express NKX6.1 and ISL1 as compared the first cell cluster. In some cases, the second cell cluster comprises a lower percentage of cells that are negative for NKX6.1 and C-peptide as compared the first cell cluster. In some cases, the second cell cluster comprises a lower percentage of cells that express SOX2 as compared the first cell cluster. In some cases, the second in vitro cell cluster comprises a lower percentage of cells that express SOX9 as compared the first cell cluster.

In some cases, the medium comprises a thyroid hormone signaling pathway activator and a transforming growth factor β (TGF-β) signaling pathway inhibitor. In some cases, the medium comprises a) serum, and b) one or both of a thyroid hormone signaling pathway activator and a TGF-β signaling pathway inhibitor. In some cases, the medium for reaggregation as provided herein (reaggregation medium) can comprise no small molecule compounds. For example, the reaggregation medium can comprise no thyroid hormone signaling pathway activator. In some cases, the reaggregation medium does not comprise triiodothyronine (T3), or merely a trace amount of T3. The reaggregation medium can comprise no TGFβ signaling pathway inhibitor. In some cases, the reaggregation medium does not comprise an Alk5 inhibitor (Alk5i), or merely a trace amount of Alk5i.

In some embodiments, compositions and methods disclosed herein improve cell recovery or yield after they are dissociated and subsequently reaggregated. Increased cell recovery can comprise an increased number or percentage of viable cells. Increased cell recovery can comprise an increased number or percentage of a population of interest (e.g., ISL1-positive and NKX6.1 positive cells or another population disclosed herein). In some embodiments, increased cell recovery is achieved after contacting the cells with a combination of agents disclosed herein. In some embodiments, cell recovery is increased when the cells are contacted with the agents prior to dissociation. In some embodiments, cell recovery is increased when the cells are contacted with the agents after dissociation.

In some embodiments, at least 25%, at least 30%, at least 35%, at least 40%, at least 45%, at least 50%, at least 55%, or at least 60% of total viable cells present before dissociation are recovered as viable cells when evaluated after re-aggregation, for example, when evaluated after about 1 day, 2, days, 3, days, 4 days, 5 days, 6 days, 7 days, 8 days, 9 days, 10 days, 11 days, 12 days, 13 days, or 14 days after dissociation, or about 1 day, 2, days, 3, days, 4 days, 5 days, 6 days, 7 days, 8 days, 9 days, 10 days, 11 days, 12 days, 13 days, or 14 days after thawing dissociated and cryopreserved cells.

In some embodiments, the yield of total viable cells obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher after re-aggregation compared to a method that not utilize an agent or combination of agents as disclosed herein, for example, when evaluated after about 1 day, 2, days, 3, days, 4 days, 5 days, 6 days, 7 days, 8 days, 9 days, 10 days, 11 days, 12 days, 13 days, or 14 days after dissociation, or 1 day, 2, days, 3, days, 4 days, 5 days, 6 days, 7 days, 8 days, 9 days, 10 days, 11 days, 12 days, 13 days, or 14 days after thawing dissociated and cryopreserved cells. In some embodiments, the yield of total viable cells is increased when the cells are contacted with an agent or combination of agents of the disclosure prior to dissociation of the earlier cluster. In some embodiments, the yield of total viable cells is increased when the cells are contacted with an agent or combination of agents of the disclosure after dissociation of the earlier cluster.

In some embodiments, at least 25%, at least 30%, at least 35%, at least 40%, at least 45%, at least 50%, at least 55%, or at least 60% of cells of a population of the disclosure (e.g., NKX6.1-opositive and ISL1-positive cells, or another population disclosed herein) present before dissociation are recovered as viable cells when evaluated after re-aggregation, for example, when evaluated after about 1 day, 2, days, 3, days, 4 days, 5 days, 6 days, 7 days, 8 days, 9 days, 10 days, 11 days, 12 days, 13 days, or 14 days after dissociation, or 1 day, 2, days, 3, days, 4 days, 5 days, 6 days, 7 days, 8 days, 9 days, 10 days, 11 days, 12 days, 13 days, or 14 days after thawing dissociated and cryopreserved cells.

In some embodiments, the yield of cells of a population of the disclosure (e.g., NKX6.1-poositive and ISL1-positive cells, or another population disclosed herein) obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher compared to a method that not utilize an agent or combination of agents as disclosed herein.

In some embodiments, the yield of cells of a population of the disclosure (e.g., NKX6.1-opositive and ISL1-positive cells, or another population disclosed herein) obtained by a method of the disclosure is at least 5%, at least 10%, at least 20%, at least 30%, at least 40%, at least 50%, at least 60%, at least 70%, at least 80%, at least 90%, at least 2-fold, at least 2.5-fold, at least 3-fold, at least 4 fold, or at least 5 fold higher after re-aggregation compared to a method that not utilize an agent or combination of agents as disclosed herein, for example, when evaluated after about 1 day, 2, days, 3, days, 4 days, 5 days, 6 days, 7 days, 8 days, 9 days, 10 days, 11 days, 12 days, 13 days, or 14 days after dissociation, or 1 day, 2, days, 3, days, 4 days, 5 days, 6 days, 7 days, 8 days, 9 days, 10 days, 11 days, 12 days, 13 days, or 14 days after thawing dissociated and cryopreserved cells. In some embodiments, the yield is increased when the cells are contacted with the agent or combination of agents prior to dissociation of an earlier cluster. In some embodiments, the yield is increased when the cells are contacted with the agent or combination of agents after dissociation of the earlier cluster.

In some embodiments, compositions and methods disclosed herein increase the percent of cells in a cluster expressing both NKX6.1 and ISL1 after dissociation and reaggregation. For example, at least about 10%, 20%, 30%, 35%, 38%, 40%, 42%, 44%, 46%, 50%, 60%, 70%, 80%, 90%, 95%, or 99% cells in a cell cluster can express NKX6.1 and ISL1 after dissociation and reaggregation as disclosed herein. In some cases, at least about 35% cells in the cell cluster can express NKX6.1 and ISL1. In some cases, at least about 38% cells in the cell cluster can express NKX6.1 and ISL1. In some cases, at least about 40% cells in the cell cluster can express NKX6.1 and ISL1. In some cases, at least about 45% cells in the cell cluster can express NKX6.1 and ISL1. In some cases, at least about 50% cells in the cell cluster can express NKX6.1 and ISL1. In some embodiments, the percent of cells in a re-aggregated cluster expressing both NKX6.1 and ISL1 is increased when the cells are contacted with an agent or combination of agents of the disclosure prior to dissociation of an earlier cluster. In some embodiments, the percent of cells in a re-aggregated cluster is increased when the cells are contacted an agent or combination of agents of the disclosure after dissociation of an earlier cluster.

Dissociating of the first cell cluster can be performed using methods known in the art. Non-limiting exemplary methods for dissociating cell clusters include physical forces (e.g., mechanical dissociation such as cell scraper, trituration through a narrow bore pipette, fine needle aspiration, vortex disaggregation and forced filtration through a fine nylon or stainless steel mesh), enzymatic dissociation using enzymes such as trypsin, collagenase, TrypLE™, and the like, or a combination thereof. After dissociation, cells from the first cell cluster can be in a cell suspension, e.g., a single cell suspension. The term “suspension” as used herein can refer to cell culture conditions in which cells are not attached to a solid support. Cells proliferating in suspension can be stirred while proliferating using apparatus well known to those skilled in the art.

In some embodiments, the disclosure provides for a composition comprising dissociated cells. In some embodiments, the disclosure provides for a composition comprising a plurality of dissociated insulin-positive endocrine progenitor cells. In some embodiments, the dissociated cells are Ngn3-positive. In some embodiments, the dissociated cells are PDX.1 positive. In some embodiments, the dissociated cells are NKX6.1 positive. In some embodiments, the disclosure provides for a composition comprising dissociated cells (e.g., dissociated insulin-positive endocrine progenitor cells) and a BMP signaling pathway inhibitor. In some embodiments, the BMP signaling pathway inhibitor is LDN193189 or a derivative thereof. In some embodiments, the disclosure provides for a composition comprising a plurality of dissociated cells (e.g., dissociated insulin-positive endocrine progenitor cells) and a ROCK inhibitor. In some embodiments, the ROCK inhibitor is thiazovivin, Y-27632, Fasudil/HA1077, or 14-1152, or derivatives thereof. In some embodiments, the disclosure provides for a composition comprising a plurality of dissociated cells (e.g., dissociated insulin-positive endocrine progenitor cells) and a histone methyltransferase inhibitor. In some embodiments, the histone methyltransferase inhibitor is 3-Deazaneplanocin A hydrochloride, or a derivative thereof. In some embodiments, the disclosure provides for a composition comprising a plurality of dissociated cells (e.g., dissociated insulin-positive endocrine progenitor cells) and zinc. In some embodiments, the zinc is in the form of ZnSO₄. In some embodiments, the disclosure provides for a composition comprising a plurality of dissociated cells (e.g., dissociated insulin-positive endocrine progenitor cells) and a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a composition comprising a plurality of dissociated cells (e.g., dissociated insulin-positive endocrine progenitor cells) and a lipid. In some embodiments, the lipid is a saturated fatty acid. In some embodiments, the saturated fatty acid is palmitate. In some embodiments, the lipid is a unsaturated fatty acid. In some embodiments, the unsaturated fatty acid is oleic acid, linoleic acid, or palmitoleic acid.

Provided herein are compositions comprising isolated insulin-positive endocrine cells that have been contacted with an agent that inhibits expression or function of monoglyceride lipase (MGLL) in vitro and exhibit an increased ratio of monoglycerides to free fatty acids compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit a decreased ratio of free fatty acids to monoglycerides compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit a decreased level of free fatty acids compared to a corresponding population of isolated insulin-positive endocrine cells that have not been contacted with an agent that inhibits expression or function of monoglyceride lipase (MGLL) in vitro.

Provided herein are compositions comprising isolated insulin-positive endocrine cells that have been contacted with an agent that inhibits expression or function of monoglyceride lipase (MGLL) in vitro and exhibit an increased level of monoglycerides compared to a corresponding population of isolated insulin-positive endocrine cells that have not been contacted with an agent that inhibits expression or function of monoglyceride lipase (MGLL) in vitro.

Provided herein are compositions comprising a population of insulin-positive endocrine cells and an agent that inhibits the conversion of monoglycerides to free fatty acids.

In some embodiments, the composition further comprises a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the composition comprises a TGF-β pathway inhibitor. In some embodiments, the TGF-β pathway inhibitor is Alk5i (SB505124), or a derivative thereof. In some embodiments, the composition comprises a thyroid hormone signaling pathway activator. In some embodiments, the thyroid hormone signaling pathway activator is GC-1 or T3, or a derivative thereof. In some embodiments, the composition comprises a ROCK inhibitor. In some embodiments, the ROCK inhibitor is thiazovivin. In some embodiments, the composition comprises a histone methyltransferase inhibitor. In some embodiments, the histone methyltransferase inhibitor is 3-Deazaneplanocin A hydrochloride. In some embodiments, the composition comprises a protein kinase inhibitor. In some embodiments, the protein kinase inhibitor is staurosporine. In some embodiments, the composition comprises vitamin C. In particular embodiments, the composition is in vitro. In some embodiments, the composition does not comprise a γ secretase inhibitor (e.g., XXI). In some embodiments, the dissociated insulin-positive endocrine progenitor cells were previously frozen.

In some embodiments, the disclosure provides for a composition comprising a plurality of cell clusters. In some embodiments, the disclosure provides for a composition comprising a plurality of cell clusters; wherein the cell clusters comprise insulin-positive cells; wherein at least 30%, at least 35%, at least 40%, at least 45%, at least 50%, at least 55%, at least 60%, or at least 65% of the cells in the composition are viable following 11 days in culture in vitro.

In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter.

In some embodiments, the disclosure provides for a composition comprising a plurality of cell clusters; wherein the cell clusters comprise insulin-positive cells; wherein at least about 40%, 50%, 60%, 70%, 80%, or 90% of the cell clusters have a diameter of about 80-150, about 100-150, about 120-150, about 140-150, about 80-130, about 100-130, about 120-130, about 80-120, about 90-120, or about 100-120 μm.

In some embodiments, the disclosure provides for a composition comprising a plurality of cell clusters; wherein the cell clusters comprise insulin-positive cells; wherein the cell clusters have a mean or median diameter of at most 120, at most 130, at most 140, at most 150, at most 160, or at most 170 μm.

In some embodiments, the disclosure provides for a composition comprising a plurality of cell clusters; wherein the cell clusters comprise insulin-positive cells; wherein the cell clusters have a mean or median diameter of about 80-150, about 100-150, about 120-150, about 140-150, about 80-130, about 100-130, about 120-130, about 80-120, about 90-120, or about 100-120 μm.

In some embodiments, the disclosure provides for a composition comprising a plurality of cell clusters; wherein the cell clusters comprise insulin-positive cells; wherein at least about 50%, at least about 60%, at least about 70%, or at least about 80% of the cell clusters have a diameter of less than 150 μm.

In some embodiments, the disclosure provides for a composition comprising a plurality of cell clusters; wherein the cell clusters comprise insulin-positive cells; wherein at least about 50%, at least about 60%, at least about 70%, or at least about 80% of the cell clusters have a diameter of less than 140 μm.

In some embodiments, the disclosure provides for a composition comprising a plurality of cell clusters; wherein the cell clusters comprise insulin-positive cells; wherein at least about 50%, at least about 60%, at least about 70%, or at least about 80% of the cell clusters have a diameter of less than 150 μm.

In some embodiments, the disclosure provides for a composition comprising a plurality of cell clusters; wherein the cell clusters comprise insulin-positive cells; wherein at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, the cell clusters comprise C-peptide positive cells. In some embodiments, the cell clusters comprise somatostatin positive cells. In some embodiments, the cell clusters comprise glucagon positive cells. In some embodiments, at least 30%, at least 35%, at least 40%, at least 45%, at least 50%, at least 55%, at least 60%, or at least 65% of the cells in the composition are viable following 11 days in culture in vitro.

In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition are 90-140 μm, 90-130 μm, 90-120 μm, 90-110 μm, 100-140 μm, 100-130 μm, 100-120 μm, 100-110 μm in diameter.

In some embodiments, at least 10%, at least 20%, at least 30%, at least 40%, at least 50%, at least 55%, at least 60%, at least 65%, at least 70%, at least 75%, at least 80%, at least 85%, at least 90%, at least 95% of the cell clusters in the composition exhibit a glucose-stimulated insulin secretion (GSIS) stimulation index of 1.5-4.5, 1.5-4.0, 1.5-3.5, 1.5-3.0, 1.5-2.5, 1.5-2.5, 1.5-2.0, 2.0-4.5, 2.0-4.0, 2.0-3.5, 2.0-3.0, 2.0-2.5, 2.5-4.5, 2.5-4.0, 2.5-3.5, 2.5-3.0, 3.0-4.5, 3.0-4.0, 3.0-3.5, 3.5-4.5, 3.5-4.0, or 4.0-4.5. In some embodiments, at least 2, 3, 4, 5, 10, 50, 100, 1000, 10000, 100000, or 1000000 cell clusters. In some embodiments, the composition is prepared in accordance with any of the methods disclosed herein. In some embodiments, the disclosure provides for a device comprising the any of the cell compositions disclosed herein. In some embodiments, the disclosure provides for a method of treating a subject with a disease characterized by high blood sugar levels over a prolonged period of time (e.g., diabetes), the method comprising administering any of the compositions disclosed herein or any of the devices disclosed herein to the subject.

In some embodiments, the disclosure provides for a method comprising the step of contacting a plurality of dissociated insulin-positive endocrine progenitor cells with a BMP signaling pathway inhibitor. In some embodiments, the BMP signaling pathway inhibitor is LDN193189 or a derivative thereof. In some embodiments, the disclosure provides for a method comprising the step of contacting a plurality of dissociated insulin-positive endocrine progenitor cells with a ROCK inhibitor. In some embodiments, the ROCK inhibitor is thiazovivin, Y-27632, Fasudil/HA1077, or 14-1152, or derivatives thereof. In some embodiments, the disclosure provides for a method comprising the step of contacting a plurality of dissociated insulin-positive endocrine progenitor cells with a histone methyltransferase inhibitor. In some embodiments, the histone methyltransferase inhibitor is 3-Deazaneplanocin A hydrochloride, or a derivative thereof. In some embodiments, the disclosure provides for a method comprising the step of contacting a plurality of dissociated insulin-positive endocrine progenitor cells with zinc. In some embodiments, the zinc is in the form of ZnSO₄. In some embodiments, the disclosure provides for a method comprising the step of contacting a plurality of dissociated insulin-positive endocrine progenitor cells with a monoglyceride lipase (MGLL) inhibitor. In some embodiments, the MGLL inhibitor is JJKK048, KML29, NF1819, JW642, JZL184, JZL195, JZP361, pristimerin, or URB602, or a derivative of any of the foregoing. In some embodiments, the disclosure provides for a method comprising the step of contacting a plurality of dissociated insulin-positive endocrine progenitor cells with a lipid. In some embodiments, the lipid is a saturated fatty acid. In some embodiments, the saturated fatty acid is palmitate. In some embodiments, the lipid is an unsaturated fatty acid. In some embodiments, the unsaturated fatty acid is oleic acid, linoleic acid, or palmitoleic acid.

In some embodiments, the method comprises contacting the plurality of dissociated insulin-positive endocrine progenitor cells with a serum albumin protein. In some embodiments, the serum albumin protein is a human serum albumin protein. In some embodiments, the composition comprises 0.01%-1%, 0.03-1%, 0.03-0.9%, 0.03-0.08%, 0.03-0.06%, 0.03-0.05%, 0.04-0.8%, 0.04-0.7%, 0.04-0.6%, 0.04-0.5%, 0.04-0.4%, 0.04-0.3%, 0.04-0.2%, 0.04-0.1%, 0.04-0.09%, 0.04-0.8%, 0.04-0.07%, 0.04-0.06%, 0.04-0.05%, 0.05-1%, 0.05-0.9%, 0.05-0.8%, 0.05-0.7%, 0.05-0.6%, 0.05-0.5%, 0.05-0.4%, 0.05-0.3%, 0.05-0.2%, 0.05-0.1%, 0.05-0.09%, 0.05-0.8%, 0.05-0.07%, or 0.05-0.06% serum albumin protein. In some embodiments, less than 90%, less than 85%, less than 80%, less than 75%, less than 70%, less than 65%, less than 60%, less than 55%, less than 50%, less than 45%, less than 40%, less than 35%, less than 30%, less than 35%, less than 30%, less than 25%, less than 20%, less than 15%, less than 10%, less than 5%, or less than 1%, of the cells in the composition are in cell clusters. In some embodiments, the method comprises contacting the plurality of dissociated insulin-positive endocrine progenitor cells with a TGF-β pathway inhibitor. In some embodiments, the TGF-β pathway inhibitor is Alk5i (SB505124), or a derivative thereof. In some embodiments, the method comprises contacting the plurality of dissociated insulin-positive endocrine progenitor cells with a thyroid hormone signaling pathway activator. In some embodiments, the thyroid hormone signaling pathway activator is GC-1 or T3, or a derivative thereof. In some embodiments, the method comprises contacting the plurality of dissociated insulin-positive endocrine progenitor cells with a protein kinase inhibitor. In some embodiments, the protein kinase inhibitor is staurosporine. In some embodiments, the method comprises contacting the plurality of dissociated insulin-positive endocrine progenitor cells with vitamin C. In some embodiments, the method does not comprise the step of contacting the plurality of dissociated insulin-positive endocrine cells with a γ secretase inhibitor (e.g., XXI). In some embodiments, the dissociated insulin-positive endocrine progenitor cells were previously frozen. In some embodiments, the method is performed over the course of 1-10 days, 1-9 days, 1-8 days, 1-7 days, 1-6 days, 1-5 days, 1-4 days, 1-3 days, 1-2 days, 2-10 days, 2-9 days, 2-8 days, 2-7 days, 2-6 days, 2-5 days, 2-4 days, 2-3 days, 3-10 days, 3-9 days, 3-8 days, 3-7 days, 3-6 days, 3-5 days, 3-4 days, 4-10 days, 4-9 days, 4-8 days, 4-7 days, 4-6 days, or 4-5 days. In some embodiments, the method results in the reaggregation of the dissociated cells into a plurality of cell clusters. In some embodiments, at least about 40%, 50%, 60%, 70%, 80%, or 90% of the plurality of cell clusters have a diameter from about 50 μm to about 250 μm, from about 75 μm to about 250 μm, or from about 100 μm to about 200 μm. In some embodiments, at least about 40%, 50%, 60%, 65%, 70%, 75%, 80%, 85%, 95%, or 99% of the cells of the plurality of cell clusters of the second cell population are viable. In some embodiments, the method results in the reaggregation of the dissociated cells into at least 2, 3, 4, 5, 10, 50, 100, 1000, 10000, 100000, or 1000000 cell clusters.

In some cases, the method provided herein does not comprise an active cell sorting process, e.g., flow cytometry. In some cases, a cell cluster as described herein can be an unsorted cell cluster. In some cases, a method provided herein does not rely on an active cell sorting for the enrichment or elimination of a particular type of cells in the first cell cluster. In some cases, a method merely requires dissociating the first cell cluster and culturing the plurality of cells dissociated from the first cell cluster in a medium, thereby allowing formation of a second cell cluster.

In some cases, the method provided herein can be applied to dissociate a cell cluster and reaggregate into a new cluster for more than once. For instance, a first cell cluster can be dissociated and reaggregated to form a second cell cluster according to the method provided herein, and the second cell cluster can be further dissociated and reaggregated to form a third cell cluster, and so on. Reaggregation as provided herein can be performed sequentially to a cell cluster for at least 1, 2, 3, 4, 5, 6, 7, 8, 9, or 10 times.

Cell sorting as described herein can refer to a process of isolating a group of cells from a plurality of cells by relying on differences in cell size, shape (morphology), surface protein expression, endogenous signal protein expression, or any combination thereof. In some cases, cell sorting comprises subjecting the cells to flow cytometry. Flow cytometry can be a laser- or impedance-based, biophysical technology. During flow cytometry, one can suspend cells in a stream of fluid and pass them through an electronic detection apparatus. In one type of flow cytometry, fluorescent-activated cell sorting (FACS), based on one or more parameters of the cells' optical properties (e.g., emission wavelength upon laser excitation), one can physically separate and thereby purify cells of interest using flow cytometry. As described herein, an unsorted cell cluster can be cell cluster that formed by a plurality of cells that have not been subject to an active cell sorting process, e.g., flow cytometry. An unsorted cell cluster, in some cases referred to as “reaggregated cell cluster,” can be formed by a plurality of cells that are dissociated from an existing cell cluster, and before their reaggregation into the new cell cluster, there can be no active cell sorting process, e.g., flow cytometry or other methods, to isolate one or more particular cell types for the reaggregation as provided herein. In some cases, flow cytometry as discussed herein can be based on one or more signal peptides expressed in the cells. For example, a cell cluster can comprise cells that express a signal peptide (e.g., a fluorescent protein, e.g., green fluorescent protein (GFP) or tdTomato). In some cases, the signal peptide is expressed as an indicator of insulin expression in the cells. For instance, a cell cluster can comprise cell harboring an exogenous nucleic acid sequence coding for GFP under the control of an insulin promoter. The insulin promoter can be an endogenous or exogenous promoter. In some cases, the expression of GFP in these cells can be indicative of insulin expression in said cells. The GFP signal can thus be a marker of a pancreatic β cell. In some cases, cell sorting as described herein can comprise magnetic-activated flow cytometry, where magnetic antibody or other ligand is used to label cells of different types, and the differences in magnetic properties can be used for cell sorting.

The cells dissociated from the first cell cluster can be cultured in a medium for re-aggregating to a second cell cluster. The medium can comprise Connought Medical Research Laboratories 1066 supplemented islet media (CMRLS). In some cases, the suitable culture medium comprises a component of CMRLS (e.g., supplemental zinc). The CMRLS can be supplemented, e.g., with serum (e.g., human serum, human platelet lysate, fetal bovine serum, or serum replacements such as Knockout Serum Replacement).

The medium can comprise one or more compounds that regulate certain signaling pathways in cells. For example, the medium can comprise a thyroid hormone signaling pathway activator, a transforming growth factor β (TGF-β) signaling pathway inhibitor, or both.

The thyroid hormone signaling pathway activator in the medium used herein can be triiodothyronine (T3). In some cases, the thyroid hormone signaling pathway activator can be an analog or derivative of T3. Non-limiting exemplary analogs of T3 include selective and non-selective thyromimetics, TRO selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-2115 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine (T4) and triiodothyronine (T3) alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (T1AM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3′-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA). In some cases, the thyroid hormone signaling pathway activator is a prodrug or prohormone of T3, such as T4 thyroid hormone (e.g., thyroxine or L-3,5,3′,5′-tetraiodothyronine). The thyroid hormone signaling pathway activator can also be an iodothyronine composition described in U.S. Pat. No. 7,163,918, which is incorporated by reference herein in its entirety.

The concentration of the thyroid hormone signaling pathway activator in the medium can be in a range suitable for cell aggregation. In some cases, the concentration of the thyroid hormone signaling pathway activator in the medium is from about 0.1 μM to about 10 μM, such as from about 0.5 μM to about 2 μM, from about 0.8 μM to about 1.5 μM, from about 0.9 μM to about 1.5 μM, from about 0.9 μM to about 1.2 μM, or from about 0.9 μM to about 1.2 μM. In some cases, the contraction of the thyroid hormone signaling pathway activator in the medium is at least about 0.1 μM, 0.2 μM, 0.4 μM, 0.8 μM, 0.9 μM, 1 μM, 1.1 μM, 1.2 μM, 1.3 μM, 1.4 μM, 1.5 μM, 2 μM, 3 μM, 4 μM, 5 μM, 6 μM, 7 μM, 8 μM, 9 μM, or 10 μM. In some case, the contraction of the thyroid hormone signaling pathway activator (e.g., T3) in the medium is about 1 μM.

The TGF-β signaling pathway inhibitor used in the medium herein can be an inhibitor of TGF-β receptor type I kinase (TGF-β RI) signaling. The TGF-β signaling pathway inhibitor can be an activin receptor-like kinase-5 (Alk5) inhibitor, e.g., ALK5 inhibitor II (CAS 446859-33-2, an ATP-competitive inhibitor of TGF-β RI kinase, also known as RepSox, IUPAC Name: 2-[5-(6-methylpyridin-2-yl)-1H-pyrazol-4-yl]-1,5-naphthyridine). In some cases, the TGF-β signaling pathway inhibitor is an analog or derivative of ALK5 inhibitor II, including those described in in U.S. Patent Publication Nos. 2012/0021519, 2010/0267731, 2009/0186076, and 2007/0142376, which are incorporated by reference herein in their entireties. In some cases, examples of TGF-β signaling pathway inhibitor that can be used in the medium herein also include D 4476, SB431542, A-83-01, also known as 3-(6-Methyl-2-pyridinyl)-N-phenyl-4-(4-quinolinyl)-1H-pyrazole-1-carbothioamide; 2-(3-(6-Methylpyridin-2-yl)-1H-pyrazol-4-yl)-1, 5-naphthyridine, Wnt3a/BIO, BMP4, GW788388 (-(4-[3-(pyridin-2-yl)-1H-pyrazol-4-yl]pyridm-2-yl}-N-(tetrahydro-2H-pyran-4-yl)benzamide), SMI 6, ΛN-1 130 (3-((5-(6-methylpyridin-2-yl)-4-(quinoxalin-6-yl)-1H-imidazol-2-yl)methyl)benzamide, GW6604 (2-phenyl-4-(3-pyridin-2-yl-1H-pyrazol-4-yl)pyridine), SB-505124 (2-(5-benzo[1,3]dioxol-5-yl-2-tert-butyl-3H-imidazol-4-yl)-6-methylpyridine hydrochloride), SU5416, lerdelimumab (CAT-152), metelimumab (CAT-192), GC-1008, ID1 1, AP-12009, AP-1 1014, LY550410, LY580276, LY364947, LY2109761, SD-208, SM16, NPC-30345, KÏ26894, SB-203580, SD-093, ALX-270-448, EW-7195, SB-525334, ΛN-1233, SKI2162, Gleevec, 3,5,7,2′,4′-pentahydroxyfiavone (Morin), activin-M108A, P144, soluble TBR2-Fc, pyrimidine derivatives and indolinones. Inhibition of the TGF-β/activin pathway can have similar effects. Thus, any inhibitor (e.g., upstream or downstream) of the TGF-β/activin pathway can be used in combination with, or instead of, TGF-β/ALK5 inhibitors as described herein. Exemplary TGF-β/activin pathway inhibitors include, but are not limited to, TGF-β receptor inhibitors, inhibitors of SMAD 2/3 phosphorylation, inhibitors of the interaction of SMAD 2/3 and SMAD 4, and activators/agonists of SMAD 6 and SMAD 7. Furthermore, the categorizations described herein are merely for organizational purposes and one of skill in the art would know that compounds can affect one or more points within a pathway, and thus compounds may function in more than one of the defined categories. TGF-β receptor inhibitors may include any inhibitors of TGF signaling in general or inhibitors specific for TGF-β receptor (e.g., ALK5) inhibitors, which can include antibodies to, dominant negative variants of, and siRNA and antisense nucleic acids that suppress expression of, TGF-β receptors.

The concentration of the TGF-β signaling pathway inhibitor in the medium can be in a range suitable for cell aggregation. In some cases, the concentration of the TGF-β signaling pathway inhibitor in the medium is from about 1 μM to about 50 μM, such as from about 5 μM to about 15 μM, from about 8 μM to about 12 μM, or from about 9 μM to about 11 μM. In some cases, the contraction of the TGF-β signaling pathway inhibitor in the medium is at least about 1 μM, 5 μM, 8 μM, 9 μM, 10 μM, 11 μM, 12 μM, 13 μM, 14 μM, 15 μM, 20 μM, 25 μM, 30 μM, 35 μM, 40 μM, 45 μM, or 50 μM. In some case, the contraction of the TGF-β signaling pathway inhibitor (e.g., Alk5 inhibitor II) in the medium is about 10 μM.

The medium used to culture the cells dissociated from the first cell cluster can be xeno-free. A xeno-free medium for culturing cells and/or cell clusters of originated from an animal can have no product from other animals. In some cases, a xeno-free medium for culturing human cells and/or cell clusters can have no products from any non-human animals. For example, a xeno-free medium for culturing human cells and/or cell clusters can comprise human platelet lysate (PLT) instead of fetal bovine serum (FBS). For example, a medium can comprise from about 1% to about 20%, from about 5% to about 15%, from about 8% to about 12%, from about 9 to about 11% serum. In some cases, medium can comprise about 10% of serum. In some cases, the medium can be free of small molecules and/or FBS. For example, a medium can comprise MCDB131 basal medium supplemented with 2% BSA. In particular embodiments, the medium is serum-free. In some examples, a medium can comprise no exogenous small molecules or signaling pathway agonists or antagonists, such as, growth factor from fibroblast growth factor family (FGF, such as FGF2, FGF8B, FGF 10, or FGF21), Sonic Hedgehog Antagonist (such as Sant1, Sant2, Sant 4, Sant4, Cur61414, forskolin, tomatidine, AY9944, triparanol, cyclopamine, or derivatives thereof), Retinoic Acid Signaling agonist (e.g., retinoic acid, CD1530, AM580, TTHPB, CD437, Ch55, BMS961, AC261066, AC55649, AM80, BMS753, tazarotene, adapalene, or CD2314), inhibitor of Rho-associated, coiled-coil containing protein kinase (ROCK) (e.g., Thiazovivin, Y-27632, Fasudil/HA1077, or 14-1152), activator of protein kinase C (PKC) (e.g., phorbol 12,13-dibutyrate (PDBU), TPB, phorbol 12-myristate 13-acetate, bryostatin 1, or derivatives thereof), antagonist of TGF beta super family (e.g., Alk5 inhibitor II (CAS 446859-33-2), A83-01, SB431542, D4476, GW788388, LY364947, LY580276, SB505124, GW6604, SB-525334, SD-208, SB-505124, or derivatives thereof), inhibitor of Bone Morphogenic Protein (BMP) type 1 receptor (e.g., LDN193189 or derivatives thereof), thyroid hormone signaling pathway activator (e.g., T3 or derivatives thereof), gamma-secretase inhibitor (e.g., XXI, DAPT, or derivatives thereof), activator of TGF-β signaling pathway (e.g., WNT3a or Activin A) growth factor from epidermal growth factor (EGF) family (e.g., betacellulin or EGF), broad kinase (e.g., staurosporine or derivatives thereof), non-essential amino acids, vitamins or antioxidants (e.g., cyclopamine, vitamin D, vitamin C, vitamin A, or derivatives thereof), or other additions like N-acetyl cysteine, zinc sulfate, or heparin. In some cases, the reaggregation medium can comprise no exogenous extracellular matrix molecule. In some cases, the reaggregation medium does not comprise Matrigel™ In some cases, the reaggregation medium does not comprise other extracellular matrix molecules or materials, such as, collagen, gelatin, poly-L-lysine, poly-D-lysine, vitronectin, laminin, fibronectin, PLO laminin, fibrin, thrombin, and RetroNectin and mixtures thereof, for example, or lysed cell membrane preparations.

A person of ordinary skill in the art will appreciate that that the concentration of BSA supplemented into the medium, if desired, may vary. For example, a medium (e.g., MCDB131) can comprise about 0.01%, 0.05%, 0.1%, 1%, about 2%, about 3%, about 4%, about 5%, about 10%, or about 15% BSA. In particular embodiments, the media does not comprise any BSA. The medium used (e.g., MCDB131 medium) can contain components not found in traditional basal media, such as trace elements, putrescine, adenine, thymidine, and higher levels of some amino acids and vitamins. These additions can allow the medium to be supplemented with very low levels of serum or defined components. The medium can be free of proteins and/or growth factors, and may be supplemented with EGF, hydrocortisone, and/or glutamine.

The medium can comprise one or more extracellular matrix molecules (e.g., extracellular proteins). Non-limiting exemplary extracellular matrix molecules used in the medium can include collagen, placental matrix, fibronectin, laminin, merosin, tenascin, heparin, heparin sulfate, chondroitin sulfate, dermatan sulfate, aggrecan, biglycan, thrombospondin, vitronectin, and decorin. In some cases, the medium comprises laminin, such as LN-332. In some cases, the medium comprises heparin.

The medium can be changed periodically in the culture, e.g., to provide optimal environment for the cells in the medium. When culturing the cells dissociated from the first cell cluster for re-aggregation, the medium can be changed at least or about every 4 hours, 12 hours, 24 hours, 48 hours, 3 days or 4 days. For example, the medium can be changed about every 48 hours.

Cells dissociated from the first cell cluster can be seeded in a container for re-aggregation. The seeding density can correlate with the size of the re-aggregated second cell cluster. The seeding density can be controlled so that the size of the second cell cluster can be similar to an endogenous pancreatic islet. In some cases, the seeding density is controlled so that the size of the second cell cluster can be from about 75 μm to about 250 μm. Cells dissociated from the first cell cluster can be seeded at a density of from about 0.1 million cells per mL to about 10 million cells per mL, e.g., from about 0.5 million cells per mL to about 1.5 million cells per mL, from about 0.8 million cells per mL to about 1.2 million cells per mL, from about 0.9 million cells per mL to about 1.1 million cells per mL, from about 2 million cells per mL to about 3 million cells per mL. In some cases, the cells dissociated from the first cell cluster can be seeded at a density of about 1 million cells per mL. In some cases, the cells dissociated from the first cell cluster can be seeded at a density of about 1.5 million cells per mL. In some cases, the cells dissociated from the first cell cluster can be seeded at a density of about 2 million cells per mL. In some cases, the cells dissociated from the first cell cluster can be seeded at a density of about 2.5 million cells per mL. In some cases, the cells dissociated from the first cell cluster can be seeded at a density of about 3 million cells per mL.

The cells dissociated from the first cell cluster can be cultured in a culture vessel. The culture vessel can be suitable for culturing a suspension of culture of cells. The culture vessel used for culturing the cells or cell clusters herein can include, but is not limited to: flask, flask for tissue culture, dish, petri dish, dish for tissue culture, multi dish, micro plate, micro-well plate, multi plate, multi-well plate, micro slide, chamber slide, tube, tray, culture bag, and roller bottle, stir tank bioreactors, or polymer (e.g., biopolymer or gel) encapsulation as long as it is capable of culturing the cells therein. The cells and/or cell clusters can be cultured in a volume of at least or about 0.2 ml, 0.5 ml, 1 ml, 5 ml, 10 ml, 20 ml, 30 ml, 40 ml, 50 ml, 100 ml, 150 ml, 200 ml, 250 ml, 300 ml, 350 ml, 400 ml, 450 ml, 500 ml, 600 ml, 800 ml, 1000 ml, 1500 ml, 2000 ml, 3000 ml or any range derivable therein, depending on the needs of the culture.

In some cases, cells can be cultured under dynamic conditions (e.g., under conditions in which the cells are subject to constant movement or stirring while in the suspension culture). For dynamic culturing of cells, the cells can be cultured in a container (e.g., an non-adhesive container such as a spinner flask (e.g., of 200 ml to 3000 ml, for example 250 ml; of 100 ml; or in 125 ml Erlenmeyer), which can be connected to a control unit and thus present a controlled culturing system. In some cases, cells can be cultured under non-dynamic conditions (e.g., a static culture) while preserving their proliferative capacity. For non-dynamic culturing of cells, the cells can be cultured in an adherent culture vessel. An adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, PLO laminin, fibrin, thrombin, and RetroNectin and mixtures thereof, for example, Matrigel™, and lysed cell membrane preparations.

Medium in a dynamic cell culture vessel (e.g., a spinner flask) can be stirred (e.g., by a stirrer). The spinning speed can correlate with the size of the re-aggregated second cell cluster. The spinning speed can be controlled so that the size of the second cell cluster can be similar to an endogenous pancreatic islet.

The cells dissociated from the first cell cluster can be cultured for a period of time to allow them for re-aggregating. The cells dissociated from the first cell cluster can be cultured for at least 12 hours, 24 hours, 1 day, 2 days, 3 days, 4 days, 5 days, 6 days, 7 days 8 days, 9 days 10 days, 15 days, 20 days, 25 days, or 30 days. In some cases, the cells dissociated from the first cell cluster can be cultured for at least 4 days.

The methods herein can also be used to enrich cells resembling endogenous cells, e.g., endogenous mature pancreatic β cells in a cell cluster. The methods can comprise dissociating a first cell cluster and re-aggregating the cells from the first cluster to a second cluster. The second cluster can comprise more cells resembling endogenous mature pancreatic β cells compared to the first cluster. The dissociating and re-aggregating can be performed using any methods and reagents disclosed through the application.

After re-aggregation, the second cell cluster can comprise more cells expressing one or more markers of an endogenous cell compared to the first cell cluster. For example, the second cluster can comprise more cells expressing one or more markers of an endogenous mature pancreatic β cell, the markers including insulin, C-peptide, PDX1, NKX6.1, CHGA, MAFA, ZNT8, PAX6, NEUROD1, glucokinase (GCK), SLC2A, PCSK1, KCNJ11, ABCC8, SLC30A8, SNAP25, RAB3A, GAD2, and PTPRN, compared to the first cell cluster. In some cases, the second cluster can comprise more cells expressing CHGA. In some cases, the second cluster can comprise more cells expressing NKX6.1. In some cases, the second cluster can comprise more cells expressing C-peptide. In some cases, the second cluster can comprise more cells expressing NKX6.1 and C-peptide. In some cases, the second cluster can comprise more cells expressing CHGA, NKX6.1 and C-peptide.

After re-aggregation, the second cell cluster can have a smaller size (e.g., a smaller diameter) compared to the first cell cluster. The smaller size can allow better exchange of molecules between the cell cluster and the surrounding environment. For example, a smaller size can allow better diffusion of molecules (e.g., reagents, gas, and/or nutrition) from the medium to the cells in a cell cluster. Thus, being in a smaller size, the second cell cluster can exchange molecules with the surrounding environment in a more efficient way compared to the first cell cluster. Thus, the second cell cluster can have fewer dead cells (e.g., cells died due to insufficient nutrition and/or gas) compared to the first cell cluster.

A method provided herein can enrich endocrine cells, e.g., cells expressing chromogranin A (CHGA). For examples, a percentage of cells in the second cell cluster that express chromogranin A is at least 1.2, at least 1.3, at least 1.4, or at least 1.5 times more than a percentage of cells in the first cell cluster that express chromogranin A. In some cases, the second cell cluster comprises at least about 70%, at least about 80%, at least about 90%, at least about 95%, at least about 99%, or 100% cells expressing CHGA. In some cases, at least about 85% cells in the second cell cluster can express CHGA. In some cases, the second cell cluster can comprise about 90% cell expressing CHGA. In some cases, the second cell cluster can comprise about 95% cells expressing CHGA. In certain cases, all cells in the second cell cluster can express CHGA.

A method provided herein can generate or enrich pancreatic β cell. For example, the second cell cluster comprises at least one pancreatic β cell, e.g., at least one non-native pancreatic β cell. For examples, a percentage of cells in the second cell cluster that express both NKX6.1 and C-peptide is at least 1.5, at least 1.75, or at least 2 times more than a percentage of cells in the first cell cluster that express both NKX6.1 and C-peptide. In some cases, the second cell cluster comprises at least about 35%, at least about 40%, at least about 50%, at least about 60%, at least about 70%, at least about 80%, at least about 90%, at least about 95%, at least about 99%, or 100% cells expressing NKX6.1 and C-peptide. In some cases, at least about 35% cells in the second cell cluster can express NKX6.1 and C-peptide. In some cases, a cell cluster can comprise about 60% cells expressing NKX6.1 and C-peptide. In some cases, the second cell cluster can comprise about 75% cell expressing NKX6.1 and C-peptide. In some cases, all cells in the second cell cluster can express NKX6.1 and C-peptide. In some cases, at least about 70% of the at least one non-native pancreatic β cell in the second cell cluster express chromogranin A as measured by flow cytometry. In some cases, at least about 25% of the at least one non-native pancreatic β cell in the second cell cluster express NKX6.1 and C-peptide as measured by flow cytometry.

A method provided herein can reduce or eliminate stem cells or precursor cells of a pancreatic endocrine cell. In some cases, a percentage of cells in the second cell cluster that express SOX2 is at least 2, at least 3, at least 5, or at least 10 times lower than a percentage of cells in the first cell cluster that express LIN28, Ki67, SOX2, or SOX9. For example, the second cell cluster can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing LIN28. In some examples, the second cell cluster as provided herein can comprise at most about 5% cells, at most about 5% cells, at most about 5% cells, at most about 5% cells, at most about 5% cells, at most about 2% cells, at most about 1% cells, at most about 0.5% cells, at most about 0.1% cells, at most about 0.05% cells, at most about 0.01% cells, or no cells expressing Ki67. For example, the second cell cluster can comprise at most 3% cells, at most about 2% cells, at most about 1% cells, at most about 0.5% cells, at most about 0.1% cells, at most about 0.05% cells, at most about 0.01% cells, or no cells expressing SOX2. In some cases, the second cell cluster can comprise about 1% cells expressing SOX2. In some cases, the second cell cluster can comprise about 0.6% cells expressing SOX2. In some cases, the second cell cluster can comprise about 0.3% cells expressing SOX2. In some cases, the second cell cluster can comprise about 0.1% cells expressing SOX2. For examples, the second cell cluster can comprise at most 10% cells, at most about 8% cells, at most about 6% cells, at most about 5% cells, at most about 2% cells, at most about 1% cells, at most about 0.5% cells, at most about 0.1% cells, at most about 0.05% cells, at most about 0.01% cells, or no cells expressing SOX9. In some cases, the second cell cluster can comprise about 2% cells expressing SOX9. In some cases, the second cell cluster can comprise about 6% cells expressing SOX9. In some cases, the second cell cluster can comprise about 1.2% cells expressing SOX9.

The second cell cluster can also function more similarly to an endogenous pancreatic islet compared to the first cell cluster. The second cell cluster can have a higher insulin content than the first cell cluster, for instance, at least 1.1, at least 1.25 or at least 1.5 times higher insulin content as compared to the first cell cluster. The second cluster can exhibit a greater in vitro GSIS than the first cell cluster, as measured by stimulation indexes. The second cluster can also exhibit a greater in vivo GSIS than the first cell cluster, as measured by stimulation indexes. In some cases, the second cluster can exhibit a greater in vitro GSIS and a greater in vivo GSIS compared to the first cell cluster, as measured by stimulation indexes. For example, the second cell cluster can secrete more insulin than the first cell cluster under the same stimulation conditions. The second cell cluster can also exhibit insulin secretion response to a potassium challenge (K⁺), e.g., a concentration of KCl, e.g., 30 mM KCl.

In some cases, the method provided herein can retain a large percentage of cells from the first cell cluster in the second cell cluster, e.g., pancreatic β cells or endocrine cells. For example, at least about 95%, at least about 98%, or at least about 99% of cells that express both NKX6.1 and C-peptide in the first cell cluster can be retained in the second in vitro cell cluster. In some cases, at most about 5%, at most about 2%, at most about 1%, at most about 0.5%, or at most about 0.1% of cells that express both NKX6.1 and C-peptide in the first cell cluster are lost during the dissociation and reaggregation process.

In some embodiments, a method provided herein provides a population of SC-β cells with increased stability or shelf life. For example, in some embodiments, a method of the discourse provides a population of cells that has at least 15%, at least 20%, at least 25%, at least 20%, at least 35%, at least 38%, at least 40%, at least 45%, or at least 50% ISL1-positive, NKX6.1-positive cells after 4 days, 7 days, or 10 days in culture.

In some cases, the cell cluster as described herein is generated from any starting cell population in vitro. For example, the starting cell can include, without limitation, insulin-positive endocrine cells (e.g., chromogranin A-positive cells) or any precursor thereof, such as a Nkx6.1-positive pancreatic progenitor cell, a Pdx1-positive pancreatic progenitor cell, and a pluripotent stem cell, an embryonic stem cell, and induced pluripotent stern cell. In some cases, the method includes differentiation of a reprogrammed cell, a partially reprogrammed cell (e.g., a somatic cell, e.g., a fibroblast which has been partially reprogrammed such that it exists in an intermediate state between an induced pluripotency cell and the somatic cell from which it has been derived), a transdifferentiated cell. In some cases, the cell cluster comprising the pancreatic β cell disclosed herein can be differentiated in vitro from an insulin-positive endocrine cell or a precursor thereof. In some cases, the cell cluster comprising the pancreatic β cell is differentiated in vitro from a precursor selected from the group consisting of a NKX6.1-positive pancreatic progenitor cell, a Pdx1-positive pancreatic progenitor cell, and a pluripotent stem cell. In some cases, the pluripotent stem cell is selected from the group consisting of an embryonic stem cell and induced pluripotent stem cell. As discussed above, the non-native pancreatic R cells can also be referred to as stem cell-derived β cells (SC-β cells) as they can be derived from stem cells in vitro. In some cases, the SC-β cell or the pluripotent stem cell from which the SC-β cell is derived is human. In some cases, the SC-β cell is human.

One aspect of the present disclosure provides a method of generating non-native pancreatic β cells. In some cases, the method can be any currently available protocol, such as those described in U.S. Published Patent Application Nos. US 2015-0240212, US 2015-0218522 and US-2021-0238553, each of which is incorporated herein by its entirety. Aspects of the disclosure involve definitive endoderm cells, Definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells (stage 1) are further differentiated, e.g., to primitive gut tube cells (stage 2), Pdx1-positive pancreatic progenitor cells (stage 3), NKX6.1-positive pancreatic progenitor cells (stage 4), or Ngn3-positive endocrine progenitor cells or insulin-positive endocrine cells (stage 5), followed by induction or maturation to SC-β cells (stage 6).

### Pharmaceutical Compositions

In some embodiments, the present disclosure provides pharmaceutical compositions that can utilize non-native pancreatic beta cell populations and cell components and products in various methods for treatment of a disease (e.g., diabetes). Certain cases encompass pharmaceutical compositions comprising live cells (e.g., non-native pancreatic beta cells alone or admixed with other cell types). Other cases encompass pharmaceutical compositions comprising non-native pancreatic beta cell components (e.g., cell lysates, soluble cell fractions, conditioned medium, ECM, or components of any of the foregoing) or products (e.g., trophic and other biological factors produced by non-native pancreatic beta cells or through genetic modification, conditioned medium from non-native pancreatic beta cell culture). In either case, the pharmaceutical composition may further comprise other active agents, such as anti-inflammatory agents, exogenous small molecule agonists, exogenous small molecule antagonists, anti-apoptotic agents, antioxidants, and/or growth factors known to a person having skill in the art.

Pharmaceutical compositions of the present disclosure can comprise non-native pancreatic beta cell, or components or products thereof, formulated with a pharmaceutically acceptable carrier (e.g., a medium or an excipient). The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are suitable for use in contact with the tissues of human beings and animals without excessive toxicity, irritation, allergic response, or other complication. Suitable pharmaceutically acceptable carriers can include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical compositions comprising cellular components or products, but not live cells, can be formulated as liquids. Pharmaceutical compositions comprising living non-native pancreatic beta cells can be formulated as liquids, semisolids (e.g., gels, gel capsules, or liposomes) or solids (e.g., matrices, scaffolds and the like).

Pharmaceutical compositions may comprise auxiliary components as would be familiar to a person having skill in the art. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other anti-oxidant agents suitable for renal administration, e.g., ascorbic acid and its salts or sulfite or sodium metabisulfite may also be employed.

A buffering agent may be used to maintain the pH of formulations in the range of about 4.0 to about 8.0; so as to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Pharmaceutical compositions comprising cells, cell components or cell products may be delivered to the kidney of a patient in one or more of several methods of delivery known in the art. In some cases, the compositions are delivered to the kidney (e.g., on the renal capsule and/or underneath the renal capsule). In another embodiment, the compositions may be delivered to various locations within the kidney via periodic intraperitoneal or intrarenal injection. Alternatively, the compositions may be applied in other dosage forms known to those skilled in the art, such as pre-formed or in situ-formed gels or liposomes.

Pharmaceutical compositions comprising live cells in a semi-solid or solid carrier may be formulated for surgical implantation on or beneath the renal capsule. It should be appreciated that liquid compositions also may be administered by surgical procedures. In particular cases, semi-solid or solid pharmaceutical compositions may comprise semi-permeable gels, lattices, cellular scaffolds and the like, which may be non-biodegradable or biodegradable. For example, in certain cases, it may be desirable or appropriate to sequester the exogenous cells from their surroundings, yet enable the cells to secrete and deliver biological molecules (e.g., insulin) to surrounding cells or the blood stream. In these cases, cells may be formulated as autonomous implants comprising living non-native pancreatic beta cells or cell population comprising non-native pancreatic beta cell surrounded by a non-degradable, selectively permeable barrier that physically separates the transplanted cells from host tissue. Such implants are sometimes referred to as “immunoprotective,” as they have the capacity to prevent immune cells and macromolecules from killing the transplanted cells in the absence of pharmacologically induced immunosuppression.

In other cases, various degradable gels and networks can be used for the pharmaceutical compositions of the present disclosure. For example, degradable materials particularly suitable for sustained release formulations include biocompatible polymers, such as poly(lactic acid), poly (lactic-co-glycolic acid), methylcellulose, hyaluronic acid, collagen, and the like.

In other cases, it may be desirable or appropriate to deliver the cells on or in a biodegradable, preferably bioresorbable or bioabsorbable, scaffold or matrix. These typically three-dimensional biomaterials contain the living cells attached to the scaffold, dispersed within the scaffold, or incorporated in an extracellular matrix entrapped in the scaffold. Once implanted into the target region of the body, these implants become integrated with the host tissue, wherein the transplanted cells gradually become established.

Examples of scaffold or matrix (sometimes referred to collectively as “framework”) material that may be used in the present disclosure include nonwoven mats, porous foams, or self-assembling peptides. Nonwoven mats, for example, may be formed using fibers comprising a synthetic absorbable copolymer of glycolic and lactic acids (PGA/PLA), foams, and/or poly(epsilon-caprolactone)/poly(glycolic acid) (PCL/PGA) copolymer.

In another embodiment, the framework is a felt, which can be composed of a multifilament yarn made from a bioabsorbable material, e.g., PGA, PLA, PCL copolymers or blends, or hyaluronic acid. The yarn is made into a felt using standard textile processing techniques consisting of crimping, cutting, carding and needling. In another embodiment, cells are seeded onto foam scaffolds that may be composite structures. In many of the abovementioned cases, the framework may be molded into a useful shape. Furthermore, it will be appreciated that non-native pancreatic beta cells may be cultured on pre-formed, non-degradable surgical or implantable devices.

The matrix, scaffold or device may be treated prior to inoculation of cells in order to enhance cell attachment. For example, prior to inoculation, nylon matrices can be treated with 0.1 molar acetic acid and incubated in polylysine, PBS, and/or collagen to coat the nylon. Polystyrene can be similarly treated using sulfuric acid. The external surfaces of a framework may also be modified to improve the attachment or growth of cells and differentiation of tissue, such as by plasma coating the framework or addition of one or more proteins (e.g., collagens, elastic fibers, reticular fibers), glycoproteins, glycosaminoglycans (e.g., heparin sulfate, chondroitin-4-sulfate, chondroitin-6-sulfate, dermatan sulfate, keratin sulfate), a cellular matrix, and/or other materials such as, but not limited to, gelatin, alginates, agar, agarose, and plant gums, among others.

In one aspect, the present disclosure provided devices comprising a cell cluster comprising at least one pancreatic β cell. A device provided herein can be configured to produce and release insulin when implanted into a subject. A device can comprise a cell cluster comprising at least one pancreatic β cell, e.g., a non-native pancreatic β cell. A cell cluster in the device can exhibit in vitro GSIS. A device can further comprise a semipermeable membrane. The semipermeable membrane can be configured to retain the cell cluster in the device and permit passage of insulin secreted by the cell cluster. In some cases of the device, the cell cluster can be encapsulated by the semipermeable membrane. The encapsulation can be performed by any technique available to one skilled in the art. The semipermeable membrane can also be made of any suitable material as one skilled in the art would appreciate and verify. For example, the semipermeable membrane can be made of polysaccharide or polycation. In some cases, the semipermeable membrane can be made of poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyphosphazene, polyamino acids, polyortho esters, polyacetals, polycyanoacrylates, biodegradable polyurethanes, albumin, collagen, fibrin, polyamino acids, prolamines, alginate, agarose, agarose with gelatin, dextran, polyacrylates, ethylene-vinyl acetate polymers and other acyl-substituted cellulose acetates and derivatives thereof, polyurethanes, polystyrenes, polyvinyl chloride, polyvinyl fluoride, poly(vinyl imidazole), chlorosulphonated polyolefins, polyethylene oxide, or any combinations thereof. In some cases, the semipermeable membrane comprises alginate. In some cases, the cell cluster is encapsulated in a microcapsule that comprises an alginate core surrounded by the semipermeable membrane. In some cases, the alginate core is modified, for example, to produce a scaffold comprising an alginate core having covalently conjugated oligopeptides with an RGD sequence (arginine, glycine, aspartic acid). In some cases, the alginate core is modified, for example, to produce a covalently reinforced microcapsule having a chemoenzymatically engineered alginate of enhanced stability. In some cases, the alginate core is modified, for example, to produce membrane-mimetic films assembled by in-situ polymerization of acrylate functionalized phospholipids. In some cases, microcapsules are composed of enzymatically modified alginates using epimerases, In some cases, microcapsules comprise covalent links between adjacent layers of the microcapsule membrane. In some embodiment, the microcapsule comprises a subsieve-size capsule comprising alginate coupled with phenol moieties. In some cases, the microcapsule comprises a scaffold comprising alginate-agarose. In some cases, the SC-β cell is modified with PEG before being encapsulated within alginate. In some cases, the isolated populations of cells, e.g., SC-β cells are encapsulated in photoreactive liposomes and alginate. It should be appreciated that the alginate employed in the microcapsules can be replaced with other suitable biomaterials, including, without limitation, polyethylene glycol (PEG), chitosan, polyester hollow fibers, collagen, hyaluronic acid, dextran with ROD, BHD and polyethylene glycol-diacrylate (PEGDA), poly(MPC-co-n-butyl methacrylate-co-4-vinylphenyl boronic acid) (PMBV) and poly(vinyl alcohol) (PVA), agarose, agarose with gelatin, and multilayer cases of these.

In some embodiments, a composition comprising a population of in vitro differentiated cells described herein are housed in a device that is implanted in a subject. In some embodiments, a composition comprising a population of in vitro differentiated cells described herein are housed in a device suitable for implantation into a subject. In some embodiments, the device upon implantation in a subject releases insulin while retaining the cells in the device, and facilitates tissue vascularization in and around the device. Exemplary devices are described, for example in U.S. Patent Publication Nos. US20190201323A1, US20200289407A1, US20210016073A1, US20220143374A1, and US20220175511A1, each of which is incorporated-by-reference in its entirety. In some embodiments, a subject is not administered an immune suppression agent during the implantation or vascularization of the device. In some embodiments, the device has a thickness of at least about 300 μm. In some embodiments, the device comprises a membrane comprising a plurality of nodes interconnected by a plurality of fibrils.

In some embodiments, the device comprises a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment having a surface area to volume ratio of at least about 40 cm-1, and wherein the enclosed compartment provides a volume for housing a cell within the device.

In some embodiments, the enclosed compartment comprises a single continuous open chamber. In some embodiments, the volume is about 8 μL to about 1,000 μL. In some embodiments, the device has at least one of a length and a width of about 0.25 cm to about 3 cm. In some embodiments, the device has a thickness of at least about 300 μm.

In some embodiments, the plurality of channels is generally perpendicular with respect to the first membrane. In some embodiments, the plurality of channels is arranged in a rectilinear array. In some embodiments, the plurality of channels is arranged in a polar array. In some embodiments, the channel has an average diameter of about 400 μm to about 3,000 μm. In some embodiments, the diameter is measured at a narrowest point in the channel. In some embodiments, a center of each channel is separated from the center of another channel by a distance of about 75 μm to about 500 μm. In some embodiments, the channel has a height to diameter ratio of at least about 0.2. In some embodiments, the device has a number of channels per area along a transverse plane, and in some embodiments the number is greater than about 50/cm2.

In some embodiments, at least one of the first membrane and the second membrane comprise a plurality of nodes interconnected by a plurality of fibrils. In some embodiments, at least one of the first membrane and the second membrane comprise PVDF, PTFE, ePTFE, PCL, PE/PES, PP, PS, PMMA, PLGA, PLLA, or any combination thereof. In some embodiments, the device further comprises an opening through the first membrane and/or the second membrane within the channel. In some embodiments, the opening has a concentricity with respect to the channel of at most about 25% the diameter of the channel. In some embodiments is a frame configured to receive the device described herein. In some embodiments, the frame is configured to receive a plurality of cell housing devices. In some embodiments, the frame comprises a flexing mechanism configured to prevent buckling of the cell housing device.

### Methods of Treatment

Further provided herein are methods for treating or preventing a disease in a subject. A composition comprising the cell clusters resembling endogenous pancreatic islets can be administered into a subject to restore a degree of pancreatic function in the subject. For example, the cell clusters resembling endogenous pancreatic islets can be transplanted to a subject to treat diabetes.

In some embodiments, a composition comprising cell clusters prepared according to methods disclosed herein achieve improved clinical outcomes when administered to a subject. For example, in some cases, viability of the transplanted pancreatic islets is increased compared to alternative compositions (e.g., prepared by alternate methods). In some embodiments, reduced immune infiltration is observed in response to the transplant compared to alternative compositions (e.g., prepared by alternate methods). In some embodiments, any of the cells disclosed herein are administered in a device (e.g., any of the devices disclosed herein).

The methods can comprise transplanting the cell cluster disclosed in the application to a subject, e.g., a subject in need thereof. The terms “transplanting” and “administering” can be used interchangeably and can refer to the placement of cells or cell clusters, any portion of the cells or cell clusters thereof, or any compositions comprising cells, cell clusters or any portion thereof, into a subject, by a method or route which results in at least partial localization of the introduced cells or cell clusters at a desired site. The cells or cell clusters can be implanted directly to the pancreas, or alternatively be administered by any appropriate route which results in delivery to a desired location in the subject where at least a portion of the implanted cells or cell remain viable. The period of viability of the cells or cell clusters after administration to a subject can be as short as a few hours, e.g., twenty-four hours, to a few days, to as long as several years. In some instances, the cells or cell clusters, or any portion of the cells or cell clusters thereof, can also be transadministered at a non-pancreatic location, such as in the liver or subcutaneously, for example, in a capsule (e.g., microcapsule) to maintain the implanted cells or cell clusters at the implant location and avoid migration.

A subject that can be treated by the methods herein can be a human or a non-human animal. In some cases, a subject can be a mammal. Examples of a subject include but are not limited to primates, e.g., a monkey, a chimpanzee, a bamboo, or a human. In some cases, a subject is a human. A subject can be non-primate animals, including, but not limited to, a dog, a cat, a horse, a cow, a pig, a sheep, a goat, a rabbit, and the like. In some cases, a subject receiving the treatment is a subject in need thereof, e.g., a human in need thereof.

As used herein, the term “treating” and “treatment” can refer to administering to a subject an effective amount of a composition (e.g., cell clusters or a portion thereof) so that the subject as a reduction in at least one symptom of the disease or an improvement in the disease, for example, beneficial or desired clinical results. For purposes of this disclosure, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (e.g., partial or total), whether detectable or undetectable. Treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis.

The methods and compositions provided herein may be used to treat a subject who has, or has a risk (e.g., an increased risk) of developing a disease. In some cases, the disease is diabetes, including, but not limited to, type I diabetes, type II diabetes, type 1.5 diabetes, prediabetes, cystic fibrosis-related diabetes, surgical diabetes, gestational diabetes, and mitochondrial diabetes The disease may also be a diabetes complication, including heart and blood vessel diseases, diabetic nephropathy, diabetic neuropathy, diabetic retinopathy, foot damages, and hearing damages.

The methods can comprise transplanting the cell cluster to a subject using any means in the art. For example, the methods can comprise transplanting the cell cluster via the intraperitoneal space, renal subcapsule, renal capsule, omentum, subcutaneous space, or via pancreatic bed infusion. For example, transplanting can be subcapsular transplanting, intramuscular transplanting, or intraportal transplanting, e.g., intraportal infusion. Immunoprotective encapsulation can be implemented to provide immunoprotection to the cell clusters.

In some embodiments, any of the cells, clusters, or compositions disclosed herein may be utilized in methods other than methods of treatment. In some embodiments, the cells, clusters, or compositions have utility for assessing the development of stem, pluripotent, multipotent, or unipotent cells in vitro or in vivo. For example, in some embodiments, the disclosure provides for compositions comprising cells (e.g., PDX. 1; NKX6.1V pancreatic progenitor cells or insulin⁺ pancreatic endocrine progenitor cells) and any one or more of the reagents disclosed herein (e.g., any one or more of the metabolites disclosed herein) to study the effect (e.g., the expression of specific genes) in the cells in response to the reagents.

### Devices

In some embodiments, the disclosure provides for a device comprising any of the cells, clusters or compositions disclosed herein. Exemplary devices are described in U.S. Patent Publication Nos. US20190201323A1, US20200289407A1, US20210016073A1, US20220143374A1, and US20220175511A1, each of which is incorporated by reference herein it its entirety.

In some embodiments, the device is configured to produce and release insulin when implanted into a subject. In some embodiments, the device is a semipermeable membrane, wherein the semipermeable membrane is configured to retain any of the cells disclosed herein in the device and permit passage of insulin produced by the cells out of the device.

In a certain aspect, described herein, is a cell housing device, comprising: a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment having a surface area to the volume ratio of at least about 40 cm⁻¹, and wherein the enclosed compartment provides a volume for housing a cell within the device.

In some embodiments, the compartment comprises a single continuous open space. In some embodiments, the volume is about 8 μL to about 1,000 μL. In some embodiments, the device has at least one of a length and a width of about 0.25 cm to about 3 cm. In some embodiments, the device has a thickness of at least about 300 μm. In some embodiments, the plurality of channels are generally perpendicular with respect to the first membrane. In some embodiments, the plurality of channels are arranged in a rectilinear array. In some embodiments, the plurality of channels are arranged in a polar array. In some embodiments, the channel has an average diameter of about 400 μm to about 3,000 μm. In some embodiments, the diameter is measured at a narrowest point in the channel. In some embodiments, a center of each channel is separated from the center of another channel by a distance of about 75 μm to about 500 μm. In some embodiments, the channel has a height to diameter ratio of at least about 0.2. In some embodiments, the device has a number of channels per area along a transverse plane is greater than about 50/cm². In some embodiments, at least one of the first membrane and the second membrane comprise a plurality of nodes interconnected by a plurality of fibrils. In some embodiments, at least one of the first membrane and the second membrane comprise PVDF, PTFE, ePTFE, PCL, PE/PES, PP, PS, PMMA, PLGA, PLLA, or any combination thereof. In some embodiments, the device further comprises an opening through the first membrane and the second membrane within the channel. In some embodiments, the opening has a concentricity with respect to the channel of at most 25% the diameter of the channel. In some embodiments, the device further comprises a frame configured to receive the device. In some embodiments, the frame is configured to receive a plurality of cell housing devices. In some embodiments, the frame comprises a flexing mechanism configured to prevent buckling of the cell housing device. In some embodiments, the device further comprises a cell population. In some embodiments, the cell population is an insulin secreting population. In some embodiments, the cell population is a stem cell derived cell that are capable of glucose-stimulated insulin secretion (GSIS). In some embodiments, the device further comprises a coating comprising a hydrophilic polymer. In some embodiments, the device has an insulin diffusion coefficient of about 2×10⁻⁶ cm²/s to about 1×10⁻⁵ cm²/s. In some embodiments, the device has a maximum insulin diffusion distance of less than about 150 μm. In some embodiments, the first membrane and the second membrane are fused with a fusion peel force of at least about 0.4 N. In some embodiments, at least one of the first membrane and the second membrane are semi-permeable. In some embodiments, the semi-permeability of the first membrane, the second membrane, or both is configured to protect the cell from an immune attack. In some embodiments, the semi-permeability of the first membrane, the second membrane, or both is configured to protect the cell from an immune attack in the absence of an immune suppression therapy. In some embodiments, at least one of the first membrane and the second membrane are configured to enable vascularization of the cell within the device. In some embodiments, at least one of the first membrane and the second membrane are configured to enable vascularization of the cell within the device in absence of an immune suppression therapy.

Another aspect provided herein is a cell housing device, comprising: a first membrane having a first surface comprising a plurality of channels, and a plurality of second surfaces opposing the first surface; and a second membrane opposite and attached to the plurality of the second surfaces of the first membrane; wherein the first membrane and the second membrane form an enclosed compartment wherein the enclosed compartment provides a volume for housing 1 million to 1 billion insulin producing cells within the device and wherein said membrane allows for diffusion of insulin from the device while retaining the insulin producing cells within the device.

In some embodiments, the device is an implantable microencapsulation device. In some embodiments, the implantable macroencapsulation device comprises: a first outer membrane; a second outer membrane; and a first semipermeable membrane attached between the first outer membrane and the second outer membrane; wherein the first semipermeable membrane and the first outer membrane are connected to form a primary compartment configured to provide a primary compartment for housing a population of cells; wherein the first semipermeable membrane and the second outer membrane are connected to form a secondary compartment; wherein the population of cells comprises pancreatic progenitor cells, endocrine cells, or beta cells, or any combination thereof; and wherein the device comprises a plurality of through holes through the first outer membrane, the second outer membrane, and the first semipermeable membrane. In some embodiments, the first outer membrane, the second outer membrane, and the first semipermeable membrane are configured to block passage of said population of cells out of the device.

In some embodiments, the device further comprises a second semipermeable membrane attached between the first semipermeable membrane and the second outer membrane to form a tertiary compartment between the primary compartment and the secondary compartment. In some embodiments, a hydraulic permeability of the first semipermeable membrane is greater than a hydraulic permeability of the first outer membrane, a hydraulic permeability of the second outer membrane, or both. In some embodiments, a hydraulic permeability of the first semipermeable membrane is greater than the hydraulic permeability of the first outer membrane, the hydraulic permeability of the second outer membrane, or both by at least about 25%. In some embodiments, a hydraulic permeability of the first semipermeable membrane is greater than a hydraulic permeability of the second semipermeable membrane. In some embodiments, a hydraulic permeability of the first semipermeable membrane is less than a hydraulic permeability of the second semipermeable membrane. In some embodiments, a porosity of the first semipermeable membrane is greater than a porosity of the first outer membrane, a porosity of the second outer membrane, or both. In some embodiments, a porosity of the first semipermeable membrane is greater than the porosity of the first outer membrane, the porosity of the second outer membrane, or both by at least about 25%. In some embodiments, a porosity of the first semipermeable membrane is greater than a porosity of the second semipermeable membrane. In some embodiments, a porosity of the first semipermeable membrane is less than a porosity of the second semipermeable membrane. In some embodiments, a flux of the first semipermeable membrane for a given material and bias (e.g., concentration gradient and/or pressure differential) is greater than a flux of the first outer membrane, a flux of the second outer membrane, or both for the same material and bias. In some embodiments, the flux of the first semipermeable membrane for a given material and bias (e.g., concentration gradient and/or pressure differential) is greater than the flux of the first outer membrane, the flux of the second outer membrane, or both by at least about 25% for the same material and bias. In some embodiments, a flux of the first semipermeable membrane for a given material and bias (e.g., concentration gradient and/or pressure differential) is greater than a flux of the second semipermeable membrane for the same material and bias. In some embodiments, a flux of the first semipermeable membrane for a given material and bias (e.g., concentration gradient and/or pressure differential) is less than a flux of the second semipermeable membrane for the same material and bias. In some embodiments, the device further comprises a primary port in fluid communication with the primary compartment, a secondary port in fluid communication with the secondary compartment, or any combination thereof. In some embodiments, the device further comprises a primary port in fluid communication with the primary compartment, a secondary port in fluid communication with the secondary compartment, a tertiary port in fluid communication with the tertiary compartment, or any combination thereof. In some embodiments at least one of the primary port the secondary port, or the tertiary port are sealable or re-sealable.

Another aspect provided herein is an implantable macroencapsulation device comprising a primary compartment configured to house one or more cells, and a secondary compartment, wherein the primary compartment and the secondary compartment are separated by a first semipermeable membrane, wherein the secondary compartment and the first semipermeable membrane are configured to i) filter a filtrate from the primary compartment, or ii) provide an ancillary agent to the one or more cells within the primary compartment, or both i) and ii); and wherein said one or more cells are encapsulated within said device from a range of about 10³ to about 10⁶ cells per μL of volume.

In some embodiments, the device further comprises a tertiary compartment, wherein the tertiary compartment and the secondary compartment are separated by a second semipermeable membrane, wherein the second semipermeable membrane is configured to i) filter a filtrate from the tertiary compartment, or ii) provide an ancillary agent to the one or more cells within the tertiary compartment, or both i) and ii). In some embodiments, the device further comprises at least one of a primary port in fluid communication with the primary compartment, or a secondary port in fluid communication with the secondary compartment. In some embodiments, the device further comprises at least one of a primary port in fluid communication with the primary compartment, a secondary port in fluid communication with the secondary compartment, or a tertiary port in fluid communication with the tertiary compartment. In some embodiments at least one of the primary port, the secondary port, or the tertiary port are sealable or re-sealable. In some embodiments, wherein the device comprises a plurality of through holes extending from one side of the device to an opposing side of the device through the layered membranes.

In some embodiments, one or more of the through holes are surrounded by a bonded portion of the membranes to form a seal. In some embodiments, the device comprises three or more seals. In some embodiments, the device comprises two or more self-intersecting seals. In some embodiments, the device comprises two or more elliptical seals. In some embodiments the seal is formed by an adhesive, an epoxy, a weld, any combination thereof, and/or any other appropriate bonding methods. In some embodiments, the first semipermeable membrane is configured to block passage of said one or more cells. In some embodiments, the primary compartment and the secondary compartment are configured to block passage of said one or more cells. In some embodiments the primary compartment, the secondary compartment, and the tertiary compartment are configured to block passage of said one or more cells.

Another aspect provided herein is a method, comprising: providing a macroencapsulation device comprising a primary compartment configured to house one or more cells, and a secondary compartment, wherein the primary compartment and the secondary compartment are separated by a first semipermeable membrane, wherein the secondary compartment and the semipermeable membrane are configured to i) filter a filtrate from the primary compartment, or ii) provide an ancillary agent to the one or more cells within the primary compartment, or both i) and ii); pre-vascularizing the macroencapsulation device; loading one or more cells into the primary compartment; and applying a pressure to the secondary compartment to remove a filtrate from the primary compartment.

In some embodiments the filtrate is removed from the primary compartment. In some embodiments, the method further comprises administering an ancillary agent into the primary compartment, the secondary compartment, or both. In some embodiments the ancillary agent comprises a drug, an oxygen generating substance, an anti-coagulant, a nutrient, or any combination thereof. In some embodiments administering the ancillary agent is performed after applying a negative pressure to the secondary compartment, though any method of providing a desired pressure differential of the secondary compartment relative to another compartment of the macroencapsulation device may also be used. In some embodiments, the method further comprises, inflating the primary compartment, the secondary compartment, or both. In some embodiments inflating the primary compartment, the secondary compartment, or both is performed before the prevascularizing of the macroencapsulation device. In some embodiments, the method further comprises, sealing the primary compartment, the secondary compartment, or both. In some embodiments, the method further comprises resealing the primary port, the secondary port, or both. In some embodiments, the housing further comprises a tertiary compartment separated from the secondary compartment by a second semipermeable membrane, and wherein the method further comprises loading one or more cells into the tertiary compartment. In some embodiments, the method further comprises administering an ancillary agent into the primary compartment, the secondary compartment, or the tertiary compartment, or any combination thereof. In some embodiments, the method further comprises inflating the primary compartment, the secondary compartment, or the tertiary compartment, or any combination thereof. In some embodiments, the method further comprises sealing the primary compartment, the secondary compartment, or the tertiary compartment, or any combination thereof. In some embodiments, the method further comprises resealing the primary port, the secondary port, the tertiary compartment, or any combination thereof.

Another aspect provided herein is a method, comprising: providing a macroencapsulation device comprising: a first outer membrane; a second outer membrane; and a first semipermeable membrane attached between the first outer membrane and the second outer membrane; wherein the first semipermeable membrane and the first outer membrane are connected to form a primary compartment configured for housing a population of cells; and wherein the first semipermeable membrane and the second outer membrane are connected to form a secondary compartment; pre-vascularizing the macroencapsulation device; loading one or more cell into the primary compartment; and applying a pressure differential to the secondary compartment to remove a filtrate from the primary compartment.

In some embodiments the filtrate is removed from the primary compartment. In some embodiments, the method further comprises administering an ancillary agent into the primary compartment, or the secondary compartment, or both. In some embodiments the ancillary agent comprises a drug, an oxygen generating substance, an anti-coagulant, a nutrient, or any combination thereof. In some embodiments administering the ancillary agent is performed after the applying a negative pressure or other pressure differential to the secondary compartment. In some embodiments, the method further comprises inflating the primary compartment, or the secondary compartment, or both. In some embodiments inflating the primary compartment, or the secondary compartment, or both is performed before prevascularizing of the macroencapsulation device. In some embodiments, the method further comprises sealing the primary compartment, or the secondary compartment, or both. In some embodiments, the method further comprises resealing the primary port, the secondary port, or both. In some embodiments, the macroencapsulation device further comprises a tertiary compartment separated from the secondary compartment by a second semipermeable membrane, and wherein the method further comprises loading one or more cell into the tertiary compartment. In some embodiments, the method further comprises administering an ancillary agent into the primary compartment, the secondary compartment, or the tertiary compartment, or any combination thereof. In some embodiments the filtrate is removed from the primary compartment, the tertiary compartment, or both. In some embodiments, the method further comprises inflating the primary compartment, the secondary compartment, the tertiary compartment, or any combination thereof. In some embodiments, the method further comprises sealing the primary compartment, the secondary compartment, or the tertiary compartment, or any combination thereof. In some embodiments, the method further comprises resealing the primary port, the secondary port, the tertiary compartment, or any combination thereof.

### EXAMPLES

These examples are provided for illustrative purposes only and not to limit the scope of the claims provided herein.

### Example 1. Effects of Activin A Concentration on Differentiation of Pancreatic β Cells

This example demonstrates the effects of treatment of stem cells with Activin A at different concentrations on the differentiation of pancreatic β cells from stem cells.

An exemplary base differentiation protocol, Version A, according to the present disclosure, was used for differentiating human stem cells into mature β cells capable of releasing insulin in response to glucose challenge in vitro.

Version A protocol is a 6-stage stepwise protocol. With Version A protocol, stem cells were treated with reagents in a manner similar to the Version A described in Example 1 of U.S. Patent Publication No. US20220090020A1, which is incorporated by reference herein in its entirety. In some experiments, Version A was modified such that 100 ng/mL Activin A at Stage 1 was replaced with a different concentration of Activin A as described below.

In one experiment, differentiation of the stem cells was initiated in spinner flasks using Version A protocol, and three different concentrations of Activin A (“AA”), 100 ng/mL (100%), 10 ng/mL (10%), and 5 ng/mL (5%), applied throughout the three days of Stage 1 were tested, while the remaining differentiation steps were kept the same. On QC days, e.g., upon completion of Stage 1 (“S1C”), completion of Stage 3 (“S3C”), or completion of Stage 5 (“S5C”), the cells were examined for their morphology and cell constituents. Specifically, 5 mL of cell cluster suspension solution were taken from the spinner flask, with 1 mL suspension aliquoted into a 24 well plate. Pictures of the cell clusters in the 24-well plate were taken under brightfield microscope. The cell clusters in the remaining 4 mL suspension were dispersed with TrypLE Express (Life Technologies; 12,604,013) at 37° C. for 20 min, fixed with 4% PFA for 20 mins at 4° C., blocked for 1 hour in FACS buffer (5% Donkey Serum and 0.05% Triton-100 in PBS) at room temperature, and stained with various antibodies against cell markers. Stained cells were then measured using an Accuri 6 flow cytometer (BD Biosciences) and analyzed using FlowJo software.

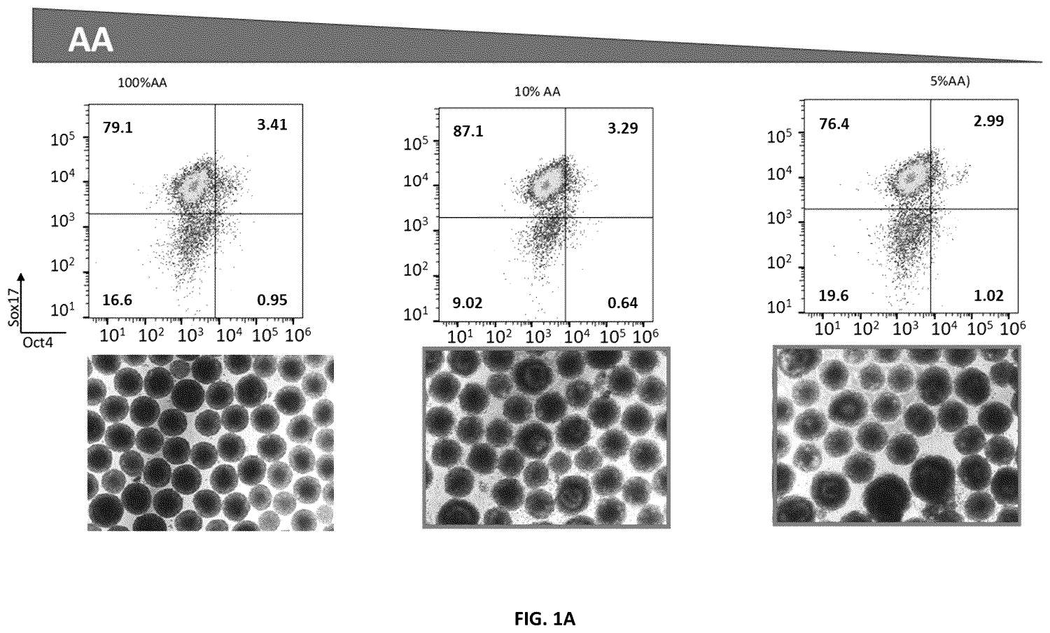

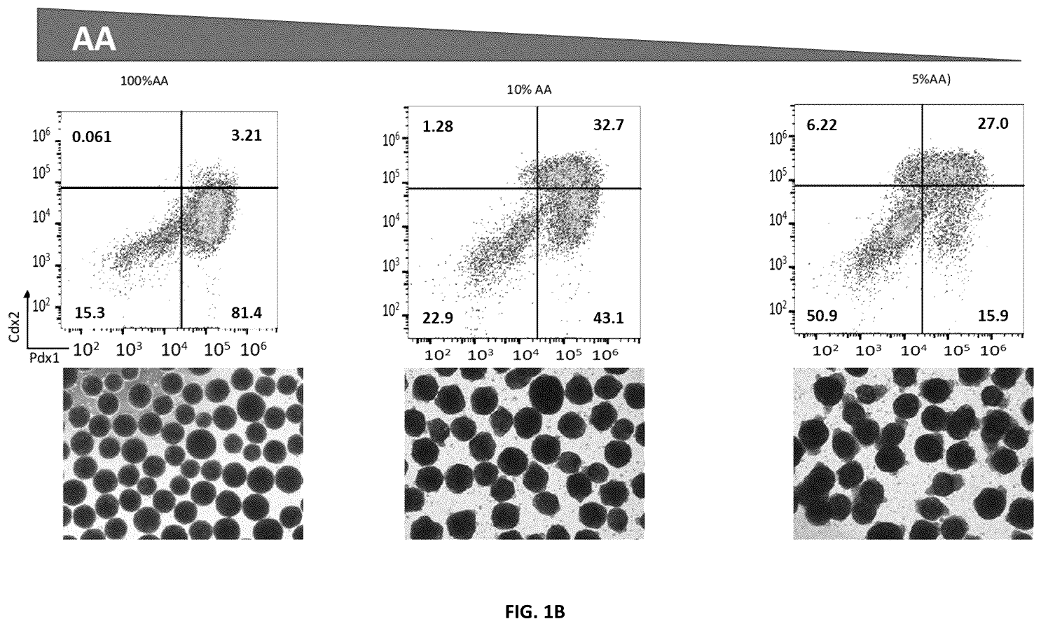

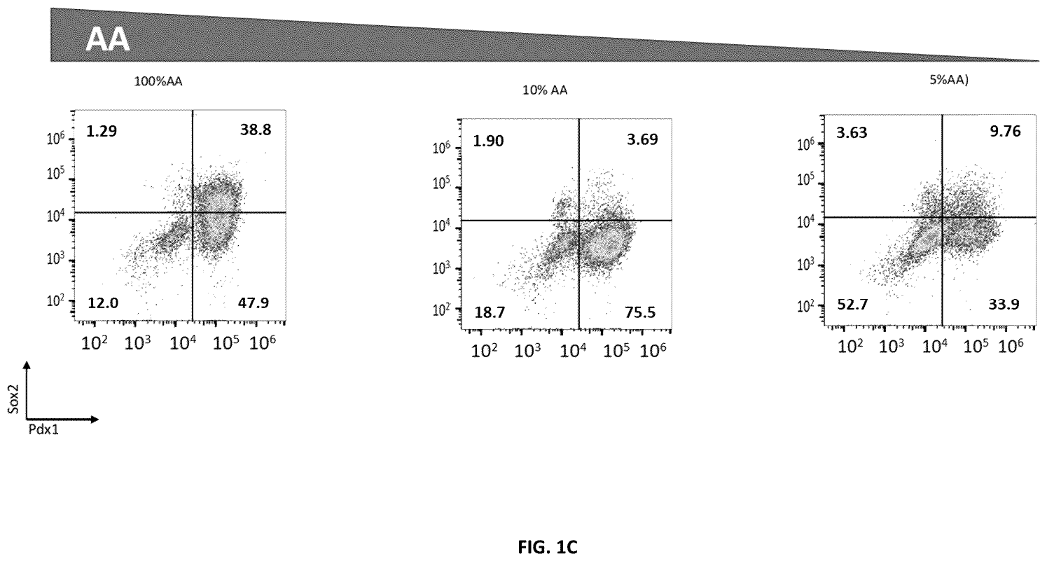

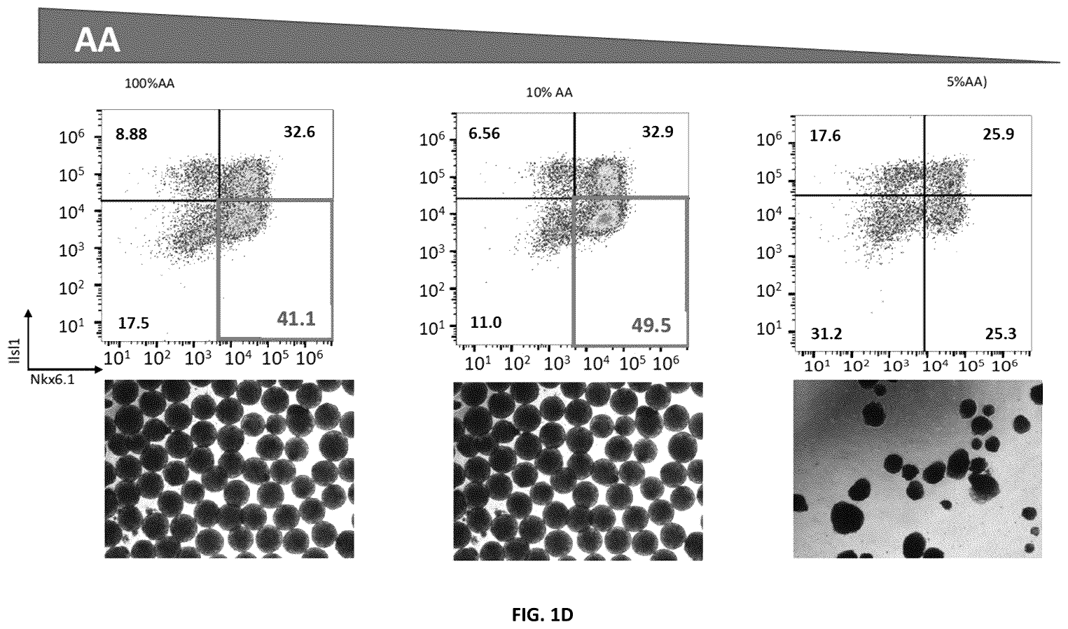

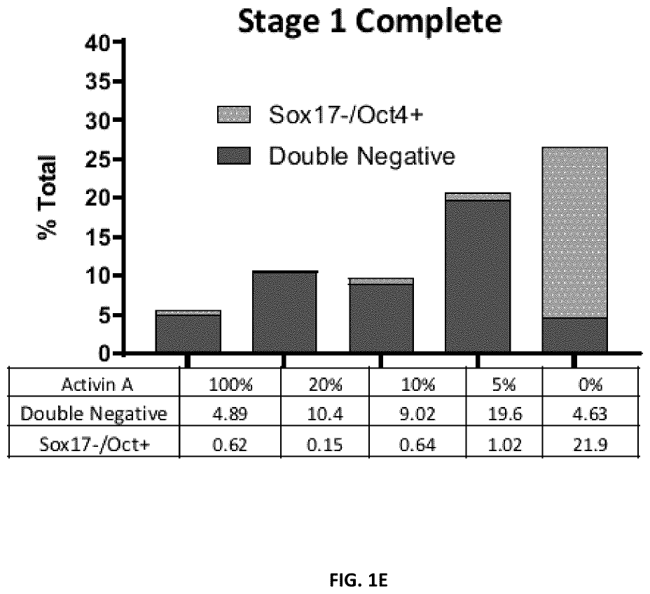

As shown in FIG. 1A, upon completion of Stage 1 differentiation, cell clusters obtained with reduced concentrations of Activin A, 10% or 5%, had altered morphology as compared to cell clusters obtained with 100% Activin A: instead of having a round shape and smooth edge, the cell clusters obtained under these conditions tend to have irregular shapes and rough edge. Flow cytometry analysis (FIG. 1A and Table 2) showed that there was not significant difference as to the percentage of cells that are negative for Oct4 staining and positive for Sox17 staining (“Oct4-negative, Sox17-positive,” or “Oct4−/Sox17”) among three conditions.

As shown in FIG. 1B, upon completion of Stage 3 differentiation, cell clusters under reduced Activin A conditions at Stage 1 (10% or 5%) continued to have altered morphology as compared to 100% Activin A. Flow cytometry analysis showed that more cells expressed Cdx2 and fewer cells expressed Sox2 under reduced concentrations of Activin A (FIGS. 1B-1C and Table 2).

FIG. 1D shows the pictures of cell clusters upon completion of Stage 5 differentiation, as well as representative flow cytometry analysis of Nkx6.1 and Isl1 expression in the cells. Cell clusters under 10% Activin A condition had similar morphology as compared to 100% Activin A at this point, while cell clusters under 5% Activin A continued to have altered morphology. Flow cytometry analysis (FIG. 1D and Table 2) showed that there were similar percentage of Nkx6.1-positive, Isl1-negative cells and Nkx6.1-positive, Isl1-positive cells between cells obtained under 10% Activin A condition and under 100% Activin A condition, while there were reduced percentage of Nkx6.1-positive cells under 5% Activin A condition.

TABLE 2
Flow Cytometry Analysis of Effects of Activin A

| Markers | 100% Activin A | 10% Activin A | 5% Activin A |
|---|---|---|---|
| S1c − Oct4−/Sox17+ | 79.1% | 87.1% | 76.4% |
| S3c − Cdx2+/Pdx1+ | 3.21% | 32.7% | 27.0% |
| S3c − Cdx2+/Pdx1− | 0.061% | 1.28% | 6.22% |
| S3c− Sox2+/Pdx1+ | 38.8% | 3.89% | 9.76% |
| S3c − Cdx2+/Pdx1− | 1.29% | 1.90% | 3.63% |
| S5c − Nkx6.1+/Isl1+ | 32.6% | 32.9% | 25.9% |
| S5c − Nkx6.1+/Isl1− | 41.1% | 49.5% | 25.3% |

In another experiment, a dose-response test was conducted to examine the effects of different concentrations of Activin A at Stage 1 on stem cell differentiation. Five different concentrations of Activin A were tested: 100 ng/mL (100%), 20 ng/mL (20%), 10 ng/mL (10%), 5 ng/mL (5%), and 0 ng/mL (0%). FIG. 1E shows a bar graph and a table summarizing the percentage of Sox7-negative, Oct4-positive cells (“Sox17−/Oct4+”), and Sox7-negative, Oct4-negative cells (“double negative”) at S1C under the five different conditions. These data show that the percentage of Sox17−/Oct4+ cells did not change significantly when Activin A concentration was reduced from 100% to 20%, 10%, or 5%, but significantly increased from under 1% with treatment with 100% Activin A to over 20% with no Activin A; with reduced Activin A (20%, 10%, and 5%), there was increase in the percentage of double negative cells, while the percentage remained similar when there was no Activin A.

### Example 2. Effects of Two Exemplary Compounds on Differentiation of Pancreatic β Cells

This example demonstrates the effects of treatment of stem cells with two exemplary compounds according to the present disclosure, GSK-690693 and BYL719, on the differentiation of pancreatic β cells from stem cells.

The same exemplary base differentiation protocol, Version A, as in EXAMPLE 1, was used subject to modifications for experiments described below.

In one experiment, four different conditions for Stage 1 differentiation were tested: 100% Activin A, 10% Activin A, 10% Activin A plus GSK-690693 (“GSK”), or 10% Activin A plus BYL719 (“BYL”). Characterization of cell morphology and cell constituents were conducted similarly as described in EXAMPLE 1. The experiment was repeated for four times. Table 3 summarizes the quantification of flow cytometry analysis results at S1C, S3C, and S5C, and total viable cell yield at S5C for the four runs. In all the four runs, 0.1 μM GSK-609063 or 0.04 μM BYL719 was applied for 3 days of Stage 1, except that in the last two runs, Run 4 and Run 5, 0.04 μM of BYL719 was applied for only 2 days (days S1d1 and S1d2).

TABLE 3
Effects of GSK-690693 and BYL719 on Cell Differentiation

| | | S1C | | S5C | St5C |
|---|---|---|---|---|---|
| | | Sox17+/ | S3C | Nkx6.1+/ | yield |
| | | Oct4− | Pdx1 | Isl1+ | (×10⁶/ml) |
| Run 2 | 100% AA | 84.5 | | | |
| | 10% AA | 73.8 | | | |
| | GSK + 10% AA | 60 | | | |
| | BYL + 10% AA | 74.8 | | | |
| Run 3 | 100% AA | 78.2 | 72.7 | 31.5 | |
| | 10% AA | 61.6 | 56.9 | 25.2 | |
| | GSK + 10% AA | 69 | 57.5 | 31.1 | |
| | BYL + 10% AA | 69.1 | 65.9 | 30.1 | |
| Run 4 | 100% AA | 88.2 | 95.3 | 36.6 | 1.51 |
| | 10% AA | 80.5 | 93.3 | 38.7 | 2.56 |
| | GSK + 10% AA | 84.9 | 91.1 | 35.6 | 3.02 |
| | BYL + 10% AA | 77.3 | 84.8 | 27 | 4.19 |
| Run 5 | 100% AA | 79.1 | 84.61 | 32.6 | 4.4544 |
| | 10% AA | 87.1 | 75.8 | 32.9 | 4.1968 |
| | GSK + 10% AA | 91.6 | 89.4 | 28.5 | 5.4416 |
| | BYL + 10% AA | 86.2 | 85.8 | 29.3 | 5.0608 |

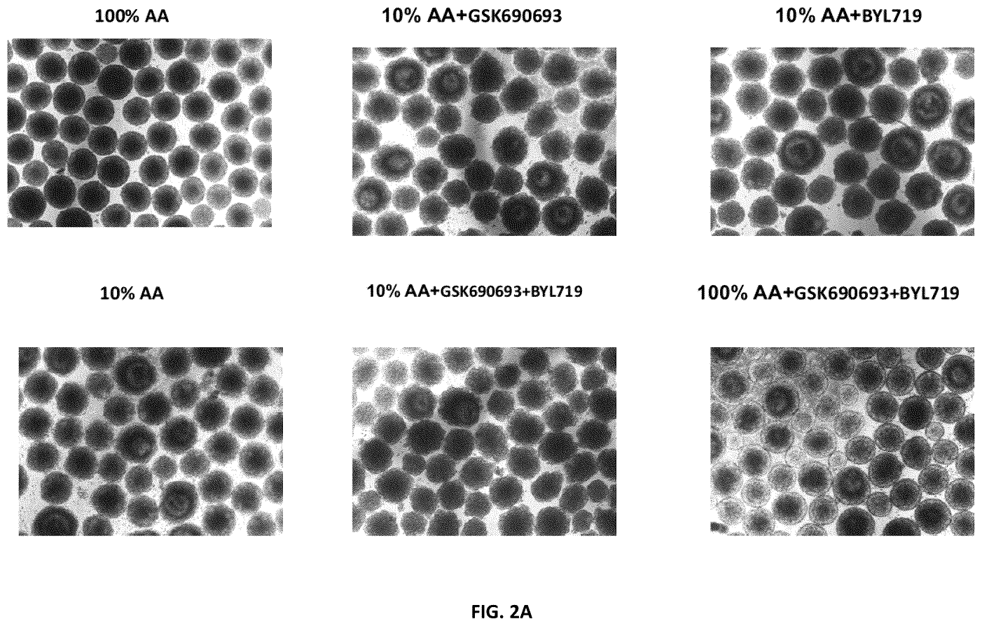

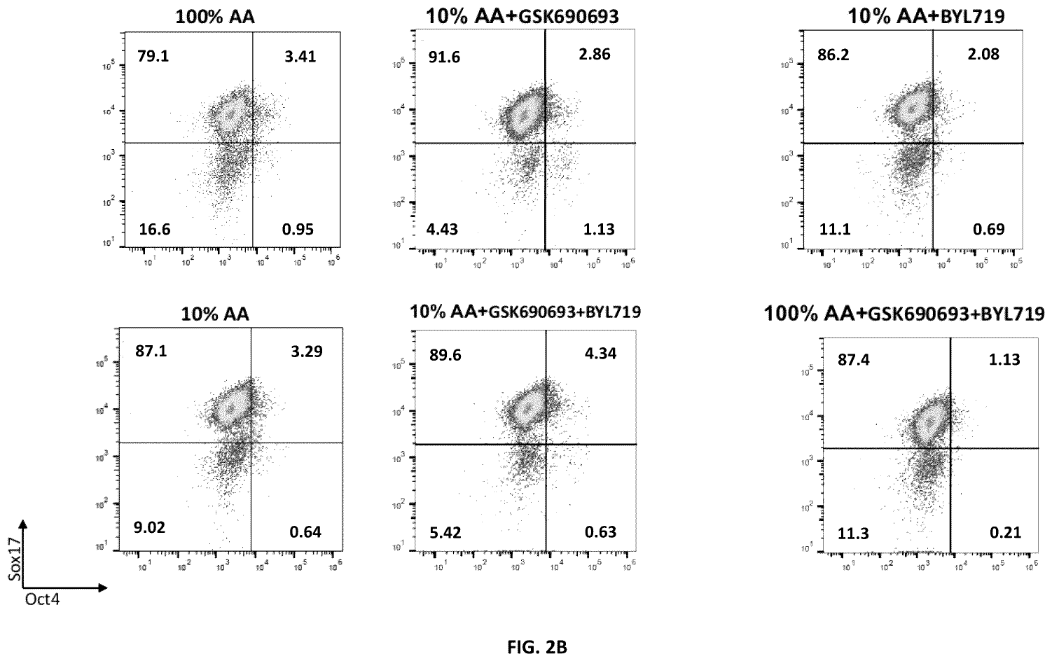

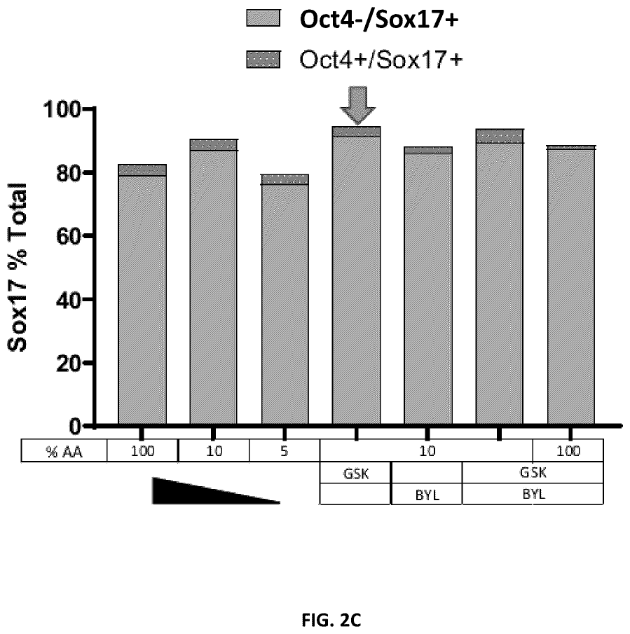

FIG. 2A shows pictures of cell clusters under the six different conditions upon completion of Stage 1. FIG. 2B shows results of flow cytometry analysis of S1c cells for expression of Oct 4 and Sox17, which is summarized in the bar graph in FIG. 2C.

### Example 3. Combination of Exemplary Compounds

This example demonstrates the effects of treatment of stem cells with a combination of two exemplary compounds, GSK-690693 and BYL719, on the differentiation of pancreatic R cells from stem cells.

The same exemplary base differentiation protocol, Version A, as in EXAMPLE 1, was used subject to modifications for experiments described below.

In one experiment, six different conditions for Stage 1 differentiation were tested: 100% Activin A, 10% Activin A, 10% Activin A plus GSK-690693, 10% Activin A plus BYL719, 10% Activin A plus GSK-690693 and BYL719, or 100% Activin A plus GSK-690693 and BYL719. Characterization of cell morphology and cell constituents were conducted similarly as described in EXAMPLE 1. FIGS. 2A-5E and Table 4 summarize the characterizations of the cells obtained under the six differentiation conditions at different stages.

FIG. 2A shows pictures of cell clusters under the six different conditions upon completion of Stage 1. FIG. 2B shows results of flow cytometry analysis of S1C cells for expression of Oct4 and Sox17, which are summarized in the bar graph in FIG. 2C.

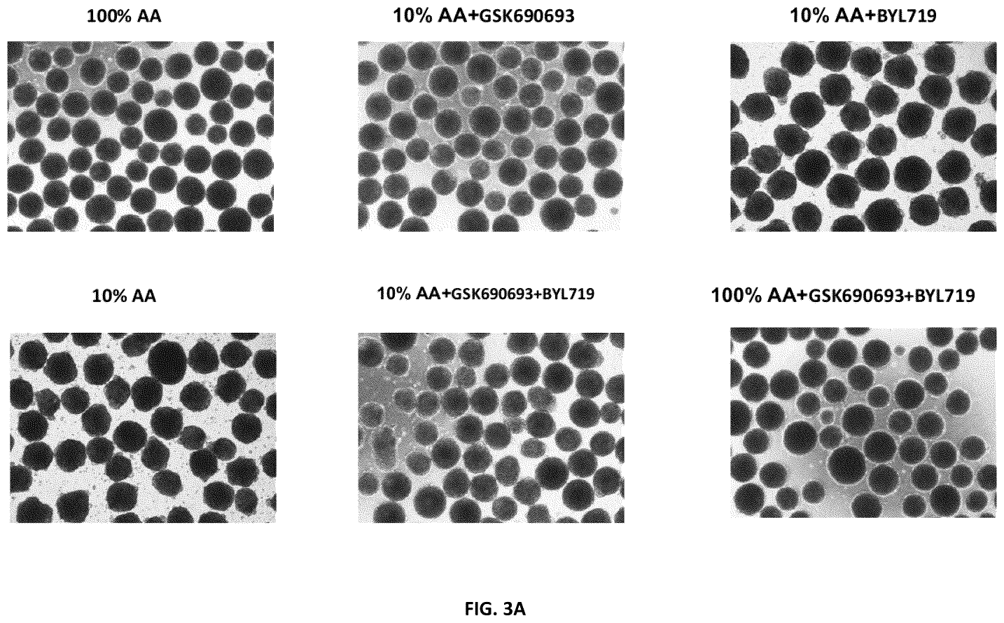

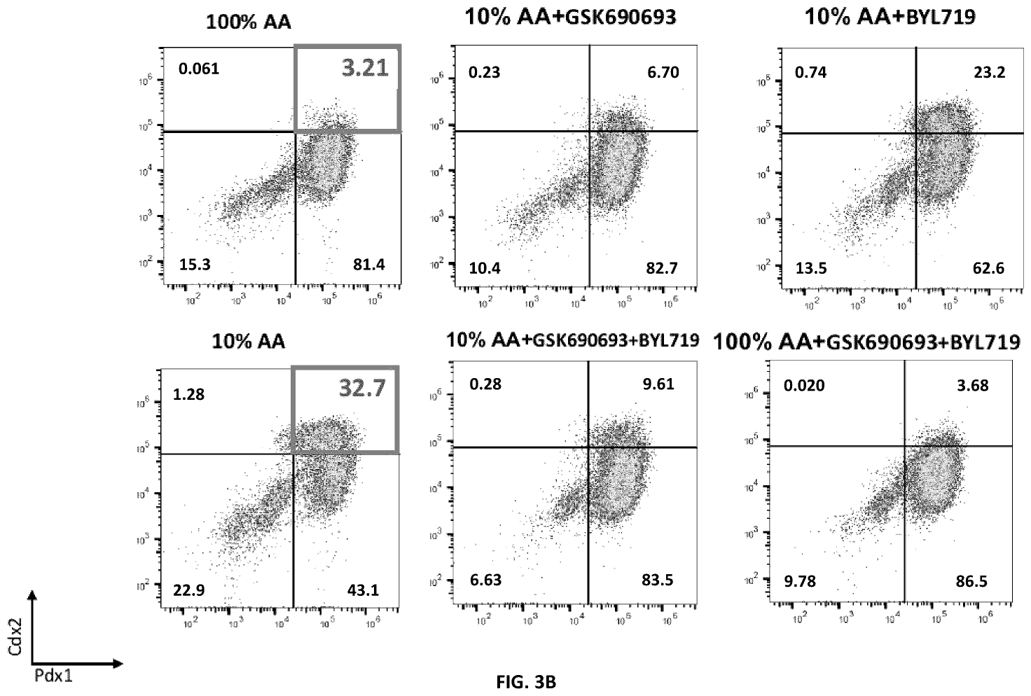

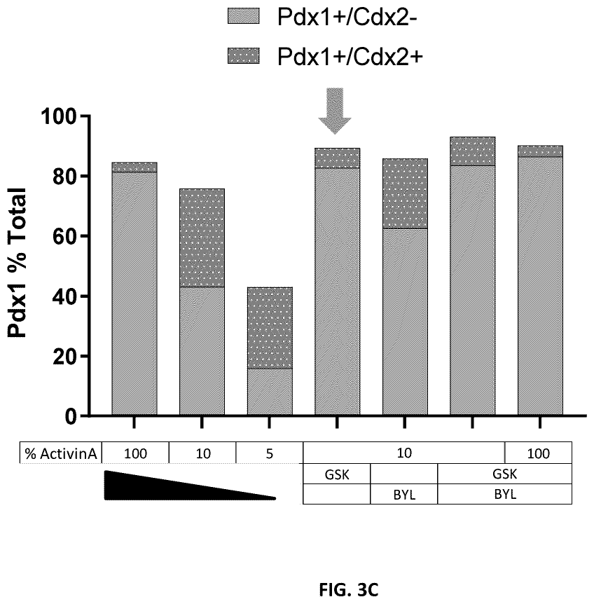

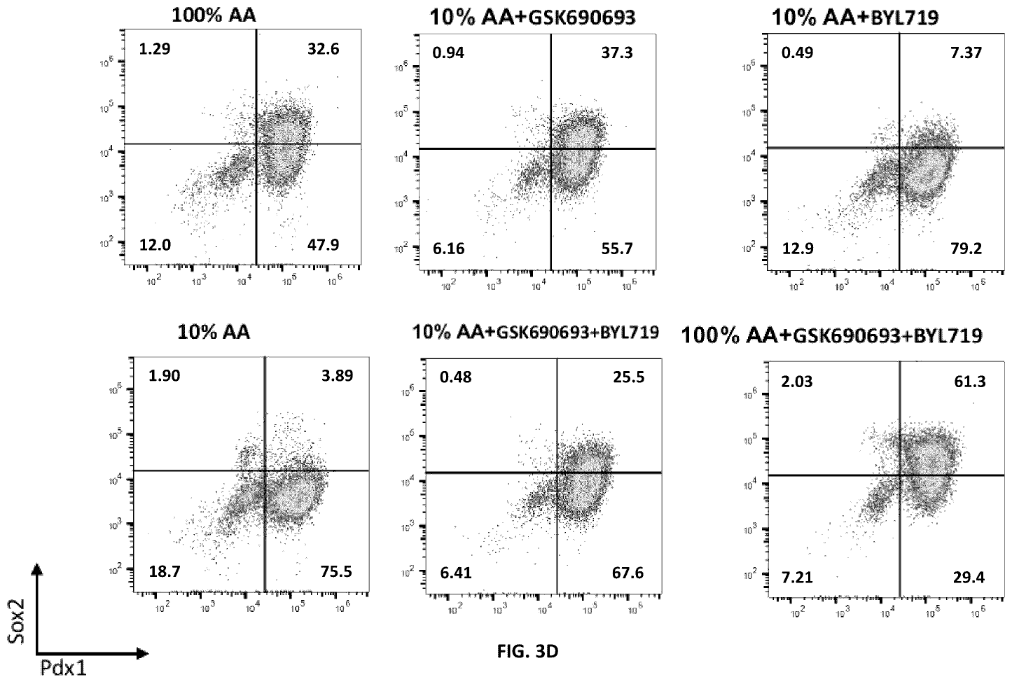

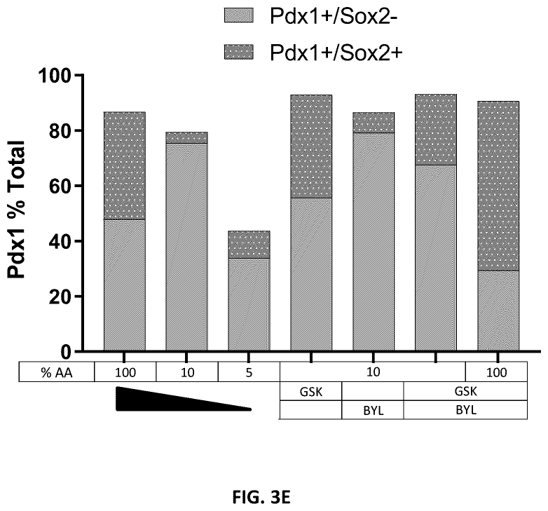

FIG. 3A shows pictures of cell clusters at S3C. FIGS. 3B and 3D shows results of flow cytometry analysis of S3C cells for expression of Cdx2, Sox2, and Pdx1, which are summarized in the bar graphs in FIGS. 3C and 3E, respectively.

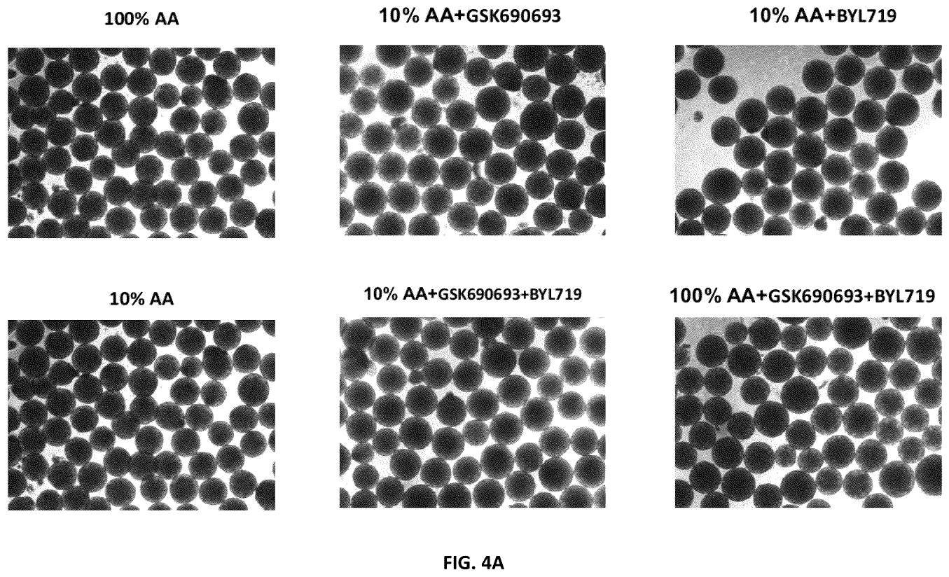

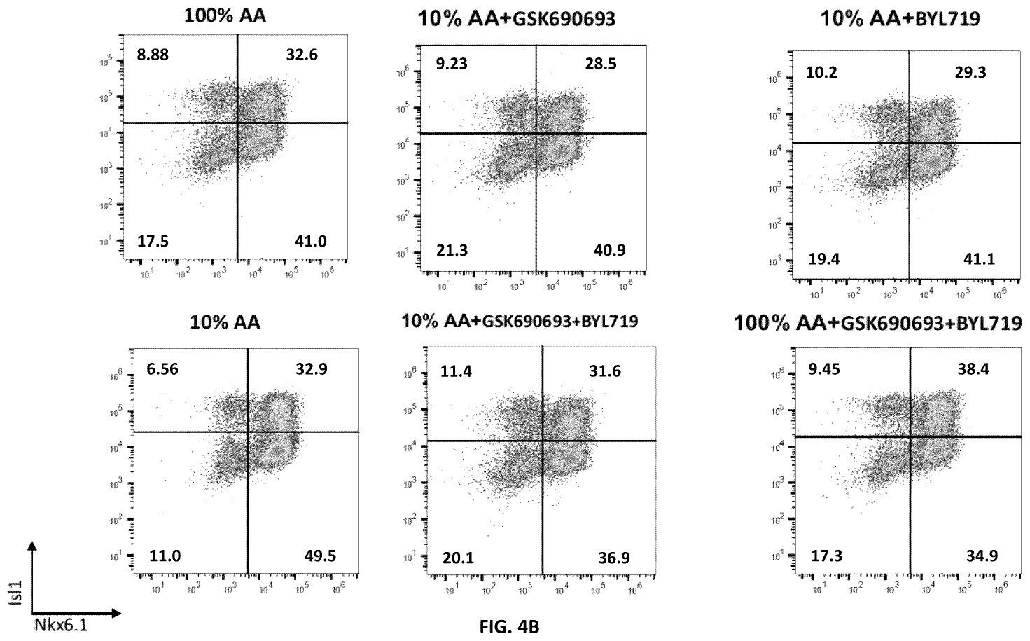

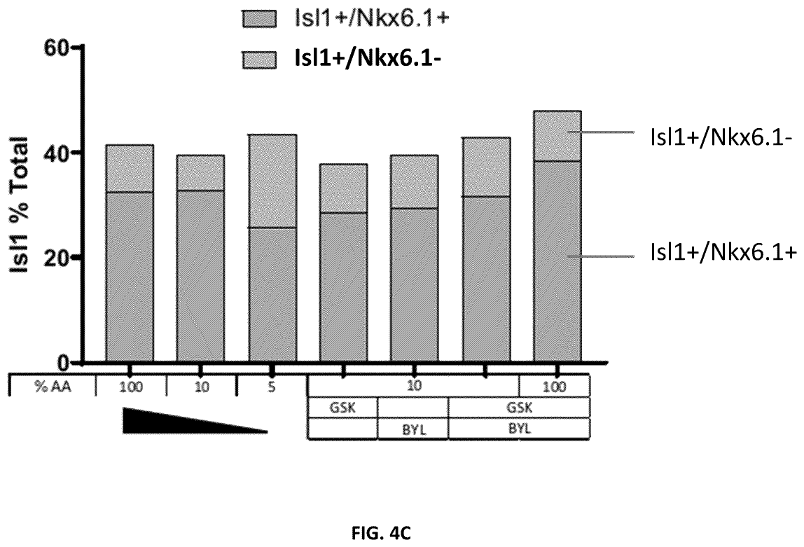

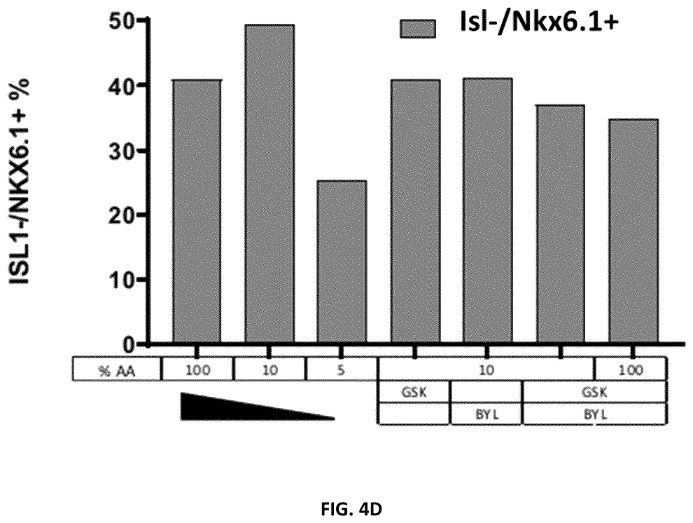

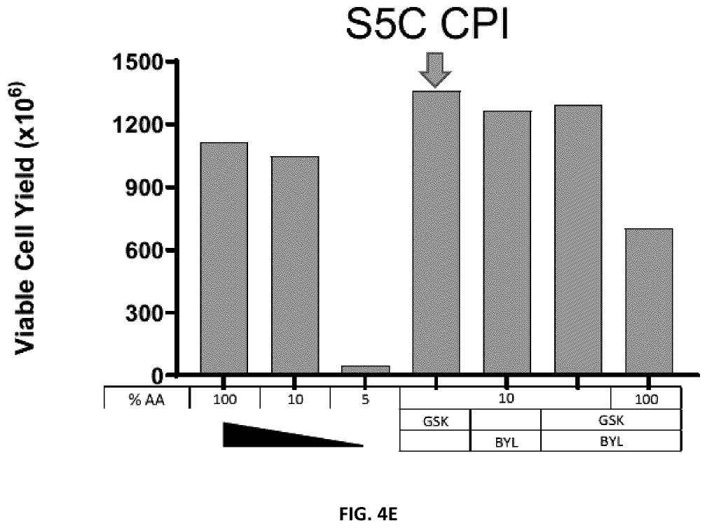

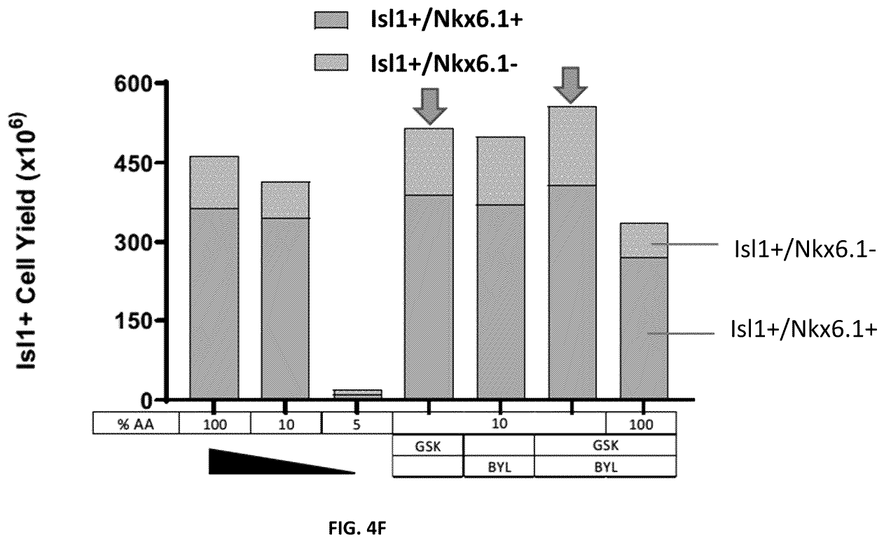

FIG. 4A shows pictures of cell clusters at S5C. FIG. 4B shows results of flow cytometry analysis of S5C cells for expression of Nkx6.1 and Isl1, which is summarized in the bar graphs in FIGS. 4C-4D. FIGS. 4E-4F show bar graphs summarizing the viable cell yield and the yield of Isl1-positive cells at S5C under the six different conditions, respectively.

TABLE 4
Effects of Treatment of GSK-690693, BYL719, or Both on Cell Differentiation

| | | 100% | 10% | 10% AA + | 10% AA + | 10% AA + | 100% AA + |
|---|---|---|---|---|---|---|---|
| Stage | Markers | AA | AA | GSK | BYL | GSK + BYL | GSK + BYL |
| S1C | Sox17 + /Oct4− | 79.1% | 87.1% | 91.6% | 86.2% | 89.6% | 87.4% |
| | Sox17 + /Oct4+ | 3.41% | 3.29% | 2.86% | 2.08% | 4.34% | 1.13% |
| S3C | Pdx1 + /Cdx2+ | 3.21% | 32.7% | 6.70% | 23.2% | 9.61% | 3.68% |
| | Pdx1 + /Sox2+ | 38.8% | 3.89% | 37.3% | 7.37% | 25.5% | 61.3% |
| S5C | Nkx6.1 + /Isl1+ | 32.6% | 32.9% | 28.5% | 29.3% | 31.6% | 38.4% |
| S6d7 | Nkx6.1 + /Isl1+ | 37.3% | 35.4% | 34.8% | 33.7% | 35.4% | 41.4% |

In another experiment, the effects of the aforementioned six different conditions for Stage 1 differentiation on Stage 6 differentiation were tested under two Stage 6 medium regimens: regimen 1 (Table 5) and regimen 2 (Table 6).

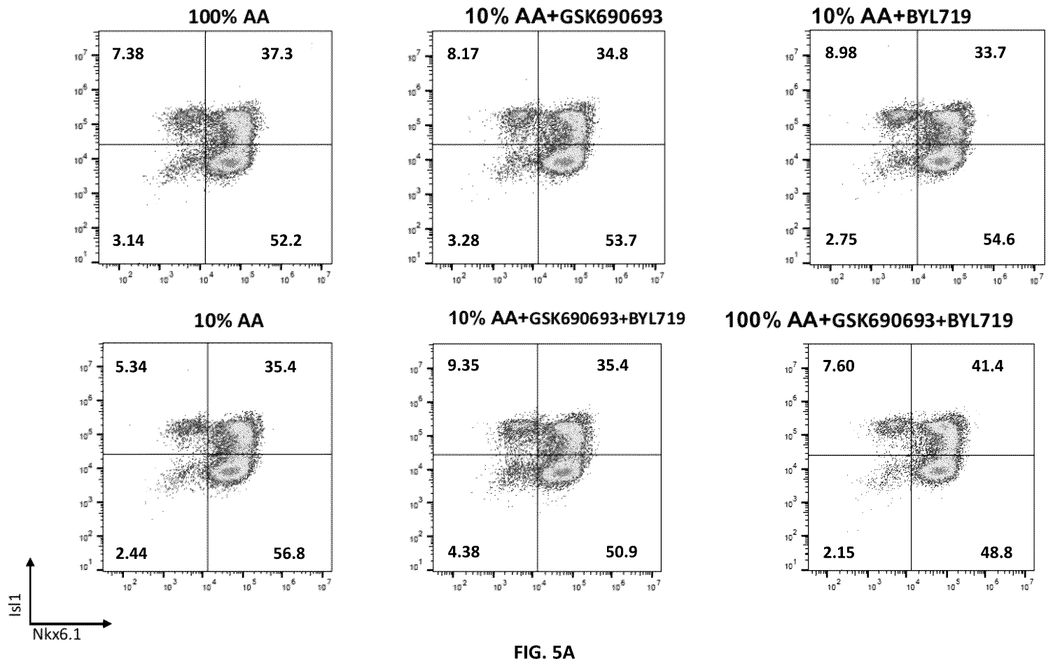

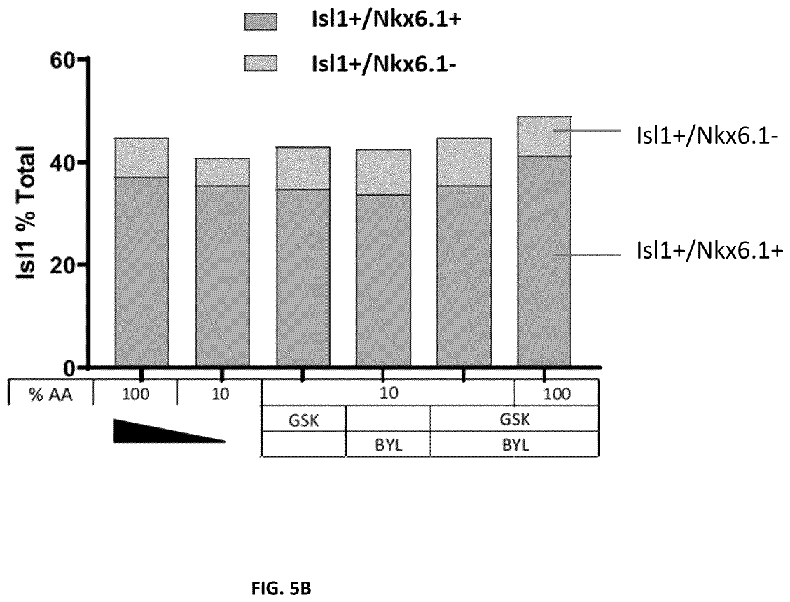

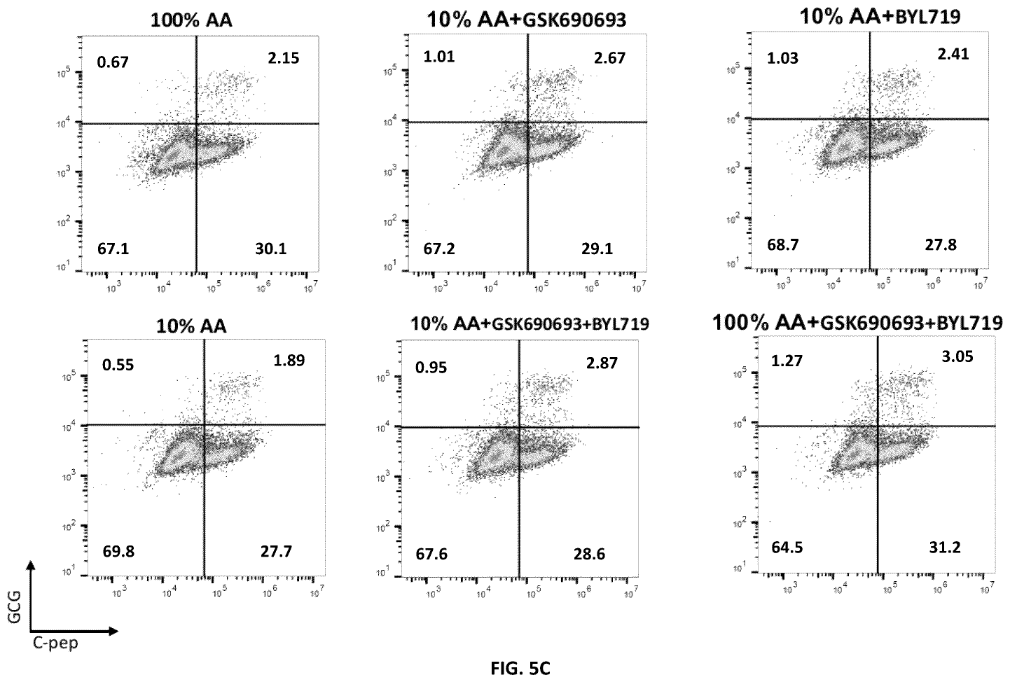

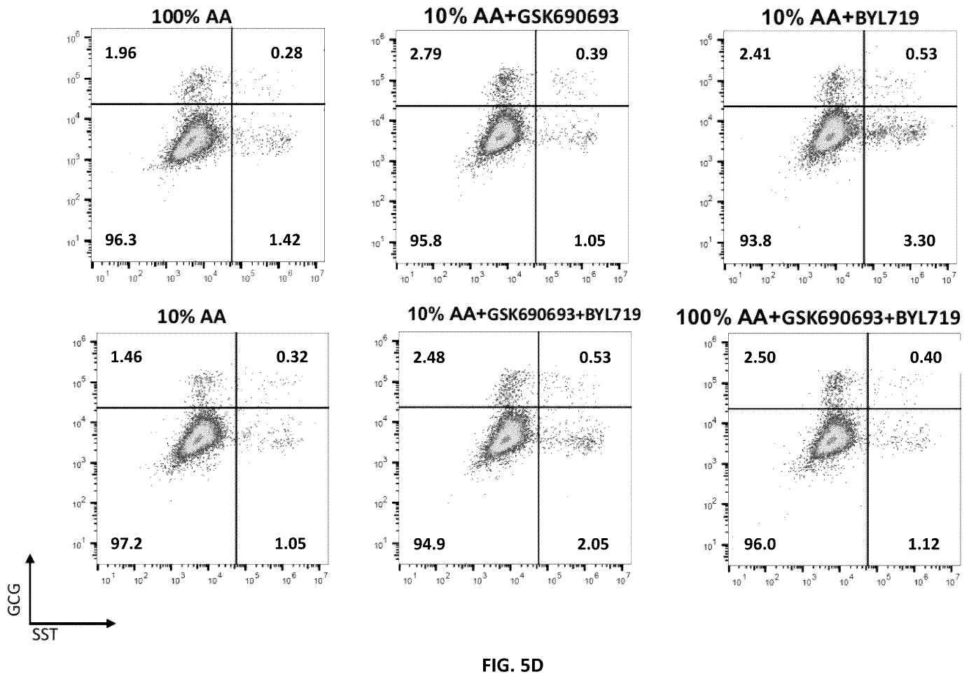

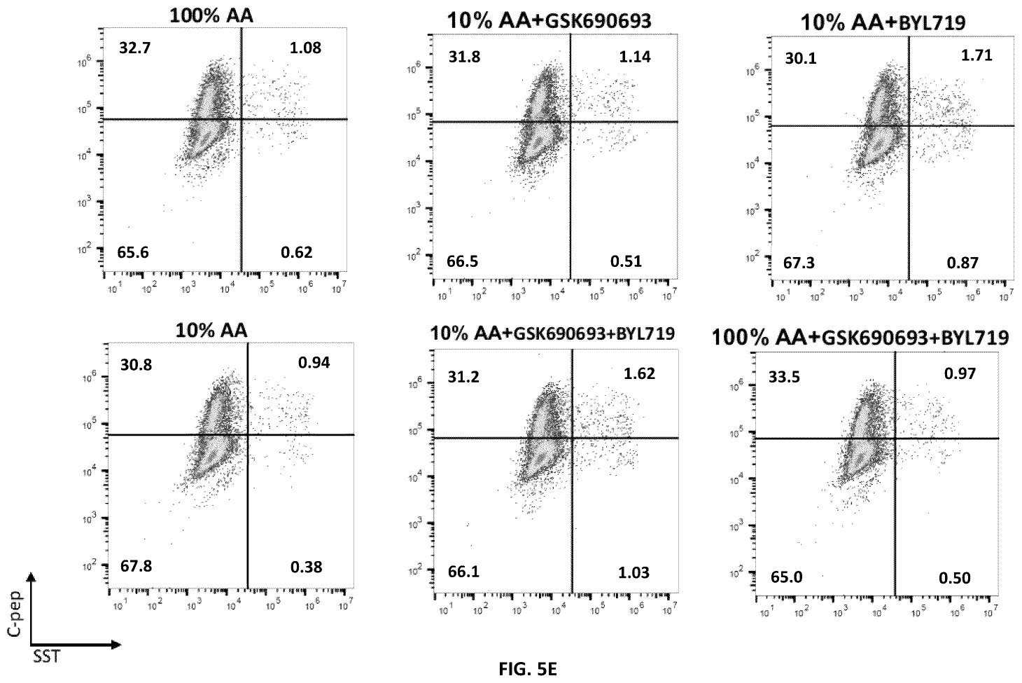

FIGS. 5A, 5C-5E show results of flow cytometry analysis of cells on day 7 of Stage 6 (S6d7) under regimen 1 and the six differentiation conditions for their respective expression of markers for staining β cells (Nkx6.1 and Isl1, FIG. 5A), α cells (glucagon and C-peptide, FIGS. 5C-5D), and δ cells (somatostatin, FIGS. 5D-5E). FIG. 5B shows a bar graph summarizing the percentages of Isl1-positive, Nkx6.1-positive cells and Isl1-positive, Nkx6.1-negative cells at S6d7 under the six differentiation conditions.

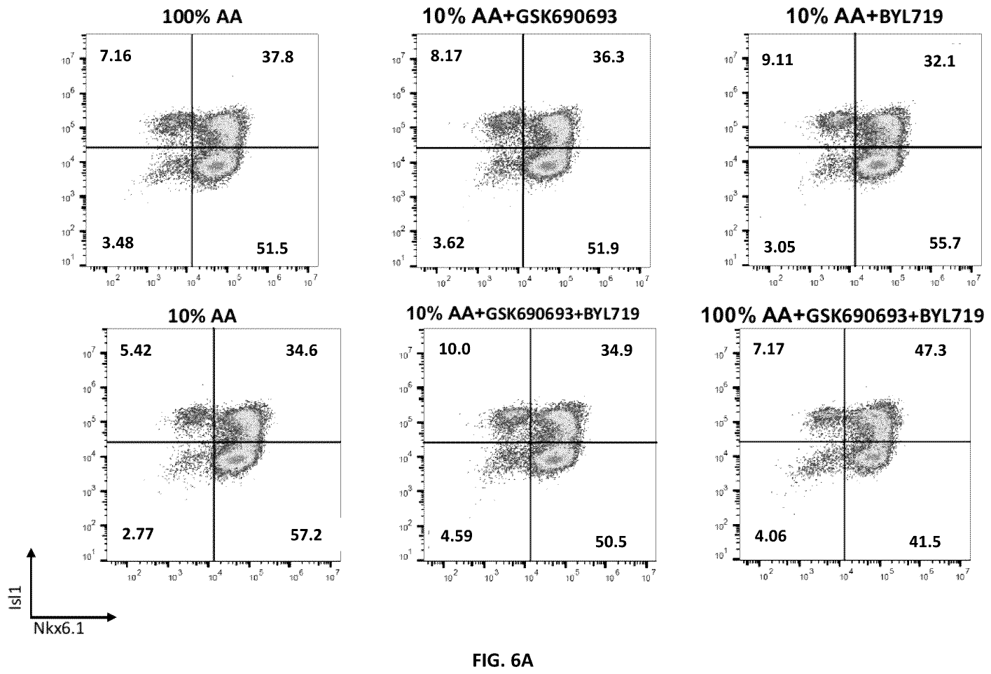

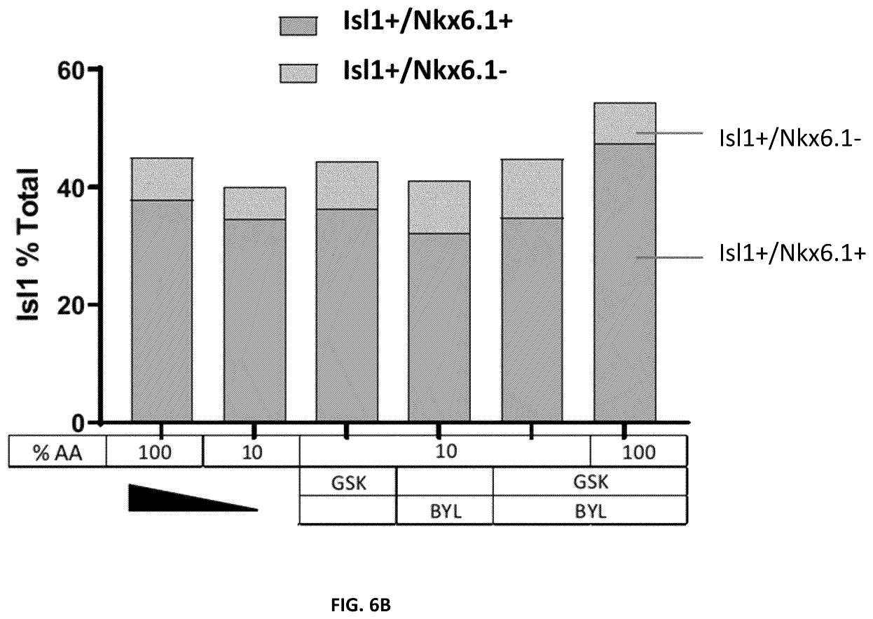

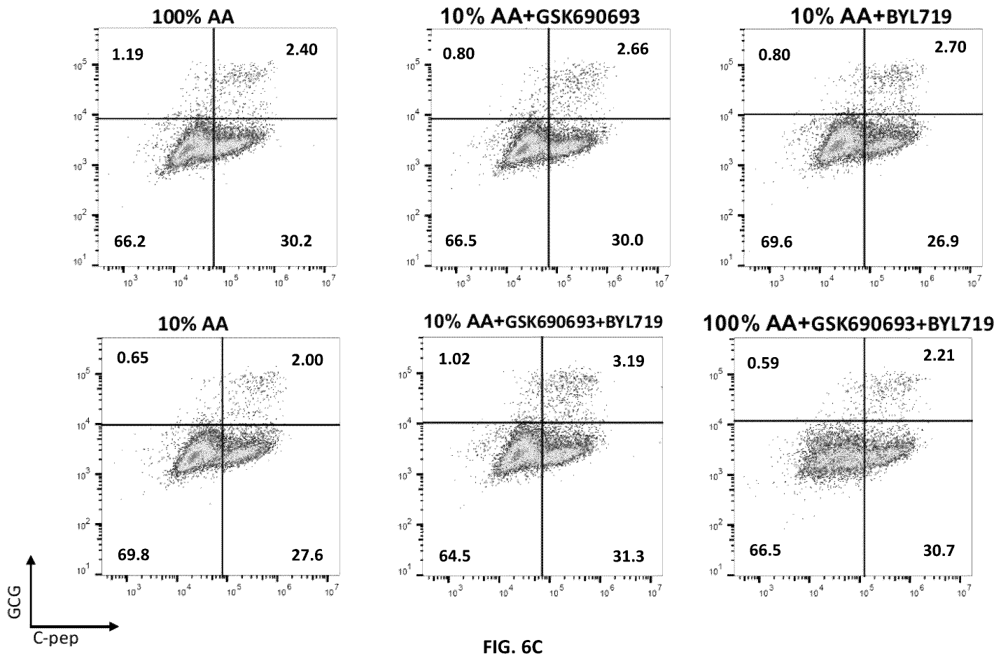

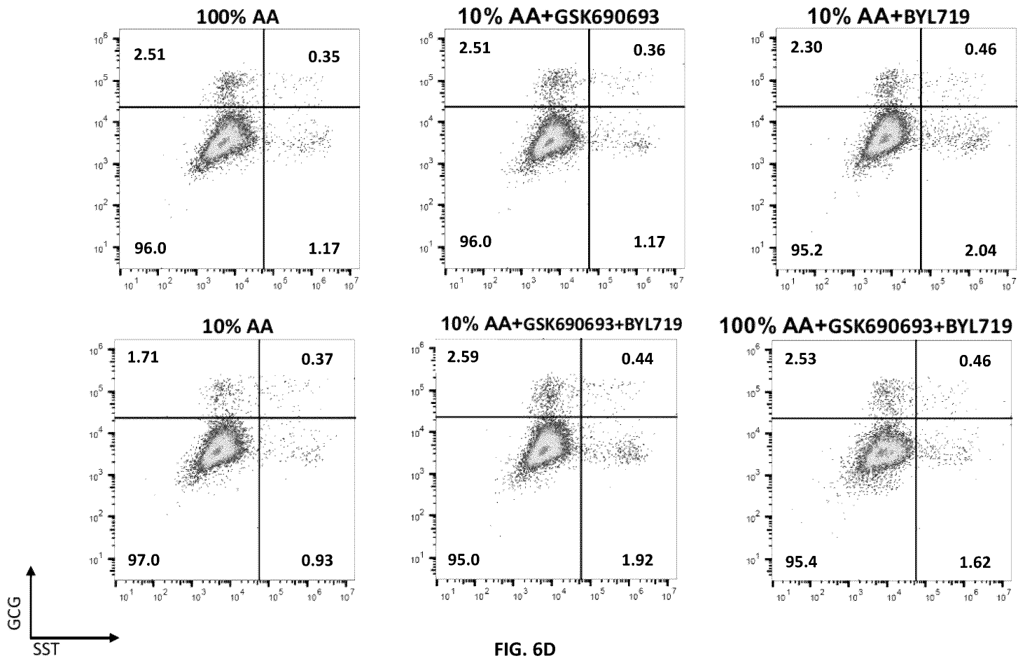

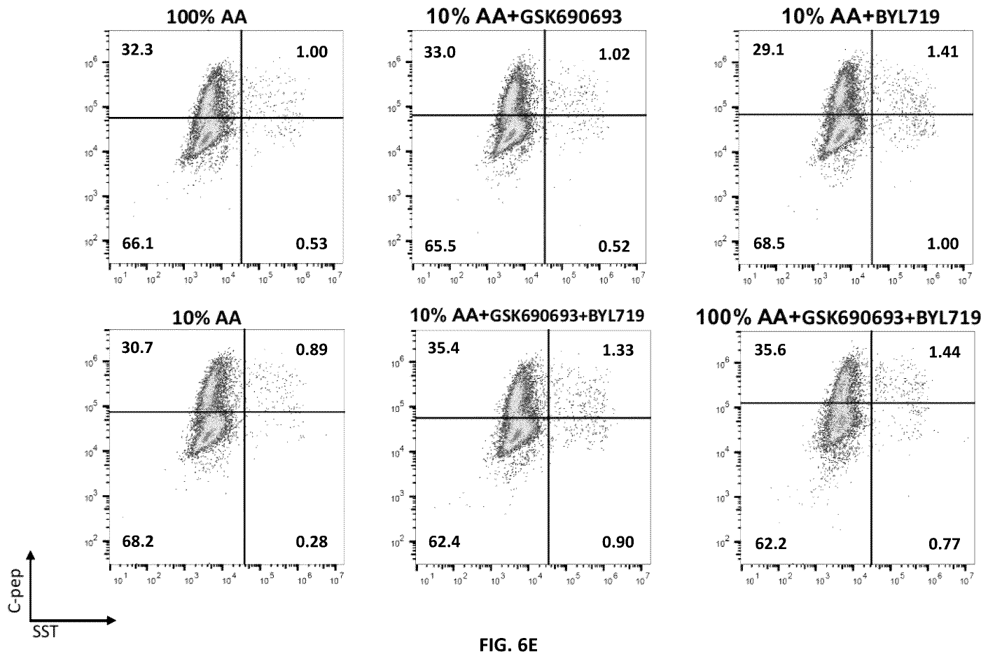

FIGS. 6A, 6C-6E show results of flow cytometry analysis of cells on day 7 of Stage 6 (S6d7) under regimen 2 and the six differentiation conditions for their respective expression of markers for staining β cells (Nkx6.1 and Isl1, FIG. 6A), α cells (glucagon and C-peptide, FIGS. 6C-6D), and δ cells (somatostatin, FIGS. 6D-6E). FIG. 6B shows a bar graph summarizing the percentages of Isl1-positive, Nkx6.1-positive cells and Isl1-positive, Nkx6.1-negative cells at S6d7 under the six differentiation conditions.

TABLE 5
Regimen 1 for Stage 6 Differentiation

| | | Stage 6 days 1-4 | Stage 6 days 4-7 |
|---|---|---|---|
| Basal media | | DMEM/F12 | DMEM/F12 |
| | | w/glutamine | w/glutamine |
| Media | HSA | 0.05% | 1% |
| supplements | ZnSO4 | 10 μM | 10 μM |
| Small molecules | Alk5i | 10 μM | — |
| | GC-1 | 1 μM | — |
| | LDN-193189 | 100 nM | — |
| | thiazovinin | 2.5 μM | — |
| | SSP | 3 nM | — |
| | DZNEP | 100 nM | — |
| Metabolites | L-glutamate | 0.5 mM | 0.5 mM |
| | L-carnitine | 40 μM | 40 μM |
| | Taurine | 90 μM | 90 μM |
| | Acetate | 160 nM | 160 nM |
| | B-hydroxybutyrate | 200 nM | 200 nM |
| | Biotin | 800 nM | 800 nM |
| | Formate | 50 μM | 50 μM |

TABLE 6
Regimen 2 for Stage 6 Differentiation

| | | Stage 6 days 1-4 | Stage 6 days 4-7 |
|---|---|---|---|
| Basal media | | DMEM/F12 | DMEM/F12 |
| | | w/glutamine | w/glutamine |
| Media | HSA | 0.05% | 1% |
| supplements | ZnSO4 | 10 μM | 10 μM |
| Small molecules | Alk5i | 10 μM | — |
| | GC-1 | 1 μM | — |
| | LDN-193189 | 100 nM | — |
| | thiazovinin | 2.5 μM | — |
| | SSP | 3 nM | — |
| | DZNEP | 100 nM | — |
| Metabolites | L-glutamate | 0.5 mM | — |
| | L-carnitine | 40 μM | — |
| | Taurine | 90 μM | — |
| | Acetate | 160 nM | — |
| | B-hydroxybutyrate | 200 nM | — |
| | Biotin | 800 nM | — |
| | Formate | 50 μM | — |

TABLE 7
Effects of Treatment of GSK-690693, BYL719, or Both on Stage 6

| | Markers on | 100% | 10% | 10% AA + | 10% | 10% AA + | 100% AA + |
|---|---|---|---|---|---|---|---|
| | S6d7 | AA | AA | GSK | AA + BYL | GSK + BYL | GSK + BYL |
| Regimen 1 | Isl1 + /Nkx6.1+ | 37.3 | 35.4 | 34.8 | 33.7 | 35.4 | 41.4 |
| | Isl1 + /Nkx6.1− | 7.38 | 5.34 | 8.17 | 8.98 | 9.35 | 7.60 |
| Regimen 2 | Isl1 + /Nkx6.1+ | 37.8 | 34.6 | 36.3 | 32.1 | 34.9 | 47.3 |
| | Isl1 + /Nkx6.1− | 7.16 | 5.42 | 8.17 | 9.11 | 10.0 | 7.17 |

### Example 4. Examination of Small Molecule Compounds for Pancreatic β Cells Differentiation

This example demonstrates the effects of a large number of small molecule compounds, including exemplary compounds according to the present disclosure, on the differentiation of pancreatic β cells from stem cells.

In one experiment, more than 2100 small molecule compounds (“test compounds”) from a small molecule chemogenomic library were tested for their effects on Stage 1 differentiation from stem cells to Sox17-positive definitive endoderm cells. Stem cells were seeded at a density of 45k cells per well in 384-well plates, and were treated with Stage 1 conditions that are same as Version A protocol in Example 1 except for 100 ng/mL Activin A (100% AA; “positive control”), modified Stage 1 condition with no AA (“negative condition”), modified Stage 1 condition with 10 ng/mL AA (10% AA; “neutral condition”), or modified Stage 1 condition with 10 ng/mL AA and one of the test compounds (“test condition”). Each test compound was tested at about 8 different concentrations. The resulting cell population in each well was then examined by immunostaining and image analysis to determine the percentage of cells expressing Sox17 and/or Oct14 (via immunostaining of Sox17 and Oct 14), and total cell count (via DAPI staining). Out of the total 2100+ test compounds, 56 test compounds met the following criteria and were considered as active compounds: (a) the resulting cell population has more than 50% Sox17-positive cells as compared to 100% AA condition; and (b) the resulting cell population has Z-Score of DAPI staining within 3 units of Stimulator Control (i.e., 100% AA). Fifteen (15) top active compounds among the initial 56 active compounds: GSK-690693, BAY 41-2272, SCHEMBL12114705, IPI-3063, AZD8055, Omipalisib, GNE-477, VS-5584, DDR1-IN-1, IWR-1-endo, BYL319, YM201636, PI4KIIIbeta-IN-10, and Nemiralisib. These 15 top active compounds were found to increase signal in Sox17 channel and decrease signal in Oct 4 channel both in a dose-responsive manner, and were found upon visual inspection not to induce nuclear blebbing in the resulting cell population. Quantification thresholds correspond to staining signal and were defined as low, normal, or high. Low indicates a low threshold was set for dectection of Sox17+ staining (i.e., easy to detect Sox17+) and high would be only highest expressing Sox17+ cells are detected. Table 8 shows ten of these active compounds and the percentage of Sox17-positive cells normalized by the percentage with 100 ng/mL AA. Without wishing to be bound by a certain theory, the “Target/Pathway” for each active compound in the table shows one of the signaling pathways that the respective compound may target (e.g., inhibit activity of the indicated signaling pathway).

In another experiment, eleven of the fifteen top active compounds were tested for differentiation of stem cells under 3D culture condition. Similar to the 384-well plate experiment, Version A protocol was used to treat the cells with the modification that reduced the concentration of Activin A from 100 ng/mL to 10 ng/mL and added one of the test compounds at two test concentrations. Three of 11 compounds passed QC criteria for DE population (Stage 1 complete), 3 of 3 compounds passed QC for PP1 population (Stage 3 complete), 2 of 3 compounds passed QC criteria for SC-β cells (Stage 5 complete). QC pass criteria were determined based on similarity to Version A protocol.

TABLE 8
Exemplary Active Compounds

| Compound | Target/Pathway | Relative to 100 ng/mL AA |
|---|---|---|
| GSK-690693 | AKT1/2/3/AMPK/DAPK3 | 119% |
| BAY 41-2272 | Soluble Guanylate Cyclase | 145% |
| FT113 | FASN | 64% |
| IPI-3063 | PI3K/p110d | 130% |
| AZD8055 | mTOR | 117% |
| Omipalisib | P110a/b/g/d/mTORC1/2 | 75% |
| GNE-477 | PI3K/mTOR | 160% |
| VS-5584 | PI3K/mTOR | 131% |
| DDR1-IN-1 | DDR1 | 120% |
| Apitolisib | PI3Ka/b/g/d/mTOR | 168% |

### Example 5. Composition of Stem Cell-Derived Pancreatic Cells

This example shows the composition of stem cell-derived pancreatic cells following different differentiation protocols according to some embodiments of the present disclosure.

Two different protocols were utilized for inducing differentiation of stem cells into pancreatic cells: Version B (“VB”) and a modified version Version B.1 (VB.1). Both protocols are 6-stage protocols. In Version B protocol, the reagents are added to the culture medium from stage 1 (three days in total, from Stage 1 Day 1 (“S1D1”) to Stage 1 Day 3 (“S1D3”)), stage 2 (3 days in total), stage 3 (2 days in total), stage 4 (4 days in total), until stage 5 (7 days in total), according to Table 9 below. Stage 6 conditions of VB followed “Regimen 2” as described in Table 6 above. VB.1 protocol is identical to VB protocol except that in stage 1 of VB.1 protocol, a combination of 0.1 μM GSK-690693 (also referred to as “GSK690693”) and 10 ng/ml Activin A is used to replace Activin A (100 ng/mL) in the VB protocol.

TABLE 9
Stage 1 through stage 5 conditions of Version B protocol

| | |
|---|---|
| S1D1 | Ascorbic Acid (44 ng/ml) |
| | Activin A (100 ng/ml) |
| | CHIR99021 (3 μM) |
| | PVA80 |
| S1D2-3 | Ascorbic Acid (44 ng/ml) |
| | Activin A (100 ng/ml) |
| | PVA80 |
| S2D1-3 | Ascorbic Acid (44 ng/ml) |
| | KGF (50 ng/ml) |
| | PVA80 |
| S3D1 | Ascorbic Acid (44 ng/mL) |
| | KGF (50 ng/ml) |
| | PDBU (500 nM) |
| | Sant-1 (250 nM) |
| | Retinoic Acid (2 μM) |
| | Activin A (20 ng/ml) |
| | Thiazovinin (2.5 μM) |
| | DMH-1 (250 nM) |
| | PVA80 |
| S3D2 | Ascorbic Acid (44 ng/ml) |
| | KGF (50 ng/ml) |
| | PDBU (500 nM) |
| | Sant-1 (250 nM) |
| | Retinoic Acid (2 μM) |
| | Activin A (20 ng/ml) |
| | Thiazovinin (2.5 μM) |
| | PVA80 |
| S4D1-4 | Ascorbic Acid (44 ng/ml) |
| | KGF (50 ng/ml) |
| | Sant-1 (250 nM) |
| | Thiazovinin (2.5 μM) |
| | Activin A (5 ng/ml) |
| | Retinoic Acid (100 nM) |
| | PVA80 |
| S4D5-6 | Ascorbic Acid (44 ng/ml) |
| | KGF (50 ng/ml) |
| | Sant-1 (250 nM) |
| | Thiazovinin (2.5 μM) |
| | Activin A (5 ng/ml) |
| | Retinoic Acid (100 nM) |
| | PDBU (500 nM) |
| | AS1842856 (1 μM) |
| | XXI (2 μM) |
| | PVA80 |
| S5D1-3 | Ascorbic Acid (44 ng/ml) |
| | Sant-1 (250 nM) |
| | Betacellulin (20 ng/ml) |
| | XXI (2 μM) |
| | Alk5i (10 μM) |
| | GC-1 (1 μM) |
| | LDN-193189 (100 nM) |
| | Thiazovinin (2.5 μM) |
| | Staurosporine (3 nM) |
| | DZNEP (100 nM) |
| | Retinoic Acid (50 nM) |
| | PDBU (500 nM) |
| | NVP-TNKS656 (2 μM) |
| | L-glutamine (4 mM) |
| | Formate (50 M) |
| | Taurine (90 μM) |
| | Acetate (1 mM) |
| | β-hydroxybutyrate (200 nM) |
| | Biotin (800 nM) |
| | PVA 87-89 |
| S5D4-7 | Ascorbic Acid (44 ng/ml) |
| | XXI (2 μM) |
| | Alk5i (10 μM) |
| | GC-1 (1 μM) |
| | LDN-193189 (100 nM) |
| | Thiazovinin (2.5 μM) |
| | Staurosporine (3 nM) |
| | DZNEP (100 nM) |
| | L-glutamine (4 mM) |
| | Formate (50 μM) |
| | Taurine (90 M) |
| | Acetate (1 mM) |
| | β-hydroxybutyrate (200 nM) |
| | Biotin (800 nM) |
| | PVA 87-89 |

Both differentiation protocols were tested on cell cultures at different scale: three different vessels were used, Biott® Bioreactors (“Biotts,” 30 mL in volume), spinners (“Spinners,” 300 mL in volume), and large-scale bioreactors (“Bioreactors,” 3 L in volume).

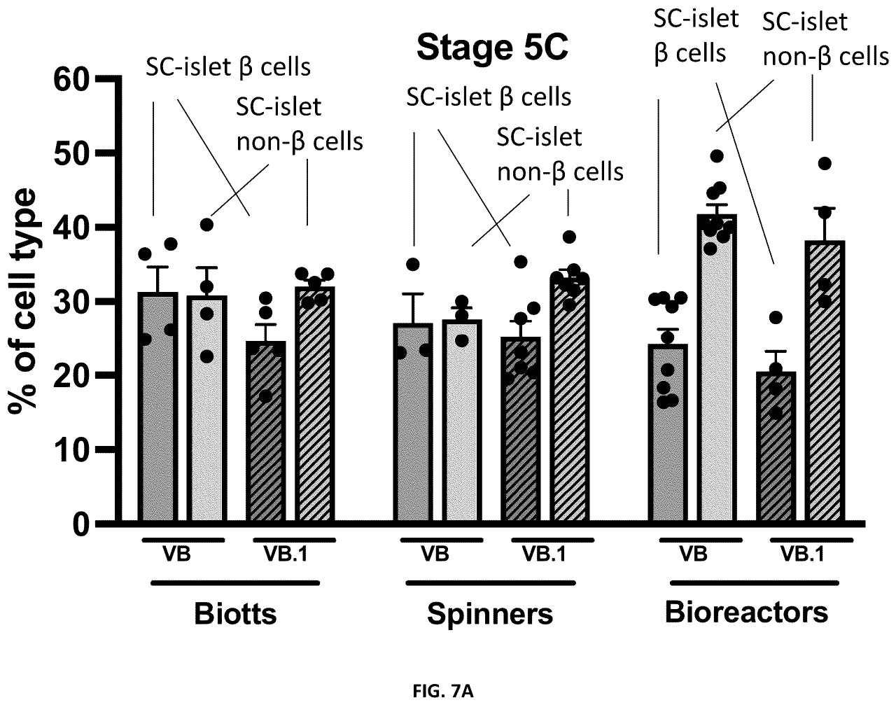

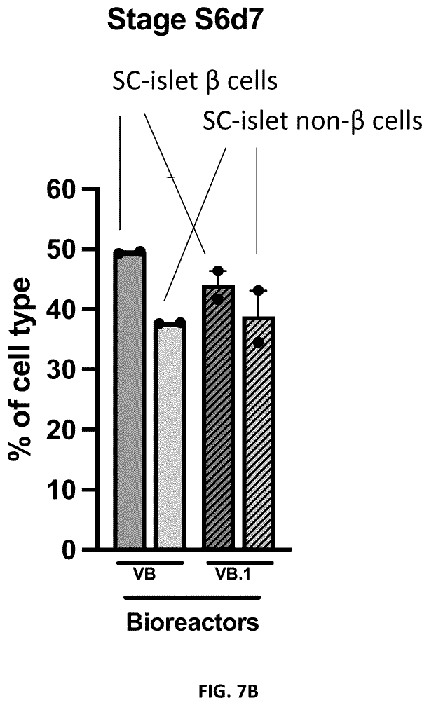

FIG. 7A shows a graph summarizing the percentage of SC-iselt β cells and the percentage of SC-islet non-β cells in the cell compositions at the completion of stage 5 differentiation (“S5C”) under different culture conditions. As shown in the figure, in all the tested culture vessels, the percentage of SC-islet β cells (NKX6.1-positive, ISL1-positive cells) and the percentage of SC-islet non-0 cells (NKX6.1-negative, ISL1-positive cells) were similar between S5C compositions generated using VB protocol and S5C compositions generated using VB.1 protocol. In Biotts, under VB protocol, about 30% of the S5C cells were SC-islet β cells on average and about 30% were SC-islet non-β cells on average; under VB.1 protocol, about 25% of the S5C cells were SC-islet β cells on average and about 30% were SC-islet non-β cells on average. In Spinners, under VB protocol, about 27% of the S5C cells were SC-islet β cells on average and about 28% were SC-islet non-β cells on average; under VB.1 protocol, about 26% of the S5C cells were SC-islet β cells on average and about 34% were SC-islet non-β cells on average. In Bioreactors, under VB protocol, about 25% of the S5C cells were SC-islet β cells on average and about 42% were SC-islet non-β cells on average; under VB.1 protocol, about 20% of the S5C cells were SC-islet β cells on average and about 38% were SC-islet non-β cells on average.

FIG. 7B shows a graph summarizing the percentage of SC-iselt β cells and the percentage of SC-islet non-β cells in the cell compositions on day 7 of Stage 6 (“S6d7”). Only S6d7 cells generated using Bioreactors were examined for this experiment, As shown in the figure, the percentage of SC-islet β cells and the percentage of SC-islet non-β cells in the S6d7 composition were similar between S6d7 cell compositions generated using VB protocol and S6d7 cell compositions generated using VB.1 protocol. Under VB protocol, about 50% of the S5C cells were SC-islet β cells on average and about 37% were SC-islet non-β cells on average; under VB.1 protocol, about 44% of the S5C cells were SC-islet β cells on average and about 38% were SC-islet non-β cells on average.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure can be employed in practicing the present disclosure. It is intended that the following claims define the scope of the present disclosure and that methods and structures within the scope of these claims and their equivalents be covered thereby.

What is claimed is:

1. An in vitro composition comprising Sox17-positive cells and an inhibitor of PI3K/Akt/mTOR signaling.

2. The composition of claim 1, wherein the composition further comprises stem cells.

3. The composition of claim 1 or 2, wherein the composition further comprises a growth factor from the TGF-β superfamily.

4. An in vitro composition comprising stem cells, an inhibitor of PI3K/Akt/mTOR signaling, and a growth factor from TGF-β superfamily.

5. The composition of claim 3 or 4, wherein the growth factor from TGF-β superfamily is selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11).

6. The composition of claim 3 or 4, wherein the growth factor from TGF-β superfamily comprises Activin A, GDF8, or both.

7. The composition of claim 2 or 6, wherein the composition comprises at most about 100 ng/mL, at most about 80 ng/mL, at most about 60 ng/mL, at most about 50 ng/mL, at most about 25 ng/mL, at most about 20 ng/mL, at most about 15 ng/mL, at most about 10 ng/mL, at most about 5 ng/mL, or at most about 2 ng/mL of Activin A.

8. The composition of claim 2 or 6, wherein the composition comprises from 0.5 ng/mL to 500 ng/mL, 1 ng/mL to 250 ng/mL, 10 ng/mL to 200 ng/mL, 20 ng/mL to 150 ng/mL, 50 ng/mL to 120 ng/mL, 1 ng/mL to 50 ng/mL, 2 ng/mL to 25 ng/mL, or 5 ng/mL to 20 ng/mL of Activin A.

9. The composition of claim 2 or 6, wherein the composition comprises about 1 ng/mL, about 2 ng/mL, about 3 ng/mL, about 4 ng/mL, about 5 ng/mL, about 6 ng/mL, about 7 ng/mL, about 8 ng/mL, about 9 ng/mL, about 10 ng/mL, about 12 ng/mL, about 14 ng/mL, about 15 ng/mL, about 18 ng/mL, about 20 ng/mL, about 25 ng/mL, about 30 ng/mL, or about 50 ng/mL of Activin A.

10. An in vitro composition comprising stem cells and an inhibitor of PI3K/Akt/mTOR signaling.

11. The composition of any one of claims 1-10, wherein the inhibitor of PI3K/Akt/mTOR signaling comprises an inhibitor of a PI3K protein, an inhibitor of an Akt protein, an inhibitor of mTOR, or any combination thereof.

12. The composition of any one of claims 1-11, wherein the inhibitor of PI3K/Akt/mTOR signaling comprises one or more of: GSK-690693, IPI-3063, AZD8055, Omipalisib, GNE-477, VS-5584, BYL319, YM201636, PI4KIIIbeta-IN-10, Nemiralisib, BYL719, FT113, or Apitolisib, or any analog or derivative thereof.

13. The composition of any one of claims 1-11, wherein the composition comprises an inhibitor of a PI3K protein and an inhibitor of an Akt protein.

14. The composition of any one of claims 1-13, wherein the composition comprises GSK-690693, an analog or a derivative thereof.

15. The composition of any one of claims 1-14, wherein the composition comprises BYL719, an analog or a derivative thereof.

16. The composition of any one of claims 1-14, wherein the composition comprises BYL319, an analog or a derivative thereof.

17. The composition of any one of claims 1-9, wherein the composition comprises GSK-690693, or an analog or a derivative thereof, and BYL319, or an analog or a derivative thereof.

18. The composition of any one of claims 1-9, wherein the composition comprises GSK-690693, or an analog or a derivative thereof, and BYL719, or an analog or a derivative thereof.

19. The composition of any one of claims 1-18, wherein the composition comprises from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693, or an analog or a derivative thereof.

20. The composition of any one of claims 1-18, wherein the composition comprises about 0.01 μM, about 0.02 μM, about 0.04 μM, about 0.06 μM, about 0.08 μM, about 0.1 μM, about 0.12 μM, about 0.15 μM, about 0.2 μM, about 0.3 μM, about 0.4 μM, about 0.5 μM, about 0.6 μM, about 0.8 μM, or about 1 μM of GSK-690693.

21. The composition of any one of claims 1-20, wherein the composition comprises from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719, or an analog or a derivative thereof.

22. The composition of any one of claims 1-20, wherein the composition comprises about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719.

23. The composition of any one of claims 1-22, wherein the composition comprises about 0.08 μM to about 0.12 μM of GSK-690693 and about 35 nM to about 50 nM of BYL719.

24. The composition of any one of claims 1-23, wherein the composition further comprises an activator of WNT signaling pathway.

25. The composition of claim 24, wherein the activator of WNT signaling pathway comprises one or more of Wnt3a, CHIR99021, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC693868, SB 216763, SB 415286, TC-G 24, TCS 2002, TCS 21311, TWS 119, and analogs or derivatives thereof.

26. The composition of any one of claims 1-25, wherein the composition further comprises a GSK3 inhibitor.

27. The composition of any one of claims 1-25, wherein the composition further comprises from 0.5 μM to 50 μM, 0.6 μM to 30 μM, 0.8 μM to 20 μM, 1 μM to 10 μM, or 2 μM to 5 μM of CHIR99021.

28. The composition of any one of claims 1-25, wherein the composition further comprises about 0.5 μM, 0.6 μM, 0.8 μM, 1 μM, 2 μM, 3 μM, 4 μM, 5 μM, 6 μM, 8 μM, 10 μM, 15 μM, 20 μM, 25 μM, or 30 μM of CHIR99021.

29. The composition of any one of claims 2-28, wherein the stem cells comprise embryonic stem cells.

30. The composition of any one of claims 2-28, wherein the stem cells comprise induced pluripotent stem cells.

31. The composition of any one of claims 2-30, wherein the stem cells are human cells.

32. The composition of any one of claims 2-31, wherein the stem cells are genetically modified.

33. The composition of any one of claims 1-3 or 6-32, wherein the composition comprises a population of cells that comprises Sox17-positive, Oct4-negative cells.

34. The composition of claim 33, wherein the population of cells comprises at least about 50%, 60%, 65%, 70%, 75%, 80%, or 85% Sox17-positive, Oct4-negative cells.

35. The composition of claim 33, wherein the population of cells comprises from about 50% to about 90%, about 60% to about 90%, about 65% to about 90%, about 70% to about 90%, about 75% to about 90%, about 80% to about 90%, or about 75% to about 85% Sox17-positive, Oct4-negative cells.

36. An in vitro composition comprising a plurality of FOXA2-positive, PDX1-negative cells and an inhibitor of PI3K/Akt/mTOR signaling.

37. The composition of claim 36, wherein the composition further comprises one or more agents selected from the group consisting of: a protein kinase C activator, a bone morphogenetic protein signaling pathway inhibitor, a growth factor from fibroblast growth factors (FGF) family, a retinoic acid (RA) signaling pathway activator, a Rho-associated, coiled-coil containing protein kinase (ROCK) inhibitor, and a sonic hedgehog (SHH) pathway inhibitor.

38. The composition of claim 36, wherein the composition further comprises:
(a) a protein kinase C activator selected from the group consisting of: phorbol 12,13-dibutyrate (PDBU), TPB, phorbol 12-myristate 13-acetate, and bryostatin 1;
(b) a bone morphogenetic protein signaling pathway inhibitor comprising LDN193189 or DMH-1;
(c) a growth factor from fibroblast growth factors (FGF) family selected from the group consisting of: keratinocyte growth factor (KGF), FGF2, FGF10, FGF21, and FGF8B;
(d) a sonic hedgehog pathway inhibitor selected from the group consisting of SANT1, SANT2, SANT4, Cur6l4l4, forskolin, tomatidine, AY9944, triparanol, and cyclopamine;
(e) a retinoic acid signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; and/or
(f) a ROCK inhibitor selected from the group consisting of Thiazovivin, Y-27632, Fasudil/HA1077, and 14-1152.

39. The composition of any one of claims 36-38, wherein the composition further comprises a growth factor from transformation growth factor β (TGF-β) superfamily.

40. The composition of claim 39, wherein the growth factor from the TGF-β superfamily is selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11).

41. The composition of claim 39, wherein the growth factor from the TGF-β superfamily comprises Activin A, GDF8, or both.

42. The composition of claim 39, wherein the composition comprises at most about 20 ng/mL Activin A.

43. The composition of claim 39, wherein the composition comprises at most about 10 ng/mL, at most about 5 ng/mL, at most about 1 ng/mL, at most about 0.5 ng/mL, or at most about 0.1 ng/mL Activin A.

44. The composition of claim 39, wherein the composition comprises about 20 ng/mL, about 10 ng/mL, about 5 ng/mL, about 1 ng/mL, about 0.5 ng/mL, or about 0.1 ng/mL Activin A.

45. The composition of any one of claims 36-44, wherein the composition further comprises PDX1-positive and NKX6.1-negative cells.

46. An in vitro composition comprising a plurality of PDX1-positive and NKX6.1-negative cells and an inhibitor of PI3K/Akt/mTOR signaling.

47. The composition of claim 46, wherein the composition further comprises one or more agents selected from the group consisting of: a growth factor from fibroblast growth factors (FGF) family, a retinoic acid (RA) signaling pathway activator, a Rho-associated, coiled-coil containing protein kinase (ROCK) inhibitor, a protein kinase C activator, and a sonic hedgehog (SHH) pathway inhibitor.

48. The composition of claim 46, wherein the composition further comprises:
(a) a growth factor from the transformation growth factor β (TGF-β) superfamily selected from the group consisting of: an Inhibin, an Activin, a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11);
(b) a growth factor from fibroblast growth factors (FGF) family selected from the group consisting of: keratinocyte growth factor (KGF), FGF2, FGF10, FGF21, and FGF8B;
(c) a retinoic acid (RA) signaling pathway activator selected from the group consisting of: retinoic acid, CD1530, AM580, TTHRB, CD437, Ch55, BMS961, AC261066, AC55649, AM80, BMS753, tazarotene, adapalene, and CD2314;
(d) a ROCK inhibitor selected from the group consisting of Thiazovivin, Y—27632, Fasudil/HA1077, and 14-1152;
(e) a protein kinase C activator selected from the group consisting of: phorbol 12,13-dibutyrate (PDBU), TPB, phorbol 12-myristate 13-acetate, and bryostatin 1;
(f) a sonic hedgehog (SHH) pathway inhibitor selected from the group consisting of SANT1, SANT2, SANT4, Cur6l4l4, forskolin, tomatidine, AY9944, triparanol, and cyclopamine; and/or
(g) a FoxO1 inhibitor, optionally wherein the FoxO1 inhibitor is AS1842856.

49. The composition of any one of claims 46-48, wherein the composition further comprises a notch signaling inhibitor, optionally wherein the notch signaling inhibitor is XXI or DAPI.

50. The composition of any one of claims 46-49, wherein the composition further comprises a growth factor from transformation growth factor β (TGF-β) superfamily.

51. The composition of claim 50, wherein the growth factor from the TGF-β superfamily is selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11).

52. The composition of claim 50, wherein the growth factor from the TGF-β superfamily comprises Activin A, GDF8, or both.

53. The composition of claim 50, wherein the composition comprises at most about 5 ng/mL Activin A.

54. The composition of claim 50, wherein the composition comprises at most about 2.5 ng/mL, at most about 1 ng/mL, at most about 0.5 ng/mL, at most about 0.1 ng/mL, or at most about 0.05 ng/mL Activin A.

55. The composition of claim 50, wherein the composition comprises about 5 ng/mL, about 2.5 ng/mL, about 1 ng/mL, about 0.5 ng/mL, about 0.1 ng/mL, or about 0.05 ng/mL Activin A.

56. The composition of any one of claims 46-55, wherein the composition further comprises PDX1-positive and NKX6.1-positive cells.

57. The composition of any one of claims 36-56, wherein the inhibitor of PI3K/Akt/mTOR signaling comprises an inhibitor of a PI3K protein, an inhibitor of an Akt protein, an inhibitor of mTOR, or any combination thereof.

58. The composition of any one of claims 36-57, wherein the inhibitor of PI3K/Akt/mTOR signaling comprises one or more of: GSK-690693, IPI-3063, AZD8055, Omipalisib, GNE-477, VS-5584, BYL319, YM201636, PI4KIIIbeta-IN-10, Nemiralisib, BYL719, FT113, Apitolisib, or any analog or derivative thereof.

59. The composition of any one of claims 36-57, wherein the composition comprises an inhibitor of a PI3K protein and an inhibitor of an Akt protein.

60. The composition of any one of claims 36-59, wherein the composition comprises GSK-690693, an analog or a derivative thereof.

61. The composition of any one of claims 36-60, wherein the composition comprises BYL719, an analog or a derivative thereof.

62. The composition of any one of claims 36-60, wherein the composition comprises BYL319, an analog or a derivative thereof.

63. The composition of any one of claims 36-56, wherein the composition comprises GSK-690693, or an analog or a derivative thereof, and BYL319, or an analog or a derivative thereof.

64. The composition of any one of claims 36-56, wherein the composition comprises GSK-690693, or an analog or a derivative thereof, and BYL719, or an analog or a derivative thereof.

65. The composition of any one of claims 36-64, wherein the composition comprises from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693, or an analog or a derivative thereof.

66. The composition of any one of claims 36-64, wherein the composition comprises about 0.01 μM, 0.02 μM, 0.04 μM, 0.06 μM, 0.08 μM, 0.1 μM, 0.12 μM, 0.15 μM, 0.2 μM, 0.3 μM, 0.4 μM, 0.5 μM, 0.6 μM, 0.8 μM, or 1 μM of GSK-690693.

67. The composition of any one of claims 36-66, wherein the composition comprises from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719, or an analog or a derivative thereof.

68. The composition of any one of claims 36-66, wherein the composition further comprises a water-soluble synthetic polymer.

69. The composition of claim 68, wherein the water-soluble synthetic polymer comprises polyvinyl alcohol, poloxamer, polyvinylpyrrolidone, polyethylene glycol (PEG), PEG copolymers, poly(N-isopropylacrylamide), or polyacrylamide.

70. The composition of claim 68, wherein the water-soluble synthetic polymer comprises polyvinyl alcohol.

71. The composition of any one of claims 68-70, wherein the water-soluble synthetic polymer is present at a concentration of about 0.005% to about 0.5% (w/v), about 0.01% to about 0.2% (w/v), about 0.02% to about 0.1% (w/v), or about 0.03% to about 0.08% (w/v) in the culture medium.

72. The composition of any one of claims 68-70, wherein the water-soluble synthetic polymer is present at a concentration of about 0.04% to about 0.06% (w/v) in the culture medium.

73. The composition of any one of claims 68-70, wherein the water-soluble synthetic polymer is present at a concentration of about 0.05% (w/v) in the culture medium.

74. The composition of any one of claims 68-73, wherein the water-soluble synthetic polymer comprises polyvinyl alcohol that is less than 85% hydrolyzed.

75. The composition of any one of claims 68-73, wherein the water-soluble synthetic polymer comprises polyvinyl alcohol that is about 80% hydrolyzed.

76. The composition of any one of claims 36-75, wherein the composition comprises about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719.

77. The composition of any one of claims 36-75, wherein the composition comprises about 0.08 μM to about 0.12 μM of GSK-690693 and about 35 nM to about 50 nM of BYL719.

78. The composition of any one of claims 1-77, wherein the composition has a liquid volume of about 500 mL to about 50 L, about 1 L to about 10 L, about 2 L to about 5 L, about 3 L to about 4 L, about 2 L to about 30 L, or about 10 L to about 20 L.

79. The composition of any one of claims 1-77, wherein the composition has a liquid volume of about 10 mL to about 1000 mL, about 10 mL to about 100 mL, about 20 mL to about 50 mL, about 30 mL to about 40 mL, about 20 mL to about 30 mL, or about 10 mL to about 20 mL.

80. A method, comprising contacting a plurality of stem cells in vitro with an inhibitor of PI3K/Akt/mTOR signaling.

81. The method of claim 80, wherein the inhibitor of PI3K/Akt/mTOR signaling comprises an inhibitor of a PI3K protein, an inhibitor of an Akt protein, an inhibitor of mTOR, or any combination thereof.

82. The method of claim 80, wherein the inhibitor of PI3K/Akt/mTOR signaling comprises one or more of: GSK-690693, IPI-3063, AZD8055, Omipalisib, GNE-477, VS-5584, BYL319, YM201636, PI4KIIIbeta-IN-10, Nemiralisib, BYL719, FT113, Apitolisib, or any analog or derivative thereof.

83. The method of claim 80, wherein the inhibitor of PI3K/Akt/mTOR signaling comprises an inhibitor of a PI3K protein and an inhibitor an Akt protein.

84. The method of any one of claims 80-83, wherein the inhibitor of PI3K/Akt/mTOR signaling comprises GSK-690693, an analog or a derivative thereof.

85. The method of any one of claims 80-84, wherein the inhibitor of PI3K/Akt/mTOR signaling comprises BYL719, an analog or a derivative thereof.

86. The method of claim 80, wherein the inhibitor of PI3K/Akt/mTOR signaling comprises GSK-690693 and BYL719.

87. The method of any one of claims 80-86, wherein the contacting comprises contacting the plurality of stem cells with from about 0.01 μM to about 1 μM, about 0.02 μM to about 0.8 μM, about 0.05 μM to about 0.5 μM, about 0.06 μM to about 0.2 μM, about 0.07 μM to about 0.15 μM, or about 0.08 μM to about 0.12 μM of GSK-690693.

88. The method of any one of claims 80-86, wherein the contacting comprises contacting the plurality of stem cells with about 0.01 μM, 0.02 μM, 0.04 μM, 0.06 μM, 0.08 μM, 0.1 μM, 0.12 μM, 0.15 μM, 0.2 μM, 0.3 μM, 0.4 μM, 0.5 μM, 0.6 μM, 0.8 μM, or 1 μM of GSK-690693.

89. The method of any one of claims 80-88, wherein the contacting comprises contacting the plurality of stem cells with from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719.

90. The method of any one of claims 80-88, wherein the contacting comprises contacting the plurality of stem cells with about 1 nM, 4 nM, 8 nM, 10 nM, 15 nM, 20 nM, 25 nM, 30 nM, 35 nM, 40 nM, 45 nM, 50 nM, 55 nM, 60 nM, 65 nM, 70 nM, 80 nM, 90 nM, 100 nM, 200 nM, or 400 nM of BYL719.

91. The method of any one of claims 80-90, wherein the contacting comprises contacting the plurality of stem cells with from about 0.01 μM to about 1 μM, about 0.02 μM to about 0.8 μM, about 0.05 μM to about 0.5 μM, about 0.06 μM to about 0.2 μM, or about 0.07 μM to about 0.15 μM of GSK-690693, and from about 1 nM to about 500 nM, about 5 nM to about 250 nM, about 10 nM to about 200 nM, about 15 nM to about 150 nM, about 20 nM to about 100 nM, about 30 nM to about 80 nM, about 30 nM to about 60 nM, or about 35 nM to about 50 nM of BYL719.

92. The method of any one of claims 80-90, wherein the contacting comprises contacting the plurality of stem cells with about 0.08 μM to about 0.12 μM of GSK-690693 and about 35 nM to about 50 nM of BYL719.

93. The method of any one of claims 80-92, wherein the method comprises contacting the plurality of stem cells with the inhibitor of PI3K/Akt/mTOR signaling and a growth factor from TGF-β superfamily.

94. The method of claim 93, wherein the growth factor from TGF-β superfamily comprises Activin A, GDF8, or both.

95. The method of claim 93, wherein the method comprises contacting the plurality of stem cells with from about 0.5 ng/mL to about 500 ng/mL, about 1 ng/mL to about 250 ng/mL, about 10 ng/mL to about 200 ng/mL, about 20 ng/mL to about 150 ng/mL, about 50 ng/mL to about 120 ng/mL, about 1 ng/mL to about 50 ng/mL, about 2 ng/mL to about 25 ng/mL, or about 5 ng/mL to about 20 ng/mL of Activin A.

96. The method of claim 93, wherein the method comprises contacting the plurality of stem cells with about 1 ng/mL, 2 ng/mL, 3 ng/mL, 4 ng/mL, 5 ng/mL, 6 ng/mL, 7 ng/mL, 8 ng/mL, 9 ng/mL, 10 ng/mL, 12 ng/mL, 14 ng/mL, 15 ng/mL, 18 ng/mL, 20 ng/mL, 25 ng/mL, 30 ng/mL, or 50 ng/mL of Activin A.

97. The method of any one of claims 80-96, wherein the method comprises contacting the plurality of stem cells with the inhibitor of PI3K/Akt/mTOR signaling for from about 24 hours to about 96 hours, from about 36 hours to about 84 hours, from about 48 hours to about 84 hours, from about 60 hours to about 84 hours, or about three days.

98. The method of any one of claims 80-96, wherein the method comprises contacting the plurality of stem cells also with an activator of WNT signaling pathway.

99. The method of claim 98, wherein the activator of WNT signaling pathway comprises one or more of Wnt3a, CHIR99021, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC693868, SB 216763, SB 415286, TC-G 24, TCS 2002, TCS 21311, TWS 119, and analogs or derivatives of any of these.

100. The method of claim 98, wherein the activator of WNT signaling pathway comprises a GSK3 inhibitor.

101. The method of claim 98, wherein the method comprises contacting the plurality of stem cells with from about 0.5 μM to about 50 μM, about 0.6 μM to about 30 μM, about 0.8 μM to about 20 μM, about 1 μM to about 10 μM, or about 2 μM to about 5 μM of CHIR99021.

102. The method of claim 98, wherein the method comprises contacting the plurality of stem cells with about 0.5 μM, 0.6 μM, 0.8 μM, 1 μM, 2 μM, 3 μM, 4 μM, 5 μM, 6 μM, 8 μM, 10 μM, 15 μM, 20 μM, 25 μM, or 30 μM of CHIR99021.

103. The method of any one of claims 98-102, wherein the method comprises culturing the plurality of stem cells in a first composition comprising the inhibitor of PI3K/Akt/mTOR signaling and the activator of WNT signaling pathway for from 12 hours to 48 hours, from 12 hours to 36 hours, from 18 hours to 30 hours, or about one day.

104. The method of claim 103, wherein the method further comprises after the culturing in the first composition, culturing at least part of resulting cells in a second composition that comprises the inhibitor of PI3K/Akt/mTOR signaling for from 12 hours to 72 hours, from 24 hours to 72 hours, from 36 hours to 72 hours, or about two days.

105. The method of claim 104, wherein the second composition does not comprise the activator of WNT signaling pathway.

106. The method of claim 104 or 105, wherein the second composition comprises the same concentration of the inhibitor of PI3K/Akt/mTOR signaling as the first composition.

107. The method of any one of claims 80-106, wherein the stem cells comprise embryonic stem cells.

108. The method of any one of claims 80-106, wherein the stem cells comprise induced pluripotent stem cells.

109. The method of any one of claims 80-108, wherein the stem cells are human cells.

110. The method of any one of claims 80-109, wherein the stem cells are genetically modified.

111. The method of any one of claims 80-110, wherein the contacting the plurality of stem cells in vitro with the inhibitor of PI3K/Akt/mTOR signaling results in generation of a population of cells comprising Sox17-positive cells.

112. The method of claim 111, wherein the population of cells comprises at least about 50%, 60%, 65%, 70%, 75%, 80%, or 85% Sox17-positive, Oct4-negative cells.

113. The method of claim 111, wherein the population of cells comprises from about 50% to about 90%, about 60% to about 90%, about 65% to about 90%, about 70% to about 90%, about 75% to about 90%, about 80% to about 90%, or about 75% to about 85% Sox17-positive, Oct4-negative cells.

114. The method of any one of claims 111-113, wherein the method results in generation of the population of cells that comprises a percentage of Sox17-positive, Oct4-negative cells that is equivalent to a percentage of Sox17-positive, Oct4-negative cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

115. The method of any one of claims 111-114, wherein the method further comprises differentiating the Sox17-positive cells into pancreatic β cells; NKX6.1-positive, ISL1-positive cells; PDX1-positive, NKX6.1-positive cells; PDX1-positive, NKX6.1-negative cells; FOXA2-positive, PDX1-negative cells; or any combination thereof.

116. The method of any one of claims 111-114, wherein the method further comprises contacting cells in the population of cells comprising Sox17-positive cells with a growth factor from fibroblast growth factors (FGF) family.

117. The method of claim 116, wherein the growth factor from fibroblast growth factors (FGF) family is selected from the group consisting of: keratinocyte growth factor (KGF), FGF2, FGF10, FGF21, and FGF8B.

118. The method of claim 116 or 117, wherein the method comprises culturing cells in the population of cells a third composition that comprises the growth factor from fibroblast growth factors (FGF) family for 1 to 5 days, or 2 to 4 days, or about 1, 2, 3, 4, or 5 days.

119. The method of any one of claims 116-118, wherein the contacting with the growth factor from fibroblast growth factors (FGF) family results in generation of a population of cells comprising FOXA2-positive, PDX1-negative cells.

120. The method of claim 119, wherein the population of cells comprising FOXA2-positive, PDX1-negative cells has a percentage of FOXA2-positive, PDX1-negative cells that is equivalent to a percentage of FOXA2-positive, PDX1-negative cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

121. The method of claim 119 or 120, wherein the method further comprises contacting cells in the population of cells comprising FOXA2-positive, PDX1-negative cells with one or more agents selected from the group consisting of: a protein kinase C activator, a growth factor from transformation growth factor β (TGF-β) superfamily, a bone morphogenetic protein signaling pathway inhibitor, a growth factor from fibroblast growth factors (FGF) family, a retinoic acid (RA) signaling pathway activator, a Rho-associated, coiled-coil containing protein kinase (ROCK) inhibitor, and a sonic hedgehog (SHH) pathway inhibitor.

122. The method of claim 121, wherein the method comprises contacting the cells in the population of cells comprising FOXA2-positive, PDX1-negative cells with:
(a) a protein kinase C activator selected from the group consisting of: phorbol 12,13-dibutyrate (PDBU), TPB, phorbol 12-myristate 13-acetate, and bryostatin 1;
(b) a growth factor from the transformation growth factor β (TGF-β) superfamily selected from the group consisting of: an Inhibin, an Activin (e.g., activin A), a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11);
(c) a bone morphogenetic protein signaling pathway inhibitor comprising LDN193189 or DMH-1;
(d) a growth factor from fibroblast growth factors (FGF) family selected from the group consisting of: keratinocyte growth factor (KGF), FGF2, FGF10, FGF21, and FGF8B;
(e) a sonic hedgehog pathway inhibitor selected from the group consisting of SANT1, SANT2, SANT4, Cur6l4l4, forskolin, tomatidine, AY9944, triparanol, and cyclopamine;
(f) a retinoic acid signaling pathway activator selected from the group consisting of: retinoic acid, CD1530, AM580, TTHRB, CD437, Ch55, BMS961, AC261066, AC55649, AM80, BMS753, tazarotene, adapalene, and CD2314; and/or
(g) a ROCK inhibitor selected from the group consisting of Thiazovivin, Y-27632, Fasudil/HA1077, and 14-1152.

123. The method of claim 121 or 122, wherein the method comprises culturing the cells in the population of cells comprising FOXA2-positive, PDX1-negative cells in a fourth composition for 4 to 8 days, or 5 to 7 days, or about 4, 5, 6, 7, or 8 days, and wherein the fourth composition comprises the one or more agents selected from the group consisting of: a protein kinase C activator, a growth factor from transformation growth factor β (TGF-β) superfamily, a bone morphogenetic protein signaling pathway inhibitor, a growth factor from fibroblast growth factors (FGF) family, a retinoic acid (RA) signaling pathway activator, a Rho-associated, coiled-coil containing protein kinase (ROCK) inhibitor, and a sonic hedgehog (SHH) pathway inhibitor.

124. The method of any one of claims 121-123, wherein the contacting the cells in the population of cells comprising FOXA2-positive, PDX1-negative cells results in generation of a population of cells comprising PDX1-positive, NKX6.1-negative cells.

125. The method of claim 124, wherein the population of cells comprising PDX1-positive, NKX6.1-negative cells has a percentage of PDX1-positive, NKX6.1-negative cells that is equivalent to a percentage of PDX1-positive, NKX6.1-negative cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

126. The method of claim 124 or 125, wherein the method further comprises contacting cells in the population of cells comprising PDX1-positive, NKX6.1-negative cells with one or more agents selected from the group consisting of: a growth factor from transformation growth factor β (TGF-β) superfamily, a growth factor from fibroblast growth factors (FGF) family, a retinoic acid (RA) signaling pathway activator, a Rho-associated, coiled-coil containing protein kinase (ROCK) inhibitor, a protein kinase C activator, a FoxO1 inhibitor, a sonic hedgehog (SHH) pathway inhibitor, and a notch signaling inhibitor.

127. The method of claim 126, wherein the method comprises contacting the cells in the population of cells comprising PDX1-positive, NKX6.1-negative cells with:
(a) a growth factor from the transformation growth factor β (TGF-β) superfamily selected from the group consisting of: an Inhibin, an Activin, a Mullerian inhibiting substance (MIS), a bone morphogenic protein (BMP), decapentaplegic (dpp), Vg-1, monoclonal nonspecific suppressor factor (MNSF), growth differentiating factor 8 (GDF8), and growth differentiating factor 11 (GDF11);
(b) a growth factor from fibroblast growth factors (FGF) family selected from the group consisting of: keratinocyte growth factor (KGF), FGF2, FGF10, FGF21, and FGF8B;
(c) a retinoic acid (RA) signaling pathway activator selected from the group consisting of: retinoic acid, CD1530, AM580, TTHRB, CD437, Ch55, BMS961, AC261066, AC55649, AM80, BMS753, tazarotene, adapalene, and CD2314;
(d) a ROCK inhibitor selected from the group consisting of Thiazovivin, Y-27632, Fasudil/HA1077, and 14-1152;
(e) a protein kinase C activator selected from the group consisting of: phorbol 12,13-dibutyrate (PDBU), TPB, phorbol 12-myristate 13-acetate, and bryostatin 1;
(f) a FoxO1 inhibitor, optionally wherein the FoxO1 inhibitor is AS1842856;
(g) a sonic hedgehog (SHH) pathway inhibitor selected from the group consisting of SANT1, SANT2, SANT4, Cur6l4l4, forskolin, tomatidine, AY9944, triparanol, and cyclopamine; and/or
(h) a notch signaling inhibitor, optionally wherein the notch signaling inhibitor is XXI or DAPI.

128. The method of claim 126 or 127, wherein the method comprises culturing the cells in the population of cells comprising PDX1-positive, NKX6.1-negative cells in a fifth composition for 4 to 8 days, or 5 to 7 days, or about 4, 5, 6, 7, or 8 days, and wherein the fifth composition comprises the one or more agents selected from the group consisting of: a growth factor from transformation growth factor β (TGF-β) superfamily, a growth factor from fibroblast growth factors (FGF) family, a retinoic acid (RA) signaling pathway activator, a Rho-associated, coiled-coil containing protein kinase (ROCK) inhibitor, a protein kinase C activator, a FoxO1 inhibitor, a sonic hedgehog (SHH) pathway inhibitor, and a notch signaling inhibitor.

129. The method of any one of claims 126-128, wherein the contacting the cells in the population of cells comprising PDX1-positive, NKX6.1-negative cells results in differentiation of PDX1-positive, NKX6.1-negative cells into PDX1-positive, NKX6.1-positive cells, thereby generating a population of cells comprising PDX1-positive, NKX6.1-positive cells.

130. The method of claim 129, wherein the population of cells comprising PDX1-positive, NKX6.1-positive cells has a percentage of PDX1-positive, NKX6.1-positive cells that is equivalent to a percentage of PDX1-positive, NKX6.1-positive cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

131. The method of any one of claims 103-106, 118, 123, or 128, wherein the first composition, the second composition, the third composition, the fourth composition, or the fifth composition further comprises a water-soluble synthetic polymer.

132. The method of claim 131, wherein the water-soluble synthetic polymer comprises polyvinyl alcohol, poloxamer, polyvinylpyrrolidone, polyethylene glycol (PEG), PEG copolymers, poly(N-isopropylacrylamide), or polyacrylamide.

133. The method of claim 131, wherein the water-soluble synthetic polymer comprises polyvinyl alcohol.

134. The method of any one of claims 131-133, wherein the water-soluble synthetic polymer is present at a concentration of about 0.005% to about 0.5% (w/v), about 0.01% to about 0.2% (w/v), about 0.02% to about 0.1% (w/v), or about 0.03% to about 0.08% (w/v).

135. The method of any one of claims 131-133, wherein the water-soluble synthetic polymer is present at a concentration of about 0.05% (w/v) in the culture medium.

136. The method of any one of claims 131-135, wherein the water-soluble synthetic polymer comprises polyvinyl alcohol that is less than 85% hydrolyzed.

137. The method of any one of claims 131-135, wherein the water-soluble synthetic polymer comprises polyvinyl alcohol that is about 80% hydrolyzed.

138. The method of any one of claims 129-137, wherein the method further comprises contacting cells in the population of cells comprising PDX1-positive, NKX6.1-positive cells with one or more agents selected from the group consisting of: a protein kinase C activator, a TGF-β signaling pathway inhibitor, a thyroid hormone signaling pathway activator, an epigenetic modifying compound, a growth factor from epidermal growth factor (EGF) family, a retinoic acid (RA) signaling pathway activator, a sonic hedgehog (SHH) pathway inhibitor, a γ-secretase inhibitor, a protein kinase inhibitor, a Rho-associated, coiled-coil containing protein kinase (ROCK) inhibitor, a bone morphogenetic protein (BMP) signaling pathway inhibitor, and a Wnt signaling pathway inhibitor.

139. The method of claim 138, wherein the method comprises contacting the cells in the population of cells comprising PDX1-positive, NKX6.1-positive cells with:
(a) a TGF-β signaling pathway inhibitor selected from the group consisting of: Alk5i II, A83-01, SB431542, D4476, GW788388, LY364947, LY580276, SB505124, GW6604, SB-525334, SD-208, or SB-505124;
(b) a thyroid hormone signaling pathway activator comprising T3 or GC-1;
(c) an epigenetic modifying compound selected from the group consisting of: 3-deazaneplanocin A (DZNep), GSK126, EPZ6438, KD5170, MC1568, and TMP195;
(d) a growth factor from the epidermal growth factor family comprising betacellulin or EGF;
(e) a retinoic acid signaling pathway activator selected from the group consisting of: retinoic acid, CD1530, AM580, TTHRB, CD437, Ch55, BMS961, AC261066, AC55649, AM80, BMS753, tazarotene, adapalene, and CD2314;
(f) a sonic hedgehog pathway inhibitor selected from the group consisting of SANT1, SANT2, SANT4, Cur6l4l4, forskolin, tomatidine, AY9944, triparanol, and cyclopamine;
(g) a γ-secretase inhibitor comprising XXI or DAPT;
(h) a protein kinase inhibitor comprising staurosporine, Ro-31-8220, a bisindolylmaleimide (Bis) compound, 10′-{5″-[(methoxycarbonyl)amino]-2″-methyl}-phenylaminocarbonylstaurosporine, or a staralog;
(i) a ROCK inhibitor selected from the group consisting of Thiazovivin, Y-27632, Fasudil/HA1077, and 14-1152;
(j) a protein kinase C activator selected from the group consisting of: phorbol 12,13-dibutyrate (PdBU), TPB, phorbol 12-myristate 13-acetate, and bryostatin 1;
(k) a bone morphogenetic protein signaling pathway inhibitor comprising LDN193189 or DMH-1; and/or
(l) a Wnt signaling pathway inhibitor comprising NVP-TNKS656.

140. The method of claim 138 or 139, wherein the method comprises culturing the cells in the population of cells comprising PDX1-positive, NKX6.1-positive cells in a sixth composition for 5 to 10 days, or 6 to 9 days, or about 5, 6, 7, 8, 9, or 10 days, and wherein the sixth composition comprises the one or more agents selected from the group consisting of: a protein kinase C activator, a TGF-β signaling pathway inhibitor, a thyroid hormone signaling pathway activator, an epigenetic modifying compound, a growth factor from epidermal growth factor (EGF) family, a retinoic acid (RA) signaling pathway activator, a sonic hedgehog (SHH) pathway inhibitor, a γ-secretase inhibitor, a protein kinase inhibitor, a Rho-associated, coiled-coil containing protein kinase (ROCK) inhibitor, a bone morphogenetic protein (BMP) signaling pathway inhibitor, and a Wnt signaling pathway inhibitor.

141. The method of claim 140, wherein the sixth composition further comprises one or more of an acetyl CoA-related metabolite (e.g., acetate), a vitamin (e.g., biotin), histone deacetylase inhibitor (HDACi) (e.g., β-hydroxybutyrate), a redox homeostasis regulator (e.g., taurine), a one carbon metabolism pathway intermediate (e.g., formate), and/or glutamine (e.g., L-glutamine).

142. The method of any one of claims 138-141, wherein the contacting the cells in the population of cells results in generation of a population of cells comprising NKX6.1-positive, ISL1-positive cells.

143. The method of claim 142, wherein the population of cells comprising NKX6.1-positive, ISL1-positive cells has a percentage of NKX6.1-positive, ISL1-positive cells that is equivalent to a percentage of NKX6.1-positive, ISL1-positive cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

144. The method of claim 142 or 143, wherein the method further comprises differentiating the NKX6.1-positive, ISL1-positive cells into a population of cells comprising pancreatic R cells.

145. The method of claim 144, wherein the method comprises contacting cells in the population of cells comprising NKX6.1-positive, ISL1-positive cells with a seventh composition comprising one or more agents selected from the group consisting of: a transformation growth factor β (TGF-β) signaling pathway inhibitor, a thyroid hormone signaling pathway activator, an epigenetic modifying compound, a growth factor from epidermal growth factor (EGF) family, a retinoic acid (RA) signaling pathway activator, a sonic hedgehog (SHH) pathway inhibitor, a γ-secretase inhibitor, a protein kinase inhibitor, a Rho-associated, coiled-coil containing protein kinase (ROCK) inhibitor, and a bone morphogenetic protein (BMP) signaling pathway inhibitor.

146. The method of claim 144 or 145, wherein the population of cells comprising pancreatic R cells has a percentage of pancreatic β cells that is equivalent to a percentage of pancreatic β cells in a population of cells generated by a reference method, wherein the reference method comprises contacting the plurality of stem cells with about 100 ng/mL Activin A but not the inhibitor of PI3K/Akt/mTOR signaling, but is otherwise identical to the method.

147. The method of claim 140 or 145, wherein the sixth composition or the seventh composition further comprises a water-soluble synthetic polymer.

148. The method of claim 147, wherein the water-soluble synthetic polymer comprises polyvinyl alcohol, poloxamer, polyvinylpyrrolidone, polyethylene glycol (PEG), PEG copolymers, poly(N-isopropylacrylamide), or polyacrylamide.

149. The method of claim 147, wherein the water-soluble synthetic polymer comprises polyvinyl alcohol.

150. The method of any one of claims 147-149, wherein the water-soluble synthetic polymer is present at a concentration of about 0.005% to about 0.5% (w/v), about 0.01% to about 0.2% (w/v), about 0.02% to about 0.1% (w/v), or about 0.03% to about 0.08% (w/v).

151. The method of any one of claims 147-149, wherein the water-soluble synthetic polymer is present at a concentration of about 0.05% (w/v) in the culture medium.

152. The method of any one of claims 147-151, wherein the water-soluble synthetic polymer comprises polyvinyl alcohol that is more than 85% hydrolyzed.

153. The method of any one of claims 147-151, wherein the water-soluble synthetic polymer comprises polyvinyl alcohol that is about 87% to 89% hydrolyzed.

154. A device comprising the composition of any one of claims 1-79, a population of cells obtained from the composition of any one of claims 1-79, or cells generated according to the method of any one of claims 80-153.

155. The device of claim 154, wherein the device is configured to produce and release insulin when implanted into a subject.

156. The device of claim 154 or 155, wherein the cells are encapsulated.

157. The device of any one of claims 154-156, further comprising a semipermeable membrane, wherein the semipermeable membrane is configured to retain the cells in the device and permit passage of insulin.

158. A method of treating a subject with a disease characterized by high blood sugar levels over a prolonged period of time, the method comprising administering the composition of any one of claims 1-79, a population of cells obtained from the composition of any one of claims 1-79, or cells generated according to the method of any one of claims 80-153, or implanting the device of any one of claims 154-157, to the subject.

159. The method of claim 158, wherein the disease is diabetes, optionally type I diabetes.


SEQUENCE LISTING (17 sequences)

SEQ ID NO: 1 - protein, 116 aa, Homo sapiens [sequence omitted]

SEQ ID NO: 2 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 3 - protein, 130 aa, Homo sapiens
ARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 4 - protein, 115 aa, synthetic construct
  REGION 1..115: note = Unidentified
GLECDGKVNICCKKQFFVSFKDIGWNDWIIAPSGYHANYCEGECPSHIAGTSGSSLSFHSTVINHYACGHSPFANLKSCCVPTKLRPMSMLYYDDGQNIIKKDIQNMIVEECGCS

SEQ ID NO: 5 - protein, 426 aa, Homo sapiens; identical to SEQ ID NO: 2

SEQ ID NO: 6 - protein, 424 aa, synthetic construct
  REGION 1..424: note = Unidentified
MPLLWLRGFLLASCWIIVRSSPTPGSEGHGSAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 7 - protein, 424 aa, synthetic construct
  REGION 1..424: note = Unidentified
MPLLWLRGFLLASCWIIVRSSPTPGSEGHGAAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDMGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 8 - protein, 424 aa, synthetic construct
  REGION 1..424: note = Unidentified
MPLLWKRGFLLVICWIIVRSSPTPGSEGHSSVADCPSCALTTLSKDVPSSQPEMVEAVKKHILNMLHLRDRPNITQPVPKAALLNATKKLHVGKVGDDGYVEIEDDVGRRAEMNEVVEQTSEIITFAESGTPKKTLHFEISKEGSELSVVEHAEVWLFLKVSKANRSRTKVTIRLFQQQRQPKGNSEAAEDMEDMGLKGERSETLISEKAVDARKSTWHIFPISSSVQRLLDQGQSSLDVRIACDLCQETGASLVLLGKKKKKEDDGEGKEKDGGELTGEEEKEQSHRPFLMMLARHSEDRQHRRRERGLECDGKVNICCKKQFFVSFKDIGWSDWIIAPTGYHANYCEEECPSHIAGTSGSSLSFHSTVINHYRMRGHSPFANLKSCCVPTKLRPMSMLYYDDGQNIIKKDIQNMIVEECGCS

SEQ ID NO: 9 - protein, 425 aa, synthetic construct
  REGION 1..425: note = Unidentified
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKKKEEEGEGKKRDGEGGAGGDEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 10 - protein, 426 aa, synthetic construct
  REGION 1..426: note = Unidentified
MPLLWLRGFLLASCWIIVKSSPTPGSEGHSAAPNCPSCALATLPKDVPNAQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRSKVTIRLLQQQKHPQGSSDTREEAEEADLMEERSEQLISEKVVDARKSTWHIFPVSSSIQRLLDQGKSSLDIRIACDQCHETGASLVLLGKKKKKEEEGEGKKKDGGEAGAGVDEEKEQSHRPFLMLQARQSEDHPHRRRRRGLECDGKVNICCKKQFFVSFKDIGWNDWIIAPSGYHANYCEGECPSHIAGTSGSSLSFHSTVINQYRLRGHNPFANLKSCCVPTKLRPMSMLYYDDGQNIIKKDIQNMIVEECGCS

SEQ ID NO: 11 - protein, 424 aa, synthetic construct
  REGION 1..424: note = Unidentified
MPLLWLRGFLLASCWIIVRSSPTPGSGGHSAAPDCPSCALATLPKDVPNSQPEMVEAVKKHILNMLHLKKRPDVTQPVPKAALLNAIRKLHVGKVGENGYVELEDDIGRRAEMNELMEQTSEIITFAEAGTARKTLRFEISKEGSDLSVVERAEIWLFLKVPKANRTRTKVSIRLFQQQRRPQGSADAGEEAEDVGFPEEKSEVLISEKVVDARKSTWHIFPVSSSIQRLLDQGKSALDIRTACEQCHETGASLVLLGKKKKKEEEAEGRKRDGEGAGVDEEKEQSHRPFLMLQARQSEEHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 12 - protein, 425 aa, synthetic construct
  REGION 1..425: note = Unidentified
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISQEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKRKEEEGEGKKRDGEGGAGGDEEKEQSHRPFLMLQARQSEDHPHRRRRRGLECDGKVNICCKKQFYVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 13 - protein, 424 aa, synthetic construct
  REGION 1..424: note = Unidentified
MPLLWLRGFLLASCWIIVRSSPTPGSEGPGAAPDCPSCALATLPKDVPNSQPEMVEAVKKHILNMLHLKKRPEVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIQLLQKQPQGGVDAGEEAEEMGLMEERNEVLISEKVVDARKSTWHIFPVSSSIQRLLDQGKSSLDVRIACEQCHETGASLVLLGKKKKKEEEGEGKKKDGGDGGAGADEDKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 14 - protein, 395 aa, synthetic construct
  REGION 1..395: note = Unidentified
MSPLPLLSGILLLLIRSCSLSAMVTKGSLPMSEQQAGATVCPSCALARFRKGVSESEDEGAQQDVVEAVKRHILNMLHLQERPNITHPVPRAALLNAIRKVHVGRVAKDGSVLIEDEASNRAETEQAEQTEIITFAETGEAPGIVNFLISKEGGEMSVVDQANVWIFLRLPKGNRTRANVNIRLLLQQGAGEKILAEKSVDTRRSGWHTFPASESVQSLLQRGGSTLSLRVSCPLCADARATPVLVSPGGSEREQSHRPFLMAVVRQMDELSLRRRRKRGLECDGKARVCCKRQFYVNFKDIGWNDWIIAPSGYHANYCEGDCASNVASITGNSLSFHSTVISHYRIRGYSPFTNIKSCCVPTRLRAMSMLYYNEEQKIVKKDIQNMIVEECGCS

SEQ ID NO: 15 - protein, 404 aa, synthetic construct
  REGION 1..404: note = Unidentified
MSSLTLVNRGTAALRLFVRGLLTHSSREWLSGDGEPDDPVTPCPSCALAQRQKDSEEQTDMVEAVKRHILNMLHLNTRPNVTHPVPRAALLNAIRRLHVGRVGEDGTVEMEEDGGGLGEHREQSEEQPFEIITFAEPGDAPDIMKFDISMEGNTLSVVEQANVWLLLKVAKGSRGKGKVSVQLLQHGKADPGSADGPQEAVVSEKTVDTRRSGWHTLPVSRTVQTLLDGDSSMLSLRVSCPMCAEAGAVPILVPTESNKGKEREQSHRPFLMVVLKPAEEHPHRRSKRGLECDGKIRVCCKRQFYVNFKDIGWSDWIIAPSGYHANYCEGDCPSHVASITGSALSFHSTVINHYRMRGYSPFNNIKSCCVPTRLRAMSMLYYNEEQKIIKKDIQNMIVEECGCS

SEQ ID NO: 16 - protein, 194 aa, synthetic construct
  REGION 1..194: note = Unidentified
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 17 - protein, 176 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETN